(19) United States
(12) Patent Application Publication
(10) Pub. No.: US 2003/0215819 A1
(43) Pub. Date: Nov. 20, 2003

(54) Compositions and methods for inferring a response to statin

(76) Inventor: Tony N. Frudakis, Bradenton, FL (US)

Correspondence Address: LISA A. HAILE, J.D., PH.D., GRAY CARYWARE & FREIDENRICH LLP, Suite 1100, 4365 Executive Drive, San Diego, CA, 92121-2133, US

(21) Appl. No.: 10/188,359

(22) Filed: Jul. 1, 2002

Related U.S. Application Data
(60) Provisional application No. 60/322,478, filed on Sep. 13, 2001.
(60) Provisional application No. 60/310,783, filed on Aug. 7, 2001.
(60) Provisional application No. 60/301,867, filed on Jun. 29, 2001.

(51) Int. Cl. C12Q 1/68; C07H 21/04

(52) U.S. Cl. 435/6; 536/24.3

(57) ABSTRACT

Methods for inferring a statin response of a human subject from a nucleic acid sample of the subject are provided, as are reagents such as oligonucleotide probes, primers, and primer pairs, which can be used to practice such methods. A method of inferring a statin response can be performed, for example, by identifying in a nucleic acid sample from a subject, a nucleotide occurrence of at least one statin response-related single nucleotide polymorphism (SNP) and/or at least one statin response-related haplotype in a cytochrome P450 gene and/or an HMG Co-A reductase gene.

This application claims the benefit under 35 USC §119(e) of U.S. application Ser. No. 60/301,867 filed Jun. 29, 2001, Ser. No. 60/310,783 filed Aug. 7, 2001, and Ser. No. 60/322,478 filed Sep. 13, 2001. This disclosure of the prior applications is considered part of and is incorporated by reference in the disclosure of this application.

### BACKGROUND OF THE INVENTION

1. Field of the Invention

The invention relates generally to methods for inferring a statin response, and more specifically to methods of detecting single nucleotide polymorphisms and combinations thereof in a nucleic acid sample that provide an inference as to a response to statins.

2. Background Information

Heart attacks are the leading cause of death in the United States today. An increased risk of heart attack is linked with abnormally high blood cholesterol levels. Patients with abnormally high cholesterol levels are frequently prescribed a class of drugs called statins to reduce cholesterol levels, thereby reducing the risk of heart attack. However, these drugs are not effective in all patients. Furthermore, in some patients, adverse reactions such as increased liver transaminase levels are observed. Recently, it has been reported that patients taking statins are much more likely to have peripheral neuropathy. Such an adverse response may require that a patient discontinue treatment or switch drugs.

It is likely that these variable statin responses can be explained, at least in part, by genetic differences of patients who take statins. Human beings differ by up to 0.1% of the 3 billion letters of DNA present in the human genome. Though we are 99.9% identical in genetic sequence, it is the 0.1% that determines our uniqueness. Though our individuality is apparent from visual inspection—anyone can recognize that we have facial features, heights and colors, and that these features are, to an extent, heritable (i.e. sons and daughters tend to resemble their parents more than strangers)—our individuality extends to our ability to respond to and metabolize commonly used drugs such as statins.

However, identifying the precise molecule details that are responsible for our individuality is a challenging task. The human genome project resulted in the sequencing of the human genome. However, this sequencing was the result of sampling taken from a small number of individuals. Therefore, while this sequencing was an important scientific milestone, the initial sequencing of the human genome does not provide adequate information regarding genetic differences between individuals to allow identification of markers on the genome that are responsible for our individuality, such as whether an individual will respond to statins. If the genetic markers that were responsible for different statin responses between people were identified, then an individual's genotype for key markers could be determined, and this information could be used by a physician to decide whether to prescribe statins and which statins to prescribe. This would result in a better response rate with lower adverse reactions in patients treated with statins.

Thus, there is a need for methods and compositions that allow an inference of statin response based on an individual's genotype for key markers. The invention satisfies this need, and provides additional advantages.

### SUMMARY OF THE INVENTION

The present invention relates to compositions and methods useful for inferring a statin response of a subject from a nucleic acid sample of the subject. The invention is based, in part, on a determination that single nucleotide polymorphisms (SNPs), including haploid or diploid SNPs, and haplotype alleles (i.e., combinations of two or more SNPs in a single gene, e.g., a cytochrome P450 gene and/or a 3-hydroxy-3-methylglutaryl-coenzymeA reductase (HMGCR) gene), including haploid or diploid haplotype alleles, allows an inference to be drawn as to whether a subject, particularly a human subject, will have a positive response to treatment with a statin, for example, by exhibiting a decrease in total cholesterol or in low density lipoprotein levels, or will have an adverse response, for example, liver damage. The statin can be any statin, including, for example, Atorvastatin or Simvastatin.

In one embodiment, the invention relates to a method for inferring a statin response of a human subject from a nucleic acid sample of the subject, for example, by identifying, in the nucleic acid sample, at least one haplotype allele indicative of a statin response. Haplotype alleles indicative of a statin response in a human subject are exemplified herein by haplotype alleles of cytochrome P450 and HMGCR genes that are associated with a decrease in total cholesterol or low density lipoprotein in response to a statin in a subject. In one aspect, such haplotype alleles are exemplified by nucleotides of the cytochrome p450 3A4 (CYP3A4) gene, corresponding to a CYP3A4A haplotype, which includes nucleotide 808 of SEQ ID NO:8 {CYP3A4E10-5_{—}292}, and nucleotide 227 of SEQ ID NO:9 {CYP3A4E12_{—}76}; or corresponding to a CYP3A4B haplotype, which includes nucleotide 1311 of SEQ ID NO:7 {CYP3A4E7_{—}243}, nucleotide 808 of SEQ ID NO:8 {CYP-3A4E10-5_{—}292}, and nucleotide 227 of SEQ ID NO:9 {CYP3A4E12_{—}76}; or corresponding to a CYP3A4C haplotype, which includes nucleotide 425 of SEQ ID NO:10 {CYP3A4E3-5_{—}249}, nucleotide 1311 of SEQ ID NO:7 {CYP3A4E7_{—}243}, nucleotide 808 of SEQ ID NO:8 {CYP3A4E10-5_{—}292}, and nucleotide 227 of SEQ ID NO:9 {CYP3A4E12_{—}76}. In another aspect, haplotype alleles indicative of a positive statin response are exemplified by nucleotides of the HMGCR gene, corresponding to an HMGCRA haplotype, which includes nucleotide 1757 of SEQ ID NO:2 {HMGCRE7E11-3_{—}472}, and nucleotide 1430 of SEQ ID NO:3 {HMGCRDBSNP_{—}45320}; corresponding to an HMGCRB haplotype, which includes nucleotide 519 of SEQ ID NO:11 {HMGCRE5E6-3_{—}283}, nucleotide 1757 of SEQ ID NO:2 {HMGCRE7E11-3_{—}472}, nucleotide 1430 of SEQ ID NO:3 {HMGCRDBSNP_{—}45320}, and nucleotide 1421 of SEQ ID NO:12 {HMGCRE16E18_{—}99}; or corresponding to a HMGCRC haplotype, which includes nucleotide 1757 of SEQ ID NO:2 {HMGCRE7E11-3_{—}472}, nucleotide 1430 of SEQ ID NO:3 {HMGCRDBSNP_{—}45320}, and nucleotide 1421 of SEQ ID NO:12 {HMGCRE16E18_{—}99}.

The haplotype allele can include a CYP3A4A haplotype allele, a CYP3A4B haplotype allele, a CYP3A4C haplotype allele, or a combination of the CYP gene haplotype alleles; or can include an HMGCRA haplotype allele, an HMGCRB haplotype allele, or a combination of the HMGCR haplotype alleles; or can include a combination of such CYP gene and HMGCR gene haplotype alleles. In addition, a method of the invention can include identifying a diploid pair of haplotype alleles, i.e., the corresponding haplotype alleles on both chromosomes, for example, a diploid pair of CYP3A4A haplotype alleles, CYP3A4B haplotype alleles, or CYP3A4C haplotype alleles; or a diploid pair of HMGCRA haplotype alleles, HMGCRB haplotype alleles, or HMGCRC haplotype alleles; or any combination of diploid pairs of such haplotype alleles. Thus, for example, a method of the invention can identify at least one CYP3A4C haplotype allele and at least one HMGCRB haplotype allele; or a diploid pair of CYP3A4C haplotype alleles; a diploid pair of HMGCRB haplotype alleles; or a diploid pair of CYP3A4C haplotype alleles and a diploid pair of HMGCRB haplotype alleles. For example, a diploid pair of CYP3A4C haplotype alleles can be ATGC/ATGC or ATGC/ATAC; and a diploid pair of HMGCRB haplotype alleles can be CGTA/CGTA or CGTA/TGTA; e.g., the diploid pair of CYP3A4C haplotype alleles can be ATGC/ATGC, and the diploid pair of HMGCRB haplotype alleles can be CGTA/CGTA or CGTA/TGTA.

The method of the invention can also identify at least one CYP3A4C haplotype allele and at least one HMGCRC haplotype allele, or a diploid pair of HMGCR haplotype alleles, or a diploid pair of HMGCR haplotype alleles and a diploid pair of CYP3A4C haplotype alleles. For example, a diploid pair of CYP3A4C haplotype alleles can be ATGC/ATGC or ATGC/ATAC; and a diploid pair of HMGCRC haplotype alleles can be GTA/GTA; e.g., the diploid pair of CYP3A4C haplotype alleles can be ATGC/ATGC, and the diploid pair of HMGCRC haplotype alleles can be GTA/GTA.

Where a diploid pair of haplotype alleles is identified, the haplotype alleles can be major haplotype alleles, which occur in a relatively larger percent of a population, for example, a population of Caucasian individuals; can be minor haplotype alleles, which occur in a relatively smaller percent of a population; or can be a combination of a minor haplotype allele and a major haplotype allele. For example, a diploid pair of CYP3A4C haplotypes alleles can include a one minor and one major haplotype allele, or can be a diploid pair of minor haplotype alleles. Similarly, a diploid pair of HMGCRB haplotype alleles can be a diploid pair of major haplotype alleles or a diploid pair of minor haplotype alleles.

A diploid pair of CYP3A4C haplotype alleles is exemplified by ATGC/ATGC, ATGC/ATAC, ATAC/ATAC, ATGC/AGAC, AGAC/AGAC, ATAC/AGAC, ATGC/AGAT, AGAT/AGAT, AGAT/ATAC, AGAT/AGAC, ATGC/ATAT, ATAT/ATAT, ATAT/ATAC, ATAT/AGAC, ATAT/AGAT, ATGC/TGAC, TGAC/TGAC, TGAC/ATAC, TGAC/AGAC, TGAC/AGAT, TGAC/ATAT, ATGC/AGAT, AGAT/AGAT, AGAT/ATAC, AGAT/AGAC, AGAT/AGAT, AGAT/ATAT, or AGAT/TGAC, and, more particularly, by ATGC/ATGC, ATGC/ATAC, ATGC/AGAC, ATGC/AGAT, ATGC/ATAT, ATGC/TGAC, and ATGT/AGAT. A diploid pair of HMGCRB haplotype alleles is exemplified by CGTA/CGTA, CGTA/TGTA, CGTA/CGTA, CGTA/CGCA, CGCA/CGCA, CGCA/CGTA, CGTA/CGTC, CGTC/CGTC, CGTC/CGCA, CGTC/CGTA, CGTA/CATA, CATA/CATA, CATA/TGTA, CATA/CGTA, CATA/CGCA, or CATA/CGTC, and, more particularly, by CGTA/CGTA, CGTA/TGTA, CGTA/CGCA, CGTA/CGTC, and CGTA/CATA.

The haplotype allele also can include at least one CYP3A4A haplotype allele and/or at least one HMGCRA haplotype allele; and can include a diploid pair of CYP3A4A haplotype alleles; a diploid pair of HMGCRA haplotype alleles; or a diploid pair of CYP3A4A haplotype alleles and a diploid pair of HMGCRA haplotype alleles. A diploid pair of CYP3A4A haplotype alleles that allows an inference as to whether a subject will have a positive statin response can be, for example, GC/GC; and such a diploid pair of HMGCRA haplotype alleles is exemplified by TG/TG. For example, the human subject can have the diploid pair of CYP3A4A haplotype alleles, GC/GC, and the diploid pair of HMGCRA haplotype alleles, TG/TG. The diploid pair of CYP3A4A haplotypes and/or HMGCR haplotype alleles can be a diploid pair of major haplotype alleles or a diploid pair of minor haplotype alleles.

A method of inferring a positive statin response also can include identifying at least one CYP3A4B haplotype allele and/or at least one HMGCRA haplotype allele, including, for example, a diploid pair of CYP3A4B haplotype alleles; a diploid pair of HMGCRA haplotype alleles; or a diploid pair of CYP3A4B haplotype alleles and a diploid pair of HMGCRA haplotype alleles. Such a diploid pair of CYP3A4B haplotype alleles is exemplified by TGC/TGC, and such a diploid pair of HMGCRA haplotype alleles is exemplified TG/TG. As such, a subject can have, for example, the diploid pair of CYP3A4B haplotype alleles, TGC/TGC, and the diploid pair of HMGCRA haplotype alleles, TG/TG. The diploid pair of CYP3A4B haplotype alleles or HMGCRA haplotype alleles can be a diploid pair of major haplotype alleles or a diploid pair of minor haplotype alleles.

A method of the invention also allows an inference to be drawn as to whether a subject will have an adverse statin response, for example, liver damage. Such a method can be performed, for example, by identifying, in a nucleic acid sample from a subject, a haplotype allele of a cytochrome p450 2D6 (CYP2D6) gene corresponding to a CYP2D6A haplotype, which includes nucleotide 1159 of SEQ ID NO:4 {CYP2D6PE1_{—}2}, nucleotide 1093 of SEQ ID NO:5 {CYP2D6PE7_{—}150}, and nucleotide 1223 of SEQ ID NO:6 {CYP2D6PE7_{—}286}. The presence of such a haplotype, particularly where the haplotype allele is other than CTA, is associated with an increase in one or more hepatocytes stress indicators, for example serum glutamic-oxaloacetic transaminase (SGOT). The method can include identifying a diploid pair of CYP2D6A haplotype alleles.

A method for inferring a negative (or adverse) statin response also can be performed by identifying, in a nucleic acid sample from a subject, a diploid pair of nucleotides of the CYP2D6 gene, at a position corresponding to nucleotide 1274 of SEQ ID NO:1 {CYP2D6E7_{—}339}, whereby a diploid pair of nucleotides, particularly a diploid pair other than C/C, is indicative of an adverse hepatocellular response. For example, the diploid pair of nucleotides can be C/A, which is indicative of an adverse hepatocellular effect.

In another embodiment, the invention relates to a method for inferring a statin response of a human subject from a nucleic acid sample of the subject by identifying, in the nucleic acid sample, at least one statin response related SNP. In one aspect, the method allows an inference to be drawn that a subject will have a positive statin response, for example, a decrease in total cholesterol or low density lipoprotein in response to administration of a statin, by identifying s statin response related SNP corresponding to nucleotide 1757 of SEQ ID NO:2 {HMGCRE7E11-3_{—}472}, nucleotide 1430 of SEQ ID NO:3 {HMGCRDBSNP_{—}45320}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283 }, or nucleotide 1421 of SEQ ID NO:12 {HMGCRE16E18_{—}99}. In another aspect, the method allows an inference to be drawn as to whether the subject will have an adverse statin response by identifying, in a nucleic acid sample from the subject, a nucleotide occurrence of at least one statin response related SNP corresponding to nucleotide 1274 of SEQ ID NO:1 {CYP2D6E7_{—}339}, nucleotide 1159 of SEQ ID NO:4 {CYP2D6PE1_{—}2}, nucleotide 1093 of SEQ ID NO:5 {CYP2D6PE7_{—}150}, or nucleotide 1223 of SEQ ID NO:6 {CYP2D6PE7_{—}286}.

Such a method for inferring a statin response by identifying at least one statin response related SNP in a nucleic acid sample from a subject can be performed, for example, by incubating the nucleic acid sample with an oligonucleotide probe or primer that selectively hybridizes to or near, respectively, a nucleic acid molecule comprising the nucleotide occurrence of the SNP, and detecting selective hybridization of the primer or probe. Selective hybridization of a probe can be detected, for example, by detectably labeling the probe, and detecting the presence of the label using a blot type analysis such as Southern blot analysis. Selective hybridization of a primer can be detected, for example, by performing a primer extension reaction, and detecting a primer extension reaction product comprising the primer. If desired, the primer extension reaction can be performed as a polymerase chain reaction.

The method can include identifying a nucleotide occurrence of each of at least two (e.g., 2, 3, 4, 5, 6, or more) statin response related SNPs, which can, but need not comprise one or more haplotype alleles, and can, but need not be in one gene. The nucleotide occurrence of the at least one statin response related SNP can be a minor nucleotide occurrence, i.e., a nucleotide present in a relatively smaller percent of a population including the subject, or can be a major nucleotide occurrence. Where a haplotype allele is determined, the haplotype allele can be a major haplotype allele, or a minor haplotype allele.

The present invention also relates to an isolated human cell, which contains, in an endogenous HMGCR gene or in an endogenous CYP gene or in both, a first minor nucleotide occurrence of at least a first statin response related SNP. Accordingly, in one embodiment, the invention provides an isolated human cell, which contains an endogenous HMGCR gene, which includes a first minor nucleotide occurrence of at least a first statin response related SNP. For example, the minor nucleotide occurrence can be at a position corresponding to

nucleotide 519 of SEQ ID NO:11 {HMGCRE5E6-3_{—}283},

nucleotide 1430 of SEQ ID NO:3 {HMGCRDBSNP_{—}45320},

nucleotide 1757 of SEQ ID NO:2 {HMGCRE7E11-3_{—}472}, or nucleotide 1421 of SEQ ID NO:12 {HMGCRE16E18_{—}99}.

The endogenous HMGCR gene in an isolated cell of the invention can further contain a minor nucleotide occurrence of a second statin response related SNP, which, for example, in combination with the first minor nucleotide occurrence of the first statin response related SNP comprises a minor haplotype allele of an HMGCR haplotype, for example, an HMGCRA or HMGCRB haplotype. The endogenous HMGCR gene of the isolated cell also can further contain a major nucleotide occurrence of a second statin response related SNP, which, for example, in combination with the first minor nucleotide occurrence of the first statin response related SNP can comprise a haplotype allele, which can be a minor haplotype allele of an HMGCR haplotype.

The isolated cell of the invention can also further contain a second minor nucleotide occurrence of the first statin response related SNP, thereby providing a diploid pair of minor nucleotide occurrences of the HMGCR gene. In addition, an isolated human cell of the invention can further contain a major nucleotide occurrence of the first statin response related SNP, thereby providing a diploid pair of nucleotide occurrences comprising a major nucleotide occurrence and a minor nucleotide occurrence. An isolated human cell of the invention also can contain an endogenous cytochrome p450 gene having a minor nucleotide occurrence of a statin response related SNP.

In another embodiment, the invention provides an isolated human cell, which contains an endogenous CYP3A4 gene that includes a thymidine residue at nucleotide 425 of SEQ ID NO:10 {CYP3A4E3-5_{—}249} or a first minor nucleotide occurrence at a position corresponding to nucleotide 1311 of SEQ ID NO:7 {CYP3A4E7_{—}243}, nucleotide 808 of SEQ ID NO:8 {CYP3A4E10-5_{—}292}, or nucleotide 227 of SEQ ID NO:9 {CYP3A4E12_{—}76}.

The endogenous CYP3A4 gene in an isolated cell of the invention can further contain a minor nucleotide occurrence of a second statin response related SNP, which, for example, in combination with the first nucleotide occurrence of the first statin response related SNP comprises a minor haplotype allele of an CYP3A4 haplotype, for example, a CYP3A4A, CYP3A4B or CYP3A4C haplotype. The endogenous CYP3A4 gene of the isolated cell also can further contain a major nucleotide occurrence of a second statin response related SNP, which, for example, in combination with the first minor nucleotide occurrence of the first statin response related SNP can comprise a haplotype allele which can be a minor haplotype allele of an CYP3A4 haplotype.

The isolated cell of the invention can also further contain a second minor nucleotide occurrence of the first statin response related SNP or a second thymidine residue at nucleotide 425 of SEQ ID NO:10 {CYP3A4E3-5_{—}249}, thereby providing a diploid pair of nucleotide occurrences of the CYP3A4 gene. In addition, an isolated human cell of the invention can further contain a major nucleotide occurrence of the first statin response related SNP, thereby providing a diploid pair of nucleotide occurrences comprising a major nucleotide occurrence and a minor nucleotide occurrence. An isolated human cell of the invention also can contain an endogenous HMGCR gene having a minor nucleotide occurrence of a statin response related SNP, and also can contain an endogenous CYP2D6 gene having a minor nucleotide occurrence of a statin response-related SNP.

In another embodiment, the invention provides an isolated human cell, which contains an endogenous CYP3A4 gene, which includes a first minor nucleotide occurrence of at least a first statin response related SNP. For example, the minor nucleotide occurrence can be at a position corresponding

nucleotide 425 of SEQ ID NO:10 {CYP3A4E3-5_{—}249},

nucleotide 1311 of SEQ ID NO:7 {CYP3A4E7_{—}243},

nucleotide 808 of SEQ ID NO:8 {CYP3A4E10-5_{—}292}, or

nucleotide 227 of SEQ ID NO:9 {CYP3A4E12_{—}76}.

In another embodiment, the invention provides an isolated human cell, which contains an endogenous CYP2D6 gene, which includes a first minor nucleotide occurrence of at least a first statin response related SNP. For example, the minor nucleotide occurrence can be at a position corresponding

nucleotide 1159 of SEQ ID NO:4 {CYP2D6PE1_{—}2}, a

nucleotide 1093 of SEQ ID NO:5 {CYP2D6PE7_{—}150}, or a

nucleotide 1223 of SEQ ID NO:6 {CYP2D6PE7_{—}286}.

The endogenous CYP2D6 gene in an isolated cell of the invention can further contain a minor nucleotide occurrence of a second statin response related SNP, which, for example, in combination with the first minor nucleotide occurrence of the first statin response related SNP comprises a minor haplotype allele of an CYP2D6 haplotype, for example, a CYP2D6A haplotype. The endogenous CYP2D6 gene of the isolated cell also can further contain a major nucleotide occurrence of a second statin response related SNP, which, for example, in combination with the first minor nucleotide occurrence of the first statin response related SNP can comprise a haplotype allele, which can be a minor haplotype allele of an CYP2D6 haplotype.

The isolated cell of the invention can also further contain a second minor nucleotide occurrence of the first statin response related SNP, thereby providing a diploid pair of minor nucleotide occurrences of the CYP2D6 gene. In addition, an isolated human cell of the invention can further contain a major nucleotide occurrence of the first statin response related SNP, thereby providing a diploid pair of nucleotide occurrences comprising a major nucleotide occurrence and a minor nucleotide occurrence. An isolated human cell of the invention also can contain an endogenous HMGCR gene having a minor nucleotide occurrence of a statin response related SNP, and also can contain an endogenous CYP3A4 gene having a minor nucleotide occurrence of a statin response-related SNP.

In certain preferred embodiments, the isolated cell of the present invention has a minor allele of a HMGCRB haplotype, a minor allele of a CY3A4C haplotype, and/or a minor allele of a CY32D6A haplotype. The specific nucleotide occurrences of such minor alleles are listed herein.

The present invention also relates to a plurality of isolated human cells, which includes at least two (e.g., 2, 3, 4, 5, 6, 7, 8, or more) populations of isolated cells, wherein the isolated cells of one population contain at least one nucleotide occurrence statin response related SNP or at least one statin response related haplotype allele that is different from the isolated cells of at least one other population of cells of the plurality. Accordingly, in one embodiment, the invention provides a plurality of isolated human cells, which includes a first isolated human cell, which comprises an endogenous HMGCR gene comprising a first minor nucleotide occurrence of a first statin response related single nucleotide polymorphism (SNP), and at least a second isolated human cell, which comprises an endogenous HMGCR gene comprising a nucleotide occurrence of the first statin response related SNP different from the minor nucleotide occurrence of the first statin response related SNP of the first cell.

A plurality of isolated human cells of the invention can include, for example, at least a second isolated human cell (generally a population of such cells) that contains a second minor nucleotide occurrence of the first statin response related SNP, wherein the second minor nucleotide occurrence of the first statin response related SNP is different from the first minor nucleotide occurrence of the first statin response related SNP. The endogenous HMGCR gene of the first isolated cell can, but need not, further contain a minor nucleotide occurrence of a second statin response related SNP, which, in combination with the first minor nucleotide occurrence of the first statin response related SNP can, but need not, comprise a minor haplotype allele of an HMGCR haplotype, for example, an HMGCRA haplotype, or can comprise a major haplotype allele of an HMGCRA haplotype.

In another embodiment, the invention provides a plurality of isolated human cells, which includes a first isolated human cell, which comprises an endogenous CYP3A4 gene that includes a first nucleotide occurrence of a statin response-related SNP that includes a thymidine residue at nucleotide 425 of SEQ ID NO:10 {CYP3A4E3-5_{—}249} or a first minor nucleotide occurrence at a position corresponding to nucleotide 1311 of SEQ ID NO:7 {CYP3A4E7_{—}243}, nucleotide 808 of SEQ ID NO:8 {CYP3A4E10-5_{—}292}, or nucleotide 227 of SEQ ID NO:9 {CYP3A4E12_{—}76}, and at least a second isolated human cell, which comprises an endogenous CYP3A4 gene comprising a nucleotide occurrence of the first statin response related SNP different from the nucleotide occurrence of the first statin response related SNP of the first cell.

A plurality of isolated human cells of the invention can include, for example, at least a second isolated human cell (generally a population of such cells) that contains a second minor nucleotide occurrence of the first statin response related SNP, wherein the second minor nucleotide occurrence of the first statin response related SNP is different from the first minor nucleotide occurrence of the first statin response related SNP. The endogenous CYP3A4 gene of the first isolated cell can, but need not, further contain a minor nucleotide occurrence of a second statin response related SNP, which, in combination with the first minor nucleotide occurrence of the first statin response related SNP to form a minor haplotype allele of an CYP3A4A, CYP3A4B, or CYP3A4C haplotype.

In another embodiment, the invention provides a plurality of isolated human cells, which includes a first isolated human cell, which comprises an endogenous CYP2D6 gene comprising a first minor nucleotide occurrence of a first statin response related single nucleotide polymorphism (SNP), and at least a second isolated human cell, which comprises an endogenous CYP2D6 gene comprising a nucleotide occurrence of the first statin response related SNP different from the minor nucleotide occurrence of the first statin response related SNP of the first cell.

A plurality of isolated human cells of the invention can include, for example, at least a second isolated human cell (generally a population of such cells) that contains a second minor nucleotide occurrence of the first statin response related SNP, wherein the second minor nucleotide occurrence of the first statin response related SNP is different from the first minor nucleotide occurrence of the first statin response related SNP. The endogenous CYP2D6 gene of the first isolated cell can, but need not, further contain a minor nucleotide occurrence of a second statin response related SNP, which, in combination with the first minor nucleotide occurrence of the first statin response related SNP to form a minor haplotype allele of an CYP2D6A.

The present invention further relates to a method for classifying an individual as being a member of a group sharing a common characteristic by identifying a nucleotide occurrence of a SNP in a polynucleotide of the individual, wherein the nucleotide occurrence of the SNP corresponds to a thymidine residue at nucleotide 425 of SEQ ID NO:10 {CYP3A4E3-5_{—}249}, or a minor nucleotide occurrence of at least one of nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 519 of SEQ ID NO:11 {HMGCRE5E6-3_{—}283}, and nucleotide 1421 of SEQ ID NO:12 {HMGCRE16E18_{—}99}, or any combination thereof.

The present invention further relates to a method for classifying an individual as being a member of a group sharing a common characteristic by identifying a nucleotide occurrence of a SNP in a polynucleotide of the individual, wherein the nucleotide occurrence of the SNP corresponds to a thymidine residue at nucleotide 425 of SEQ ID NO:10 {CYP3A4E3-5_{—}249}, or a minor nucleotide occurrence of at least one of nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283 }, and nucleotide 1421 of SEQ ID NO:12 {HMGCRE16E18_{—}99}, or any combination thereof.

In addition, the present invention relates to a method for detecting a nucleotide occurrence for a SNP in a polynucleotide by incubating a sample containing the polynucleotide with a specific binding pair member, wherein the specific binding pair member specifically binds at or near a polynucleotide suspected of being polymorphic, and wherein the polynucleotide includes a thymidine residue at nucleotide 425 of SEQ ID NO:10 {CYP3A4E3-5_{—}249}, or a minor nucleotide occurrence corresponding to at least one of nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}, nucleotide 519 of SEQ ID NO:11 {HMGCRE5E6-3_{—}283}, and nucleotide 1421 of SEQ ID NO:12 {HMGCRE16E18_{—}99}, or any combination thereof; and detecting selective binding of the specific binding pair member, wherein selective binding is indicative of the presence of the nucleotide occurrence. Such methods can be performed, for example, by a primer extension reaction or an amplification reaction such as a polymerase chain reaction, using an oligonucleotide primer that selectively hybridizes upstream, or an amplification primer pair that selectively hybridizes to nucleotide sequences flanking and in complementary strands of the SNP position, respectively; contacting the material with a polymerase; and identifying a product of the reaction indicative of the SNP.

In addition, the present invention relates to a method for detecting a nucleotide occurrence for a SNP in a polynucleotide by incubating a sample containing the polynucleotide with a specific binding pair member, wherein the specific binding pair member specifically binds at or near a polynucleotide suspected of being polymorphic, and wherein the polynucleotide includes a minor nucleotide occurrence corresponding to at least one of nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283 }, and nucleotide 1421 of SEQ ID NO:12 {HMGCRE16E18_{—}99}, or any combination thereof; and detecting selective binding of the specific binding pair member, wherein selective binding is indicative of the presence of the nucleotide occurrence. Such methods can be performed, for example, by a primer extension reaction or an amplification reaction such as a polymerase chain reaction, using an oligonucleotide primer that selectively hybridizes upstream, or an amplification primer pair that selectively hybridizes to nucleotide sequences flanking and in complementary strands of the SNP position, respectively; contacting the material with a polymerase; and identifying a product of the reaction indicative of the SNP.

Accordingly, the present invention also relates to an isolated primer pair, which can be useful for amplifying a nucleotide sequence comprising a SNP in a polynucleotide, wherein a forward primer of the primer pair selectively binds the polynucleotide upstream of the SNP position on one strand and a reverse primer selectively binds the polynucleotide upstream of the SNP position on a complementary strand, wherein the polynucleotide includes a thymidine residue at nucleotide 425 of SEQ ID NO:10 {CYP3A4E3-5_{—}249}, or a minor nucleotide occurrence corresponding to at least one of nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}, nucleotide 519 of SEQ ID NO:11 {HMGCRE5E6-3_{—}283}, and nucleotide 1421 of SEQ ID NO:12 {HMGCRE16E18_{—}99}.

The isolated primer pair can include a 3′ nucleotide that is complementary to one nucleotide occurrence of the statin response-related SNP. Accordingly, the primer can be used to selectively prime an extension reaction to polynucleotides wherein the nucleotide occurrence of the SNP is complementary to the 3′ nucleotide of the primer pair, but not polynucleotides with other nucleotide occurrences at a position corresponding to the SNP.

In another embodiment the present invention provides an isolated probe for determining a nucleotide occurrence of a single nucleotide polymorphism (SNP) in a polynucleotide, wherein the polynucleotide includes a thymidine residue at nucleotide 425 of SEQ ID NO:10 {CYP3A4E3-5_{—}249}, or a minor nucleotide occurrence corresponding to at least one of nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}, nucleotide 519 of SEQ ID NO:11 {HMGCRE5E6-3_{—}283}, and nucleotide 1421 of SEQ ID NO:12 {HMGCRE16E18_{—}99}.

In another embodiment the present invention provides an isolated probe for determining a nucleotide occurrence of a single nucleotide polymorphism (SNP) in a polynucleotide, wherein the polynucleotide includes a thymidine residue at nucleotide 425 of SEQ ID NO:10 {CYP3A4E3-5_{—}249}, or a minor nucleotide occurrence corresponding to at least one of nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}, nucleotide 519 of SEQ ID NO:11 {HMGCRE5E6-3_{—}283 }, and nucleotide 1421 of SEQ ID NO:12 {HMGCRE16E18_{—}99}.

In another embodiment the present invention provides an isolated probe for determining a nucleotide occurrence of a single nucleotide polymorphism (SNP) in a polynucleotide, wherein the probe selectively binds to a polynucleotide comprising a minor nucleotide occurrence of a statin response-related SNP. The polynucleotide includes a minor nucleotide occurrence of a SNP corresponding to

nucleotide 1274 of SEQ ID NO:1 {CYP2D6E7_{—}339},

nucleotide 1757 of SEQ ID NO:2 {HMGCRE7E11-3_{—}472},

nucleotide 1159 of SEQ ID NO:4 {CYP2D6PE1_{—}2},

nucleotide 1093 of SEQ ID NO:5 {CYP2D6PE7_{—}150},

nucleotide 1223 of SEQ ID NO:6 {CYP2D6PE7_{—}286},

nucleotide 1311 of SEQ ID NO:7 {CYP3A4E7_{—}243},

nucleotide 808 of SEQ ID NO:8 {CYP3A4E10-5_{—}292},

nucleotide 227 of SEQ ID NO:9 {CYP3A4E12_{—}76};

nucleotide 425 of SEQ ID NO:10 {CYP3A4E3-5_{—}249},

nucleotide 519 of SEQ ID NO:11 {HMGCRE5E6-3_{—}283}, and

nucleotide 1421 of SEQ ID NO:12 {HMGCRE16E18_{—}99}.

In another embodiment, the present invention provides an isolated primer for extending a polynucleotide. The isolated polynucleotide includes a single nucleotide polymorphism (SNP), wherein the primer selectively binds the polynucleotide upstream of the SNP position on one strand. The polynucleotide includes a thymidine residue at nucleotide 425 of SEQ ID NO:10 {CYP3A4E3-5_{—}249}, or a minor nucleotide occurrence corresponding to at least one of

nucleotide 1274 of SEQ ID NO:1 {CYP2D6E7_{—}339},

nucleotide 1757 of SEQ ID NO:2 {HMGCRE7E11-3_{—}472},

nucleotide 1159 of SEQ ID NO:4 {CYP2D6PE1_{—}2},

nucleotide 1093 of SEQ ID NO:5 {CYP2D6PE7_{—}150},

nucleotide 1223 of SEQ ID NO:6 {CYP2D6PE7_{—}286},

nucleotide 1311 of SEQ ID NO:7 {CYP3A4E7_{—}243},

nucleotide 808 of SEQ ID NO:8 {CYP3A4E10-5_{—}292},

nucleotide 227 of SEQ ID NO:9 {CYP3A4E12_{—}76},

nucleotide 519 of SEQ ID NO:11 {HMGCRE5E6-3_{—}283 }, and

nucleotide 1421 of SEQ ID NO:12 {HMGCRE16E18_{—}99}.

In another embodiment, the present invention provides an isolated primer for extending a polynucleotide. The isolated polynucleotide includes a single nucleotide polymorphism (SNP), wherein the primer selectively binds the polynucleotide upstream of the SNP position on one strand. The polynucleotide includes a thymidine residue at nucleotide 425 of SEQ ID NO:10 {CYP3A4E3-5_{—}249}, or a minor nucleotide at a position corresponding to at least one of

nucleotide 1274 of SEQ ID NO:1 {CYP2D6E7_{—}339},

nucleotide 1757 of SEQ ID NO:2 {HMGCRE7E11-3_{—}472},

nucleotide 1159 of SEQ ID NO:4 {CYP2D6PE1_{—}2},

nucleotide 1093 of SEQ ID NO:5 {CYP2D6PE7_{—}150},

nucleotide 1223 of SEQ ID NO:6 {CYP2D6PE7_{—}286},

nucleotide 1311 of SEQ ID NO:7 {CYP3A4E7_{—}243},

nucleotide 808 of SEQ ID NO:8 {CYP3A4E10-5_{—}292},

nucleotide 227 of SEQ ID NO:9 {CYP3A4E12_{—}76};

nucleotide 425 of SEQ ID NO:10 {CYP3A4E3-5_{—}249},

nucleotide 519 of SEQ ID NO:11 {HMGCRE5E6-3_{—}283 }, and

nucleotide 1421 of SEQ ID NO:12 {HMGCRE16E18_{—}99}.

The present invention further relates to an isolated specific binding pair member, which can be useful for determining a nucleotide occurrence of a SNP in a polynucleotide, wherein the specific binding pair member specifically binds to a polynucleotide that includes a thymidine residue at nucleotide 425 of SEQ ID NO:10 {CYP3A4E3-5_{—}249}, or a minor nucleotide occurrence at a position corresponding to at least one of nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283}, and nucleotide 1421 of SEQ ID NO:12 {HMGCRE16E18_{—}99}.

The present invention further relates to an isolated specific binding pair member, which can be useful for determining a nucleotide occurrence of a SNP in a polynucleotide, wherein the specific binding pair member specifically binds to a minor nucleotide occurrence of the polynucleotide at or near a position corresponding to nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283}, and nucleotide 1421 of SEQ ID NO:12 {HMGCRE16E18_{—}99}. The specific binding pair member can be, for example, an oligonucleotide or an antibody. Where the specific binding pair member is an oligonucleotide, it can be a substrate for a primer extension reaction, or can be designed such that is selectively hybridizes to a polynucleotide at a sequence comprising the SNP as the terminal nucleotide.

The present invention also relates to a kit, which contains one or more components useful for identifying at least one statin response related SNP. For example, the kit can contain an isolated primer, primer pair, or probe of the invention, or a combination of such primers and/or primer pairs and/or probes. The kit also can contain one or more reagents useful in combination with another component of the kit. For example, reagents for performing an amplification reaction can be included where the kit contains one or more primer pairs of the invention. Similarly, at least one detectable label, which can be used to label an oligonucleotide probe, primer, or primer pair contained in the kit, or that can be incorporated into a product generated using a component of the kit, also can be included, as can, for example, a polymerase, ligase, endonuclease, or combination thereof.

The kit can further contain at least one polynucleotide that includes a minor nucleotide occurrence at a position corresponding to a statin response-related SNP.

The kit of the invention can include an isolated primer according of the invention and an isolated primer pair of the invention.

The present invention also relates to an isolated polynucleotide, which contains at least about 30 nucleotides and a minor nucleotide occurrence of a SNP of an HMGCR gene, in at least one position corresponding to nucleotide 519 of SEQ ID NO:11 {HMGCRE5E6-3_{—}283}, nucleotide 1757 of SEQ ID NO:2 {HMGCRE7E11-3_{—}472}, nucleotide corresponding to nucleotide 1430 of SEQ ID NO:3 {HMGCRDBSNP_{—}45320}, and nucleotide corresponding to nucleotide 1421 of SEQ ID NO:12 {HMGCRE16E18_{—}99}. The isolated polynucleotide can further include a minor nucleotide occurrence at a second statin-related SNP corresponding to nucleotide 519 of SEQ ID NO:11 {HMGCRE5E6-3_{—}283}, nucleotide 1757 of SEQ ID NO:2 {HMGCRE7E11-3_{—}472}, and nucleotide 1421 of SEQ ID NO:12 {HMGCRE16E18_{—}99}. The isolated polynucleotide can include a minor HMGCRB haplotype allele.

A polynucleotide of the present invention, in another embodiment, can include at least 30 nucleotides of the human cytochrome p450 3A4 (CYP3A4) gene, wherein the polynucleotide comprises in at least one minor nucleotide occurrence of a first statin response-related SNP corresponding to nucleotide 425 of SEQ ID NO:10 {CYP3A4E3-5_{—}249}, nucleotide 1311 of SEQ ID NO:7 {CYP3A4E7_{—}243}, nucleotide 808 of SEQ ID NO:8 {CYP3A4E10-5_{—}292}, and nucleotide 227 of SEQ ID NO:9 {CYP3A4E12_{—}76}. The polynucleotide can further include a minor nucleotide occurrence at a second statin-related SNP corresponding to nucleotide 425 of SEQ ID NO:10 {CYP3A4E3-5_{—}249}, nucleotide 1311 of SEQ ID NO:7 {CYP3A4E7_{—}243}, nucleotide 808 of SEQ ID NO:8 {CYP3A4E10-5_{—}292}, and nucleotide 227 of SEQ ID NO:9 {CYP3A4E12_{—}76}. The isolated polynucleotide can include a minor CYP3A4A, CYP3A4B, or CYP3A4C haplotype allele.

In another embodiment, the present invention provides an isolated polynucleotide that includes at least 30 nucleotides of the cytochrome p450 2D6 (CYP2D6) gene. The polynucleotide includes in at least a first minor nucleotide occurrence of at least a first statin response related single nucleotide polymorphism (SNP), wherein said minor nucleotide occurrence is at a position corresponding to nucleotide 1159 of SEQ ID NO:4 {CYP2D6PE1_{—}2}, a nucleotide 1093 of SEQ ID NO:5 {CYP2D6PE7_{—}150}, and a nucleotide 1223 of SEQ ID NO:6 {CYP2D6PE7_{—}286}. The isolated polynucleotide can further include a minor nucleotide occurrence at a second statin-related SNP corresponding to nucleotide 1159 of SEQ ID NO:4 {CYP2D6PE1_{—}2}, a nucleotide 1093 of SEQ ID NO:5 {CYP2D6PE7_{—}150}, and a nucleotide 1223 of SEQ ID NO:6 {CYP2D6PE7_{—}286}. Furthermore, the isolated polynucleotide can include a minor CYP2D6A haplotype allele.

The isolated polynucleotides of the present invention can be at least 50, at least 100, at least 200, at least 250, at least 500, or at least 1000 nucleotides in length. 193.

In another embodiment the present invention provides a vector containing one or more of the isolated polynucleotides disclosed above. In another embodiment, the present invention provides an isolated cell containing one or more of the isolated polynucleotides disclosed above, or one or more of the vectors disclosed in the preceding sentence.

In another embodiment, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the SNPs listed in Table 9-1, Table 9-2, Table 9-3, Table 9-4, Table 9-5, Table 9-6, Table 9-7, Table 9-8, Table 9-9, Table 9-10, Table 9-11, and Table 9-12. The nucleotide occurrence is associated with a statin response. Thereby an inference of the statin response of the subject is provided.

In another embodiment, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-1 and Table 9-2, whereby the nucleotide occurrence is associated with a decrease in low density lipoprotein in response to administration of Atorvastatin, thereby inferring the statin response of the subject. The method can be performed wherein the SNP occurs in one of the genes listed in Table 9-1 and Table 9-2 that includes at least two statin response-related SNPs.

In another embodiment, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) listed in Table 9-1 and Table 9-2, whereby the nucleotide occurrence is associated with a decrease in low density lipoprotein in response to administration of Atorvastatin. Thereby, identification of the nucleotide occurrence of the SNP provides an inference of the statin response of the subject. In one example, the subject is Caucasian and the statin response-related SNP is at least one SNP listed in Table 9-2.

In another aspect the present invention provides, a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-3 and Table 9-4, whereby the nucleotide occurrence is associated with a decrease in total cholesterol in response to administration of Atorvastatin. Thereby, identification of the nucleotide occurrence of the SNP provides an inference of the statin response of the subject. In one aspect, the SNP occurs in one of the genes listed in Table 9-3 and Table 9-4 comprising at least two statin response-related SNPs.

In another aspect the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) listed in Table 9-3 and Table 9-4, whereby the nucleotide occurrence is associated with a decrease in total cholesterol in response to administration of Atorvastatin. Thereby, identification of the nucleotide occurrence of the SNP provides an inference of the statin response of the subject. In one aspect, the subject is Caucasian and the statin response-related SNP is at least one SNP listed in Table 9-4.

In another aspect the present invention provides, a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-5 and Table 9-6, whereby the nucleotide occurrence is associated with an increase in SGOT readings in response to administration of Atorvastatin. Thereby, identification of the nucleotide occurrence of the SNP provides an inference of the statin response of the subject. In one aspect, the SNP occurs in one of the genes listed in Table 9-5 and Table 9-6 comprising at least two statin response-related SNPs.

In another aspect, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) listed in Table 9-5 and Table 9-6, whereby the nucleotide occurrence is associated with an increase in SGOT readings in response to administration of Atorvastatin. Thereby, identification of the nucleotide occurrence of the SNP provides an inference of the statin response of the subject. In one aspect, the subject is Caucasian and the statin response-related SNP is at least one SNP listed in Table 9-6.

In another aspect the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-7 and Table 9-8, whereby the nucleotide occurrence is associated with an increase in ALTGPT readings in response to administration of Atorvastatin. Thereby, identification of the nucleotide occurrence of the SNP provides an inference of the statin response of the subject. In one aspect, the SNP occurs in one of the genes listed in Table 9-7 and Table 9-8 comprising at least two statin response-related SNPs.

In another aspect the present invention provides, a method for inferring a statin response of a human subject from a nucleic acid sample of the subject, wherein the method includes identifying, in the nucleic acid sample, a nucleotide occurrence of at least one statin response-related single nucleotide polymorphism (SNP) listed in Table 9-7 and Table 9-8, whereby the nucleotide occurrence is associated with an increase in ALTGPT readings in response to administration of Atorvastatin Thereby, identification of the nucleotide occurrence of the SNP provides an inference of the statin response of the subject. In one aspect, the subject is Caucasian and the statin response-related SNP is at least one SNP listed in Table 9-8.

In another embodiment, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-9 and Table 9-10, whereby the nucleotide occurrence is associated with a decrease in low density lipoprotein in response to administration of Simvastatin. Thereby, identification of the nucleotide occurrence of the SNP provides an inference of the statin response of the subject. In one aspect, the SNP occurs in one of the genes listed in Table 9-9 and Table 9-10 comprising at least two statin response-related SNPs.

In another aspect, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) listed in Table 9-9 and Table 9-10, whereby the nucleotide occurrence is associated with a decrease in low density lipoprotein in response to administration of Simvastatin. Thereby, identification of the nucleotide occurrence of the SNP provides an inference of the statin response of the subject. In one aspect, the subject is Caucasian and the statin response-related SNP is at least one SNP listed in Table 9-10.

In another embodiment, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-1 1 and Table 9-12, whereby the nucleotide occurrence is associated with a decrease in total cholesterol in response to administration of Simvastatin Thereby, identification of the nucleotide occurrence of the SNP provides an inference of the statin response of the subject. In one aspect, the SNP occurs in one of the genes listed in Table 9-1 1 and Table 9-12 comprising at least two statin response-related SNPs.

In another aspect, the present invention provides a method for inferring a statin response of a human subject from a nucleic acid sample of the subject, the method includes identifying, in the nucleic acid sample, a nucleotide occurrence of at least one statin response-related single nucleotide polymorphism (SNP) listed in Table 9-11 and Table 9-12, whereby the nucleotide occurrence is associated with a decrease in total cholesterol in response to administration of Simvastatin. Thereby, identification of the nucleotide occurrence of the SNP provides an inference of the statin response of the subject. In one aspect, the subject is Caucasian and the statin response-related SNP is at least one SNP listed in Table 9-12.

### BRIEF DESCRIPTION OF THE DRAWINGS

FIG. 1 is a haplotype cladogram for the four haplotype system of HMGCRE7E11-3_{—}472 and HMGCRDBSNP_{—}45320 loci, as follows (in order): 1)GT; 2)AT; 3)GC; and 4)AC, as discussed in Example 3.

FIG. 2 is a graph of the haplotype pairs for individual patients plotted in 2 dimensional space. Individual haplotypes are shown as lines whose coordinates are GT/GT (1,1)(1,1); GT/AT (1,1)(0,1); GT/GC (1,1)(1,0); GT/AC (1,1)(0,0). If a person had two of the same haplotypes, for Example, GT/GT, which encoded as (1,1)(1,1), they were represented as a circle rather than a line.

Solid lines or filled circles indicate individuals who did not respond to statin treatment, and dashed lines or open circles represent those that responded positively to statin treatment.

### DETAILED DESCRIPTION OF THE INVENTION

The invention relates to methods for inferring a statin response of a human subject from a nucleic acid sample of the subject. The methods of the invention are based, in part, on the identification of single nucleotide polymorphisms (SNPs) that, alone or in combination, especially when combined into haplotypes, allow an inference to be drawn as to a statin response. The statin response can be a lowering of total cholesterol or LDL, or it can be an adverse reaction. As such, the compositions and methods of the invention are useful, for example, for identifying patients who are most likely to respond to statin treatment and most likely not to suffer adverse effects of statin treatment.

In one aspect, the present invention provides a method for inferring a statin response of a human subject from a nucleic acid sample of the subject by identifying in the biological sample, a nucleotide occurrence of at least one statin response-related single nucleotide polymorphism (SNP) corresponding to nucleotide 1757 of SEQ ID NO:2 {HMGCRE7E11-3_{—}472}, nucleotide 1430 of SEQ ID NO:3 {HMGCRDBSNP_{—}45320}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283}, and nucleotide 1421 of SEQ ID NO:12 {HMGCRE16E18_{—}99}. In this aspect, the nucleotide occurrence is associated with a decrease in total cholesterol or low density lipoprotein in response to administration of the statin. Thereby, a statin response is inferred for the subject.

In one embodiment of this aspect of the invention, a nucleotide occurrence of each of at least two statin response-related SNPs is identified. For this embodiment, nucleotide occurrences of at least two of the statin response-related SNPs can comprise at least one haplotype allele.

Accordingly, another embodiment of this aspect of the invention provides a method for inferring a statin response of a human subject from a nucleic acid sample of the subject by identifying, in the nucleic acid sample, at least one haplotype allele indicative of a statin response. The haplotype allele indicative of a statin response includes:

a) nucleotides of the cytochrome p450 3A4 (CYP3A4) gene, corresponding to

i) a CYP3A4A haplotype, which includes

nucleotide 808 of SEQ ID NO:8 {CYP3A4E10-5_{—}292}, and

nucleotide 227 of SEQ ID NO:9 {CYP3A4E12_{—}76}; or

ii) a CYP3A4B haplotype, which includes

nucleotide 1311 of SEQ ID NO:7 {CYP3A4E7_{—}243},

nucleotide 808 of SEQ ID NO:8 {CYP3A4E10-5_{—}292}, and

nucleotide 227 of SEQ ID NO:9 {CYP3A4E12_{—}76}; or

iii) a CYP3A4C haplotype, which includes

nucleotide 425 of SEQ ID NO:10 {CYP3A4E3-5_{—}249},

nucleotide 1311 of SEQ ID NO:7 {CYP3A4E7_{—}243},

nucleotide 808 of SEQ ID NO:8 {CYP3A4E10-5_{—}292}, and

nucleotide 227 of SEQ ID NO:9 {CYP3A4E12_{—}76}; or

b.) nucleotides of the 3-hydroxy-3-methylglutaryl-coenzyme A reductase (HMGCR) gene, corresponding to:

i) an HMGCRA haplotype, which includes

nucleotide 1757 of SEQ ID NO:2 {HMGCRE7E11-3_{—}472}, and

nucleotide 1430 of SEQ ID NO:3 {HMGCRDBSNP_{—}45320};

ii) an HMGCRB haplotype, which includes

nucleotide 519 of SEQ ID NO:11 {HMGCRE5E6-3_{—}283},

nucleotide 1757 of SEQ ID NO:2 {HMGCRE7E11-3_{—}472},

nucleotide 1430 of SEQ ID NO:3 {HMGCRDBSNP_{—}45320}, and

nucleotide 1421 of SEQ ID NO:12 {HMGCRE16E18_{—}99}; or

iii) an HMGCRC haplotype, which includes

nucleotide 1757 of SEQ ID NO:2 {HMGCRE7E11-3_{—}472},

nucleotide 1430 of SEQ ID NO:3 {HMGCRDBSNP_{—}45320}, and

nucleotide 1421 of SEQ ID NO:12 {HMGCRE16E18_{—}99}.

As disclosed herein, the identification of at least one statin response-related haplotype allele allows an inference to be drawn as to a statin response of a human subject. An inference drawn according to a method of the invention can be strengthened by identifying a second, third, fourth or more statin response-related haplotype allele in the same, or preferably different statin response-related gene(s). Accordingly, the method can further include identifying in the nucleic acid sample at least a second statin response-related haplotype allele. The first and second haplotypes are typically found in the cytochrome p450 3A4 (CYP3A4) and 3-hydroxy-3-methylglutaryl-coenzyme A reductase (HMGCR) genes, respectively. As disclosed in the Examples included herein, and listed above, statin response-related haplotypes and haplotype alleles for these genes are provided herein. In a preferred embodiment, the CYP3A4 haplotype is CYP3A4C and the HMGCR haplotype is HMGCRB. In another embodiment the CYP3A4 haplotype is CYP3A4C and the HMGCR haplotype is HMGCRC.

Statins are a class of medications that have been shown to be effective in lowering human total cholesterol (TC) and low density lipoprotein (LDL) levels in hyperlipidemic patients. The drugs act at the step of cholesterol synthesis. By reducing the amount of cholesterol synthesized by the cell, through inhibition of the HMG Co-A Reductase gene (HMGCR), the drug initiates a cycle of events that culminates in the increase of LDL uptake by liver cells. As LDL uptake is increased, total cholesterol and LDL levels in the blood decrease. Lower blood levels of both factors are associated with lower risk of atherosclerosis and heart disease, and the Statins are widely used to reduce atherosclerotic morbidity and mortality. Nonetheless, some patients show no response to a given Statin.

Methods of the present invention provide an inference of a statin response after administration of statins to a subject. The inference of the present invention assumes that statins are administered at an effective dosage, for example, using FDA approved guidelines including dosages, for those statins that are FDA approved. An effective dosage is a dosage where a statin has been shown to reduce serum cholesterol in the general population without respect to HMGCR or CYP3A4 genotype.

It will be understood that any method of the present invention, or SNP identified herein, will be useful not only for predicting a positive response to statins, but for predicting a negative response as well.

Drugs such as statins are called xenobiotics because they are chemical compounds that are not naturally found in the human body. Xenobiotic metabolism genes make proteins whose sole purpose is to detoxify foreign compounds present in the human body, and they evolved to allow humans to degrade and excrete harmful chemicals present in many foods (such as tannins and alkaloids from which many drugs are derived). The CYP3A4 gene is the primary gene in the human body responsible for metabolism of both drugs.

Examples of statins include, but are not limited to, Fluvastatin (Lescol™), Atorvastatin (Lipitor™), Lovastatin (Mevacor™), Pravastatin (Pravachol™), Simvastatin (Zocor™), Cerivastatin (Baycol™). The chemical structure of these statins are known and widely available. For example, Atorvastatin calcium is {R-(R*,R*) }-2-(4-fluorophenyl)-b,d-dihydroxy-5-(1-methylethyl)-3-phenyl-4{(phenylamino)carbonyl}-1H-pyrrole-1-heptanoic acid, calcium salt (2:1) trihydrate. The empirical formula of atorvastatin calcium is (C₃₃H₃₄FN₂O₅)2Ca.3H2O and its molecular weight is 1209.42. Simvastatin is butanoic acid, 2,2-dimethyl-,1,2,3,7,8,8a-hexahydro-3,7-dimethyl-8-{2-(tetrahydro-4-hydroxy-6-oxo-2H-pyran-2-yl)-ethyl}-1-naphthalenyl ester, {1S*-{1a,3a,7b,8b(2S*,4S),-8ab}}. The empirical formula of Simvastatin is C₂₅H₃₈O₅ and its molecular weight is 418.57. Pravastatin sodium is designated chemically as 1-Naphthalene-heptanoic acid, 1,2,6,7,8,8a-hexahydro-b, d,6-trihydroxy-2-methyl-8-(2-methyl-1-oxobutoxy)-, monosodium salt, {1S-{1a(bS*, d S*),2a,6a,8b(R*),8aa}}-. Formula C₂₃H₃₅NaO₇, Molecular Weight is 446.52.

For the statin response-related genes of this aspect of the invention wherein the statin response-related SNPs are located in the CYP3A4 and/or the HMGCR genes, the statin response is typically statin efficacy (i.e. lowering of serum cholesterol levels). This is also referred to herein as a positive response to statins or a favorable response to statins. Statin efficacy can be determined by a cholesterol test to determine whether cholesterol levels are lowered as a result of statin administration. Such tests include total cholesterol (TC) and/or low density lipoprotein (LDL) measurements, as illustrated in Examples 3, 5, 6, and 7. Methods, such as those disclosed in Examples 3, 5, 6, and 7 are widely used in clinical practice today, for determining levels of TC and LDL in blood, especially serum samples, and for interpreting results of such tests.

A cholesterol test is often performed to evaluate risks for heart disease. As is known in the art, cholesterol is an important normal body constituent, used in the structure of cell membranes, synthesis of bile acids, and synthesis of steroid hormones. Since cholesterol is water insoluble, most serum cholesterol is carried by lipoproteins (chylomicrons, VLDL, LDL, and HDL). The term “LDL” means LDL-cholesterol and “HDL” means HDL-cholesterol. The term “cholesterol” means total cholesterol (VLDL+LDL+HDL).

Excess cholesterol in the blood has been correlated with cardiovascular disease. LDL is sometimes referred to as “bad” cholesterol, because elevated levels of LDL correlate most directly with coronary heart disease. HDL is sometimes referred to as “good” cholesterol since high levels of HDL reduce risk for coronary heart disease.

Preferably, cholesterol is measured after a patient has fasted. In 2001, guidelines from the National Cholesterol Education Panel recommended that all lipid tests be performed fasting and should measure total cholesterol, HDL, LDL and triglycerides. The total cholesterol measurement, as with all lipid measurements, is typically reported in milligrams per deciliter (mg/dL). Typically, the higher the total cholesterol, the more at risk a subject is for heart disease. A value of less than 200 mg/dL is a “desirable” level and places the subject in a group at less risk for heart disease. Levels over 240 mg/dL may put a subject at almost twice the risk of heart disease as compared to someone with a level less than 200 mg/dL. High LDL cholesterol levels may be the best predictor of risk of heart disease.

The statin response-related SNPs and haplotypes of the present invention can be used to infer whether a patient's cholesterol levels are more likely to be reduced by statin treatment. A patient whose cholesterol levels, e.g. LDL levels or TC levels, are reduced by statin treatment can be referred to as responders. However, for classification of a subject as a Responder, a cutoff cholesterol reduction minimum can be set. For example, a subject can be classified as a Responder if TC or LDL or both TC and LDL are reduced by at least 1%, or reduced by at least 20%.

As used herein, the term “at least one”, when used in reference to a gene, SNP, haplotype, or the like, means 1, 2, 3, 4, 5, 6, 7, 8, 9, 10, etc., up to and including all of the exemplified statin response-related haplotype alleles, statin response-related genes, or statin response-related SNPs. Reference to “at least a second” gene, SNP, or the like, for example, a statin response-related gene, means two or more, i.e., 2, 3, 4, 5, 6, 7, 8, 9, 10, etc., statin response-related genes.

The term “haplotypes” as used herein refers to groupings of two or more nucleotide SNPs present in a gene. The term “haplotype alleles” as used herein refers to a non-random combination of nucleotide occurrences of SNPs that make up a haplotype. Haplotype alleles are much like a string of contiguous sequence bases, except the SNPs are not adjacent to one another on a chromosome. For example, SNPs can be included as part of the same haplotype, even if they are thousands of base pairs apart from one another on a genome. Typically, SNPs that make up a haplotype are from the same gene.

Penetrant statin response-related haplotype alleles are haplotype alleles whose association with a statin response is strong enough to be detected using simple genetics approaches. Corresponding haplotypes of penetrant statin response-related haplotype alleles, are referred to herein as “penetrant statin response-related haplotypes.” Similarly, individual nucleotide occurrences of SNPs are referred to herein as “penetrant statin response-related SNP nucleotide occurrences” if the association of the nucleotide occurrence with a statin response is strong enough on its own to be detected using simple genetics approaches, or if the SNP loci for the nucleotide occurrence make up part of a penetrant haplotype. The corresponding SNP loci are referred to herein as “penetrant statin response-related SNPs.” Haplotype alleles of penetrant haplotypes are also referred to herein as “penetrant haplotype alleles” or “penetrant genetic features.” Penetrant haplotypes are also referred to herein as “penetrant genetic feature SNP combinations.” The SNPs disclosed herein, and listed in Tables 1 and 2 below, include both penetrant and latent (see below) statin response-related SNPs, and make up statin response-related penetrant haplotypes. since they were identified using simple genetics approaches.

Tables 1 and 3A-B identifies and provides information regarding SNPs disclosed herein that are associated with a statin response. Tables 1 and 3 set out the marker name, a SEQ ID NO: for the SNP and surrounding nucleotide sequences in the genome, and the position of the SNP within the sequence listing entry for that SNP and surrounding sequences. From this information, the SNP loci can be identified within the human genome. Table 2 identifies and provides information regarding haplotypes of the present invention that are related to a statin response. Additionally, the sequence listing provides flanking sequences, and Table 3A-B provides the variable nucleotide occurrence, and additional information regarding the statin response-related SNPs of the present invention including the name and marker numbers for the SNP, a Genbank accession number of the gene from which a SNP occurs, and information regarding whether the SNP is within a coding region or intron of the gene for some of the SNPs of the present invention.

It will be recognized that the 5′ and 3′ flanking sequences exemplified herein, provide sufficient information to identify the SNP location within the human genome. However, due to variability in the human genome, in addition to the statin response-related SNPs disclosed herein, as well as sequencing inaccuracy and inaccuracy of information available in public databases, the 5′ and 3′ flanking sequences disclosed herein may not be 100% identical to a database entry, but need not be 100% identical to effectively identify the location of the SNP within a database sequence. However, when the flanking sequences are used to search a database of human genome sequences, it is expected that the highest match in terms of sequence identity will be the entry in the database that corresponds to the location within the human genome that includes the SNP surrounded by those flanking sequences.

TABLE 1
Statin response-related SNPs of the present invention

| SEQ | POSITION | | | |
|---|---|---|---|---|
| ID | of SNP in | | MARKER | |
| NO: | SEQ ID | MARKER NAME | NUMBER | EXAMPLE |
| 1 | | CYP2D6E7_339 | 554368 | 1 |
| 2 | | HMGCRE7E11_472 | 712050 | 3, 5, 6 |
| 3 | | HMGCRDBSNP_45320 | 712044 | 3, 5, 6 |
| 4 | | CYP2D6PE1_2 | 554371 | 4 |
| 5 | | CYP2D6PE7_150 | 554363 | 4 |
| 6 | | CYP2D6PE7_286 | 554365 | 4 |
| 7 | | CYP3A4E7_243 | 664803 | 5, 6 |
| 8 | | CYP3A4E10-5_292 | 712037 | 5, 6 |
| 9 | | CYP3A4E12_76 | 869772 | 5, 6 |
| 10 | | CYP3A4E3-5_249 | 809114 | 6 |
| 11 | | HMGCRE5E6-3_283, | 809125 | 6 |
| 12 | | HMGCRE16E18_99 | 664793 | 6 |

TABLE 2

| SEQ | | | | |
|---|---|---|---|---|
| ID | | | | |
| NO: | Haplotype | MARKER | EXAMPLE | STATIN RESPONSE |
| 1 | | CYP2D6E7_339 | 1 | Adverse hepatocellular |
| | | | | response |
| 2 | | HMGCRE7E11_472 | 3 | Efficacy |
| 3 | | HMGCRDBSNP_45320 | 3 | Efficacy |
| 4 | HMGCRA | HMGCRE7E11_472 | 3 | Efficacy |
| | | HMGCRDBSNP_45320 | | |
| 5 | CYP2D6A | CYP2D6PE1_2, | 4 | Adverse hepatocellular |
| | | CYP2D6PE7_150, | | response |
| | | CYP2D6PE7_286 | | |
| 6 | HMGCRA | HMGCRE7E11_472, | 5 | Efficacy |
| | | HMGCRDBSNP_45320 | | |
| 7 | CYP3A4A | CYP3A4E10-5_292, | 5 | Efficacy |
| | | CYP3A4E12_76 | | |
| 9 | CYP3A4A and | HMGCRDBSNP_45320, | 5 | Efficacy |
| | HMGCRA | HMGCRE7E11_472, | | |
| | | CYP3A4E10-5_292, | | |
| | | CYP3A4E12_76 | | |
| 10 | CYP3A4B and | HMGCRDBSNP_45320, | 5 | Efficacy |
| | HMGCRA | HMGCRE7E11_472, | | |
| | | CYP3A4E10-5_292, | | |
| | | CYP3A4E12_76, | | |
| | | CYP3A4E7_243 | | |
| 11 | CYP3A4C | CYP3A4E3-5_249, | 6, 7 | Efficacy |
| | | CYP3A4E7_243, | | |
| | | CYP3A4E10-5_292, | | |
| | | CYP3A4E12_76 | | |
| 12 | HMGCRB | HMGCRE5E6-3_283 | 6, 7 | Efficacy |
| | | (809125), HMGCRE7E11- | | |
| | | 3_472 (712050), | | |
| | | HMGCRDBSNP_45320(71 | | |
| | | 2044), and | | |
| | | HMGCRE16E18_99 | | |
| | | (664793) | | |
| 13 | HMGCRB, | Combine CYP3As (root) | 6, 7 | Efficacy |
| | CYP3A4C | above with HMGCRs above | | |

TABLE 3A
Exemplary SNPs for an inference of a statin response

| GENE | SNPNAME | MARKER | LOCATION | GEN-BANK | Variant |
|---|---|---|---|---|---|
| CYP3A4 | CYP3A4E7_243 | 664803 | 15871 | AF209389 | K |
| CYP3A4 | CYP3A4E3-5_249 | 809114 | 6165 | AF209389 | W |
| CYP3A4 | CYP3A4E10-5_292 | 712037 | 20338 | AF209389 | R |
| CYP3A4 | CYP3A4E12_76 | 869772 | 23187 | AF209389 | Y |
| HMGCR | HMGCRE16E18_99 | 664793 | 42021 | AC008897 | M |
| HMGCR | HMGCRDBSNP_45320 | 712044 | 45320 | AC008897 | Y |
| HMGCR | HMGCRE7E11-3_472 | 712050 | 51597 | AC008897 | R |
| HMGCR | HMGCRE5E6-3_283 | 809125 | 55959 | AC008897 | Y |
| CYP2D6 | CYP2D6E7_339 | 554368 | 5054 | M33388 | M |
| CYP2D6 | CYP2D6PE1_2 | 554371 | 1719 | M33388 | Y |
| CYP2D6 | CYP2D6E7_150 | 554363 | 4873 | M33388 | Y |
| CYP2D6 | CYP2D6E7_286 | 554265 | 5003 | M33388 | M |

TABLE 3B

| SNPNAME | SOURCE | TYPE | INTEGRITY |
|---|---|---|---|
| CYP2D6E7_339 | RESEQ | INTRON | POLY |
| HMGCRE7E11-3_472 | RESEQ | INTRON | POLY |
| HMGCRDBSNP_45320 | DBSNP | ILE_VAL | POLY |
| CYP2D6PE1_2 | RESEQ | PRO_SER | POLY |
| CYP2D6E7_150 | RESEQ | SILENT | POLY |
| CYP2D6E7_286 | RESEQ | INTRON | POLY |
| HMGCRDBSNP_45320 | DBSNP | ILE_VAL | POLY |
| HMGCRE7E11-3_472 | RESEQ | INTRON | POLY |
| CYP3A4E10-5_292 | RESEQ | INTRON | POLY |
| CYP3A4E12_76 | RESEQ | INTRON | POLY |
| CYP3A4E7_243 | RESEQ | INTRON | POLY |

TABLE 4
Primer and probe sequences for CYP3A4 and HMGCR Statin response-
related SNPs.

| | Primer/Pro | Primer/Pro | SEQ ID | |
|---|---|---|---|---|
| SNP Name | be type | be # | NO: | SEQUENCE |
| CYP3A4E | PCRU | 1994442 | 19 | TATTCTGGAAACTTCCATTGGATAGA |
| 3-5_249 | | | | |
| CYP3A4E | PCRL | 1994443 | 20 | CAAATAAATATCTTCTTCTTTCAGAGAACTTC |
| 3-5_249 | | | | |
| CYP3A4E | Probe | | 35 | AGCCTCTTGGGATRAAGCTC |
| 3-5_249 | | | | |
| CYP3A4E | PCRU | 1547571 | 21 | CATYGACTCTCTCAACAATCCAC |
| 7_243 | | | | |
| CYP3A4E | PCRL | 1547572 | 22 | ACATGGTGATTTATATCTCAATAAAGCAG |
| 7_243 | | | | |
| CYP3A4E | Probe | | 36 | TATTTCCTTTAATTTATCTT |
| 7_243 | | | | |
| CYP3A4E | PCRU | 1654506 | 23 | TGCAGGAGGAAATTGATGC |
| 10-5_292 | | | | |
| CYP3A4E | PCRL | 1654507 | 24 | ATAAAAATTYTCCTGGGAAGTGGT |
| 10-5_292 | | | | |
| CYP3A4E | Probe | | 37 | CCCAATAAGGTGAGTGGATG |
| 10-5_292 | | | | |
| CYP3A4E | PCRU | 1989145 | 25 | CCKAAGTAAGAAACCCTAACATGTAACTC |
| 12_76 | | | | |
| CYP3A4E | PCRL | 1989146 | 26 | GTCCACTTCCAAAGGGTGTGTA |
| 12_76 | | | | |
| CYP3A4E | Probe | | 38 | ACTTTTTAAAAATCTACCAA |
| 12_76 | | | | |
| HMGCRE | PCRU | 1994475 | 27 | TACAGGGGACTGTTCCTGGG |
| 5E6-3_283 | | | | |
| HMGCRE | PCRL | 1994476 | 28 | GAATAGTATTCCTTTTTTCAGTTTACATTAATAGG |
| 5E6-3_283 | | | | |
| HMGCRE | Probe | | 39 | AATCTTGTGCTATGAAGAAA |
| 5E6-3_283 | | | | |
| HMGCRE | PCRU | 1654541 | 29 | TTACTCTTCTACTAGTGCCATATGTAAGAATTG |
| 7E11- | | | | |
| 3_472 | | | | |
| HMGCRE | PCRL | 1654542 | 30 | CTTGAAATTATGTGCTGCTTTGG |
| 7E11- | | | | |
| 3_472 | | | | |
| HMGGRE | Probe | | 40 | AAAGTCATGAACACGAAGTA |
| 7E11- | | | | |
| 3_472 | | | | |
| HMGCRD | PCRU | 1654527 | 31 | TTACCTTTGAAATCATGTTCATCCC |
| BSNP_453 | | | | |
| 20 | | | | |
| HMGCRD | PCRL | 1654528 | 32 | CTTTGCATCTTTTATTTATAGATTTGCAC |
| BSNP_453 | | | | |
| 20 | | | | |
| HMGCRD | Probe | | 41 | ATAAAGGTTGCGTCCAGCTA |
| BSNP_453 | | | | |
| 20 | | | | |
| HMGCRE | PCRU | 2039700 | 33 | GCTCTCTTCATCTACTTTCTTA TCTAACCA |
| 16E18_99 | | | | |
| HMGCRE | PCRL | 2039701 | 34 | TCTATCTGAGAYTATGTATCACTCACCTCTATT |
| 16E18_99 | | | | |
| HMGCRE | Probe | | 42 | ATGGATTAGGCTGATATGAC |
| 16E18_99 | | | | |

Table 4. PCRU is a forward primer and PCRL is a reverse primer.

Polymorphisms are allelic variants that occur in a population. The polymorphism can be a single nucleotide difference present at a locus, or can be an insertion or deletion of one or a few nucleotides. As such, a single nucleotide polymorphism (SNP) is characterized by the presence in a population of one or two, three or four nucleotides (i.e., adenosine, cytosine, guanosine or thymidine), typically less than all four nucleotides, at a particular locus in a genome such as the human genome. Accordingly, it will be recognized that, while the methods of the invention are exemplified primarily by the detection of SNPs, the disclosed methods or others known in the art similarly can be used to identify other polymorphisms in the exemplified genes or other statin response-related genes.

In methods of the present invention, the haplotype allele can include a) a CYP3A4A haplotype alleles, a CYP3A4B haplotype allele, or a CYP3A4C haplotype allele; b) an HMGCRA haplotype allele, an HMGCRB haplotype allele, or an HMGCRC haplotype allele; or c) a combination of a) and b).

In methods of the present invention, at least one CYP3A4C haplotype allele and at least one HMGCRB haplotype allele can be identified. As illustrated in Examples 6 and 7, the combination of both CYP3A4C and HMGCRB haplotype alleles can improve the accuracy of the inference of statin response. In methods of the present invention, at least one CYP3A4C haplotype allele and at least one HMGCRC haplotype allele can be identified.

In methods of the present invention, a diploid pair of alleles can be identified, and the diploid pair of haplotype alleles can include a) a diploid pair of CYP3A4A haplotype alleles, CYP3A4B haplotype alleles, or CYP3A4C haplotype alleles; b) a diploid pair of HMGCRA haplotype alleles, HMGCRB haplotype alleles or HMGCRC haplotype alleles; or c) a combination of a) and b).

In methods of the present invention, a diploid pair of alleles can be identified, and the diploid pair of haplotype alleles can include a diploid pair of CYP3A4C haplotype alleles; a diploid pair of HMGCRB haplotype alleles; or a diploid pair of CYP3A4C haplotype alleles and a diploid pair of HMGCRB haplotype alleles. As illustrated in Examples 6 and 7, the combination of both CYP3A4C and HMGCRB haplotype alleles can improve the accuracy of the inference of statin response.

In methods in which a diploid pair of CYP3A4C alleles are identified, the diploid pair of CYP3A4C haplotype alleles can be ATGC/ATGC or ATGC/ATAC. As illustrated in Table 6-3, statins such as Lipitor™ are more likely to be effective in individuals with an ATGC/ATGC or ATGC/ATAC CYP3A4C haplotypes.

In methods in which a diploid pair of HMGCR alleles are identified, a diploid pair of HMGCRB haplotype alleles can be CGTA/CGTA or CGTA/TGTA. As illustrated in Table 6-5, statins such as Lipitor™ are more likely to be effective in individuals with CGTA/CGTA or CGTA/TGTA HMGCRB haplotypes.

In methods in which a diploid pair of HMGCR alleles are identified, a diploid pair of HMGCRC haplotype alleles can be GTA/GTA. As illustrated in Table 6-5, statins such as Lipitor™ are more likely to be effective in individuals with GTA/GTA diploid haplotype alleles.

In methods in which a diploid pair of both CYP3A4C alleles and HMGCRB alleles are determined, the diploid pair of CYP3A4C haplotype alleles can be ATGC/ATGC, and the diploid pair of HMGCRB haplotype alleles can be CGTA/CGTA or CGTA/TGTA. As illustrated in Example 6, this combination of haplotype alleles improves the power of the inference of statin (e.g. Lipitor™) response. The statin whose response is inferred by these embodiments can be any statin, but in certain preferred examples is Simvastatin, and in certain most preferred examples, is Atorvastatin (i.e. Lipitor™).

In methods in which a diploid pair of both CYP3A4C alleles and HMGCRB alleles are determined, the diploid pair of CYP3A4C haplotype alleles can be ATGC/ATGC, and the diploid pair of HMGCRC haplotype alleles can be GTA/GTA.

Simple genetic approaches for discovering penetrant statin response-related haplotype alleles include analyzing allele frequencies in populations with different phenotypes for a statin response being analyzed, to discover those haplotypes that occur more or less frequently in individuals with a certain statin response, for example, decreased LDL levels. In such simple genetics methods SNP nucleotide occurrences are scored and distribution frequencies are analyzed. The Examples provide illustrations of using simple genetics approaches to discover statin response-related haplotypes, and disclose methods that can be used to discover other statin response-related haplotypes and their alleles, and other statin response-related SNPs.

Haplotypes can be inferred from genotype data corresponding to certain SNPs using the Stephens and Donnelly algorithm (Am J. Hum. Genet. 68:978-989, 2001). Haplotype phases (i.e., the particular haplotype alleles in an individual) can also be determined using the Stephens and Donnelly algorithm (Am. J. Hum. Genet. 68:978-989, 2001). Software programs are available which perform this algorithm (e.g., The PHASE program, Department of Statistics, University of Oxford).

In one example, called the Haploscope method (See U.S. patent applcation Ser. No. 10/120,804 entitled “METHOD FOR THE IDENTIFICATION OF GENETIC FEATURES FOR COMPLEX GENETICS CLASSISFIERS,” filed Apr. 11, 2002) a candidate SNP combination is selected from a plurality of candidate SNP combinations for a gene associated with a genetic trait. Haplotype data associated with this candidate SNP combination are read for a plurality of individuals and grouped into a positive-responding group and a negative-responding group based on whether predetermined trait criteria, such as a statin response, for an individual are met. A statistical analysis (as discussed below) on the grouped haplotype data is performed to obtain a statistical measurement associated with the candidate SNP combination. The acts of selecting, reading, grouping, and performing are repeated as necessary to identify the candidate SNP combination having the optimal statistical measurement. In one approach, all possible SNP combinations are selected and statistically analyzed. In another approach, a directed search based on results of previous statistical analysis of SNP combinations is performed until the optimal statistical measurement is obtained. In addition, the number of SNP combinations selected and analyzed may be reduced based on a simultaneous testing procedure.

As used herein, the term “infer” or “inferring”, when used in reference to a statin response, means drawing a conclusion about a statin response using a process of analyzing individually or in combination, nucleotide occurrence(s) of one or more statin response-related SNP(s) in a nucleic acid sample of the subject, and comparing the individual or combination of nucleotide occurrence(s) of the SNP(s) to known relationships of nucleotide occurrence(s) of the statin response-related SNP(s). As disclosed herein, the nucleotide occurrence(s) can be identified directly by examining nucleic acid molecules, or indirectly by examining a polypeptide encoded by a particular gene, for example, a CYP3A4 gene, wherein the polymorphism is associated with an amino acid change in the encoded polypeptide.

Methods of performing such a comparison and reaching a conclusion based on that comparison are exemplified herein (see Example 6). The inference typically can involve using a complex model that involves using known relationships of known alleles or nucleotide occurrences as classifiers. The comparison can be performed by applying the data regarding the subject's statin response-related haplotype allele(s) to a complex model that makes a blind, quadratic discriminate classification using a variance-covariance matrix. Various classification models are discussed in more detail herein.

To determine whether haplotypes are useful in an inference of a statin response, numerous statistical analyses can be performed. Allele frequencies can be calculated for haplotypes and pair-wise haplotype frequencies estimated using an EM algorithm (Excoffier and Slatkin, Mol Biol Evol. 1995 Sep;12(5):921-7). Linkage disequilibrium coefficients can then be calculated. In addition to various parameters such as linkage disequilibrium coefficients, allele and haplotype frequencies, chi-square statistics and other population genetic parameters such as Panmitic indices can be calculated to control for ethnic, ancestral or other systematic variation between the case and control groups.

Markers/haplotypes with value for distinguishing the case matrix from the control, if any, can be presented in mathematical form describing any relationship and accompanied by association (test and effect) statistics. A statistical analysis result which shows an association of a SNP marker or a haplotype with a statin response with at least 80%, 85%, 90%, 95%, or 99%, most preferably 95% confidence, or alternatively a probability of insignificance less than 0.05, can be used to identify haplotypes. These statistical tools may test for significance related to a null hypothesis that an on-test SNP allele or haplotype allele is not significantly different between the groups. If the significance of this difference is low, it suggests the allele is not related to a statin response. The discovery of haplotype alleles can be verified and validated as genetic features for statin response using a nested contingency analysis of haplotype cladograms.

It is beneficial to express polymorphisms in terms of multi-locus haplotypes because, as disclosed in the Examples provided herein, far fewer haplotypes exist in the world population than would be predicted based on the expectations from random allele combinations. For example, as disclosed in Example 6, for the four disclosed polymorphic loci within the CYP3A4 gene for haplotype CYP3A4C, CYP3A4E3-5_{—}249, CYP3A4E7_{—}243, CYP3A4E10-5_{—}292, CYP3A4E12_{—}76, there would be 2⁴=16 possible haplotype combinations observed in the population. With the first letter in each haplotype allele corresponding to the first SNP, CYP3A4E3-5_{—}249, the second letter corresponding to the nucleotide occurrence of the second SNP (CYP3A4E7_{—}243) in the haplotype, the third letter corresponding to the nucleotide occurrence of the third SNP (CYP3A4E10-5_{—}292), and the fourth letter corresponding to the nucleotide occurrence of the fourth SNP (CYP3A4E12_{—}76) of the haplotype. The various haplotype alleles exemplified above can be considered possible or potential “flavors” of the CYP3A4 gene in the population. However, for the CYP3A4 SNPs listed above, seven haplotypes or “flavors” have been observed in real data from people of the world—ATGC, ATAC, AGAT, AGAC, ATAT, ATGT, and TGAC. The observance of a number of haplotypes in nature that is far fewer than the number of haplotypes possible is common and appreciated as a general principle among those familiar with the state of the art, and it is commonly accepted that haplotypes offer enhanced statistical power for genetic association studies. This phenomenon is caused by systematic genetic forces such as population bottlenecks, random genetic drift, selection, and the like, which have been at work in the population for millions of years, and have created a great deal of genetic “pattern” in the present population. As a result, working in terms of haplotypes offers a geneticist greater statistical power to detect associations, and other genetic phenomena, than working in terms of disjointed genotypes. For larger numbers of polymorphic loci the disparity between the number of observed and expected haplotypes is larger than for smaller numbers of loci.

In diploid organisms such as humans, somatic cells, which are diploid, include two alleles for each haplotype. As such, in some cases, the two alleles of a haplotype are referred to herein as a genotype or as a diploid pair, and the analysis of somatic cells, typically identifies the alleles for each copy of the haplotype. Methods of the present invention can include identifying a diploid pair of haplotype alleles. These alleles can be identical (homozygous) or can be different (heterozygous). The haplotypes of a subject can be symbolized by representing alleles on the top and bottom of a slash (e.g., ATG/CTA or GTT/AGA), where the sequence on the top of the slash represents the combination of polymorphic alleles on the maternal chromosome and the other, the paternal (or vice versa).

For certain haplotypes, one allele or a small number of alleles, are much more prevalent in the population than other alleles for that haplotype. Typically, major haplotypes alleles represent at least 25%, preferably at least 50%, more preferably at least 75%, of the allele occurrences in a population for a haplotype. For example, as illustrated in Example 4, for the CYP2D6 haplotype, CTA is much more prevalent in the population than other CYP2D6 alleles. Therefore, for CYP2D6, CTA is the major allele. For example as illustrated in Example 6, for the CYP3A4C haplotype, the ATGC allele is much more prevalent in the population than other CYP3A4C haplotype alleles. Therefore, for the CYP3A4C haplotype, ATGC is a major allele. For example as illustrated in Example 6, for the HMGCRB haplotype, the CGTA allele is much more prevalent in the population than other HMGCRB haplotype alleles. Therefore, for the HMGCRB haplotype, the CGTA allele is a major allele. For example, from the data shown in Table 6-7, 72 out of a total of 84 (86%) haplotype occurrences of HMGCRB haplotypes (2×42 diploid pairs of HMGCRB haplotypes) found in the population, were CGTA alleles.

For methods of the present invention that analyze diploid pairs of CYP3A4C or HMGCRB haplotypes alleles, the diploid pairs can include one minor and one major haplotype allele, a diploid pair of minor haplotype alleles, or a diploid pair of major haplotype alleles. As illustrated in the attached Examples, such as Example 6, the major allele of CYP3A4C, ATGC, and the major allele of HBGCRB, CGTA, especially homozygous diploid pairs of major alleles for these two haplotypes, are associated with a higher likelihood that a statin will be efficacious, for example decreasing LDL or TC levels.

In certain embodiments of the present invention, the diploid pair of CYP3A4C haplotype alleles is ATGC/ATGC, ATGC/ATAC, ATGC/AGAC, ATGC/AGAT, ATGC/ATAT, ATGC/TGAC or ATGT/AGAT. These are diploid pairs that were found in the population, as illustrated in Example 6. In certain embodiments of the present invention, the diploid pair of HMGCRB haplotype alleles is CGTA/CGTA, CGTA/TGTA, CGTA/CGCA, CGTA/CGTC, or CGTA/CATA. These are diploid pairs that were observed in the population, as illustrated in Example 6.

In certain embodiments of the present invention, the diploid pair can include every possible diploid pair for the haplotype alleles observed in the population. These diploid pairs can include for the CYP3A4C haplotype, ATGC/ATGC, ATGC/ATAC, ATAC/ATAC, ATGC/AGAC, AGAC/AGAC, ATAC/AGAC, ATGC/AGAT, AGAT/AGAT, AGAT/ATAC, AGAT/AGAC, ATGC/ATAT, ATAT/ATAT, ATAT/ATAC, ATAT/AGAC, ATAT/AGAT, ATGC/TGAC, TGAC/TGAC, TGAC/ATAC, TGAC/AGAC, TGAC/AGAT, TGAC/ATAT, ATGC/AGAT, AGAT/AGAT, AGAT/ATAC, AGAT/AGAC, AGAT/AGAT, AGAT/ATAT, or AGAT/TGAC. These diploid pairs can include for the HMGCRB haplotype, CGTA/CGTA, CGTA/TGTA, CGTA/CGTA, CGTA/CGCA, CGCA/CGCA, CGCA/CGTA, CGTA/CGTC, CGTC/CGTC, CGTC/CGCA, CGTC/CGTA, CGTA/CATA, CATA/CATA, CATA/TGTA, CATA/CGTA, CATA/CGCA, or CATA/CGTC.

For example, a specific binding pair member of the invention can be an oligonucleotide or an antibody that, under the appropriate conditions, selectively binds to a target polynucleotide at or near nucleotide 1274 of SEQ ID NO: 1 {CYP2D6E7_{—}339}, nucleotide 1757 of SEQ ID NO:2 {HMGCRE7E11-3_{—}472}, nucleotide 1430 of SEQ ID NO:3 {HMGCRDBSNP_{—}45320}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283}, and nucleotide 1421 of SEQ ID NO:12 {HMGCRE16E18_{—}99}. As such, a specific binding pair member of the invention can be an oligonucleotide probe, which can selectively hybridize to a target polynucleotide and can, but need not, be a substrate for a primer extension reaction, or an anti-nucleic acid antibody. The specific binding pair member can be selected such that it selectively binds to any portion of a target polynucleotide, as desired, for example, to a portion of a target polynucleotide containing a SNP as the terminal nucleotide.

The methods of the invention that include identifying a nucleotide occurrence in the sample for at least one statin response-related SNP, in preferred embodiments can include grouping the nucleotide occurrences of the statin response-related SNPs into one or more identified haplotype alleles of a statin response-related haplotypes. To infer the statin response of the subject, the identified haplotype alleles are then compared to known haplotype alleles of the statin response-related haplotype, wherein the relationship of the known haplotype alleles to the statin response is known.

The statin response-related haplotype allele identified in the methods of the present invention also can include at least one CYP3A4A haplotype allele and/or at least one HMGCRA haplotype allele; and can include a diploid pair of CYP3A4A haplotype alleles; a diploid pair of HMGCRA haplotype alleles; or a diploid pair of CYP3A4A haplotype alleles and a diploid pair of HMGCRA haplotype alleles.

A diploid pair of CYP3A4A haplotype alleles that allows an inference as to whether a subject will have a positive (i.e. favorable, decreased serum cholesterol levels) statin response can be, for example, GC/GC; and such a diploid pair of HMGCRA haplotype alleles is exemplified by TG/TG. For example, the human subject can have the diploid pair of CYP3A4A haplotype alleles, GC/GC, and the diploid pair of HMGCRA haplotype alleles, TG/TG. Subjects with diploid pairs GC/GC at the CCP3A4A haplotype and diploid alleles TG/TG at the HMGCRA haplotype have a high likelihood of positively responding to statin treatment, as illustrated in Example 5. In fact, as discussed in Example 5, only 4 of 73 subjects that have this diploid pair of haplotypes, do not respond to either Atorvastatin or Simvastatin. As another example, the diploid pair of CYP3A4A haplotypes and/or HMGCR haplotype alleles can be a diploid pair of major haplotype alleles (e.g. GC/GC at CYP3A4A and TG/TG at HMGCRA) or a diploid pair of minor haplotype alleles. Minor haplotype alleles of CYP3A4A and HMGCRA are disclosed in Example 5, and set out below in Table 5.

TABLE 5
Minor/Major nucleotide occurrences and haplotype alleles

| | | Allele/Nuc. |
|---|---|---|
| Haplotype | SNP | Occur. |
| CYP3A4A | | TG, cG, Ta, |
| | | ca |
| | nucleotide 808 of SEQ ID NO: 8 | T, c |
| | {CYP3A4E10-5_292} | |
| | nucleotide 227 of SEQ ID NO: 9 | G, a |
| | {CYP3A4E12_76} | |
| CYP3A4B | | TGC, TaC, gat, |
| | | gaC, Tat, TGt, |
| | | gaC |
| | nucleotide 1311 of SEQ ID NO: 7 | T, g |
| | {CYP3A4E7_243} | |
| | nucleotide 808 of SEQ ID NO: 8 | G, a |
| | {CYP3A4E10-5_292} | |
| | nucleotide 227 of SEQ ID NO: 9 | C, t |
| | {CYP3A4E12_76} | |
| CYP3A4C | | ATGC, ATaC, |
| | | Agat, AgaC, |
| | | ATat, ATGt, |
| | | tgaC |
| | nucleotide 425 of SEQ ID NO: 10 | A, t |
| | {CYP3A4E3-5_249} | |
| | nucleotide 1311 of SEQ ID NO: 7 | T, g |
| | {CYP3A4E7_243} | |
| | nucleotide 808 of SEQ ID NO: 8 | G, a |
| | {CYP3A4E10-5_292} | |
| | nucleotide 227 of SEQ ID NO: 9 | C, t |
| | {CYP3A4E12_76} | |
| HMGCRA | | GT, aT, Gc, |
| | | ac |
| | nucleotide 1757 of SEQ ID NO: 2 | G, a |
| | {HMGCRE7E11-3_472} | |
| | nucleotide 1430 of SEQ ID NO: 3 | T, c |
| | {HMGCRDBSNP_45320} | |
| HMGCRB | | CGTA, tGTA, |
| | | CGcA, CGTc, |
| | | CaTA |
| | nucleotide 519 of SEQ ID NO: 11 | C, t |
| | {HMGCRE5E6-3_283} | |
| | nucleotide 1757 of SEQ ID NO: 2 | G, a |
| | {HMGCRE7E11-3_472} | |
| | nucleotide 1430 of SEQ ID NO: 3 | T, c |
| | {HMGCRDBSNP_45320} | |
| | nucleotide 1421 of SEQ ID NO: 12 | A, c |
| | {HMGCRE16E18_99} | |
| CYP2D6A | | CTA, tTc, |
| | | tTA, CTc, |
| | | CcA |
| | nucleotide 1159 of SEQ ID NO: 4 | C, t |
| | {CYP2D6PE1_2} | |
| | nucleotide 1093 of SEQ ID NO: 5 | T, c |
| | {CYP2D6PE7_150} | |
| | nucleotide 1223 of SEQ ID NO: 6 | A, c |
| | {CYP2D6PE7_286} | |

Table 5. Capital letters indicate a major nucleotide occurrence; Small letters indicate minor nucleotide occurrence. Haplotype alleles with one or more small letters (minor nucleotide occurrences) are minor haplotypes. Haplotypes with all capital letters are major haplotypes.

In another aspect the present invention provides a method for inferring a statin response of a human subject from a nucleic acid sample of the subject, wherein the method comprising identifying a diploid pair of CYP3A4C alleles and a diploid pair of HMGCRB alleles. In a preferred embodiment, the diploid pair of CYP3A4C alleles include a diploid pair of major alleles (ATGC/ATGC), a diploid pair of alleles that include a minor allele, or ATGC/ATAC, ATGC/AGAC, ATGC/AGAT, ATGC/ATAT, ATGC/TGAC, or ATGT/AGAT. In a preferred embodiment, the diploid pair of HMGCR alleles include a diploid pair of major alleles (CGTA/CGTA), a diploid pair of alleles that include a minor allele, or CGTA/TGTA, CGTA/CGCA, CGTA/CGTC, CGTA/CATA.

As disclosed herein, major haplotype alleles, especially homozygous major haplotype alleles, and nucleotide occurrences for HMGCR and CYP3A4 are generally associated with an efficacious response to statins. As disclosed herein, major haplotype alleles, especially homozygous major haplotype alleles, and nucleotide occurrences for CYP2D6 are generally associated with no adverse reactions to statins.

A method of inferring a positive statin response also can include identifying at least one CYP3A4B haplotype allele and/or at least one HMGCRA haplotype allele, including, for example, a diploid pair of CYP3A4B haplotype alleles; a diploid pair of HMGCRA haplotype alleles; or a diploid pair of CYP3A4B haplotype alleles and a diploid pair of HMGCRA haplotype alleles. Such a diploid pair of CYP3A4B haplotype alleles is exemplified by TGC/TGC, and such a diploid pair of HMGCRA haplotype alleles is exemplified by TG/TG. As such, a subject can have, for example, the diploid pair of CYP3A4B haplotype alleles, TGC/TGC, and the diploid pair of HMGCRA haplotype alleles, TG/TG. Subjects with diploid pairs TGC/TGC at the CYP3A4B haplotype and a diploid pair of TG/TG alleles at the HMGCRA haplotype have a high likelihood of positively responding to statin treatment, as illustrated in Example 5. The diploid pair of CYP3A4B haplotype alleles or HMGCRA haplotype alleles can be a diploid pair of major haplotype alleles (e.g. TGC/TGC at CYP3A4B and TG/TG at HMGCRA) or a diploid pair of minor haplotype alleles.

The methods and compositions of the invention have numerous utilities, the most obvious of which is that they can be used to determine whether to prescribe statins to a patient with elevated serum cholesterol levels.

A sample useful for practicing a method of the invention can be any biological sample of a subject that contains nucleic acid molecules, including portions of the gene sequences to be examined, or corresponding encoded polypeptides, depending on the particular method. As such, the sample can be a cell, tissue or organ sample, or can be a sample of a biological fluid such as semen, saliva, blood, and the like. A nucleic acid sample useful for practicing a method of the invention will depend, in part, on whether the SNPs of the haplotype to be identified are in coding regions or in non-coding regions. Thus, where at least one of the SNPs to be identified is in a non-coding region, the nucleic acid sample generally is a deoxyribonucleic acid (DNA) sample, particularly genomic DNA or an amplification product thereof. However, where heteronuclear ribonucleic acid (RNA), which includes unspliced mRNA precursor RNA molecules, is available, a cDNA or amplification product thereof can be used. Where the each of the SNPs of the haplotype is present in a coding region of a gene(s), the nucleic acid sample can be DNA or RNA, or products derived therefrom, for example, amplification products. Furthermore, while the methods of the invention generally are exemplified with respect to a nucleic acid sample, it will be recognized that particular haplotype alleles can be in coding regions of a gene and can result in polypeptides containing different amino acids at the positions corresponding to the SNPs due to non-degenerate codon changes. As such, in another aspect, the methods of the invention can be practiced using a sample containing polypeptides of the subject.

It will be recognized by one skilled in the art that the invention includes methods of the present invention can identify alleles for any 1 of the statin response-related haplotypes disclosed herein, alone, or any combination of 2, 3, 4, or more, statin response-related haplotypes. In a preferred example with relatively high inference power, the method of the invention, includes identifying haplotype alleles for both CYP3A4C and HMGCRB wherein.

Numerous methods for identifying haplotype alleles in nucleic acid samples (also referred to a surveying the genome) are disclosed herein or othervise known in the art. As disclosed herein, nucleic acid occurrences for the individual SNPs that make up the haplotype alleles are determined, then, the nucleic acid occurrence data for the individual SNPs is combined to identify the haplotype alleles. For example, for the HMGCRA haplotype, both nucleotide occurrences at each SNP loci corresponding to markers HMGCRE7E11_{—}472 and HMGCRDBSNP_{—}45320 can be combined to determine the diploid pair of HMGCRA haplotype alleles of a subject. The Stephens and Donnelly algorithm (Am. J. Hum. Genet. 68:978-989, 2001, which is incorporated herein by reference) can be applied to the data generated regarding individual nucleotide occurrences in SNP markers of the subject, in order to determine the alleles for each haplotype in the subject's genotype. Other methods that can be used to determine alleles for each haplotype in the subject's genotype, for example Clarks algorithm, and an EM algorithm described by Raymond and Rousset (Raymond et al. 1994. GenePop. Ver 3.0. Institut des Siences de l'Evolution. Universite de Montpellier, France. 1994)

The attached sequence listing provides flanking nucleotide sequences for the SNPs disclosed herein. These flanking sequence serve to aid in the identification of the precise location of the SNPs in the human genome, and serve as target gene segments useful for performing methods of the invention. A target polynucleotide typically includes a SNP locus and a segment of a corresponding gene that flanks the SNP. Primers and probes that selectively hybridize at or near the target polynucleotide sequence, as well as specific binding pair members that can specifically bind at or near the target polynucleotide sequence, can be designed based on the disclosed gene sequences and information provided herein.

Latent statin response-related haplotype alleles are haplotype alleles that, in the context of one or more penetrant haplotypes, strengthen the inference of a statin response. Latent statin response-related haplotype alleles are typically alleles whose association with a statin response is not strong enough to be detected with simple genetics approaches. Latent statin response-related SNPs are individual SNPs that make up latent statin response-related haplotypes. It is possible that some of the SNPs which forms statin response-related haplotypes disclosed herein, are latent statin response-related SNPs.

The subject for the methods of the present invention can be a subject of any race. As such, the subject can be of any group of people classified together on the basis of common history, nationality, or geographic distribution. For example, the subject can be of African, Asian, Australia, European, North American, and South American descent. In certain embodiments the subject is Asian, Hispanic, African, or Caucasian. In one embodiment the subject is Caucasian.

As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in identifying a nucleotide occurrence of a SNP. It will be recognized that some amount of non-specific hybridization is unavoidable, but is acceptable provide that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989)).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42EC (moderate stringency conditions); and 0.1×SSC at about 68EC (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

The term “polynucleotide” is used broadly herein to mean a sequence of deoxyribonucleotides or ribonucleotides that are linked together by a phosphodiester bond. For convenience, the term “oligonucleotide” is used herein to refer to a polynucleotide that is used as a primer or a probe. Generally, an oligonucleotide useful as a probe or primer that selectively hybridizes to a selected nucleotide sequence is at least about 15 nucleotides in length, usually at least about 18 nucleotides, and particularly about 21 nucleotides or more in length.

A polynucleotide can be RNA or can be DNA, which can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (e.g., a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acicds Res. 22:5220-5234 (1994); Jellinek et al., Biochemistry 34:11363-11372 (1995); Pagratis et al., Nature Biotechnol. 15:68-73 (1997), each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986 (1994); Ecker and Crooke, BioTechnology 13:351360 (1995), each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide or oligonucleotide comprising nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995). Thus, the term polynucleotide as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR).

In various embodiments, it can be useful to detectably label a polynucleotide or oligonucleotide. Detectable labeling of a polynucleotide or oligonucleotide is well known in the art. Particular non-limiting examples of detectable labels include chemiluminescent labels, radiolabels, enzymes, haptens, or even unique oligonucleotide sequences.

A method of the identifying a SNP also can be performed using a specific binding pair member. As used herein, the term “specific binding pair member” refers to a molecule that specifically binds or selectively hybridizes to another member of a specific binding pair. Specific binding pair member include, for example, probes, primers, polynucleotides, antibodies, etc. For example, a specific binding pair member includes a primer or a probe that selectively hybridizes to a target polynucleotide that includes a SNP loci, or that hybridizes to an amplification product generated using the target polynucleotide as a template.

As used herein, the term “specific interaction,” or “specifically binds” or the like means that two molecules form a complex that is relatively stable under physiologic conditions. The term is used herein in reference to various interactions, including, for example, the interaction of an antibody that binds a polynucleotide that includes a SNP site; or the interaction of an antibody that binds a polypeptide that includes an amino acid that is encoded by a codon that includes a SNP site. According to methods of the invention, an antibody can selectively bind to a polypeptide that includes a particular amino acid encoded by a codon that includes a SNP site. Alternatively, an antibody may preferentially bind a particular modified nucleotide that is incorporated into a SNP site for only certain nucleotide occurrences at the SNP site, for example using a primer extension assay.

A specific interaction can be characterized by a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or greater. A specific interaction generally is stable under physiological conditions, including, for example, conditions that occur in a living individual such as a human or other vertebrate or invertebrate, as well as conditions that occur in a cell culture such as used for maintaining mammalian cells or cells from another vertebrate organism or an invertebrate organism. Methods for determining whether two molecules interact specifically are well known and include, for example, equilibrium dialysis, surface plasmon resonance, and the like.

Numerous methods are known in the art for determining the nucleotide occurrence for a particular SNP in a sample. Such methods can utilize one or more oligonucleotide probes or primers, including, for example, an amplification primer pair, that selectively hybridize to a target polynucleotide, which contains one or more statin response-related SNP positions. Oligonucleotide probes useful in practicing a method of the invention can include, for example, an oligonucleotide that is complementary to and spans a portion of the target polynucleotide, including the position of the SNP, wherein the presence of a specific nucleotide at the position (i.e., the SNP) is detected by the presence or absence of selective hybridization of the probe. Such a method can further include contacting the target polynucleotide and hybridized oligonucleotide with an endonuclease, and detecting the presence or absence of a cleavage product of the probe, depending on whether the nucleotide occurrence at the SNP site is complementary to the corresponding nucleotide of the probe.

An oligonucleotide ligation assay also can be used to identify a nucleotide occurrence at a polymorphic position, wherein a pair of probes that selectively hybridize upstream and adjacent to and downstream and adjacent to the site of the SNP, and wherein one of the probes includes a terminal nucleotide complementary to a nucleotide occurrence of the SNP. Where the terminal nucleotide of the probe is complementary to the nucleotide occurrence, selective hybridization includes the terminal nucleotide such that, in the presence of a ligase, the upstream and downstream oligonucleotides are ligated. As such, the presence or absence of a ligation product is indicative of the nucleotide occurrence at the SNP site.

An oligonucleotide also can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP site can be useful, wherein the amplification product is examined to determine the nucleotide occurrence at the SNP site. Particularly useful methods include those that are readily adaptable to a high throughput format, to a multiplex format, or to both. The primer extension or amplification product can be detected directly or indirectly and/or can be sequenced using various methods known in the art. Amplification products which span a SNP loci can be sequenced using traditional sequence methodologies (e.g., the “dideoxy-mediated chain termination method,” also known as the “Sanger Method”(Sanger, F., et al., J. Molec. Biol. 94:441 (1975); Prober et al. Science 238:336-340 (1987)) and the “chemical degradation method,” “also known as the “Maxam-Gilbert method”(Maxam, A. M., et al., Proc. Natl. Acad. Sci. (U.S.A.) 74:560 (1977)), both references herein incorporated by reference) to determine the nucleotide occurrence at the SNP loci.

Methods of the invention can identify nucleotide occurrences at SNPs using a “microsequencing” method. Microsequencing methods determine the identity of only a single nucleotide at a “predetermined” site. Such methods have particular utility in determining the presence and identity of polymorphisms in a target polynucleotide. Such microsequencing methods, as well as other methods for determining the nucleotide occurrence at a SNP loci are discussed in Boyce-Jacino, et al., U.S. Pat. No. 6,294,336, incorporated herein by reference, and summarized herein.

Microsequencing methods include the Genetic Bit Analysis method disclosed by Goelet, P. et al. (WO 92/15712, herein incorporated by reference). Additional, primer-guided, nucleotide incorporation procedures for assaying polymorphic sites in DNA have also been described (Komher, J. S. et al, Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al, Hum. Mutat. 1:159-164 (1992); Ugozzoli, L. et al., GATA 9:107-112 (1992); Nyren, P. et al., Anal. Biochem. 208:171-175 (1993); and Wallace, WO89/10414). These methods differ from Genetic Bit™. Analysis in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al. Amer. J. Hum. Genet. 52:46-59 (1993)).

Alternative microsequencing methods have been provided by Mundy, C. R. (U.S. Pat. No. 4,656,127) and Cohen, D. et al (French Patent 2,650,840; PCT Appln. No. WO91/02087) which discusses a solution-based method for determining the identity of the nucleotide of a polymorphic site. As in the Mundy method of U.S. Pat. No. 4,656,127, a primer is employed that is complementary to allelic sequences immediately 3′-to a polymorphic site.

In response to the difficulties encountered in employing gel electrophoresis to analyze sequences, alternative methods for microsequencing have been developed. Macevicz (U.S. Pat. No. 5,002,867), for example, describes a method for determining nucleic acid sequence via hybridization with multiple mixtures of oligonucleotide probes. In accordance with such method, the sequence of a target polynucleotide is determined by permitting the target to sequentially hybridize with sets of probes having an invariant nucleotide at one position, and a variant nucleotides at other positions. The Macevicz method determines the nucleotide sequence of the target by hybridizing the target with a set of probes, and then determining the number of sites that at least one member of the set is capable of hybridizing to the target (i.e., the number of “matches”). This procedure is repeated until each member of a sets of probes has been tested.

Boyce-Jacino, et al., U.S. Pat. No. 6,294,336 provides a solid phase sequencing method for determining the sequence of nucleic acid molecules (either DNA or RNA) by utilizing a primer that selectively binds a polynucleotide target at a site wherein the SNP is the most 3′ nucleotide selectively bound to the target.

In one particular commercial example of a method that can be used to identify a nucleotide occurrence of one or more SNPs, the nucleotide occurrences of statin response-related SNPs in a sample can be determined using the SNP-IT™ method (Orchid BioSciences, Inc., Princeton, N.J.). In general, SNP-IT™ is a 3-step primer extension reaction. In the first step a target polynucleotide is isolated from a sample by hybridization to a capture primer, which provides a first level of specificity. In a second step the capture primer is extended from a terminating nucleotide trisphosphate at the target SNP site, which provides a second level of specificity. In a third step, the extended nucleotide trisphosphate can be detected using a variety of known formats, including: direct fluorescence, indirect fluorescence, an indirect colorimetric assay, mass spectrometry, fluorescence polarization, etc. Reactions can be processed in 384 well format in an automated format using a SNPstream™ instrument ((Orchid BioSciences, Inc., Princeton, N.J.).

In another embodiment, a method of the present invention can be performed by amplifying a polynucleotide region that includes a statin response-related SNP, capturing the amplified product in an allele specific manner in individual wells of a microtiter plate, detecting the captured target allele.

In a specific non-limiting example of a method for identifying marker HMGCRE7E11-3_{—}472, of the HMGCRAA haplotype, a primer pair is synthesized that comprises a forward primer that hybridizes to a sequence 5′ to the SNP of SEQ ID NO:2 (the SEQ ID corresponding to this marker (see Table 1)) and a reverse primer that hybridizes to the opposite strand of a sequence 3′ to the SNP of SEQ ID NO:2. This primer pair is used to amplify a target polynucleotide that includes marker HMGCRE7E11-3_{—}472, to generate an amplification product. A third primer can then be used as a substrate for a primer extension reaction. The third primer can bind to the amplification product such that the 3′ nucleotide of the third primer (e.g., adenosine) binds to the marker HMGCRE7E11-3_{—}472 site and is used for a primer extension reaction. The primer can be designed and conditions determined such that the primer extension reaction proceeds only if the 3′ nucleotide of the third primer is complementary to the nucleotide occurrence at the SNP. For example, the third primer can be designed such that the primer extension reaction will proceed if the nucleotide occurrence of marker HMGCRE7E11-3_{—}472 is a guanidine, for example, but not if the nucleotide occurrence of the marker is adenosine.

Phase known data can be generated by inputting phase unknown raw data from the SNPstream™ instrument into the Stephens and Donnelly's PHASE program.

Accordingly, using the methods described above, the statin response-related haplotype allele or the nucleotide occurrence of the statin response-related SNP can be identified using an amplification reaction, a primer extension reaction, or an immunoassay. The statin response-related haplotype allele or the statin response-related SNP can also be identified by contacting polynucleotides in the sample or polynucleotides derived from the sample, with a specific binding pair member that selectively hybridizes to a polynucleotide region comprising the statin response-related SNP, under conditions wherein the binding pair member specifically binds at or near the statin response-related SNP. The specific binding pair member can be an antibody or a polynucleotide.

Antibodies that are used in the methods of the invention include antibodies that specifically bind polynucleotides that encompass a statin response-related or race-related haplotype. In addition, antibodies of the invention bind polypeptides that include an amino acid encoded by a codon that includes a SNP. These antibodies bind to a polypeptide that includes an amino acid that is encoded in part by the SNP. The antibodies specifically bind a polypeptide that includes a first amino acid encoded by a codon that includes the SNP loci, but do not bind, or bind more weakly to a polypeptide that includes a second amino acid encoded by a codon that includes a different nucleotide occurrence at the SNP.

Antibodies are well-known in the art and discussed, for example, in U.S. Pat. No. 6,391,589. Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Antibodies of the invention include antibody fragments that include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. The antibodies of the invention may be monospecific, bispecific, trispecific or of greater multispecificity.

The antibodies of the invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example; in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Where the particular nucleotide occurrence of a SNP, or nucleotide occurrences of a statin response-related haplotype, is such that the nucleotide occurrence results in an amino acid change in an encoded polypeptide, the nucleotide occurrence can be identified indirectly by detecting the particular amino acid in the polypeptide. The method for determining the amino acid will depend, for example, on the structure of the polypeptide or on the position of the amino acid in the polypeptide.

Where the polypeptide contains only a single occurrence of an amino acid encoded by the particular SNP, the polypeptide can be examined for the presence or absence of the amino acid. For example, where the amino acid is at or near the amino terminus or the carboxy terminus of the polypeptide, simple sequencing of the terminal amino acids can be performed. Alternatively, the polypeptide can be treated with one or more enzymes and a peptide fragment containing the amino acid position of interest can be examined, for example, by sequencing the peptide, or by detecting a particular migration of the peptide following electrophoresis. Where the particular amino acid comprises an epitope of the polypeptide, the specific binding, or absence thereof, of an antibody specific for the epitope can be detected. Other methods for detecting a particular amino acid in a polypeptide or peptide fragment thereof are well known and can be selected based, for example, on convenience or availability of equipment such as a mass spectrometer, capillary electrophoresis system, magnetic resonance imaging equipment, and the like.

In another embodiment, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the SNPs listed in Table 9-1, Table 9-2, Table 9-3, Table 9-4, Table 9-5, Table 9-6, Table 9-7, Table 9-8, Table 9-9, Table 9-10, Table 9-11, and 9-12. These SNPs are found in SEQ ID NOS:43-234. The nucleotide occurrence is associated with a statin response, thereby proving an inference of the statin response of the subject.

For example, in one aspect the nucleotide occurrence, also referred to as allele herein, is in SNP 756 listed in Table 9-1. From Table 9-14 it is seen that this SNP corresponds to SEQ ID NO:43. The position of the SNP within this sequence, nucleotide 398, is given in the sequence listing (See marker 756 identified within the sequence listing), and can be visualized in FIG. 3, in the section related to marker 756. This SNP can include an A or a T at position 398. Therefore, for this aspect of the invention, the method can identify a nucleotide occurrence at position 398 of SEQ ID NO:43. Likewise, it will be recognized that from the Tables provided herein in Example 14, as well as the sequence listing, the SEQ ID NO: and position within that SEQ ID NO: of all of the SNPs of the present invention can be determined.

In another embodiment, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-1 and Table 9-2, whereby the nucleotide occurrence is associated with a decrease in low density lipoprotein in response to administration of Atorvastatin, thereby inferring the statin response of the subject. The method can be performed wherein the SNP occurs in one of the genes listed in Table 9-1 and Table 9-2 that includes at least two statin response-related SNPs.

Example 19 discloses numerous genes that include SNPs whose nucleotide occurrence is related to a statin response. It will be understood that using the methods disclosed herein, other SNPs related to a statin response could be identified in these genes. The tables and text of Example 9 discloses genes from which statin response-related SNPs were identified.

The genes in which the SNPs of SEQ ID NOS:43-234 are located can be determined using the sequences provided herein. The gene name is provided in the sequence listing, or can be determined by the first portion of the marker name in the sequence listing, and in Table 9-14. Furthermore, by using these sequences in a search, such as a BLAST search, of human genome sequences, the location of the sequences provided within the human genome can be determined. Therefore, it will be recognized that the genes wherein the SNPs of the present invention occur, can be readily identified.

In another embodiment, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) listed in Table 9-1 and Table 9-2, whereby the nucleotide occurrence is associated with a decrease in low density lipoprotein in response to administration of Atorvastatin. Thereby, identification of the nucleotide occurrence of the SNP provides an inference of the statin response of the subject. In one example, the subject is Caucasian and the statin response-related SNP is at least one SNP listed in Table 9-2.

In another aspect the present invention provides, a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-3 and Table 9-4, whereby the nucleotide occurrence is associated with a decrease in total cholesterol in response to administration of Atorvastatin. Thereby, identification of the nucleotide occurrence of the SNP provides an inference of the statin response of the subject. In one aspect, the SNP occurs in one of the genes listed in Table 9-3 and Table 9-4 comprising at least two statin response-related SNPs.

In another aspect the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) listed in Table 9-3 and Table 9-4, whereby the nucleotide occurrence is associated with a decrease in total cholesterol in response to administration of Atorvastatin. Thereby, identification of the nucleotide occurrence of the SNP provides an inference of the statin response of the subject. In one aspect, the subject is Caucasian and the statin response-related SNP is at least one SNP listed in Table 9-4.

In another embodiment, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-9 and Table 9-10, whereby the nucleotide occurrence is associated with a decrease in low density lipoprotein in response to administration of Simvastatin. Thereby, identification of the nucleotide occurrence of the SNP provides an inference of the statin response of the subject. In one aspect, the SNP occurs in one of the genes listed in Table 9-9 and Table 9-10 comprising at least two statin response-related SNPs.

In another aspect, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) listed in Table 9-9 and Table 9-10, whereby the nucleotide occurrence is associated with a decrease in low density lipoprotein in response to administration of Simvastatin. Thereby, identification of the nucleotide occurrence of the SNP provides an inference of the statin response of the subject. In one aspect, the subject is Caucasian and the statin response-related SNP is at least one SNP listed in Table 9-10.

In another embodiment, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-11 and Table 9-12, whereby the nucleotide occurrence is associated with a decrease in total cholesterol in response to administration of Simvastatin Thereby, identification of the nucleotide occurrence of the SNP provides an inference of the statin response of the subject. In one aspect, the SNP occurs in one of the genes listed in Table 9-11 and Table 9-12 comprising at least two statin response-related SNPs.

In another aspect, the present invention provides a method for inferring a statin response of a human subject from a nucleic acid sample of the subject, the method includes identifying, in the nucleic acid sample, a nucleotide occurrence of at least one statin response-related single nucleotide polymorphism (SNP) listed in Table 9-11 and Table 9-12, whereby the nucleotide occurrence is associated with a decrease in total cholesterol in response to administration of Simvastatin. Thereby, identification of the nucleotide occurrence of the SNP provides an inference of the statin response of the subject. In one aspect, the subject is Caucasian and the statin response-related SNP is at least one SNP listed in Table 9-12.

In another aspect, the present invention provides methods for inferring a statin response, wherein the statin response is an adverse reaction, for example, hepatocellular stress that can include liver damage. Such a method can be performed, for example, by identifying, in a nucleic acid sample from a subject, a haplotype allele of a cytochrome p450 2D6 (CYP2D6) gene corresponding to a CYP2D6A haplotype, which includes nucleotide 1159 of SEQ ID NO:4 {CYP2D6PE1_{—}2}, nucleotide 1093 of SEQ ID NO:5 {CYP2D6PE7_{—}150}, and nucleotide 1223 of SEQ ID NO:6 {CYP2D6PE7_{—}286}. The presence of such a haplotype, particularly where the haplotype allele is other than CTA, is associated with an increase in serum glutamic oxaloacetate (SGOT), which is indicative of hepatocellular stress and possibly liver damage. CTA is a major allele of this haplotype. Other alleles that are identified herein include TTC, TTA, CTC, and CCA. The method can include identifying a diploid pair of CYP2D6A haplotype alleles.

A method for inferring a negative (or adverse) statin response also can be performed by identifying, in a nucleic acid sample from a subject, a diploid pair of nucleotides of the CYP2D6 gene, at a position corresponding to nucleotide 1274 of SEQ ID NO:1 {CYP2D6E7_{—}339}, whereby a diploid pair of nucleotides, particularly a diploid pair other than C/C, is indicative of an adverse hepatocellular response. For example, the diploid pair of nucleotides can be C/A, which is indicative of an adverse hepatocellular effect.

The human subject for certain embodiments of the present invention is Caucasian. The statin in certain embodiments of this aspect of the invention is Atorvastatin.

In another aspect, the method allows an inference to be drawn as to whether the subject will have an adverse statin response by identifying, in a nucleic acid sample from the subject, a nucleotide occurrence of at least one statin response-related SNP corresponding to nucleotide 1274 of SEQ ID NO:1 {CYP2D6E7_{—}339}, nucleotide 1159 of SEQ ID NO:4 {CYP2D6PE1_{—}2}, nucleotide 1093 of SEQ ID NO:5 {CYP2D6PE7_{—}150}, or nucleotide 1223 of SEQ ID NO:6 {CYP2D6PE7_{—}286}.

The method can include identifying a nucleotide occurrence of each of at least two (e.g., 2, 3, 4, 5, 6, or more) statin response-related SNPs, which can, but need not comprise one or more haplotype alleles, and can, but need not be in one gene. The nucleotide occurrence of the at least one statin response-related SNP can be a minor nucleotide occurrence, i.e., a nucleotide present in a relatively smaller percent of a population including the subject, or can be a major nucleotide occurrence. Minor nucleotide occurrences are generally associated with a higher probability of an adverse response, as illustrated in Example 4. Where a haplotype allele is determined, the haplotype allele can be a major haplotype allele, or a minor haplotype allele. The presence of a major haplotype allele, which in Caucasian populations appears to be CTA, is associated with a lower chance of an adverse response, as illustrated in Example 4.

A variety of commonly prescribed medications cause what are commonly considered to be “benign” side effects. Though surrogate markers of adverse response for many FDA approved drugs usually self resolve and are thought to be of little consequence for long term health, there may be more sinister relationships between aberrant surrogate marker test results and long term health than originally thought (Baker et al., 2001; Amacher et al., 2001).

About 3% of patients who take Statins develop symptoms of hepatocellular (liver) injury. A greater percent of patients exhibit myalgia or muscle pain. Prolonged use in those individuals that exhibit adverse response to Statins can, and does lead to permanent disease. For example, clinical trials showed that about 1% of Baycol patients (similar to other Statins), experienced muscle discomfort and/or creatine kinase elevations in response to treatment. Nonetheless, it took several years of post-trial drug use to illustrate that the relatively high frequency of minor complaints and surrogate marker abnormalities were part of a continuum of clinical pathology that extends, in its extreme, to myonecrosis and even death.

The incidence of Statin induced hepatocellular stress may likewise portend a serious health risks in the Statin patient population (Rienus, 2000). Though Statin induced hepatic stress usually resolves on its own, in some patients it worsens to hepatic injury indicated by decreases in liver weight, jaundice, hepatitis or even death.

An “adverse statin response” is any negative response to statins, most particularly hepatic stress, possibly accompanied by liver damage. A negative hepatocellular response according to the present invention is inferred by identifying nucleotide occurrences, and optionally haplotypes, of the CYP2D6 gene.

Approximately 0.7% of patients taking Atorvastatin exhibit persistent and dose-dependent indications of hepatic stress, the most commonly observed being an elevation in serum transaminase (SGOT, ALTGPT) levels. These and other indications of hepatic stress are indicators of an adverse statin response according to this aspect of the invention. Because drug induced hepatocellular damage is preceded by elevations in liver function tests, physicians routinely perform these tests prior to, at 12 weeks and periodically following the initiation of (or increase in dosage of) Statins and discontinue treatment if the elevations persist. Though clinical trials have shown that only a minor proportion of patients exhibit what are considered “dangerous” SGOT and GPT elevations (the classification of which is entirely arbitrary), it is common knowledge that a significantly higher proportion of patients (up to 30%, unpublished observations) exhibit more modest, but significant elevations greater than 20% of baseline. Additionally, For the average individual, an increase in the SGOT level to 37 or higher, or an increase in the GPT level above 56 signifies an adverse hepatocellular response. However, these thresholds are relevant to the average human, without regard to their race, sex or age. Creatine kinase is another enzyme whose increased levels are indicative of adverse response to statins. About 20% of patients who take statins complain of muscle ache, and elevated creatine kinase levels are indicative of myalgia (muscle injury).

Because the incidence of aberrant surrogate marker levels in response to drugs like Statins is not small, various laboratories have investigated whether drug pretreatment regimens diminish the severity of adverse hepatocellular injury caused by some drugs by decreasing oxidative stress and lipoperoxidation. The results of these studies indicate that direct measures of hepatocellular health, such as hepatocellular regeneration or DNA fragmentation, are often left unaffected by these pretreatments (Ferrali et al., 1997). The results further suggest that a potential drug-based resolution of Statin induced hepatocellular stress may not always proceed without sequelae, and that genetic tests to match patients with Statins may be more effective-modality of prophylaxis.

Before the present invention, it was not possible to predict which hepatocellular stressed patients will progress along the continuum of hepatocellular pathology, or to define the risks of this progression in terms of the magnitude of surrogate indicator levels. As such, it may be more logical to find ways to avoid the risk altogether by matching patients with drugs based on their genetic constitution. To this end, the present studies were directed to investigating whether common haplotypes in various pharmaco-relevant human genes can be associated with unwanted hepatocellular side affects.

Statin induced hepatocellular toxicity is thought to occur via cytochrome P450-mediated oxidation to pathophysiologically reactive metabolites, which are known to react with hepatic proteins and lipids to form covalent adducts. These adducts can render hepatic cells more susceptible to oxidation damage, which, in turn, can result in further modification of cellular lipids and proteins, DNA degradation, apoptosis and hepatic necrosis (Reid and Bornheim 2001, Boularis et al., 2000; Ulrich et al, 2001; Reid et al., 2001). The wide distribution, interethnic variability and intraethnic frequency of these types of adverse effects within geographical regions suggest that hepatocellular toxicity is a function of aberrant chemical side reactions and individual genetic constitution.

Tests using model systems show striking individual and species variability in hepatic toxicity to the same drug and dose, suggesting that individual or species differences in any step along a particular drug metabolism pathway can result in “idiosyncratic responses (Ulrich et al., 2001). Because variant xenobiotic modifier isoforms have different substrate specificities as compared to the wild-type form (Wennerholm et al., 1998), it is possible that unique haplotype variants of the commonly studied xenobiotic metabolizers (i.e. the phase I and phase II enzymes) explain a large part of the variance in adverse events for a variety of drugs. These genetic differences may, but need not necessarily, be extended to explain other idiosyncratic responses that follow from variations in drug metabolism, including effects on drug efficacy, drug interactions and other collateral effects on mitochondrial function, nutritional status, general health or underlying disease.

Because of the complexities of the major and minor metabolic pathways involved, and the extent of genetic variation at most xenobiotic modifier loci, haplotypes associated with cytochrome P450 mediated side reactions may or may not be deterministically or genetically linked to previously defined aberrant metabolizer alleles (Vandel et al., 1999). Further, the current knowledge base of polymorphisms within the major cytochrome P450s is not yet complete and therefore, there is not yet an understanding of how genetic variation in the cytochrome P450 can explain variable drug metabolism and response. For example, the strength of the concordance between CYP2D6 metabolizer phenotypes and poor metabolizer genotypes depends on the drug and population; debrisoquine metabolism among Tanzanians has been found to be slower than expected from the CYP2D6 genotype (Wennerholm et al., 1998), and patients with an extensive metabolizer (EM) genotype sometimes phenotype as poor metabolizers (PM) in absence of competing drugs in their blood stream (O'Neil et al., 2000). This point is particularly easy to appreciate when it is considered that CYP2D6 (and other CYP) metabolizer genotypes have been documented with respect to a limited set of highly penetrant variants, a limited set of compounds, measured against a limited set of end points (often efficacy) in a limited number of generalized ethnic classes (Kalow, 1992). In particular, little is known about the biochemistry and genetics of minor CYP2D6 metabolic pathways affected by variants because they are often more difficult to measure than major pathways.

For virtually all cytochrome P450s, including CYP2D6, little is known about interactions of alleles between genes (epistasis) or to what extent pharmacogenomic concepts can be integrated with haploid sets of SNPs and environmental components to explain variance in drug response. The expansion of the new field of pharmacogenomics promises to help us more systematically define the role of drug metabolizer variants in drug response. It is hoped that systematic candidate gene approaches (involving multiple genes per project), multiple markers within each gene, and intensely annotated patient databanks can be economically screened to find new and/or complimentary pharmacogenomics marker sets that explain a greater percent of drug reaction trait variability in the population than previously found.

Polymorphisms in the CYP2D6 gene have been previously discovered by others to be deterministic for undesirable reaction to a variety of commonly prescribed medications (Kalow, Pergamon Press, Pharmacogenetics of Drug Metabolism). Catastrophic, Mendelian mutations in this gene have also been associated with various adverse events associated with the use of various drugs. Until the present studies were performed, however, nothing was known about how natural variation in this gene is related to variable efficacy of the Statins, or commonly observed adverse hepatocellular and muscle responses to the statin class of anti-cholesterol drugs.

The human genome project has resulted in the generation of a human polymorphism database containing the location and identity of variants (SNPs) for many of the 30,000 or so human genes (dbSNP). However, only a few SNPs exist in this database for the CYP2D6 gene, and a total of 18 polymorphisms are known from the literature. How, or if, these polymorphisms, or any as of yet undiscovered polymorphisms are related to statin response has heretofore been unknown. Because of our limited understanding of idiosyncratic drug responses, and our limited knowledge of extant genetic variation at most xenobiotic modifier loci, the problem was approached from a fresh viewpoint. As disclosed herein, rather than focus on small numbers of previously described SNPs with known functional relevance, numerous highly detailed SNP and haplotype maps have been built from several hundred multi-ethnic donors.

Due to several factors, the present maps are more detailed than those previously produced (see, for example, Marez et al., 1997). These maps were used to genotype individual patients within a “master” specimen databank, which contains representative and intensely annotated patient specimens for several hundred commonly prescribed, and variably efficacious drugs. The goal of this approach was to haplotype every person at every pharmaco-relevant gene for the systematic and relatively hypothesis-free identification of individual, epistatic and environmental components of variable drug response.

The present effort resulted in the discovery of 50 novel polymorphisms in the CYP2D6 gene. Several of these polymorphisms have been scored, in addition to several of the publicly available SNPs, in individuals of known statin response. Initial results as disclosed herein have identified an SNP in the CYP2D6 gene that is statistically associated adverse hepatocellular response to two commonly prescribed statins (Lipitor™ and Zocor; p=0.01; see Example 3). Furthermore, a haplotype system within the CYP2D6 gene was identified that is predictive of adverse hepatocellular response in Atorvastatin patients (Example 4). The results, which were highly specific to the SGOT response, specifically in Atorvastatin patients, are consistent with an earlier report demonstrating the role of wild-type CYP2D6 in Atorvastatin disposition (Cohen et al., 2000). As such, the present results confirm the earlier report implicating CYP2D6 as a modifier of Atorvastatin, and extend it by implicating minor CYP2D6 haplotypes as contributors towards idiosyncratic Atorvastatin response. The results also demonstrate that the present approach is of sufficient sensitivity and specificity that it can form the basis for a new pharmacogenomics test, which can help prospective Atorvastatin patients avoid undesired hepatocellular responses.

For methods of the present invention which analyze diploid pairs of CYP2D6A haplotypes alleles, the diploid pairs can include one minor and one major haplotype allele or a diploid pair of minor haplotype alleles, or a diploid pair of major haplotype alleles. As illustrated in Example 4, the major allele of CYP2D6, CTA, especially homozygous diploid pairs of the major allele for this haplotype is associated with no adverse reaction in terms of SGOT scores.

The method of the invention that include identifying a nucleotide occurrence in the sample for at least one statin response-related SNP, as discussed above, in preferred embodiments can include grouping the nucleotide occurrences of the statin response-related SNPs into one or more identified haplotype alleles of a statin response-related haplotypes. To infer the statin response of the subject, the identified haplotype alleles are then compared to known haplotype alleles of the statin response-related haplotype, wherein the relationship of the known haplotype alleles to the statin response is known.

In another aspect the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-5 and Table 9-6, whereby the nucleotide occurrence is associated with an increase in SGOT readings in response to administration of Atorvastatin. Thereby, identification of the nucleotide occurrence of the SNP provides an inference of the statin response of the subject. In one aspect, the SNP occurs in one of the genes listed in Table 9-5 and Table 9-6 comprising at least two statin response-related SNPs.

In another aspect, the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) listed in Table 9-5 and Table 9-6, whereby the nucleotide occurrence is associated with an increase in SGOT readings in response to administration of Atorvastatin. Thereby, identification of the nucleotide occurrence of the SNP provides an inference of the statin response of the subject. In one aspect, the subject is Caucasian and the statin response-related SNP is at least one SNP listed in Table 9-6.

In another aspect the present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Table 9-7 and Table 9-8, whereby the nucleotide occurrence is associated with an increase in ALTGPT readings in response to administration of Atorvastatin. Thereby, identification of the nucleotide occurrence of the SNP provides an inference of the statin response of the subject. In one aspect, the SNP occurs in one of the genes listed in Table 9-7 and Table 9-8 comprising at least two statin response-related SNPs.

In another aspect the present invention provides, a method for inferring a statin response of a human subject from a nucleic acid sample of the subject, wherein the method includes identifying, in the nucleic acid sample, a nucleotide occurrence of at least one statin response-related single nucleotide polymorphism (SNP) listed in Table 9-7 and Table 9-8, whereby the nucleotide occurrence is associated with an increase in ALTGPT readings in response to administration of Atorvastatin Thereby, identification of the nucleotide occurrence of the SNP provides an inference of the statin response of the subject. In one aspect, the subject is Caucasian and the statin response-related SNP is at least one SNP listed in Table 9-8.

The present invention also related to an isolated human cell or an isolated plurality of cells, which contain a minor nucleotide occurrence of a statin response-related SNP or a minor haplotype allele. The cells are useful for drug design, for example of new, more effective statins that exhibit fewer side effects. For example, the cells can be used to screen test agents, such as new statins, for efficacy and propensity to elicit an adverse response. Bioassays of test agents using the isolated cells can for example, screen the agent for an effect on activity, such as enzymatic activity, of a CYP3A4, HMGCR, or CYP2D6 protein. Furthermore, efficacy of an on-test agent can be determined by measuring cholesterol uptake and/or metabolism in the isolated cells. In certain preferred embodiments, the cells are cultured hepatocytes.

Methods are known in the art for testing agents such as statins, on isolated cells, including hepatocytes, for inhibition of HMGCR, CYP3A4 and/or CYP2D6 activity (See e.g., Cohen et. al. Biopharm. Drug Dispos. 21:353 (2002)). Isolated cells of the present invention can also be cultured and used to make microsomal preparations for assaying effects of agents such as statins on the activity of HMGCR, CYP3A4, and/or CYP2D6.

As illustrated in the Examples section, present statins such as Lipitor™ and Zocor™ are most effective in subjects that have a diploid pair of major CYP3A4C, CYP3A4B, or CYP3A4A alleles and a diploid pair of major HMGCRB or HMGCRA genotype alleles. Furthermore, present statins such as Lipitor™ are least likely to cause adverse statin responses in subjects with major CYP2D6A haplotype alleles. Therefore, isolated cells that include minor CYP3A4, HMGCR, or CYP2D6 SNP nucleotide occurrences, and minor haplotype alleles, are useful for identifying new statins that are effective against subjects with minor alleles of one or more of these haplotypes, for which present statins are less likely to be effective and more likely to cause an adverse reaction.

Enzyme activity for CYP3A4, HMGCR, and/or CYP2D6 after exposure to a statin, such as Atorvastatin, can be analyzed in isolated cells of the present invention, which have at least one minor nucleotide occurrence in at least one statin response-related SNP, and compared to enzyme activity after exposure to the statin of isolated cells which have a major (i.e. wild type) nucleotide occurrence in the corresponding statin response-related SNP, to identify isolated cells which exhibit a different enzymatic activity after exposure to the statin, than cells with a major nucleotide occurrence. This step can be helpful because the data presented in the Examples indicates that certain subjects with a minor nucleotide occurrence in a statin response-related SNP can exhibit an efficacious statin response and/or no adverse reactions. Therefore, it is likely that cells isolated from these subjects will likewise exhibit a wild type response with respect to CYP3A4, HMGCR, and/or CYP2D6 activity.

A method of identifying an agent can be performed, for example, by contacting an isolated cell of the present invention with at least a test agent to be examined as a potential agent for treating elevated serum cholesterol, and detecting an effect on the activity of CYP3A4, HMGCR, or CYP2D6. In certain embodiments, an effect on the activity of CYP3A4, HMGCR, or CYP2D56 can be determined by comparing the effect on isolated cells of the present invention which include a minor nucleotide occurrence of a statin response-related SNP, to cells which include a major occurrence at the statin response-related SNP.

The term “test agent” is used herein to mean any agent that is being examined for the ability to affect the activity of CYP2D6, CYP3A4, or HMGCR using isolated cells of the present invention. The method generally is used as a screening assay to identify previously unknown molecules that can act as a therapeutic agent for treating elevated cholesterol levels.

A test agent can be any type of molecule, including, for example, a peptide, a peptidomimetic, a polynucleotide, or a small organic molecule, that one wishes to examine for the ability to act as a therapeutic agent, which is a agent that provides a therapeutic advantage to a subject receiving it. It will be recognized that a method of the invention is readily adaptable to a high throughput format and, therefore, the method is convenient for screening a plurality of test agents either serially or in parallel. The plurality of test agents can be, for example, a library of test agents produced by a combinatorial method library of test agents. Methods for preparing a combinatorial library of molecules that can be tested for therapeutic activity are well known in the art and include, for example, methods of making a phage display library of peptides, which can be constrained peptides (see, for example, U.S. Pat. Nos. 5,622,699; 5,206,347; Scott and Smith, Science 249:386-390, 1992; Markland et al., Gene 109:13-19, 1991; each of which is incorporated herein by reference); a peptide library (U.S. Pat. No. 5,264,563, which is incorporated herein by reference); a peptidomimetic library (Blondelle et al., Trends Anal. Chem. 14:83-92, 1995; a nucleic acid library (O'Connell et al., supra, 1996; Tuerk and Gold, supra, 1990; Gold et al., supra, 1995; each of which is incorporated herein by reference); an oligosaccharide library (York et al., Carb. Res., 285:99-128, 1996; Liang et al., Science, 274:1520-1522, 1996; Ding et al., Adv. Expt. Med. Biol., 376:261-269, 1995; each of which is incorporated herein by reference); a lipoprotein library (de Kruif et al., FEBS Lett., 399:232-236, 1996, which is incorporated herein by reference); a glycoprotein or glycolipid library (Karaoglu et al., J. Cell Biol., 130:567-577, 1995, which is incorporated herein by reference); or a chemical library containing, for example, drugs or other pharmaceutical agents (Gordon et al., J. Med. Chem., 37:1385-1401, 1994; Ecker and Crooke, BioTechnology, 13:351-360, 1995; each of which is incorporated herein by reference). Accordingly, the present invention also provides a therapeutic agent identified by such a method, for example, a cancer therapeutic agent.

Assays that utilize these cells to screen test agents are typically performed on isolated cells of the present invention in tissue culture. The isolated cells can be cells from a cell line, passaged primary cells, or primary cells, for example. An isolated cell according to the present invention can be, for example, a hepatocyte, or a hepatocyte cell line.

The present invention also relates to an isolated human cell, which contains, in an endogenous HMGCR gene or in an endogenous CYP gene or in both, a first minor nucleotide occurrence of at least a first statin response related SNP. Accordingly, in one embodiment, the invention provides an isolated human cell, which contains an endogenous HMGCR gene, which includes a first minor nucleotide occurrence of at least a first statin response related SNP. For example, the minor nucleotide occurrence can be at a position corresponding to nucleotide 519 of SEQ ID NO: 11 {HMGCRE5E6-3_{—}283}, nucleotide 1430 of SEQ ID NO:3 {HMGCRDBSNP_{—}45320}, nucleotide 1757 of SEQ ID NO:2 {HMGCRE7E11-3_{—}472}, or nucleotide 1421 of SEQ ID NO:12 {HMGCRE16E18_{—}99}.

The endogenous HMGCR gene in an isolated cell of the invention can further contain a minor nucleotide occurrence of a second statin response related SNP, which, for example, in combination with the first minor nucleotide occurrence of the first statin response related SNP comprises a minor haplotype allele of an HMGCR haplotype, for example, an HMGCRA or HMGCRB haplotype. The endogenous HMGCR gene of the isolated cell also can further contain a major nucleotide occurrence of a second statin response related SNP, which, for example, in combination with the first minor nucleotide occurrence of the first statin response related SNP can comprise a haplotype allele, which can be a minor haplotype allele of an HMGCR haplotype.

The isolated cell of the invention can also further contain a second minor nucleotide occurrence of the first statin response related SNP, thereby providing a diploid pair of minor nucleotide occurrences of the HMGCR gene. In addition, an isolated human cell of the invention can further contain a major nucleotide occurrence of the first statin response related SNP, thereby providing a diploid pair of nucleotide occurrences comprising a major nucleotide occurrence and a minor nucleotide occurrence. An isolated human cell of the invention also can contain an endogenous cytochrome p450 gene having a minor nucleotide occurrence of a statin response related SNP.

In another embodiment, the invention provides an isolated human cell, which contains an endogenous CYP3A4 gene, which includes a thymidine residue at nucleotide 425 of SEQ ID NO:10 {CYP3A4E3-5_{—}249}, or a first minor nucleotide occurrence, of at least a first statin response related SNP. at a position corresponding nucleotide 1311 of SEQ ID NO:7 {CYP3A4E7_{—}243}, nucleotide 808 of SEQ ID NO:8 {CYP3A4E10-5_{—}292}, or nucleotide 227 of SEQ ID NO:9 {CYP3A4E12_{—}76}.

The endogenous CYP3A4 gene in an isolated cell of the invention can further contain a minor nucleotide occurrence of a second statin response related SNP, which, for example, in combination with the first minor nucleotide occurrence of the first statin response related SNP comprises a minor haplotype allele of an CYP3A4 haplotype, for example, a CYP3A4A, CYP3A4B or CYP3A4C haplotype. The endogenous CYP3A4 gene of the isolated cell also can further contain a major nucleotide occurrence of a second statin response related SNP, which, for example, in combination with the first minor nucleotide occurrence of the first statin response related SNP can comprise a haplotype allele, which can be a minor haplotype allele of an CYP3A4 haplotype.

The isolated cell of the invention can also further contain a second minor nucleotide occurrence of the first statin response related SNP, thereby providing a diploid pair of minor nucleotide occurrences of the CYP3A4 gene. In addition, an isolated human cell of the invention can further contain a major nucleotide occurrence of the first statin response related SNP, thereby providing a diploid pair of nucleotide occurrences comprising a major nucleotide occurrence and a minor nucleotide occurrence. An isolated human cell of the invention also can contain an endogenous HMGCR gene having a minor nucleotide occurrence of a statin response related SNP, and also can contain an endogenous CYP2D6 gene having a minor nucleotide occurrence of a statin response-related SNP.

In another embodiment, the invention provides an isolated human cell, which contains an endogenous CYP3A4 gene, which includes a first minor nucleotide occurrence of at least a first statin response related SNP. For example, the minor nucleotide occurrence can be at a position corresponding

nucleotide 425 of SEQ ID NO:10 {CYP3A4E3-5_{—}249},

nucleotide 1311 of SEQ ID NO:7 {CYP3A4E7_{—}243},

nucleotide 808 of SEQ ID NO:8 {CYP3A4E10-5_{—}292}, or

nucleotide 227 of SEQ ID NO:9 {CYP3A4E12_{—}76}.

In another embodiment, the invention provides an isolated human cell, which contains an endogenous CYP2D6 gene, which includes a first minor nucleotide occurrence of at least a first statin response related SNP. For example, the minor nucleotide occurrence can be at a position corresponding

nucleotide 1159 of SEQ ID NO:4 {CYP2D6PE1_{—}2}, a

nucleotide 1093 of SEQ ID NO:5 {CYP2D6PE7_{—}150}, or a

nucleotide 1223 of SEQ ID NO:6 {CYP2D6PE7_{—}286}.

The endogenous CYP2D6 gene in an isolated cell of the invention can further contain a minor nucleotide occurrence of a second statin response related SNP, which, for example, in combination with the first minor nucleotide occurrence of the first statin response related SNP comprises a minor haplotype allele of an CYP2D6 haplotype, for example, a CYP2D6A haplotype. The endogenous CYP2D6 gene of the isolated cell also can further contain a major nucleotide occurrence of a second statin response related SNP, which, for example, in combination with the first minor nucleotide occurrence of the first statin response related SNP can comprise a haplotype allele, which can be a minor haplotype allele of an CYP2D6 haplotype.

The isolated cell of the invention can also further contain a second minor nucleotide occurrence of the first statin response related SNP, thereby providing a diploid pair of minor nucleotide occurrences of the CYP2D6 gene. In addition, an isolated human cell of the invention can further contain a major nucleotide occurrence of the first statin response related SNP, thereby providing a diploid pair of nucleotide occurrences comprising a major nucleotide occurrence and a minor nucleotide occurrence. An isolated human cell of the invention also can contain an endogenous HMGCR gene having a minor nucleotide occurrence of a statin response related SNP, and also can contain an endogenous CYP3A4 gene having a minor nucleotide occurrence of a statin response-related SNP.

In certain preferred embodiments, the isolated cell of the present invention has a minor allele of a HMGCRB haplotype, a minor allele of a CY3A4C haplotype, and/or a minor allele of a CY32D6A haplotype. The specific nucleotide occurrences of such minor alleles are listed herein.

The present invention also relates to a plurality of isolated human cells, which includes at least two (e.g., 2, 3, 4, 5, 6, 7, 8, or more) populations of isolated cells, wherein the isolated cells of one population contain at least one nucleotide occurrence statin response related SNP or at least one statin response related haplotype allele that is different from the isolated cells of at least one other population of cells of the plurality. Accordingly, in one embodiment, the invention provides a plurality of isolated human cells, which includes a first isolated human cell, which comprises an endogenous HMGCR gene comprising a first minor nucleotide occurrence of a first statin response related single nucleotide polymorphism (SNP), and at least a second isolated human cell, which comprises an endogenous HMGCR gene comprising a nucleotide occurrence of the first statin response related SNP different from the minor nucleotide occurrence of the first statin response related SNP of the first cell.

A plurality of isolated human cells of the invention can include, for example, at least a second isolated human cell (generally a population of such cells) that contains a second minor nucleotide occurrence of the first statin response related SNP, wherein the second minor nucleotide occurrence of the first statin response related SNP is different from the first minor nucleotide occurrence of the first statin response related SNP. The endogenous HMGCR gene of the first isolated cell can, but need not, further contain a minor nucleotide occurrence of a second statin response related SNP, which, in combination with the first minor nucleotide occurrence of the first statin response related SNP can, but need not, comprise a minor haplotype allele of an HMGCR haplotype, for example, an HMGCRA haplotype, or can comprise a major haplotype allele of an HMGCRA haplotype.

In another embodiment, the invention provides a plurality of isolated human cells, which includes a first isolated human cell, which comprises an endogenous CYP3A4 gene comprising a first minor nucleotide occurrence of a first statin response related single nucleotide polymorphism (SNP), and at least a second isolated human cell, which comprises an endogenous CYP3A4 gene comprising a nucleotide occurrence of the first statin response related SNP different from the minor nucleotide occurrence of the first statin response related SNP of the first cell.

A plurality of isolated human cells of the invention can include, for example, at least a second isolated human cell (generally a population of such cells) that contains a second minor nucleotide occurrence of the first statin response related SNP, wherein the second minor nucleotide occurrence of the first statin response related SNP is different from the first minor nucleotide occurrence of the first statin response related SNP. The endogenous CYP3A4 gene of the first isolated cell can, but need not, further contain a minor nucleotide occurrence of a second statin response related SNP, which, in combination with the first minor nucleotide occurrence of the first statin response related SNP to form a minor haplotype allele of an CYP3A4A, CYP3A4B, or CYP3A4C haplotype.

In another embodiment, the invention provides a plurality of isolated human cells, which includes a first isolated human cell, which comprises an endogenous CYP2D6 gene comprising a first minor nucleotide occurrence of a first statin response related single nucleotide polymorphism (SNP), and at least a second isolated human cell, which comprises an endogenous CYP2D6 gene comprising a nucleotide occurrence of the first statin response related SNP different from the minor nucleotide occurrence of the first statin response related SNP of the first cell.

A plurality of isolated human cells of the invention can include, for example, at least a second isolated human cell (generally a population of such cells) that contains a second minor nucleotide occurrence of the first statin response related SNP, wherein the second minor nucleotide occurrence of the first statin response related SNP is different from the first minor nucleotide occurrence of the first statin response related SNP. The endogenous CYP2D6 gene of the first isolated cell can, but need not, further contain a minor nucleotide occurrence of a second statin response related SNP, which, in combination with the first minor nucleotide occurrence of the first statin response related SNP to form a minor haplotype allele of an CYP2D6A.

In another embodiment the present invention provides a vector containing one or more of the isolated polynucleotides disclosed herein. Many vectors are known in the art, including expression vectors. In one aspect, the vectors of the present invention include an isolated polynucleotide of the present invention that encodes a polypeptide, operatively linked to an expression control sequence such as a promoter sequence on the vector. Sambrook (1989) for example, provides examples of vectors and methods for manipulating vectors, which are well known in the art.

In another embodiment, the present invention provides an isolated cell containing one or more of the isolated polynucleotides disclosed herein, or one or more of the vectors disclosed in the preceding sentence. As such, the cell is a recombinant cell.

The present invention provides novel CYP3A4, HMGCR, and CYP2D6 alleles, and polynucleotides which include one or more novel SNP nucleotide occurrences of these novel alleles. Accordingly, the present invention further relates to a method for classifying an individual as being a member of a group sharing a common characteristic by identifying a nucleotide occurrence of a SNP in a polynucleotide of the individual, wherein the nucleotide occurrence corresponds, for example, to a thymidine residue of nucleotide 425 of SEQ ID NO: 10 {CYP3A4E3-5_{—}249}, or at least one minor allele of at least one of a

nucleotide 1274 of SEQ ID NO:1 {CYP2D6E7_{—}339},

nucleotide 1757 of SEQ ID NO:2 {HMGCRE7E11-3_{—}472},

nucleotide 1159 of SEQ ID NO:4 {CYP2D6PE1_{—}2},

nucleotide 1093 of SEQ ID NO:5 {CYP2D6PE7_{—}150},

nucleotide 1223 of SEQ ID NO:6 {CYP2D6PE7_{—}286},

nucleotide 1311 of SEQ ID NO:7 {CYP3A4E7_{—}243},

nucleotide 808 of SEQ ID NO:8 {CYP3A4E10-5_{—}292},

nucleotide 227 of SEQ ID NO:9 {CYP3A4E12_{—}76};

nucleotide 519 of SEQ ID NO: 11 {HMGCRE5E6-3_{—}283 }, and

nucleotide 1421 of SEQ ID NO:12 {HMGCRE16E18_{—}99}, or any combination thereof.

Additionally, the present invention further relates to a method for classifying an individual as being a member of a group sharing a common characteristic by identifying a nucleotide occurrence of a SNP in a polynucleotide of the individual, wherein the nucleotide occurrence is a thymidine residue at nucleotide 425 of SEQ ID NO:10 {CYP3A4E3-5_{—}249}, or a minor nucleotide occurrence at a position corresponding to at least one of nucleotide 1274 of SEQ ID NO:1 {CYP2D6E7_{—}339}, nucleotide 1757 of SEQ ID NO:2 {HMGCRE7E1 1-3_{—}472},

nucleotide 1159 of SEQ ID NO:4 {CYP2D6PE1_{—}2},

nucleotide 1093 of SEQ ID NO:5 {CYP2D6PE7_{—}150},

nucleotide 1223 of SEQ ID NO:6 {CYP2D6PE7_{—}286},

nucleotide 1311 of SEQ ID NO:7 {CYP3A4E7_{—}243},

nucleotide 808 of SEQ ID NO:8 {CYP3A4E10-5_{—}292},

nucleotide 227 of SEQ ID NO:9 {CYP3A4E12_{—}76};

nucleotide 519 of SEQ ID NO:11{HMGCRE5E6-3_{—}283}, and

nucleotide 1421 of SEQ ID NO:12 {HMGCRE16E18_{—}99}, or any combination thereof.

In addition, the present invention relates to a method for detecting a nucleotide occurrence for a SNP in a polynucleotide by incubating a sample containing the polynucleotide with a specific binding pair member, wherein the specific binding pair member specifically binds at or near a polynucleotide suspected of being polymorphic, and wherein the polynucleotide includes a thymidine residue at nucleotide 425 of SEQ ID NO:10 {CYP3A4E3-5_{—}249}, or a minor nucleotide occurrence corresponding to at least one of nucleotide 1274 of SEQ ID NO: 1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243 }, nucleotide 808 of SEQ ID NO:8 {CYP3A4E10-5_{—}292}, nucleotide 227 of SEQ ID NO:9 {CYP3A4E12_{—}76}; nucleotide 519 of SEQ ID NO:11 {HMGCRE5E6-3_{—}283}, and nucleotide 1421 of SEQ ID NO:12 {HMGCRE16E18_{—}99}, or any combination thereof; and detecting selective binding of the specific binding pair member, wherein selective binding is indicative of the presence of the nucleotide occurrence. Such methods can be performed, for example, by a primer extension reaction or an amplification reaction such as a polymerase chain reaction, using an oligonucleotide primer that selectively hybridizes upstream, or an amplification primer pair that selectively hybridizes to nucleotide sequences flanking and in complementary strands of the SNP position, respectively; contacting the material with a polymerase; and identifying a product of the reaction indicative of the SNP.

Methods according to this aspect of the invention can be used for example, for fingerprint analysis, to identify an individual. Furthermnore, methods according to this aspect of the invention can be used to screen novel statins or other xenobiotics for efficacy and toxicity to hepatocytes.

Accordingly, the present invention also relates to an isolated primer pair, which can be useful for amplifying a nucleotide sequence comprising a SNP in a polynucleotide, wherein a forward primer of the primer pair selectively binds the polynucleotide upstream of the SNP position on one strand and a reverse primer selectively binds the polynucleotide upstream of the SNP position on a complementary strand, wherein the polynucleotide includes a nucleotide occurrence corresponding to at least one of a thymidine residue at a position corresponding to nucleotide 425 of SEQ ID NO:10 {CYP3A4E3-5_{—}249}, or a minor nucleotide occurrence at a position corresponding to nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 519 of SEQ ID NO:11 {HMGCRE5E6-3_{—}283}, and nucleotide 1421 of SEQ ID NO:12 {HMGCRE16E18_{—}99}.

The present invention also relates to an isolated primer pair, which can be useful for amplifying a nucleotide sequence comprising a SNP in a polynucleotide, wherein a forward primer of the primer pair selectively binds the polynucleotide upstream of the SNP position on one strand and a reverse primer selectively binds the polynucleotide upstream of the SNP position on a complementary strand, wherein the polynucleotide includes a nucleotide occurrence corresponding to at least one of a thymidine residue at a position corresponding to nucleotide 425 of SEQ ID NO:10 {CYP3A4E3-5_{—}249}, or a minor nucleotide occurrence at nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283 }, and nucleotide 1421 of SEQ ID NO:12 {HMGCRE16E18_{—99}.}

The present invention also relates to an isolated primer pair, which can be useful for amplifying a nucleotide sequence comprising a SNP in a polynucleotide, wherein a forward primer of the primer pair selectively binds the polynucleotide upstream of the SNP position on one strand and a reverse primer selectively binds the polynucleotide upstream of the SNP position on a complementary strand, wherein the polynucleotide includes a minor nucleotide occurrences corresponding to at least one of nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283}, and nucleotide 1421 of SEQ ID NO:12 {HMGCRE16E18_{—}99}.

The isolated primer pair can include a 3′ nucleotide that is complementary to one nucleotide occurrence of the statin response-related SNP. Accordingly, the primer can be used to selectively prime an extension reaction to polynucleotides wherein the nucleotide occurrence of the SNP is complementary to the 3′ nucleotide of the primer pair, but not polynucleotides with other nucleotide occurrences at a position corresponding to the SNP.

It has been found that randomly selected primers about 20 nucleotides in length, for example, from the five prime and three-prime sequence included in the sequence listing, can be used as primers according to the present invention provided that the A/T:G/C ratios are similar within each primer.

In another embodiment the present invention provides an isolated probe for determining a nucleotide occurrence of a single nucleotide polymorphism (SNP) in a polynucleotide, wherein the probe selectively binds to a polynucleotide comprising at least one of a thymidine residue at a position corresponding to nucleotide 425 of SEQ ID NO:10 {CYP3A4E3-5_{—}249}, or a minor nucleotide occurrence at a SNP corresponding to nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283 }, and nucleotide 1421 of SEQ ID NO:12 {HMGCRE16E18_{—}99}.

In another embodiment the present invention provides an isolated probe for determining a nucleotide occurrence of a single nucleotide polymorphism (SNP) in a polynucleotide, wherein the probe selectively binds to a polynucleotide comprising at least one of a thymidine residue at a position corresponding to nucleotide 425 of SEQ ID NO:10 {CYP3A4E3-5_{—}249}, or a minor nucleotide occurrence at a SNP corresponding to nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283}, and nucleotide 1421 of SEQ ID NO:12 {HMGCRE16E18_{—}99}.

In another embodiment the present invention provides an isolated probe for determining a nucleotide occurrence of a single nucleotide polymorphism (SNP) in a polynucleotide, wherein the probe selectively binds to a polynucleotide that includes at least one of a thymidine residue at a position corresponding to nucleotide 425 of SEQ ID NO:10 {CYP3A4E3-5_{—}249}, or a minor nucleotide occurrence at of a SNP corresponding to nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283}, and nucleotide 1421 of SEQ ID NO:12 {HMGCRE16E18_{—}99}.

In another embodiment, the present invention provides an isolated primer for extending a polynucleotide. The isolated polynucleotide includes a single nucleotide polymorphism (SNP), wherein the primer selectively binds the polynucleotide upstream of the SNP position on one strand wherein the SNP position has a nucleotide occurrence corresponding to a thymidine residue at nucleotide 425 of SEQ ID NO:10 {CYP3A4E3-5_{—}249}, or a minor nucleotide occurrence at a position correspond to

nucleotide 1274 of SEQ ID NO:1 {CYP2D6E7_{—}339},

nucleotide 1757 of SEQ ID NO:2 {HMGCRE7E11-3_{—}472},

nucleotide 1159 of SEQ ID NO:4 {CYP2D6PE1_{—}2},

nucleotide 1093 of SEQ ID NO:5 {CYP2D6PE7_{—}150},

nucleotide 1223 of SEQ ID NO:6 {CYP2D6PE7_{—}286},

nucleotide 1311 of SEQ ID NO:7 {CYP3A4E7_{—}243},

nucleotide 808 of SEQ ID NO:8 {CYP3A4E10-5_{—}292},

nucleotide 227 of SEQ ID NO:9 {CYP3A4E12_{—}76};

nucleotide 519 of SEQ ID NO:11 {HMGCRE5E6-3_{—}283 }, and

nucleotide 1421 of SEQ ID NO:12 {HMGCRE16E18_{—}99}.

In another embodiment, the present invention provides an isolated primer for extending a polynucleotide. The polynucleotide includes a single nucleotide polymorphism (SNP), wherein the primer selectively binds the polynucleotide upstream of the SNP position on one strand. The polynucleotide includes one of the minor nucleotide occurrences at a position corresponding to at least one of

nucleotide 1757 of SEQ ID NO:2 {HMGCRE7E11-3_{—}472},

nucleotide 1159 of SEQ ID NO:4 {CYP2D6PE1_{—}2},

nucleotide 1093 of SEQ ID NO:5 {CYP2D6PE7_{—}150},

nucleotide 1223 of SEQ ID NO:6 {CYP2D6PE7_{—}286},

nucleotide 1311 of SEQ ID NO:7 {CYP3A4E7_{—}243},

nucleotide 808 of SEQ ID NO:8 {CYP3A4E10-5_{—}292},

nucleotide 227 of SEQ ID NO:9 {CYP3A4E12_{—}76};

nucleotide 425 of SEQ ID NO:10 {CYP3A4E3-5_{—}249},

nucleotide 519 of SEQ ID NO:11 {HMGCRE5E6-3_{—}283}, and,

nucleotide 1421 of SEQ ID NO:12 {HMGCRE16E18_{—}99}.

The present invention further relates to an isolated specific binding pair member, which can be useful for determining a nucleotide occurrence of a SNP in a polynucleotide, wherein the specific binding pair member specifically binds to a minor nucleotide occurrence of the polynucleotide at or near a position corresponding to nucleotide 1274 of SEQ ID NO:1 {CYP2D6E7_{—}339}, nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 425 of SEQ ID NO:10 {CYP3A4E3-5_{—}249}, nucleotide 519 of SEQ ID NO:11 {HMGCRE5E6-3_{—}283 }, and nucleotide 1421 of SEQ ID NO:12 {HMGCRE16E18_{—}99}. The specific binding pair member can be, for example, an oligonucleotide or an antibody. Where the specific binding pair member is an oligonucleotide, it can be a substrate for a primer extension reaction, or can be designed such that is selectively hybridizes to a polynucleotide at a sequence comprising the SNP as the terminal nucleotide.

The present invention further relates to an isolated specific binding pair member, which can be useful for determining a nucleotide occurrence of a SNP in a polynucleotide, wherein the specific binding pair member specifically binds to a thymidine residue at a position corresponding to nucleotide 425 of SEQ ID NO:10 {CYP3A4E3-5_{—}249}, or to a minor nucleotide occurrence of the polynucleotide at or near a position corresponding to nucleotide 1757 of SEQ ID NO:2 {HMGCRE7E11-3_{—}472}, nucleotide 1159 of SEQ ID NO:4 {CYP2D6PE1_{—}2}, nucleotide 1093 of SEQ ID NO:5 {CYP2D6PE7_{—}150}, nucleotide 1223 of SEQ ID NO:6 {CYP2D6PE7_{—}286}, nucleotide 1311 of SEQ ID NO:7 {CYP3A4E7_{—}243}, nucleotide 808 of SEQ ID NO:8 {CYP3A4E10-5_{—}292}, nucleotide 227 of SEQ ID NO:9 {CYP3A4E12_{—}76}; nucleotide 519 of SEQ ID NO:11 {HMGCRE5E6-3_{—}283}, and nucleotide 1421 of SEQ ID NO:12 {HMGCRE16E18_{—}99}.

For methods wherein the specific binding pair member is a substrate for a primer extension reaction, the specific binding pair member is a primer that binds to a polynucleotide at a sequence comprising the SNP as the terminal nucleotide. As discussed above, methods such as SNP-IT (Orchid BioSciences), utilize primer extension reactions using a primer whose terminal nucleotide binds selectively to certain nucleotide occurrence(s) at a SNP loci, to identify a nucleotide occurrence at the SNP loci.

The present invention also provides primers, probes, specific binding pair members and isolated polynucleotides as described herein, for SNPs disclosed in Example 19, particularly those SNPs in Example 19 whose SNPname (see Table 9-14) includes anything other than “DBSNP”. It will be recognized that a novel nucleotide occurrence at these SNPs can be identified by using the sequence disclosed herein in the sequence listing and FIG. 3 to search Genbank or DBSNP to identify a known nucleotide occurrence at that position.

The present invention also relates to an isolated polynucleotide, which contains at least about 30 nucleotides and a minor nucleotide occurrence of a SNP of an HMGCR gene, at a position corresponding to nucleotide 519 of SEQ ID NO:11 {HMGCRE5E6-3_{—}283}, nucleotide 1757 of SEQ ID NO:2 {HMGCRE7E11-3_{—}472}, nucleotide 1430 of SEQ ID NO:3 {HMGCRDBSNP_{—}45320}, or a nucleotide corresponding to nucleotide 1421 of SEQ ID NO:12 {HMGCRE16E18_{—}99}. The isolated polynucleotide can further include a minor nucleotide occurrence at a second statin-related SNP corresponding to nucleotide 519 of SEQ ID NO:11 {HMGCRE5E6-3_{—}283}, nucleotide 1757 of SEQ ID NO:2 {HMGCRE7E11-3_{—}472}, or nucleotide 1421 of SEQ ID NO:12 {HMGCRE16E18_{—}99}. The isolated polynucleotide can include a minor HMGCRB haplotype allele.

A polynucleotide of the present invention, in another embodiment, can include at least 30 nucleotides of the human cytochrome p450 3A4 (CYP3A4) gene, wherein the polynucleotide includes at least one of a thymidine residue at a position corresponding to nucleotide 425 of SEQ ID NO:10 {CYP3A4E3-5_{—}249}, and a minor nucleotide occurrence of a first statin response-related SNP corresponding to nucleotide 425 of SEQ ID NO:10 {CYP3A4E3-5_{—}249}, nucleotide 1311 of SEQ ID NO:7 {CYP3A4E7_{—}243}, nucleotide 808 of SEQ ID NO:8 {CYP3A4E10-5_{—}292}, or nucleotide 227 of SEQ ID NO:9 {CYP3A4E12_{—}76}. The polynucleotide can further include a minor nucleotide occurrence at a second statin-related SNP corresponding to nucleotide 1311 of SEQ ID NO:7 {CYP3A4E7_{—}243}, nucleotide 808 of SEQ ID NO:8 {CYP3A4E10-5_{—}292}, or nucleotide 227 of SEQ ID NO:9 {CYP3A4E12_{—}76}. The isolated polynucleotide can include a minor CYP3A4A, CYP3A4B, or CYP3A4C haplotype allele.

In another embodiment, the present invention provides an isolated polynucleotide that includes at least 30 nucleotides of the cytochrome p450 2D6 (CYP2D6) gene. The polynucleotide includes a first minor nucleotide occurrence of at least a first statin response related single nucleotide polymorphism (SNP), wherein said minor nucleotide occurrence is at a position corresponding to nucleotide 1159 of SEQ ID NO:4 {CYP2D6PE1_{—}2}, a nucleotide 1093 of SEQ ID NO:5 {CYP2D6PE7_{—}150}, or a nucleotide 1223 of SEQ ID NO:6 {CYP2D6PE7_{—}286}. The isolated polynucleotide can further include a minor nucleotide occurrence at a second statin-related SNP corresponding to nucleotide 1159 of SEQ ID NO:4 {CYP2D6PE1_{—}2}, a nucleotide 1093 of SEQ ID NO:5 {CYP2D6PE7_{—}150}, or a nucleotide 1223 of SEQ ID NO:6 {CYP2D6PE7_{—}286}. Furthermore, the isolated polynucleotide can include a minor CYP2D6A haplotype allele.

The isolated polynucleotide can be at least 50, at least 100, at least 150, at least 200, at least 250, at least 500, at least 1000, etc. nucleotides in length. In certain embodiments of this aspect of the invention, the isolated polynucleotide can be at least 50, at least 100, at least 150, at least 200, at least 250, at least 500, at least 1000, etc. nucleotides in length.

In embodiments wherein the minor nucleotide occurrence is at a position corresponding to nucleotide 519 of SEQ ID NO:11 {HMGCRE5E6-3_{—}283}, the isolated polynucleotide can comprise SEQ ID NO:11. In embodiments wherein the minor nucleotide occurrence is at a position corresponding to nucleotide 1757 of SEQ ID NO:2 {HMGCRE7E11-3_{—}472} the isolated polynucleotide can comprise SEQ ID NO:2. In embodiments wherein the minor nucleotide occurrence is at a position corresponding to nucleotide 1430 of SEQ ID NO:3 {HMGCRDBSNP_{—}45320}, the isolated polynucleotide can comprise SEQ ID NO::3. In embodiments wherein the minor nucleotide occurrence is at a position corresponding to nucleotide corresponding to nucleotide 1421 of SEQ ID NO:12 {HMGCRE16E18_{—}99}, the isolated polynucleotide can comprise SEQ ID NO::12.

In embodiments wherein the nucleotide occurrence is a thymidine residue at a position corresponding to nucleotide 425 of SEQ ID NO:10 {CYP3A4E3-5_{—}249}, the isolated polynucleotide can comprise SEQ ID NO:10. In embodiments wherein the minor nucleotide occurrence is at a position corresponding to nucleotide 1311 of SEQ ID NO:7 {CYP3A4E7_{—}243}, the isolated polynucleotide can comprise SEQ ID NO:7. In embodiments wherein the minor nucleotide occurrence is at a position corresponding to nucleotide 808 of SEQ ID NO:8 {CYP3A4E10-5_{—}292}, the isolated polynucleotide can comprise SEQ ID NO:8. In embodiments wherein the minor nucleotide occurrence is at a position corresponding to nucleotide 227 of SEQ ID NO:9 {CYP3A4E12_{—}76} the isolated polynucleotide can comprise SEQ ID NO:9.

In embodiments wherein the minor nucleotide occurrence is at a position corresponding to nucleotide 1159 of SEQ ID NO:4 {CYP2D6PE1_{—2}}, the isolated polynucleotide can include SEQ ID NO:4.

In embodiments wherein the minor nucleotide occurrence is at a position corresponding to nucleotide 1093 of SEQ ID NO:5 {CYP2D6PE7_{—}150}, the isolated polynucleotide can include SEQ ID NO:5.

In embodiments wherein the minor nucleotide occurrence is at a position corresponding to nucleotide 1223 of SEQ ID NO:6 {CYP2D6PE7_{—}286} the isolated polynucleotide can include SEQ ID NO 6.

The polynucleotides of the present invention have many uses. For example, the polynucleotides can be used in recombinant DNA technologies to produce recombinant polypeptides that can be used, for example, to determine whether a statin binds or effects activity of the polypeptide. The present invention also provides isolated polypeptides that are produced using the isolated polynucleotides of the present invention.

In another aspect, the invention provides a method for identifying genes, including statin response genes, SNPs, SNP alleles, haplotypes, and haplotype alleles that are statistically associated with a statin response. This aspect of the invention provides commercially valuable research tools, for example. The approach can be performed generally as follows:

1) Select genes from the human genome database that are likely to be involved in a statin response;

2) Identify the common genetic variations in the selected genes by designing primers to flank each promoter, exon and 3′ UTR for each of the genes; amplifying and sequencing the DNA corresponding to each of these regions in enough donors to provide a statistically significant sample; and utilize an algorithm to compare the sequences to one another in order to identify the positions within each region of each gene that are variable in the population, to produce a gene map for each of the relevant genes;

3) Use the gene maps to design and execute large-scale genotyping experiments, whereby a significant number of individuals, typically at least one hundred, more preferably at least two hundred individuals, of known statin response are scored for the polymorphisms; and

4) Use the results obtained in step 3) to identify genes, polymorphisms, and sets of polymorphisms, including haplotypes, that are quantitatively and statistically associated with a statin response.

The Examples included herein illustrate general approaches for discovering statin response-related SNPs and SNP alleles as provided above.

The invention also relates to kits, which can be used, for example, to perform a method of the invention. Thus, in one embodiment, the invention provides a kit for identifying haplotype alleles of statin response-related SNPs. Such a kit can contain, for example, an oligonucleotide probe, primer, or primer pair, or combinations thereof, of the invention, such oligonucleotides being useful, for example, to identify a SNP or haplotype allele as disclosed herein; or can contain one or more polynucleotides corresponding to a portion of a CYP3A4, CYP2D6, or HMGCR gene containing one or more nucleotide occurrences associated with a statin response, such polynucleotide being useful, for example, as a standard (control) that can be examined in parallel with a test sample. In addition, a kit of the invention can contain, for example, reagents for performing a method of the invention, including, for example, one or more detectable labels, which can be used to label a probe or primer or can be incorporated into a product generated using the probe or primer (e.g., an amplification product); one or more polymerases, which can be useful for a method that includes a primer extension or amplification procedure, or other enzyme or enzymes (e.g., a ligase or an endonuclease), which can be useful for performing an oligonucleotide ligation assay or a mismatch cleavage assay; and/or one or more buffers or other reagents that are necessary to or can facilitate performing a method of the invention. The primers or probes can be included in a kit in a labeled form, for example with a label such as biotin or an antibody.

In one embodiment, a kit of the invention includes one or more primer pairs of the invention, such a kit being useful for performing an amplification reaction such as a polymerase chain reaction (PCR). Such a kit also can contain, for example, one or reagents for amplifying a polynucleotide using a primer pair of the kit. The primer pair(s) can be selected, for example, such that they can be used to determine the nucleotide occurrence of a statin response-related SNP, wherein a forward primer of a primer pair selectively hybridizes to a sequence of the target polynucleotide upstream of the SNP position on one strand, and the reverse primer of the primer pair selectively hybridizes to a sequence of the target polynucleotide upstream of the SNP position on a complementary strand. When used together in an amplification reaction an amplification product is formed that includes the SNP loci.

In addition to primer pairs, in this embodiment the kit can further include a probe that selectively hybridizes to the amplification product of one of the nucleotide occurrences of a SNP, but not the other nucleotide occurrence. Also in this embodiment, the kit can include a third primer which can be used for a primer extension reaction across the SNP loci using the amplification product as a template. In this embodiment the third primer preferably binds to the SNP loci such that the nucleotide at the 3′ terminus of the primer is complementary to one of the nucleotide occurrences at the SNP loci. The primer can then be used in a primer extension reaction to synthesize a polynucleotide using the amplification product as a template, preferably only where the nucleotide occurrence is complementary to the 3′ nucleotide of the primer. The kit can further include the components of the primer extension reaction.

In another embodiment, a kit of the invention provides a plurality of oligonucleotides of the invention, including one or more oligonucleotide probes or one or more primers, including forward and/or reverse primers, or a combination of such probes and primers or primer pairs. Such a kit provides a convenient source for selecting probe(s) and/or primer(s) useful for identifying one or more SNPs or haplotype alleles as desired. Such a kit also can contain probes and/or primers that conveniently allow a method of the invention to be performed in a multiplex format.

The kit can also include instructions for using the probes or primers to identify a statin response-related haplotype allele.

The inference drawn according to the methods of the invention can utilize a complex classifier function. However, as illustrated in the Examples, simple classifier systems can be used with the statin response-related SNPs and haplotypes of the present invention to infer statin response. However, the methods of the invention, which draw an inference regarding a statin response of a subject can use a complex classification function. A classification function applies nucleotide occurrence information identified for a SNP or set of SNPs such as one or preferably a combination of haplotype alleles, to a set of rules to draw an inference regarding a statin response. Pending U.S. patent application Ser. No. 10/156,995, filed May 28, 2002, provides examples of complex classifier methods.

The following examples are intended to illustrate but not limit the invention.

### EXAMPLE 1

### Identification of CYP2D6 Polymorphism Associated with Statin Response

Because adverse hepatocellular response to statins pose serious long-term health risks, physicians routinely run “liver panels” on patients initiating statin therapy. Serum glutamic oxaloacetic (SGOT) and serum glutamic pyruvic transaminases (SGPT) tests are the two most common liver panel tests. Base SGOT, post SGOT, base GPT and post GPT are shown in Table 1-1 (below). These tests measure the level of liver transaminase activity in various patients before (base) and after (post) the prescription of the statin in a given patient. For the average individual, an increase in the SGOT level to 37 or higher, or an increase in the GPT level above 56 signifies an adverse hepatocellular response. However, these thresholds are relevant to the average human, without regard to their race, sex or age. A better indicator is an increase in the post (on-drug) reading relative to the base (baseline) reading greater or equal to two-fold. Adverse hepatocellular responses to statins usually result in discontinuation of the medication for the protection of the patient.

Creatine kinase is another enzyme whose increased levels are indicative of adverse response to statins. About 20% of patients who take statins complain of muscle ache, and elevated creatine kinase levels are indicative of myalgia (muscle injury).

The effect of the drug on the patients liver enzyme levels can be determined by comparing the post (prescription) level to the base level (before prescription). In the patient specimen databank used for these studies, several readings for each of the tests are available, though only the latest test before the prescription date, and the earliest test result after the date of drug prescription, are presented. Increased post prescription readings are indicated by italicized, bold numbers of large font.

Adverse hepatocellular response to statins is common in individuals of the C/A genotype at the CYP2D6E7_{—}339 locus (5/8 tests conducted, and 3/3 persons surveyed). In contrast, adverse hepatocellular response to statins is relatively “uncommon” for individual of the C/C genotype at the CYP2D6E7_{—}339 locus (only 3/41 tests conducted, and 2/20 persons surveyed). This result can be seen by noting that the number of bold print, italicized and large font numbers in Table 1-1 constitute a larger proportion of the total number of readings in persons of the C/A genotype compared to persons of the C/C genotype. These results indicate that the proclivity for a patient to develop adverse hepatocellular response to statins can be predicted, to an extent, by their genotype at the CYP2D6E7_{—}339 locus. Further, these results indicate that the CYP2D6 gene is involved in individual human responses to at least two statin drugs—Lipitor™ and Zocor™.

Table 1-1 shows two groups of data. Individuals with the C/A (the minor) genotype at the CYP2D6E7_{—}339 polymorphism are shown in the first group, and individuals with the C/C (the major) genotype at the CYP2D6E7_{—}339 locus are shown in the second (see, also, Table 2; SEQ ID NO:3). SGOT and SGPT measurements taken before the prescription of the drug are indicated as “BASE” readings. SGOT and SGPT measurements taken after the prescription of the drug are indicated as “POST” readings. The particular Statin drug the patient is prescribed is listed. The hepatocellular and creatine kinase (CKIN) response data were collected by physicians during the normal course of treatment for the patients. Adverse responses are indicated by bold, italicized numbers. Data is not available for every patient, for every test. No data is indicated by a blank space.

TABLE 1-1

| | | | | | | | |
|---|---|---|---|---|---|---|---|
| | | BASE | POST | | | | |
| PATIENT | DRUG | SGOT | SGOT | BASE GPT | POST GPT | BASE CKIN | POST CKIN |

PATIENTS WITH THE C/A GENOTYPE AT CYP2D6E7_339 (DNAP MARKER 554368)

| | | | | | | | |
|---|---|---|---|---|---|---|---|
| DNAP00003 | ZOCOR | 16 | 35 | 12 | 42 | | |
| DNAP00006 | ZOCOR | | | 10 | 18 | | |
| DNAP00072 | LIPITOR | 12 | 27 | 12 | 13 | 99 | 222 |
| DNAP00072 | ZOCOR | 13 | 14 | 5 | 10 | | |

PATIENTS WITH THE C/C GENOTYPE AT CY02D6E7_339 (DNAP MARKER 554368)

| | | | | | | | |
|---|---|---|---|---|---|---|---|
| DNAP00007 | ZOCOR | 11 | 12 | 10 | 9 | | 42 |
| DNAP00009 | ZOCOR | | 17 | 12 | 14 | | |
| DNAP00010 | LIPITOR | 24 | 24 | 15 | 18 | | 37 |
| DNAP00011 | LIPITOR | 23 | | 16 | | 67 | |
| DNAP00011 | LESCOL | 15 | | 13 | | | |
| DNAP00013 | LIPITOR | 17 | 22 | 16 | 14 | 23 | 33 |
| DNAP00014 | LIPITOR | 18 | 33 | 14 | 33 | 37 | 93 |
| DNAP00017 | ZOCOR | 28 | 30 | 27 | 37 | | |
| DNAP00017 | PRAVACHOL | 30 | 36 | 37 | 20 | | |
| DNAP00017 | ZOCOR | 36 | | 20 | | | |
| DNAP00018 | ZOCOR | 20 | 21 | | 21 | | 113 |
| DNAP00019 | ZOCOR | 13 | 24 | | 15 | 90 | 121 |
| DNAP00020 | PRAVACHOL | | | | | | |
| DNAP00020 | LIPITOR | 19 | 24 | 24 | 48 | 70 | 111 |
| DNAP00021 | ZOCOR | 26 | 20 | 22 | 16 | | |
| DNAP00022 | LIPITOR | 26 | 40 | 23 | 23 | | |
| DNAP00022 | ZOCOR | 40 | 31 | 19 | 31 | | 78 |
| DNAP00023 | LIPITOR | 18 | 20 | | | | 63 |
| DNAP00024 | ZOCOR | 19 | 21 | 21 | 20 | | |
| DNAP00025 | TRICOR | 23 | 25 | | 13 | | |
| DNAP00025 | ZOCOR | 25 | 36 | 13 | 17 | | |
| DNAP00026 | ZOCOR | 25 | 28 | 25 | 26 | 253 | 707 |
| DNAP00026 | TRICOR | 28 | 32 | | | 313 | 596 |
| DNAP00026 | ZOCOR | 24 | 29 | | 23 | 217 | 141 |
| DNAP00026 | NIASPAN | 29 | 25 | 23 | 25 | 384 | 253 |
| DNAP00027 | LIPITOR | 25 | 17 | 30 | | 154 | 133 |

These results demonstrate that not all individuals who develop an adverse hepatocellular response to statins harbor the C/A genotype at this locus. For Example, DNAP00014 harbors a C/C genotype at the CYP2D6E7_{—}339 locus, but develops an adverse response to the statin, Lipitor™. This result is not unexpected, as most traits in the human population are the function of complex gene-gene and gene-environment interactions. If a gene product is involved in the metabolism of a given drug, several different polymorphisms in this gene may impair the function of the gene product and thus, the metabolism of the drug. One person may harbor one particular debilitating polymorphism, and another person may harbor another. Thus, on a population level, it is expected that several polymorphisms in the gene can be associated with adverse events associated with use of the drug. The present results indicate that the CYP2D6E7_{—}339 polymorphism of the invention is one of the polymorphisms that impact patient hepatocellular response to this drug, and that variation at the CYP2D6E7_{—}339 locus explains, at least in part, the natural variance in hepatocellular response to statins.

Accordingly, the present invention provides compositions for detecting the CYP2D6E7_{—}339 polymorphism; methods that query other genetic variants that are genetically linked to the claimed polymorphism (CYP2D6E7_{—}339) for the determination of adverse hepatocellular response to statins; methods that query the deoxyribonucleic acid polymorphism (CYP2D6E7_{—}339) for the determination of adverse hepatocellular response to statins; methods that query the level of transcript, or variants of the (CYP2D6E7_{—}339) transcript for the determination of adverse hepatocellular response to statins, and methods that query the level of the variant CYP2D6E7_{—}339 polypeptide, or polypeptides containing this variant, for the determination of the adverse hepatocellular response to statins.

Methods

The CYP2D6E7_{—}339 polymorphism was difficult to identify due to the difficulty in specifically amplifying this member of the larger CYP family, and because there are several CYP2D6 pseudogenes that complicated studies of this gene. Humans contain up to 60 unique CYP genes. Amplifying the CYP2D6 gene specifically was crucial for discovering polymorphisms in this gene through sequence analysis. The primers that were used to find the CYP2D6E7_{—}339 polymorphism also imparted a unique specificity for the genotyping assay of this locus in the patient population.

The CYP2D6E7_{—}339 polymorphism was scored using a single-nucleotide sequencing protocol and equipment purchased and licensed from Orchid Biosciences (Orchid SNPstream 25K instrument). Briefly, primers were designed to flank the polymorphism, whereby one primer of each pair contains 5′-polythiophosphonate groups. The 5′ flanking sequence and 3′ flanking sequence of the polymorphism and the polymorphic site (indicated by “N”) are shown in SEQ ID NO:1. Since these primers were designed without regard to other CYP family members, a nested PCR strategy was used, whereby the CYP2D6 specific primers used to discover the CYP2D6E7_{—}339 polymorphisms were used in the first round of amplification. Second round amplification products, using the second set of primers, were physically attached to a solid substrate via the polythiophosphonate groups and washed using TNT buffer. Primers and amplification products were as follows:

1) Primer Set 1

| | |
|---|---|
| 5′ primer: | |
| 5′-aggcaagaaggagtgtcagg; and | (SEQ ID NO:13) |
| 3′ primer: | |
| 5′-cagtcagtgtggtggcattg. | (SEQ ID NO:14) |

2) Primer Set 2 (“P” Indicates the Primer is Phosphothionated)

| | | |
|---|---|---|
| PCRL P | 5′-GTGGGGACAGTCAGTGTGGT; and | (SEQ ID NO:15) |
| PCRU | 5′-AGCMCCTGGTGATAGCCC. | (SEQ ID NO:16) |

The amplification product created by these two primers was (the CYP2D6E7_{—}339 polymorphism is indicated with an “M” flanked by a blank space 5′ and 3′ to the M):

| | |
|---|---|
| 5′-AGCMCCTGGTGATAGCCCCAGCATGGCYACTGCCAGGTGGGCCCASTCTAGGAA M. | (SEQ ID NO:17) |
| CCTGGCCACCYAGTCCTCAATGCCACCACACTGACTGTCCCCAC. | |

The oligonucleotide used to detect the SNP in this amplification was:

| | | |
|---|---|---|
| GBAU | 5′-YACTGCCAGGTXGGCCCASTCTAGGAA. | (SEQ ID NO:18) |

One of the NCBI reference sequences for the CYP2D6 gene is M33388, which is incorporated herein by reference. The CYP2D6E7_{—}339 polymorphism is located at position 5054 in this reference sequence.

### EXAMPLE 2

### Methods for Identifying SNPS and Haplotypes Related to Statin Response

The study sample consisted of several hundred patients treated with statins. Subjects provided a blood sample after providing informed consent and completing a biographical questionnaire. Samples were processed into DNA immediately and the DNA stored at −80° C. for the duration of the project. Samples were used only as per this study design and project protocol. Biographical data was entered into an Oracle relational database system run on a Sun Enterprise 420R server.

Marker Gene Selection

Gene markers were selected based on evidence from the body of literature, or from other sources of information, that implicate them in either the hepatocellular function or hepatocellular responsiveness to statins. The Physicians Desk Reference, Online Mendelian Inheritance database (NCBI) and PubMed/Medline are Examples of sources used for this information.

SNP Discovery within Markers Genes (Data Mining)

CYP2D6E7_{—}339 was discovered using a resequencing protocol as described below. Novel polymorphisms in the CYP2D6 gene, the HMGCR gene, and the CYP3A4 gene were identified using raw human genomic data present in public data resources (NCBI database) using data mining tools. The NCBI SNP database, the Human Genome Unique Gene database (Unigene from NCBI) and a DNA sequence database generated for this and similar studies, were used as sources for this raw sequence data. Sequence files for the genes were downloaded from proprietary and public databases and saved as a text file in FASTA format and analyzed using a multiple sequence alignment tool. The text file that was obtained from this analysis served as the input for SNP/HAPLOTYPE automated pipeline discovery software system (See U.S. patent application Ser. No. 09/964,059, filed Sep. 26, 2001, incorporated herein by reference). This method finds candidate SNPs among the sequences and documents haplotypes for the sequences with respect to these SNPs. The method uses a variety of quality control metrics when selecting candidate SNPs including the use of user specified stringency variables, the use of PHRED quality control scores and others.

Resequencing

The public genome database was constructed from a relatively small collection of donors. In order to discover new SNPs that may be under-represented or biased against in the public human SNP and Unigene databases, the CYP2D6 gene was completely sequenced in a larger pool (n=500) of persons (the DNA specimens were obtained from the Coriell Institute). Specimens from this combined pool were used as a template for amplification using a combination of Pfu turbo thermostable DNA polymerase and Taq polymerase. Amplification was performed in the presence of 1.5 mM MgCl₂, 5 mM KCl, 1 mM Tris, pH 9.0, and 0.1% Triton X-100 nonionic detergent. Amplification products were cloned into a T-vector using the Clontech (Palo Alto, Calif.) PCR Cloning Kit, transformed into Calcium Chloride Competent cells (Stratagene; La Jolla Calif.), plated on LB-Ampicillin plates and grown overnight.

Clones were selected from each plate, isolated by a miniprep procedure using the Promega Wizard or Qiagen Plasmid Purification Kit, and sequenced using standard PE Applied Biosystems Big Dye Terminator Sequencing Chemistry. Sequences were deposited into an internet based relational database system, trimmed of vector sequence and quality trimmed.

Marker Genotyping

Genotypes were surveyed within the specimen cohorts by sequencing using Klenow fragment-based single base primer extension and an automated Orchid Biosciences SNPstream instrument, based on Dye linked immunochemical recognition of base incorporated during extension. Reactions were processed in 384-well format and stored into a temporary database application until transferred to a UNIX based SQL database.

Analytical Methods

The data corresponded to SNPs that are informative for distinguishing common genetic haplotypes that we have identified from public and private databases. Using algorithms, the data was used to infer haplotypes from empirically determined SNP sequences.

Allele frequencies were calculated and pair-wise haplotype frequencies estimated using an EM algorithm (Excoffier and Slatkin 1995). Linkage disequilibrium coefficients were then calculated. The analytical approach was based on the case-control study design. Genotype/biographical data matricies for each group was examined using a pattern detection algorithm. The purpose of these algorithms is to fit quantitative (or Mendelian) genetic data with continuous trait distributions (or discrete, as the case may be). In addition to various parameters such as linkage disequilibrium coefficients, allele and haplotype frequencies (within ethnic, control and case groups), chi-square statistics and other population genetic parameters (such as Panmitic indices) were calculated to control for systematic variation between the case and control groups. Markers/haplotypes with value for distinguishing the case matrix from the control, if any, were presented in mathematical form describing their relationship(s) and accompanied by association (test and effect) statistics.

### EXAMPLE 3

### Two Markers (One 2 Locus Haplotype System) for Statin Efficacy

HMG co-A reductase, encoded for by the HMGCR gene, is involved in the synthesis of cholesterol in humans. An abnormally high cholesterol level is linked with increased risk of artherosclerotic disease and heart attack. As discussed herein, a class of drugs called statins are commonly prescribed to patients with abnormally high total cholesterol, or total cholesterol/high density lipoprotein levels to reduce the risk of this disease. In some patients, adverse reactions such as increased liver transaminase levels (SGOT/GPT tests) are observed, which induce physicians to discontinue treatment or switch drugs for the patient. If these types of variable results are a function of genetic variability, and if the genetic variability responsible for the variable response could be learned, genetic tests could be developed for classifying patients prior to prescription to maximize therapeutic efficacy and minimize the probability of adverse events.

Methods for the present Example are discussed in Example 2. Probes and primers used for genotyping SNPs in this Example, are listed in Table 4. A high-density SNP (single nucleotide polymorphism) map of the HMGCR gene was developed, and individual statin patients were genotyped at each of these SNP positions in order to learn whether variable statin response is a function of HMGCR genotypes, haplotypes or haplotype pairs (see Table 3-1). The results for several individual SNPs are presented herein, and for haplotypes comprised of these SNPs that show the variable efficacy of the statin class of drugs.

Table 3-1 shows that the genotypes of patients at the two disclosed markers is associated with the extent to which statins reduced total cholesterol levels in each patient. The SAMPLE ID is an identification number for each patient in column 1. Column 2 shows the particular drug, and dose (mg/ml), and columns 3,4 and 5,6 show post prescription total cholesterol (TC) and low-density lipoprotein (LDL) levels.

TABLE 3-1

| | | | | | | HMGCRE7E11 | HMGCRDB | |
|---|---|---|---|---|---|---|---|---|
| | | | TC- | LDL- | LDL- | SNP-3_472 | SNP_45320 | HAPLO |
| SAMPLE ID | DRUG | TC-pre | post | pre | post | GENOTYPE | GENOTYPE | TYPE |
| DNAP00002 | ZOC | 190 | 228 | 80 | 124 | GA | TT | GT/AT |
| DNAP00002 | PRAV | 228 | 151 | 124 | 54 | GA | TT | GT/AT |
| DNAP00004 | ZOC10 | 281 | 243 | 204 | 137 | GA | TT | GT/AT |
| DNAP00004 | ZOC40 | 245 | 234 | 140 | 114 | GA | TT | GT/AT |
| DNAP00007 | ZOC10 | 271 | 219 | 171 | 109 | GA | TT | GT/AT |
| DNAP00007 | ZOC20 | 219 | 161 | 109 | 80 | GA | TT | GT/AT |
| DNAP00089 | LIP | 163 | 210 | 70 | 131 | GA | TT | GT/AT |
| DNAP00089 | LIP10 | 201 | 130 | | | GA | TT | GT/AT |
| DNAP00089 | LIP20 | 130 | 161 | | | GA | TT | GT/AT |
| DNAP00089 | LIP10 | 161 | 224 | 76 | 124 | GA | TT | GT/AT |
| DNAP00086 | ZOC20 | 211 | 201 | 137 | 101 | GA | TT | GT/AT |
| DNAP00021 | ZOC10 | 256 | 224 | 173 | 139 | GG | CT | GT/GC |
| DNAP00066 | ZOC10 | 243 | 300 | 158 | 113 | GG | CT | GT/GC |
| DNAP00001 | LIP10 | 256 | 184 | 151 | 95 | GA | CT | GT/AC |
| DNAP00020 | PRAV40 | 254 | 188 | 187 | 123 | GG | TT | GT/GT |
| DNAP00020 | LIP40 | 252 | 186 | 178 | 99 | GG | TT | GT/GT |
| DNAP00032 | ZOC20 | 258 | 143 | 188 | 78 | GG | TT | GT/GT |
| DNAP00052 | PRAV20 | 222 | 175 | 153 | 110 | GG | TT | GT/GT |
| DNAP00041 | ZOC20 | 241 | 160 | 188 | 78 | GG | TT | GT/GT |
| DNAP00013 | LIP10 | 246 | 248 | 116 | 123 | GG | TT | GT/GT |
| DNAP00019 | ZOC20 | 230 | 160 | 151 | 73 | GG | TT | GT/GT |
| DNAP00027 | LIP10 | 235 | 175 | 108 | 86 | GG | TT | GT/GT |
| DNAP00063 | PRAV20 | 238 | 215 | 238 | 215 | GG | TT | GT/GT |
| DNAP00021 | ZOC10 | 256 | 224 | 173 | 139 | GG | TT | GT/GT |
| DNAP00043 | LIP10 | 281 | 199 | 182 | 100 | GG | TT | GT/GT |
| DNAP00050 | ZOC20 | 309 | 207 | 191 | 95 | GG | TT | GT/GT |
| DNAP00084 | ZOC10 | 234 | 170 | 172 | 146 | GG | TT | GT/GT |
| DNAP00084 | ZOC20 | 210 | 112 | 146 | 60 | GG | TT | GT/GT |
| DNAP00005 | LIP10 | 195 | 135 | 139 | 80 | GG | TT | GT/GT |

Column 7 shows the genotype of the individual for the HMGCRE7E11-3_{—}472 marker and column 8 shows the genotype of the individual for the HMGCRDBSNP_{—}45320 marker. The diploid pair of haplotypes in each individual is shown in Column 9. Clinical test results (TC and LDL) were compiled using the latest test date for the given test before the date of drug prescription and the earliest test date for the given test after the date of drug prescription. Readings in regular print are reading pairs that show an individual patient did not respond, or did not respond adequately to statin treatment. Readings in italics and bold show test result pairs for a given test type that indicate a patient responded well to the statin treatment and readings in italics, but not bold, indicate a mediocre response.

The results in Table 3-1 demonstrate that the frequency of individuals (5/6) exhibiting a poor response to statins was increased in individuals of the GA genotype at the locus HMGCRE7E11_{—}472 locus, compared to individuals of the GG genotype at the same locus (3/15). This result is significant at the p=0.01 level. For the second marker, 2/3 individuals with the heterozygous (CT) genotype at the HMGCRDBSNP_{—}45320 locus (2/2) were poor responders. The TT homozygous genotype, alone, had little predictive value, showing about an equal number of TT poor responders and TT good responders.

A method of geometric modeling as described for analysis of the OCA2 locus (T. Frudakis, U.S. patent application Ser. No. 10/156,995, filed May 28, 2002), incorporated herein in its entirety by reference. was applied to the present loci to combine the markers into haplotypes and classification systems, to further illustrate their value as predictive markers. As is clear from the haplotypes above, there are 4 possible two locus haplotypes at the HMGCRE7E11-3_{—}472 and HMGCRDBSNP_{—}45320 loci, as follows (in order): 1)GT; 2)AT; 3)GC; and 4)AC.

An inspection of the HMGCRE7E11-3_{—}472 and HMGCRDBSNP_{—}45320 haplotype pairs with respect to statin response (specifically the reduction of Total Cholesterol or TC) in Table 3-1 revealed that individuals with two copies of the GT genotype tended to react as expected to statins (12/15 treatment events showed significant decrease in total cholesterol levels), whereas heterozygous individuals containing the GT haplotype and either the AT haplotype or GC haplotype tended to react poorly to statins (10/13 treatment events showed no significant decrease or an in total cholesterol levels).

Heterozygous individuals containing the GT haplotype along with the AC haplotype responded to statins similarly to individuals with two copies of the GT haplotype. These results indicate that, the AT haplotype and the GC haplotype are predictive for individual resistance, or inability to respond adequately to normal doses of statins.

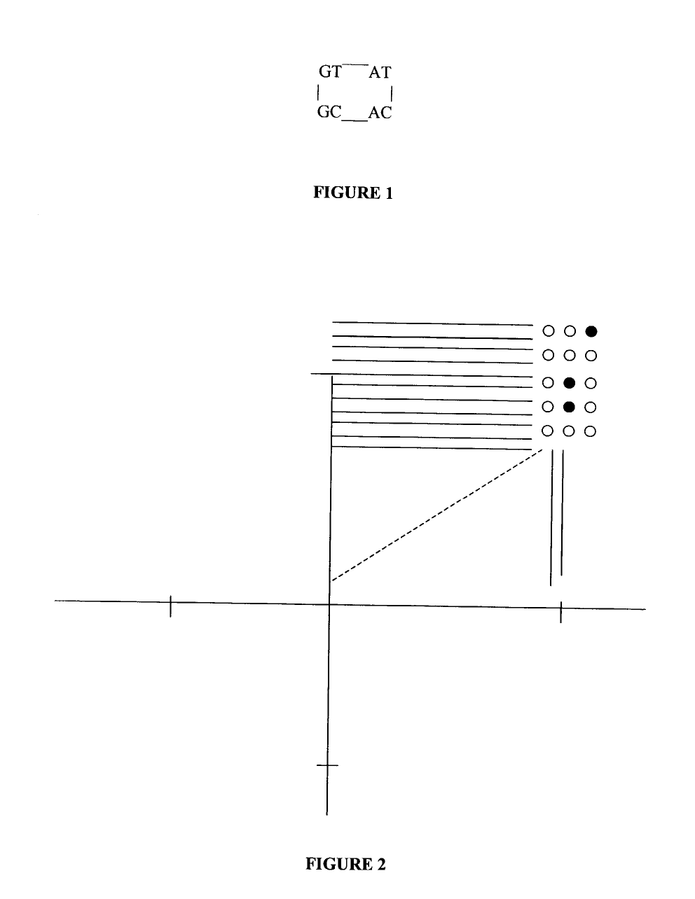

The haplotype cladogram for the four haplotype system is shown in FIG. 1.

Laying the cladogram over a grid, with values gives Table 3-2.

TABLE 3-2

| | 1 | 0 |
|---|---|---|
| 1 | GT | AT |
| 0 | GC | AC |

And the haplotype pairs can be recoded in two dimensions as:

GT/GT (1,1)(1,1)

GT/AT (1,1)(0,1)

GT/GC (1,1)(1,0)

GT/AC (1,1)(0,0)

FIG. 2 shows the haplotype pairs for individual patients plotted in 2 dimensional space. Individual haplotypes are shown as lines whose coordinates are given above in the text. If a person had two of the same haplotypes, for Example, GT/GT, which encoded as (1,1)(1,1), they were represented as a circle rather than a line. Solid lines or filled circles indicate individuals who did not respond to statin treatment, and dashed lines or open circles represent those that responded positively to statin treatment.

From FIG. 2, which is a visually informative way to represent the data shown in Table 2-1, it is clear that individuals containing the GT/GT haplotype pair, encoded as (1,1)(1,1) and shown in FIG. 5 as circles at position (1,1); or the GT/AC pair, encoded as (1,1)(1,0) and shown in FIG. 5 as a dashed line between these two coordinates, tend to respond well to statin treatment, but individuals containing GT and any other haplotype, such as AT or GC tend to not respond well to statin treatment (vertical and horizontal light lines).

The HMGCR SNPs are shown in Table 6-20 and SEQ ID NO:2 (HMGCRE7E11-3_{—}472) and SEQ ID NO:3 (HMGCRDBSNP_{—}45320). Table shows, in order, the GENE name, SNPNAME, LOCATION within the NCBI reference sequence (GENBANK), VARIANT IUB code for the polymorphic nucleotide position, FIVEPRIME flanking sequence and THREEPRIME flanking sequence is shown, in addition to the TYPE of SNP (intron, exon etc.), and the INTEGRITY (polymorphic or monomorphic).

### EXAMPLE 4

### CYP2D6 Haplotype Loci Predictive of Atorvastatin Efficacy

This Example identifies three loci (See Table 2; SEQ ID NOS:4-6) of the CYP2D6 haplotype system that are predictive of adverse responsiveness of a patient to statins.

A. Methods

Specimens

A network of primary care physician collectors was established throughout the state of Florida to provide anonymous, matching specimens and detailed biographical, drug and clinical data. The study design was approved by the appropriate investigational review boards for the hospitals working with each participating physician, and each participating patient read and signed a pan-drug informed consent form. Consent forms were retained by the treating physician to maintain anonymity. DNA was obtained from blood or buccal specimens using standard DNA isolation techniques (Promega, Madison Wis.) and quantified via spectrophotometry.

SNP discovery

A vertical resequencing of CYP2D6 encompassing the proximal promoter, exons, and 3′UTR was performed by amplifying each region from a multiethnic panel of 670 individuals. PCR was performed on this pool of 670 people with pfu Turbo, according to the manufacture's guidelines (Stratagene; La Jolla Calif.). Primers were designed so that the maximum number of relevant regions are included in the fewest possible number of conveniently sequencable amplicons, and selected the primers to not cross react with pseudo or other homologous sequences (for CYP2D6, for Example, the primers did not match the CYP2D6 pseudogene (CYP2D7) or other orthologous sequences in the human genome, including other CYP genes).

Amplification products were gel purified and subcloned into a sequencing vector, pTOPO (Invitrogen). Up to 192 insert-positive colonies were grown and plasmid DNA isolated and sequenced using one of the gene specific primers. The resulting sequences were aligned and analyzed to identify candidate SNPs based on characteristics of the alignment as well as the PHRED score of the discrepant base(s). (See U.S. patent application Ser. No. 09/964,059, filed Sep. 26, 2001, incorporated herein by reference.)

Genotyping

A first round of PCR was performed on these samples using the locus specific primers designed during re-sequencing (the SNP discovery primers described above). The resulting PCR products were checked on an agarose gel, diluted and then used as template for a second round of PCR incorporating phosphothionated primers. Genotyping was performed on individual DNA specimens using a single base primer extension protocol and an Orchid SNPstream 23K platform (Orchid Biosystems, Princeton, N.J.). This procedure was repeated for each SNP and all PCR steps used the high-fidelity DNA polymerase pfu turbo. Primers and probes for SNPs that are included in haplotypes that are useful for inferring a statin-related response, are included in Table 3,

Phenotyping

Determinations of serum glutamic oxaloacetic (SGOT) and glutamic pyruvic transaminases (SGPT), serum alkaline phosphatase (AP), bilirubin and albumin measurements were used to phenotype patients for hepatocellular response to Atorvastatin, Simvastatin and Pravastatin. Because many of the patients were taking multiple medications (an average of about 5 per patient), each was electronically phenotyped using the latest date of a given test before prescription of the drug as the baseline, and the earliest date of the test after prescription of the drug as the indicator. Subtracting the indicator from the baseline gave the best estimation of patient response to the statin for each test because the test dates most closely straddled the prescription date. Greater than 98% of the reading pairs for SGOT, ALTGPT, albumin, alkaline phosphatase and bilirubin tests were within 3 months of one another. For the creatine kinase tests, all readings were within 6 months of one another.

Data Analysis

Genotype and phenotype data were deposited and accessed from an Oracle 8i relational database system. Each patient was genotyped at every pharmaco-relevant marker in our database, and the database was randomly queried as it grew in order to automatically find and update statistically significant pharmacogenomics concepts. The pharmacogenomics discovery search engine was constructed using JAVA and queries randomly selected permutations of SNP combinations within genes and random combinations of haplotypes between genes for statistical association with certain selected drug-reaction traits. After a user defines the data set and the drug-reaction traits of interest, the software retrieves the relevant data, stores the query and automatically formats the data for input into the statistical component of the search engine. The engine utilizes various applications that culminate in the deposition of statistically significant population level comparisons (if any exist).

For the version of the software used in this study, the Stephens and Donnelly (2000) PHASE algorithm was used to infer haplotypes and the Arlequin program (Schneider et al., Arlequin ver. 2.000: A software for population genetics data analysis. Genetics and Biometry Laboratory, University of Geneva, Switzerland (2000)) to calculate population level test and effect statistics for each of the “randomly” selected phenotype comparisons. Results indicating significant population level structure for a given phenotype comparison causes the data to be kicked out to a separate subdirectory and subject to additional, more detailed analysis. Insignificant results were discarded. For the population comparisons, an average weighted pair wise F-statistic was determined. In addition, a Slatkin linearized F-statistic value (t) was calculated where t/M=FST/(1-FST) and M=2N for diploid data. Lastly, an exact test of non-differentiation between the groups was calculated assuming the null hypothesis. A comparison with significant results for two of these three tests was passed to the next step of analysis.

Allele frequencies were calculated for haplotype i using the function p₁=(x₁/n), where x₁ is the number of times that haplotype i was observed and n is the number of patients in the group. Standard deviations (sd) were measured from an unbiased estimate of the sampling variance given by V(pᵢ)=p₁(1−p₁)/(n−1). For the exact tests of non-differentiation, we used 1000 steps of the Markov chain and 1000 dememorization steps.

B. Results

Numerous cytochrome P450 polymorphisms are known to directly impact drug metabolism and disease (Kalow, W., Pharmacogenetics of drug metabolism. Pergamon Press, Elmsford, N.Y. (1992); Brown et al., Hum. Molec. Genet. 9: 1563-1566, (2000)), and virtually all of the concordance studies that aim to understand how or whether genetic variation in these genes impacts variable drug response incorporate these known alleles. Because idiosyncratic drug responses can be caused by unique gene variants, and because complete SNP maps documenting all of the common variants are not available for many of these genes, a database of all the common Cytochrome P450 (and other gene) SNPs was constructed.

An average of 30 candidate SNPs per gene were identified, and were distributed throughout the proximal promoter, each exon and the 3′UTR of each gene (Table 4-1). The number of SNPs was highly variable between regions within each gene as well as between cognate regions of different genes. Some of the SNPs have been discovered or documented before, but most were novel (particularly SNPs within intron regions, data not shown).

Table 4-1 shows the number of candidate SNPs and validated SNPs (parenthesis) found in each of 23 xenobiotic metabolizer genes that could conceivably be involved in idiosyncratic Statin responses in the population. The gene is identified in Column 1, and the number of SNPs found from the re-sequencing work described in the text is shown in Column 2. The number of SNPs known from the public SNP database (NCBI: dbSNP) and the number known from the literature are shown in Columns 3 and 4.

TABLE 4-1

| | SNPs found in: | | |
|---|---|---|---|
| GENE | DNAP dB | dbSNP | Literature |
| CYP2D6 | 56 | 6 | 74 |
| CYP3A4 | 23 | 5 | 25 |
| CYP3A5 | 12 | 5 | 8 |
| CYP3A7 | 24 | 11 | 5 |
| CYP2C9 | 24 | 17 | 12 |
| CYP1A1 | 15 | 4 | 10 |
| CYP1A2 | 29 | 4 | 13 |
| CYP2C19 | 32 | 5 | 11 |
| CYP2E1 | 23 | 5 | 13 |
| AHR | 14 | 10 | 0 |
| PON1 | 22 | 9 | 0 |
| PON3 | 14 | 2 | 0 |

Each validated SNP, in each gene was scored in a panel of 148 Caucasian statin patients, for whom detailed biographical, drug and clinical data were available. Genotypes were obtained, haplotypes inferred using the algorithm of Stephens and Donnelly (2000) and random permutations of the data analyzed in order to identify statistically significant associations (see materials and methods). A total number of 1,230 haplotype systems were queried for their ability to resolve patients in a way that was clinically meaningful. For each haplotype system inferred for a particular gene (average n=28), the patients were stratified based on hepatocellular responses to the three drugs as indicated by each of five clinical end-points: ALTGPT, SGOT, Bilirubin, Alkaline Phosphatase and Albumin. Several overlapping haplotype “systems” were observed within the CYP2D6 gene that were useful for resolving patients based on SGOT responses to Atorvastatin (Table 4-2). The most parsimonious haplotype system of this group (explaining the most phenotypic variability with the fewest SNPs) contains three bi-allelic SNP loci distributed between the first and seventh exons of the CYP2D6 locus.

TABLE 4-2

| | | | | Freq | |
|---|---|---|---|---|---|
| LOCUS | SNP name | Marker | CHANGE | (minor) | HWE |
| 1 | CYP2D6PE1-2 | 554371 | Pro to Ser | 0.282 | No |
| 2 | CYP2D6E7_150 | 554363 | Silent | 0.040 | Yes |
| 3 | CYP2D6E7_286 | 554365 | Intron | 0.440 | Yes |

Table 4-2 shows CYP2D6 SNPs, haplotypes of which are predictive for the relative risk of adverse hepatocellular response to Atorvastatin as discussed in the text. The SNP name is shown in Column 2, and the DNAPrint identification number shown in Column 3. The type of amino acid change is shown in Column 4; if the SNP is located within an exon but there is no amino acid change the change is listed as Silent and if the SNP is not located within an exon, the location of the SNP is given. The frequency of the minor allele is presented in Column 5, and whether or not the SNP alleles are in Hardy-Weinberg equilibrium is noted in Column 6.

The minor allele frequencies for the three SNPs in the Caucasian population range from under 1% to 27%, and within the Caucasian group, alleles for all three SNPs were found to be within Hardy-Weinberg proportions (HWE; Table 4-2). Only one of these three SNPs was previously described in the literature, though no functionality was ascribed. Neither of the other two SNPs appear in the literature or the public SNP database (NCBI:dbSNP). Of the 2³=8 possible haplotypes combinations possible for these three loci, only 4 haplotypes were observed in a group of 244 haplotyped Caucasians; CTA, tTc, tTA, CTc and CcA, where the sequence of letters represent the alleles at each of the 3 loci in order from 5′to 3′within the gene, and a lower case letter indicates the minor allele. In the general Caucasian population, loci 1 and 3 are in linkage disequilibrium (P<0.00001+/−0.00001), as are loci 2 and 3 (P=0.034+/−0.0006), but loci 2 and 3 are not in LD. Of the three loci, only the alleles of locus 1 are not in Hardy-Wienberg equilibrium, which may explain why loci 1 and 3 are so strongly linked.

The first test performed stratified the patients, within each drug group, on absolute increase over baseline vs. no increase (or decrease) over baseline in SGOT levels following Statin prescription. Patients within each drug group also were stratified on a 20% increase over baseline vs. no increase (or decrease) over baseline in SGOT levels. The results of these analyses showed population level structure differences in the 3-locus CYP2D6 haplotype system (as well as in 4 other overlapping haplotype systems), but not other gene (n=11) haplotype systems (n=243) using both the absolute and 20% definition of adverse SGOT response. Using the absolute increase in SGOT criteria for defining adverse responders, the P-values ranged from 0.020+/−0.003 for the exact test to 0.063+/−0.004 for the pair wise F statistic (bold print, row 2, Table 4-3). Using the 20% over baseline increase in SGOT criteria for defining adverse responders, P-values ranged from 0.014+/−0.002 for the exact test to 0.018+/−0.002 for the pair wise F statistic (bold print, row 1, Table 4-3).

No CYP2D6 (or other gene) haplotype sequence differences were observed between similarly defined elevated and non-elevated groups for the other test types (alkaline phosphatase, ALTGPT, bilirubin or albumin) within the Atorvastatin patient group or the other two drug groups in this study (data not shown). No CYP2D6 (or other gene) haplotype sequence differences were observed for SGOT elevated and non-elevated populations taking Simvastatin or Pravastatin in this study (Table 4-3), and no haplotype sequence differences were noted for any haplotype systems within the other genes shown in Table 4-1 in this study. For Example, a randomly selected haplotype system from the CYP3A4 gene (a gene that is known to be involved in the disposition of Atorvastatin) is shown in Table 4-3 and revealed no significant associations for any of the tests in any of the drug groups (Table 4-3). It is possible that haplotype sequence differences (i.e. lack of a statistically significant correlation between the occurrence of certain haplotype alleles and a change in a hepatocellular stress test) for other hepatocellular tests, other statins, or other haplotypes exist but were not observed because of the sample size, the population of subjects analyzed. Furthermore, it is possible that latent haplotype alleles exist.

TABLE 4-3

| TEST | GENE | DRUG | PW dist F | PW P value | Slatkin | Exact P |
|---|---|---|---|---|---|---|
| sgot20 | CYP2D6 | Atorvastatin | 0.148 | 0.018 +/− 0.000 | 0.174 | 0.014 +/− 0.002 |
| sgot | CYP2D6 | Atorvastatin | 0.149 | 0.063 +/− 0.024 | 0.133 | 0.020 +/− 0.003 |
| sgot20 | CYP3A4 | Atorvastatin | 0.024 | 0.559 +/− 0.040 | 0 | 0.583 +/− 0.010 |
| sgot | CYP3A4 | Atorvastatin | 0.007 | 0.306 +/− 0.045 | 0.007 | 0.136 +/− 0.006 |
| sgot20 | CYP2D6 | Simvastatin | 0.012 | 0.460 +/− 0.039 | 0 | 0.630 +/− 0.011 |
| sgot | CYP2D6 | Simvastatin | 0.018 | 0.550 +/− 0.052 | 0 | 0.279 +/− 0.008 |
| sgot20 | CYP3A4 | Simvastatin | 0.029 | 0.991 +/− 0.003 | 0 | 1.000 +/− 0.000 |
| sgot | CYP3A4 | Simvastatin | 0.035 | 0.702 +/− 0.038 | 0 | 1.000 +/− 0.000 |
| sgot20 | CYP2D6 | Pravastatin | n/s | n/s | n/s | n/s |
| sgot | CYP2D6 | Pravastatin | n/s | n/s | n/s | n/s |
| sgot20 | CYP3A4 | Pravastatin | n/s | n/s | n/s | n/s |
| sgot | CYP3A4 | Pravastatin | n/s | n/s | n/s | n/s |

Table 4-3 shows differentiation tests of haplotype-based population structure between Atorvastatin, Simvastatin and Pravastatin SGOT responder groups. Though many haplotype systems were tested for each drug, only two haplotype systems within the CYP2D6 and CYP3A4 genes are shown (Column 2). The groupings used were adverse responders (patients that exhibited an absolute elevation in SGOT test reading) and non-responders (patients that did not exhibit an absolute elevation in the reading) (indicated as “sgot” in Column 1) or adverse responders (patients that exhibited greater than 20% elevation in SGOT levels) or non-responders (those that did not) (indicated as “sgot20” in Column 1). Each test type considered is indicated in the TEST column and readings from these tests were obtained as described in the text.

Because the population structure tests indicated a significant difference in haplotype structure between the two groups of SGOT responders taking Atorvastatin, the frequencies of the various observed haplotypes in responder and non-responder groups was calculated (Table 4-4). The results showed that the wild-type haplotype, CTA was more frequent in the SGOT unchanged group relative to the adverse SGOT responder group using the 20% increase in SGOT levels over baseline definition of adverse responders (80%+/−10% versus 30%+/−10%, respectively, for absolute vs. not SGOT responders, and 80%+/−10% versus 40%30 /−10%, respectively, for 20% SGOT responders). In contrast, the four minor haplotypes, tTc, tTA, CTc and CcA, were more frequent in the SGOT elevated groups (20%+/−10%, 10%+/−<0,1%, 30%+/−10%, 10%+/−<0,1%, respectively) than in the non-adverse SGOT responder groups (10%+/−10%, not observed, 10%+/−10%, not observed, respectively). Similar results were obtained using the absolute increase in SGOT levels over baseline definition of adverse SGOT response (Table 4-4). The standard deviations for both types of SGOT comparisons indicate that the differences in major versus minor haplotype frequencies are significant. In contrast, the relative frequencies of major versus minor CYP3A4 haplotypes were not significantly different between adverse versus non-adverse SGOT responders using either definition for adverse response, for any of the three drugs. Thus, the frequency differences for CYP2D6 major and minor haplotypes accounted for the difference in population haplotype structures we observed with the pair-wise F-statistic and non-differentiation exact tests.

TABLE 4-4

| | CYP2D6 HAPLOTYPE FREQUENCIES | | | | | |
|---|---|---|---|---|---|---|
| Drug | Criteria | CTA | tTc | tTA | CTc | CcA |
| Atorvastatin | sgot up >20% | 0.3 +/− 0/1 | 0.2 +/− 0.1 | 0.1 +/− 0.0 | 0.3 +/− 0/1 | 0.1 +/− 0.0 |
| Atorvastatin | sgot not up >20% | 0.8 +/− 0.1 | 0.1 +/− 0.1 | n/s | 0.1 +/− 0.1 | n/s |
| Simvastatin | sgot up >20% | 0.4 +/− 0.1 | 0.3 +/− 0.1 | n/s | 0.2 +/− 0.1 | 0.1 +/− 0.0 |
| Simvastatin | sgot up not >20% | 0.5 +/− 0.1 | 0.2 +/− 0.1 | 0.0 +/− 0.0 | 0.2 +/− 0.0 | 0.0 +/− 0.0 |
| Pravastatin | sgot up >20% | n/s | n/s | n/s | n/s | n/s |
| Pravastatin | sgot up not >20% | n/s | n/s | n/s | n/s | n/s |
| Atorvastatin | sgot up | 0.4 +/− 0.1 | 0.2 +/− 0.1 | 0.0 +/− 0.0 | 0.3 +/− 0.1 | 0.0 +/− 0.0 |
| Atorvastatin | sgot not up | 0.8 +/− 0.1 | 0.1 +/− 0.1 | n/s | 0.1 +/− 0.1 | n/s |
| Simvastatin | sgot up | 0.5 +/− 0.1 | 0.3 +/− 0.1 | n/s | 0.2 +/− 0.1 | 0.1 +/− 0.0 |
| Simvastatin | sgot not up | 0.3 +/− 0.2 | 0.3 +/− 0.2 | 0.1 +/− 0.1 | 0.3 +/− 0.2 | n/s |
| Pravastatin | sgot up | n/s | n/s | n/s | n/s | n/s |
| Pravastatin | sgot not up | n/s | n/s | n/s | n/s | n/s |

Table 4-4. shows CYP2D6 haplotype counts in adverse versus non-adverse SGOT responder groups. Two different criteria for adverse SGOT response are shown; an individual was assigned to the “sgot up” group if they responded to Atorvastatin therapy with an absolute increase in SGOT readings and to the “sgot not up” group if they did not respond to Atorvastatin therapy with an absolute increase in SGOT readings. Similarly, individuals were assigned to the “sgot up >20%” group if they responded to Atorvastatin therapy with at least a 20% increase in SGOT readings over baseline and to the “sgot not up >20%” group if they did not respond to Atorvastatin therapy with an at least 20% increase in SGOT readings. Minor alleles are indicated by lower case letters in the top row.

To cast these results in terms of diploid pairs of haplotypes, individual haplotype pairs were counted for the SGOT elevated and not elevated groups using both criteria for response (same as above). Condensing the data into contingency tables of diploid pairs in this manner shows a clear partition of CYP2D6 genotypes in the two responder groups (see Table 4-6). Eight haplotype pairs were observed in our patient group (Column 1, Table 4-6), and these haplotype pairs were encoded as pairs of wild-type (WT) and minor haplotypes based on their frequencies in the Caucasian population (Table 4-2). The results of this analysis revealed that the WT/WT haplotype pair was most commonly observed in persons that did not respond to Atorvastatin with increased SGOT readings (73% or 67% depending on the criteria for classifying adverse responders). In contrast, the WT/WT genotype was uncommon in individuals who responded to Atorvastatin with increased SGOT readings (<1% for either criteria). In fact, virtually all of the persons who responded to treatment with increased SGOT readings had at least one minor haplotype (>99%). The results were similar when the 25% increase in SGOT reading criteria was used to group the patients, although a slightly higher frequency of WT/MINOR haplotype pairs were observed in the SGOT not elevated group.

The average change in SGOT levels was determined for individuals with the various diploid haplotype combinations (Table 4-6). Because of the low frequency of some of the minor haplotypes, not all of the possible pairings were observed. Comparing the effects between the six combinations that were observed, we noted differences in the average effect (SGOT elevations) associated with various minor haplotypes. The average effect of the minor haplotype with two minor alleles (MINOR 1) is greater than the average effect of the other two minor haplotypes that each contain only one variant. The average effect of the MINOR 1 haplotype is greater when found with another minor haplotype (average 75% SGOT increase) than with the major (WT) haplotype (average 38% SGOT increase). However, the average effect of the MINOR 3 haplotype (average 52% SGOT increase) is the same when combined with another minor haplotype or with the major (WT) haplotype.

TABLE 4-5

| | CYP3A4 HAPLOTYPE FREQUENCIES | | | | |
|---|---|---|---|---|---|
| Drug | Criteria | GC | AC | AT | GT |
| Atorvastatin | sgot up >20% | 0.8 +/− 0.1 | 0.2 +/− 0.1 | 0.1 +/− 0.0 | n/s |
| Atorvastatin | sgot not up >20% | 0.8 +/− 0.1 | 0.1 +/− 0.1 | 0.1 +/− 0.1 | 0.1 +/− 0.1 |
| Simvastatin | sgot up >20% | 0.9 +/− 0.1 | 0.1 +/− 0.1 | n/s | n/s |
| Simvastatin | sgot not up >20% | 0.9 +/− 0.1 | 0.1 +/− 0.1 | n/s | 0.0 +/− 0.0 |
| Pravastatin | sgot up >20% | n/s | n/s | n/s | n/s |
| Pravastatin | sgot not up >20% | n/s | n/s | n/s | n/s |
| Atorvastatin | sgot up | 0.8 +/− 0.1 | 0.2 +/− 0.1 | 0.0 +/− 0.0 | n/s |
| Atorvastatin | sgot not up | 0.8 +/− 0.1 | n/s | 0.1 +/− 0.1 | 0.1 +/− 0.1 |
| Simvastatin | sgot up | 0.9 +/− 0.0 | 0.1 +/− 0.0 | n/s | 0.0 +/− 0.0 |
| Simvastatin | sgot not up | 0.9 +/− 0.1 | 0.1 +/− 0.1 | n/s | n/s |
| Pravastatin | sgot up | n/s | n/s | n/s | n/s |
| Pravastatin | sgot up | n/s | n/s | n/s | n/s |

Table 4-5. shows CYP3A4 haplotype counts in adverse versus non-adverse SGOT responder groups. Two different criteria for adverse SGOT response are shown; an individual was assigned to the “sgot up” group if they responded to Atorvastatin therapy with an absolute increase in SGOT readings and to the “sgot not up” group if they did not respond to Atorvastatin therapy with an absolute increase in SGOT readings. Similarly, individuals were assigned to the “sgot up >20%” group if they responded to Atorvastatin therapy with at least a 20% increase in SGOT readings over baseline and to the “sgot not up >20%” group if they did not respond to Atorvastatin therapy with an at least 20% increase in SGOT readings. Minor alleles are indicated by lower case letters in the top row.

TABLE 4-6
Frequencies of haplotype combination between atorvastatin SGOT responders.

| HAPLOTYPE | | | | | |
|---|---|---|---|---|---|
| PAIRS | TYPE | ELEVATED | NOT ELEVATED | >25% ELEVATION | <25% ELEVATION |
| CTA/CTA | WT/WT | <0.01 | 0.73 | <0.01 | 0.67 |
| CTA/CTc | WT/MINOR 1 | 0.64 | 0.18 | 0.60 | 0.25 |
| CTA/tTc | WT/MINOR 2 | 0.09 | <0.01 | 0.10 | <0.01 |
| CTA/tTA | WT/MINOR3 | <0.01 | <0.01 | <0.01 | <0.01 |
| CTA/CcA | WT/MINOR4 | <0.01 | <0.01 | <0.01 | <0.01 |
| tTc/tTA | MINOR2/MINOR3 | 0.09 | <0.01 | 0.10 | <0.01 |
| CcA/tTc | MINOR4/MINOR2 | 0.09 | <0.01 | 0.10 | <0.01 |
| tTc/tTc | MINOR2/MINOR2 | 0.09 | 0.09 | 0.10 | 0.08 |
| | WT/WT | <0.01 | 0.73 | <0.01 | 0.67 |
| | WT/MINOR | 0.73 | 0.18 | 0.70 | 0.25 |
| | MINOR/MINOR | 0.27 | 0.09 | 0.30 | 0.08 |
| TOTAL | ALL/ALL | 1 | 1 | 1 | 1 |

Table 4-6 shows counts of haplotype pairs for patients based on their SGOT response to Atorvastatin. The haplotype pair is indicated in column 1, and these haplotypes are designated as wild type (WT) or MINOR in haplotype 2 based on their frequencies in the total population. Two 2-class groupings are presented; patients whose post-Atorvastatin reading was greater than the baseline, or not greater than baseline (columns 3 and 4, respectively), and patients whose post Atorvastatin reading was over 25% greater than baseline or not over 25% greater than baseline (columns 5 and 6, respectively).

TABLE 4-7

| | | | MINOR | MINOR | MINOR | |
|---|---|---|---|---|---|---|
| | | WT | 1 | 2 | 3 | |
| | | CTA | CTc | tTc | tTA | CcA |
| WT | CTA | (−0.23) | 0.25 (9) | 0.52 (1) | nobs | Nobs |
| | | (8) | | | | |
| MINOR | CTc | | | nobs | nobs | Nobs |
| 1 | | | | | | |
| MINOR | tTc | | | 0.59 (2) | 0.25 (1) | nobs |
| 2 | | | | | | |
| MINOR | tTA | | | | nobs | nobs |
| 3 | | | | | | |
| MINOR | CcA | | | | | nobs |
| 4 | | | | | | |

Table 4-7. shows the average SGOT increase or decrease for Atorvastatin patients with various haplotype combinations. Letters in bold indicate increases. The amount of change is indicated as the average percent of change of each individual of the haplotype class relative to their baseline.

C. Discussion

A three locus CYP2D6 haplotype system is disclosed herein that can classify patients based on their proclivity to respond to Atorvastatin with SGOT elevations. Such classifications can be obtained, for Example, by calculating the Bayesian maximum likelihood estimators of a correct classification (the posterior probability), using the frequency of each haplotype in the various classes as a prior probability. Almost half of Atorvastatin patients responded to the drug with an absolute increase in SGOT readings. The frequency of this response event was in line with the SNP and haplotype frequencies observed previously, and confirm that the presence of a minor haplotype using this 3 locus system is predictive for adverse SGOT response to Atorvastatin; the frequency of the adverse event and the associated haplotypes should be similar if the association can be used to explain most of the SGOT variation in the Atorvastatin patient population.

CYP2D6 was not previously known to be involved in the adverse disposition of Atorvastatin in humans or any model system, and the only report had implicated CYP2D6 as relevant to Atorvastatin disposition used a hepatocyte model system (Cohen et al., Cohen L H, van Leeuwen R E, van Thiel G C, van Pelt J F, Yap S H. Equally potent inhibitors of cholesterol synthesis in human hepatocytes have distinguishable effects on different cytochrome P450 enzymes. Biopharm Drug Dispos Dec. 21, 2000 (9):353-3642000). CYP3A4, not CYP2D6, is considered to be the major metabolizer of Atorvastatin. Since specific CYP2D6 variants have unique substrate specificities, and since the haplotypes disclosed herein incorporate novel CYP2D6 polymorphisms, the association between CYP2D6 haplotypes and Atorvastatin response may not have been previously observed because the component SNPs of this particular haplotype were not studied and/or they are not in linkage disequilibrium with the known CYP2D6 pharmaco-relevant alleles. Within the general population the three loci are in LD, and the present results show that haplotypes incorporating these loci are not independently distributed among the two classes of SGOT responders to Atorvastatin. That the SNP at locus 1 is a dramatic coding change (from a Proline to a Serine), suggests that the haplotype variants we describe comprise an evolutionarily related cluster of haplotypes that are functionally deterministic for the phenotypic variance in SGOT response. An alternative explanation is that the present haplotype system is tracking the presence of unseen aetiological variant(s) through linkage disequilibrium. Whether the disclosed markers are in LD with previously defined poor/ultra-metabolizer CYP2D6 alleles is not yet known. However, the presence of a dramatic coding change in the present haplotype solution indicates that new CYP2D6 variants with pharmacological relevance have been defined.

The fact that these alleles have not yet been implicated as pharmacologically relevant may follow from their irrelevance to drug efficacy, which is the benchmark end-point of most pharmacogenetic studies. In support of this position, a completely independent distribution of the haplotype isoforms described here was observed between groups of Atorvastatin (and other Statin) patients stratified based on overall total cholosterol (TC) response, clinically significant TC response, overall LDL, clinically relevant LDL, HDL and triglyceride responses. The variants disclosed herein, therefore, likely directly contribute towards a minor metabolic pathway(s) that results in a very specific idiosyncratic response in some Atorvastatin patients.

The fact that the relationship is highly specific for SGOT response in Atorvastatin patients is sensible in light of what is known about the substrate and pathway specificity of variant xenobiotic metabolizer loci. Further, the association appears to be quantitative in nature. The average increase in SGOT readings in persons with a wild-type haplotype and a minor haplotype is lower than the average increase in persons with two minor haplotypes. Considering the group of patients with a minor allele at locus 1 of the system, there is good correlation between the magnitude of SGOT elevation and the total number of minor alleles present in individual diploid pairs of haplotypes. The present results showed that individuals with haplotypes containing a minor allele at locus 1 have the most dramatic elevations in SGOT response, whereas individuals with haplotypes containing a minor allele only at locus 3 had more modest responses. It is interesting to note in light of these results that locus 1 involves a dramatic Proline to Serine substitution, while that at locus 3 is in an intron. The quantitative nature of the association, the approximate match of the frequency of adverse SGOT responders with the associated allele frequencies, and the correlation between the severity of the amino acid change and magnitude of SGOT response effect, all combine to support our conclusions and lend credence to the following assertion: the posterior probability that a patient will respond to Atorvastatin with elevated SGOT readings is a function of the composite uniqueness of that patients CYP2D6 haplotype pair, as measured within the context of the minor allclcs as disclosed herein.

In its current form, the data is strictly predictive for SGOT response to Atorvastatin in the Caucasian population. It will be informative to extend these results to other ethnic groups. The present study was a retrospective case-controlled study, which can be extended to a larger, randomized prospective study. Prospective data can define the extent to which a predictive test incorporating these markers help prospective Atorvastatin patients avoid elevated SGOT responses, and can help further define the role of these markers in more serious hepatocellular responses such as injury and/or active disease. In its present form, however, the present results can be useful for excluding prospective patients from Atorvastatin treatment based on their proclivity to respond to the drug with increased SGOT levels. Because the long term health consequences of Atorvastatin induced hepatic abnormalities are part of a continuum of hepatic pathology, patients with the minor haplotypes disclosed herein would appear to be better suited for alternative medications and/or lifestyle changes to control their total cholesterol levels and/or HDL risk.

### EXAMPLE 5

### Composite Solution for Statin Efficacy

This solution for Statin efficacy incorporates several SNPs, each of which independently show an association with the degree to which a patient responds favorably to Atorvastatin and/or Simvastatin.

In general, the methods of Example 2 were used for the present Example. In order to determine whether variable patient response to Atorvastatin (Lipitor™) and Simvastatin (Zocor™) was a function of HMGCR and CYP3A4 haplotype sequences, a “vertical” re-sequencing effort was conducted in order to identify the common SNP and haplotype variants for the two genes. Gene specific primers were designed to flank each promoter, exon and 3′UTR and used these primers to amplify these regions in 500 multi-ethnic donors; 25 and 23 SNPs were identified for the HMGCR and CYP3A4 genes, respectively (Table 5-1). Surprisingly, none of these SNPs were previously known from the literature or the NCBI dbSNP resource (Gonzalez et al., Nature 331: 442-446, (1988); Rebbeck et al., J. Natl. Cancer Inst. 90:1225 (1998); Westlind et al., Biochem. Biophys. Res. Commun. 27:201 (1999); Kuehl et al., Nat. Genet. 27:383 (2001); Sata et al., 2000; Hsieh et al., 2001. Of the 48 SNP positions surveyed for these three genes, two SNPs were identified at the HMGCR locus (Table 1, SEQ ID NO:2, and SEQ ID NO:3), and two SNPs at the CYP3A4 locus (Table 1, SEQ ID NO:8, and SEQ ID NO:9) that contain predictive value for whether a patient will respond to Atorvastatin or Simvastatin with an absolute decrease in total cholesterol (TC) levels. In addition, a third SNP at the CYP3A4 locus that improved the solution (Table 1; SEQ ID NO:7) was identified that improved the solution.

TABLE 5-1

| | | Validated | | |
|---|---|---|---|---|
| | Candidate | SNPs | Publicly | |
| Gene | SNPs | f > 0.005 | Available | Overlap |
| HMGCR | 25 | 18 | 6 | 0 |
| CYP3A4 | 23 | 16 | 21 | 0 |

Table 5-1. provides a summary of SNP discovery results from the vertical re-sequencing effort. The number of candidate SNPs identified and validated variants are shown in Columns 2 and 3. The number of SNPs available from the literature or the NCBI dbSNP database are indicated in Column 4 and the overlap between the two sets of SNPs for each gene is shown in Column 5.

Of the 189 patients genotyped at these four SNPs, 77 were Caucasians who were, or had been treated with Atorvastatin or Simvastatin, for whom clinical baseline and end point measurements were available (total cholesterol—TC, low density lipoprotein—LDL, high density lipoprotein HDL), and for whom there were no missing data for any of the four loci. Another 76 individuals were Caucasians controls for whom there were no missing genotype data (Human Polymorphism Discovery Resource, Coriell Institute, N.J.). and the combined collection of genotyped Caucasians was used to infer haplotypes using software performing the algorithm of Stephens and Donnelly (2001). Haplotypes were then counted and frequencies estimated (Table 5-2). We found that the TG haplotype was the most frequent (95%) version of the HMGCRA haplotype and the GC haplotype the most frequent CYP3A4A haplotype allele version in the Caucasian population (88%).

In order to determine whether the HMGCRA and/or CYP3A4A haplotypes were associated with Statin response, a case-controlled concordance study was conducted. The distribution of haplotypes within each gene was analyzed between responders and non-responders for each of the two genes alone and in combination. Responders were defined in terms of LDL or TC change, using two different criteria of change for each—a 1% decrease in the reading or a 20% decrease. Patients were electronically phenotyped for response to the drug using the latest relevant reading before prescription, and the earliest relevant reading after prescription, and partitioned into two groups; responders and non-responders. The population of haplotypes within the 1% or 20% decrease group (the responder group) was then statistically compared to the population of haplotypes that were not (the non-responder group).

The results for the analysis at the single gene level show that HMGCRA haplotype alleles were not independently distributed between the 20% Atorvastatin responder and non-responder groups (P=0.03814+−0.00195) (row 1, Table 5-3). In contrast, the CYP3A4 haplotype alleles were independently distributed between the same two groups (row 3, Table 5-3). For Simvastatin; CYP3A4 haplotype alleles were not independently distributed between the 1% LDL responder groups (row 8, Table 5-3). Overall, the data analyzed at the level of the single gene suggests that certain haplotypes of these two genes are associated with responders and/or non-responders. The results at the single gene level were less impressive; positive results for the 1% responder stratification did not always extend to the 20% responder comparison for the same drug.

Individuals were next considered in terms of diploid pairs of CYP3A4 and HMGCR haplotype alleles. Diploid haplotype alleles for the patients were counted for the responder and non-responder groups (using the 1% decrease criteria). The results for the HMGCR gene haplotype alleles are shown in Table 5-3, and those for the CYP3A4 haplotype alleles are shown in Table 5-4. The ratio of HMGCR TG/TG non-responders to responders was 1:2.3 for Atorvastatin patients, and was 1:4 for Simvastatin patients. The results for these counts show that most individuals with the TG haplotype allele (the major haplotype) for the HMGCRA haplotype (18/26 for Atorvastatin, 35/40 for Simvastatin) were responders (rows 1, 6, 11, Table 5-4). In contrast, individuals with one copy of a minor haplotype allele (CG or TA for the HMGCR gene, and GT, AT, AC for CYP3A4) were equally likely to be responders or non-responders using the 1% criteria. For both drugs, patients harboring only one copy of the TG haplotype (TG/CG and TG/TA) showed a reduced tendency to respond favorably to the drug. For example, 5 of 20 non responders had minor HMGCR haplotypes (rows 13,14, Column 3, Table 5-4) whereas 3 of 56 responders had minor HMGCR (same rows, Column 4, Table 5-4) haplotypes.

TABLE 5-2
Haplotype frequencies for HMGCRA and CYP3A4A haplotypes.

| GENE | HAPLOTYPE | FREQUENCY |
|---|---|---|
| HMGCR | TG | 0.95 |
| HMGCR | CG | 0.02 |
| HMGCR | TA | 0.03 |
| HMGCR | CA | n/o |
| CYP3A4 | GC | 0.88 |
| CYP3A4 | GT | <0.01 |
| CYP3A4 | AT | <0.01 |
| CYP3A4 | AC | 0.11 |

TABLE 5-3
HMGCR and CYP3A4 haplotype frequencies in the Caucasian population (n = 153).

| TEST | GENE | DRUG | PW dist F | PW P value | Slatkin | Exact P |
|---|---|---|---|---|---|---|
| LDL20 | HMGCR | Atorvastatin | 0.1707 | 0.04505 +− 0.0203 | 0.20584 | 0.03814 +− 0.00195 |
| LDL1 | HMGCR | Atorvastatin | 0.06299 | 0.14414 +− 0.0309 | 0.06722 | 0.10281 +− 0.00283 |
| LDL20 | CYP3A4 | Atorvastatin | 0.04892 | 0.10811 +− 0.0264 | 0.05144 | 0.28163 +− 0.00545 |
| LDL1 | CYP3A4 | Atorvastatin | 0.06283 | 0.14414 +− 0.0454 | 0.06704 | 0.13118 +− 0.00605 |
| LDL20 | HMGCR | Simvastatin | N/S | N/S | N/S | N/S |
| LDL1 | HMGCR | Simvastatin | N/S | N/S | N/S | N/S |
| LDL20 | CYP3A4 | Simvastatin | 0.01025 | 0.48649 +− 0.0411 | 0 | 0.28498 +− 0.00563 |
| LDL1 | CYP3A4 | Simvastatin | 0.09427 | 0.00901 +− 0.0091 | 0.10408 | 0.08077 +− 0.00212 |
| LDL20 | HMGCR | Pravastatin | N/S | N/S | N/S | N/S |
| LDL1 | HMGCR | Pravastatin | N/S | N/S | N/S | N/S |
| LDL20 | CYP3A4 | Pravastatin | N/S | N/S | N/S | N/S |
| LDL1 | CYP3A4 | Pravastatin | N/S | N/S | N/S | N/S |
| LDL20 | HMGCR | Artorv + Simv | 0.20085 | 0.00901 +− 0.0091 | 0.25132 | 0.01348 +− 0.00106 |
| LDL20 | CYP3A4 | Artorv + Simv | 0.00148 | 0.34234 +− 0.0379 | 0.00148 | 0.61056 +− 0.00446 |
| LDL1 | HMGCR | Artorv + Simv | 0.05523 | 0.05405 +− 0.0148 | 0.05845 | 0.07616 +− 0.00246 |
| LDL1 | CYP3A4 | Artorv + Simv | 0.25581 | 0.00000 +− 0.0000 | 0.34375 | 0.00105 +− 0.00022 |

Table 5-3. shows haplotype distributions between responders and non-responders for Atorvastatin, Simvastatin and Pravastatin. (as indicated in Column 3). The test is shown in Column 1 (LDL) with a number following the test to indicate the criteria for stratifying the population. For Example, for LDL1, responders were defined as individuals who exhibited a decrease in post-prescription LDL levels by greater than 1% compared to the baseline for a given patient, and non-responders were defined as individuals who did not exhibit this change in post-prescription LDL levels compared to the baseline for a given patient. The Pair Wise F—statistic is shown along with its P value in Columns 4 and 5. The Slatkin statistic is shown in Column 6 and the P value from the Exact test of non-differentiation is shown in Column 7. N/S means there was not a sufficient sample size to obtain meaningful results. Results for TC levels were essentially the same (not shown).

TABLE 5-4

TC CHANGE

| | HMGCR | UP or | |
|---|---|---|---|
| DRUG | HAPLOTYPES | SAME | DOWN |
| Atorvastatin | TG/TG | 8 | 18 |
| Atorvastatin | TG/CG | 0 | 1 |
| Atorvastatin | TG/TA | 4 | 0 |
| | ALL | 12 | 19 |
| Simvastatin | TG/TG | 5 | 35 |
| Simvastatin | TG/CG | 1 | 1 |
| Simvastatin | TG/TA | 0 | 1 |
| | ALL | 6 | 37 |
| Both | TG/TG | 15 | 53 |
| Both | TG/CG | 1 | 2 |
| Both | TG/TA | 4 | 1 |
| | ALL | 20 | 56 |

Table 5-4. shows HMGCR haplotype combinations in patients with different responses to Atorvastatin (Lipitor™) or Simvastatin (Zocor). The Drug is indicated in column one, and the haplotype counts are indicated in columns 4 and 5 for the three different haplotype combinations observed (column 2).

TABLE 5-5

TC CHANGE

| | CYP3A4 | UP or | |
|---|---|---|---|
| DRUG | HAPLOTYPES | SAME | DOWN |
| LIPITOR | GC/GC | 2 | 15 |
| LIPITOR | GT/AT | 0 | 1 |
| LIPITOR | GC/AC | 3 | 3 |
| | ALL | 5 | 19 |
| ZOCOR | GC/GC | 2 | 30 |
| ZOCOR | GT/AT | 0 | 0 |
| ZOCOR | GC/AC | 3 | 4 |
| | ALL | 5 | 34 |
| TOTAL | GC/GC | 4 | 45 |
| TOTAL | GT/AT | 0 | 1 |
| TOTAL | GC/AC | 6 | 7 |
| | ALL | 10 | 53 |

Table 5-5. shows CYP3A4 haplotype combinations in patients with different responses to Atorvastatin (Lipitor™) or Simvastatin (Zocor™). The Drug is indicated in column one, and the haplotype counts are indicated in columns 4 and 5 for the three different haplotype combinations observed (column 2).

For the CYP3A4 gene, most of the individuals with the GC CYP3A4 haplotype (15/17 for Atorvastatin (Lipitor™) and 30/32 for Simvastatin (Zocor™)) were responders (rows 1,6,11, Table 5-5). Atorvastatin and Simvastatin patients (considered together) who were homozygous for the major GC haplotype (the major haplotype) responded to the drug with decreased TC levels 92% of the time, but patients with only one copy of the GC haplotype and a copy of one of the minor haplotypes responded to the drug with decreased TC levels only 43% of the time. In all, 6 of 10 individuals with a minor CYP3A4 haplotype were non-responders for both drugs considered jointly, whereas only 8 of 53 were responders. Some predicted haplotype pairs were not observed in this analysis, presumably due to their low frequencies in the population.

When genotypes of patients is considered at both genes jointly in each patient, a very clear trend becomes apparent. The haplotypes were encoded as wild-type and minor based on their frequencies shown in Table 5-2, and then combined the results in a bivariate analysis (Table 5-6). The results of this comparison showed that, for both drugs, the presence of a diploid pair of major HMGCR haplotypes, combined with a diploid pair of major CYP3A4 haplotypes, was strongly associated with the expected therapeutic response (a decrease in TC levels) for both drugs. Table 5-5 shows the break-down for each drug, and then for both drugs combined. Nine of eleven Atorvastatin patients who did not respond to the drug contained at least one minor haplotype in either the HMGCR or CYP3A4 gene. In contrast only 2 of 18 Atorvastatin patients who did respond had a minor haplotype for either of these genes. For Simvastatin, 4 or 6 non-responders had at least one minor haplotype at one of the genes, but only 2 of 36 responders had a minor haplotype.

When considering both drugs together 13/17 non responders harbored a minor haplotype but only 4/56 responders had a minor haplotype, and 4/17 non responders harbored a diploid pair of major haplotypes, but 52/56 responders harbored a diploid pair of major haplotypes. Using the presence of a minor haplotype in either gene as a criteria for classifying an unknown individual as a potential non-responder to Atorvastatin or Simvastatin yielded an accuracy of 93% for responders and 76% for non-responders. The total accuracy of this classification tool can vary depending on the genotype of the individual but, for all genotypes, was about 90% (Table 5-9). The use both genes in the solution yielded a better result than either gene alone, as evidenced by comparing the accuracy of classification using the HMGCR gene alone (Table 5-7), the CYP3A4 gene alone (Table 5-8) or both (Table 5-9).

For calculating the effect statistics of this solution, the total number of patients (73) was used as the fixed variable. The probability of an individual containing no minor haplotype in either gene not responding to either drug is 4/73=0.0547 (confidence interval 0.0025 to 0.1069). The probability of the same individual responding (based on TC levels) to either drug is 52/73=0.7123 (CI 0.6085 to 0.8161). For individuals with one minor haplotype, the probability of not responding to these drugs (based on TC levels) is 0.1780 (CI 0.0902 to 0.2658) and the probability of the individual responding is 0.0548 (CI 0.0026 to 0.1070). The soundness of using the presence of a minor haplotype to classify individuals based on their proclivity to respond to these drugs (based on TC levels) can be measured from this data using a T test. Comparing the statistics with a T test yields a significance of P<0.0001.

Lastly, a third SNP at the CYP3A4 locus that improved the solution (Table 1; SEQ ID NO:7) was identified that improved the solution.

TABLE 5-6
CYP3A4 + HMGCR TOGETHER (NUMBER OF EVENTS)

| | | TC CHANGE | |
|---|---|---|---|
| | HMG AND 3A4 | SAME OR | DE- |
| DRUG | HAPLOTYPES | INCREASE | CREASE |
| Atorvastatin | (wt/wt and wt/wt) | 2 | 18 |
| Atorvastatin | (wt/wt) and (wt/— or —/—) | 6 | 0 |
| Atorvastatin | (wt/— or —/—) and (wt/wt) | 3 | 2 |
| Atorvastatin | (wt/— or —/—) and (wt/— or | 0 | 0 |
| | —/—) | | |
| Simvastatin | (wt/wt and wt/wt) | 2 | 34 |
| Simvastatin | (wt/wt) and (wt/— or —/—) | 3 | 0 |
| Simvastatin | (wt/— or —/—) and (wt/wt) | 1 | 2 |
| Simvastatin | (wt/— or —/—) and (wt/— or | 0 | 0 |
| | —/—) | | |
| BOTH | (wt/wt and wt/wt) | 4 | 52 |
| BOTH | (wt/wt) and (wt/— or —/—) | 9 | 0 |
| BOTH | (wt/— or —/—) and (wt/wt) | 4 | 4 |
| BOTH | (wt/— or —/—) and (wt/— or | 0 | 0 |
| | —/—) | | |
| BOTH | no minor haplotypes | 4 | 52 |
| BOTH | at least one minor haplotype | 13 | 4 |

Table 5-6. shows counts of HMGCR and CYP3A4 haplotype combinations in Atorvastatin and Simvastatin patients that showed a therapeutic response (DECREASE, Column 4) or did not show a therapeutic response (SAME OR INCREASE, Column 3). The haplotypes are encoded as wild type (wt) or minor (−−) depending on their frequencies shown in Table 5-2. The combination of haplotype pairs is shown in Column 2, with the encoded diploid genotype of haplotypes for the HMGCR gene in the first set of parentheses and the encoded diploid genotype of haplotypes for the CYP3A4 gene in the second set of parenthesis of the line. A further condensation of the data is shown in the last two rows, where patients are grouped based on the presence (or lack thereof) of a minor haplotype for either of the two genes.

TABLE 5-7
RULE: presence of HMGCR minor haplotype
TA predicts inefficacious response

correctly classified

| DRUG | count | percent |
|---|---|---|
| ZOCOR | (36/44) | 81.80% |
| LIPITOR | (21/33) | 63.60% |
| BOTH | (57/77) | 74.00% |

Table 5-7. shows the accuracy of classifying a patient as a potential non-responder based on the presence of a minor HMGCR haplotype.

TABLE 5-8
RULE: presence of CYP3A4 minor haplotype
AC predicts inefficacious response

correctly classified

| DRUG | count | percent |
|---|---|---|
| ZOCOR | (33/39) | 84.60% |
| LIPITOR | (18/23) | 78.30% |
| BOTH | (51/61) | 80.90% |

Table 5-8. shows the accuracy of classifying a patient as a potential non-responder based on the presence of a minor CYP3A4 haplotype.

TABLE 5-9
RULE: presence of HMG minor haplotype TA and/or presence of
CYP3A4 minor haplotype AC predicts inefficacious response

correctly classified

| DRUG | count | percent |
|---|---|---|
| ZOCOR | (38/42) | 90.50% |
| LIPITOR | (27/31) | 87.10% |
| BOTH | (65/73) | 89.04% |

Table 5-9. shows the accuracy of classifying a patient as a potential non-responder based on the presence of a minor HMGCR haplotype or a minor CYP3A4 haplotype.

### EXAMPLE 6

### Genetic Solution for a Lipitor™ Response

This Example identifies haplotypes in the CYP3A4 gene that are related to a response to Lipitor™. The methods used are those generally described in Example 2 along with primers as listed in Table 4 for the SNPs described herein. Briefly, a set of algorithms was used to identify the best genetic features for resolving the various trait classes, and then modeled these features in order to construct a genetic classifier. In order to find the genetic features, patients were genotyped at hundreds of single nucleotide polymorphisms (SNPs) within xenobiotic metabolism and drug target genes, haplotype systems were defined within these genes and individual haplotypes of a given haplotype system were analyzed to determine whether they were associated with Lipitor™ response. To make this determination, individual haplotypes were counted in each of two classes: non-responders=TC levels unchanged or increased; and responders=TC levels decreased. The null hypothesis that Lipitor™ response was not associated with specific haplotypes of a given haplotype system, was tested by performing a Pearson's Chi-square and Fisher's exact test on haplotype counts.

SNP combinations in 24 genes were screened for the ability of their constituent haplotype alleles to “explain” Lipitor™ response; to resolve Lipitor™ patients based on the percent increase or decrease in total cholesterol (TC) levels. Of 1,434 candidate haplotype systems defined for these 24 genes, alleles of the CYP3A4C haplotype system (Table 6-1) were found to be the best at resolving patients based on their response to Lipitor™ (percent increase or decrease in total cholesterol (TC) levels; FST P=0.036+/−0.020) (Table 6-2 and Table 6-3). The ATGC haplotype was the most frequent in the patient population. While ATGC/ATGC individuals responded to Lipitor™ with decreases (“DECR”, Table 6-2) in TC levels 34 of 40 times (85%), individuals with other haplotype combinations responded only 14 of 26 times (54%).

TABLE 6-1
The composition of the haplotype systems discussed in the text.

| HAPLOTYPE | MARKER | MARKER | MARKER | MARKER |
|---|---|---|---|---|
| SYSTEM | 1 | 2 | 3 | 4 |
| CYP3A4C | 809114 | 664803 | 712037 | 869772 |
| HMGCRB | 809125 | 712050 | 712044 | 664793 |

TABLE 6-2

| | Change in Total Cholesterol | | | | | |
|---|---|---|---|---|---|---|
| CYP3A4C | >5% | <5% | <5% | 5-10% | 10-20% | >20% |
| GENOTYPE | INCR | INCR | DECR | DECR | DECR | DECR |
| ATGC/ATGC | 4 | 2 | 5 | 3 | 8 | 18 |
| ATGC/ATAC | 1 | 1 | 1 | 1 | 1 | 3 |
| ATGC/AGAT | 2 | 0 | 0 | 0 | 0 | 2 |
| AGAC/ATGC | 1 | 0 | 1 | 2 | 1 | 0 |
| ATAT/ATGC | 0 | 0 | 0 | 0 | 0 | 1 |
| ATGT/AGAT | 0 | 0 | 0 | 0 | 0 | 1 |
| TGAC/ATGC | 1 | 0 | 0 | 0 | 0 | 0 |

Table 6-2. CYP3A4C genotype counts of Lipitor™ patients exhibiting various responses. Response is measured in terms of post-prescription total cholesterol (TC) increase (INCR) or decrease (DECR) relative to baseline. Genotypes are diploid pairs of haplotypes shown in the first column, and the various responses are shown across the top of the table from poorest response (far left; >5%INCR) to best response (>20%DECR, far right).

The significance of these counts of Total Cholesterol (TC) changes, as well as counts of Low Density Lipoprotein (LDL) changes in Lipitor™ patients was tested. For statistical analysis of the data a one-sided, paired t-test was used. The hypothesis that there is no effect of the drug in decreasing low cholesterol level (1 dl) was tested for each genotype. i. e., the mean of difference (1 dl level before drug−1 dl after drug) in cholesterol (1 dl) in each genotype group is zero (Table 6-3).

TABLE 6-3
Gene-CYP3A4, Marker: 809114|664803|712037|869772;
Drug-Lipitor ™; Test-LDL

| Genotype | n | D bar (+/−SE(d bar)) | | t₀ | p |
|---|---|---|---|---|---|
| 1. ATGC/ATGC | 38 | 32.6779 | (+/−08.3668) | 3.90* | 0.0002 |
| 2. ATGC/ATAC | 5 | 61.6000 | (+/−13.4559) | 4.58* | 0.0051 |
| 3. ATGC/AGAC | 5 | −5.2000 | (+/−05.8600) | −0.89 | 0.2125 |
| 4. ATGC/AGAT | 4 | 30.2500 | (+/−25.9049) | 1.17 | 0.1637 |
| 5. ATGC/ATAT | 1 | 09.0 | (−) | — | — |
| 6. ATGC/TGAC | 1 | −17.0 | (−) | — | — |
| 7. ATGT/AGAT | 1 | 65.0 | (−) | — | — |
| 8. Total | 55 | 30.9272 | (+/−6.5152) | 4.75* | <0.00005 |

Table 6-3. Summary statistics for Lipitor™ response (as measured by LDL change) within genotype classes of the CYP3A4C haplotype system. The genotype is shown on the far left, the number of patients in the second column (“n”), the average response in the third column, an effect statistic and associated p-value in the last two columns.

The result of this analysis indicate that there is an effect of the drug Lipitor™ in decreasing LDL cholesterol level in individuals with the ATGC/ATGC and ATGC/ATAC genotypes only. The effect on all patients is highly significant (<0.00005, row 8, Table 6-3), but the response seems to be focused in individuals of ATGC/ATGC and ATGC/ATAC genotypes. The mean of difference (before test date-after test date) in LDL cholesterol for individuals of the ATGC/ATGC and ATGC/ATAC genotypes are 32.6779 and 61.6000 respectively indicating that the LDL reductions are highly significant. In the case of other genotypes, ATGC/AGAT, ATGC/AGAT and ATGT/AGAT the decrease is not significant, and in the case of ATGC/AGAC and ATGC/TGAC, the average LDL response is actually an increase. (*=significant.)

Next, the null hypothesis that there is no effect of drug in decreasing total cholesterol level (TC) in each genotype was tested. In other words, whether the mean of difference in TC levels (TC level before Lipitor™−TC level post Lipitor™) was zero for each genotype group (H0=d bar=0 against H1:d bar>0) (Table 6-4) was tested.

TABLE 6-4
Gene-CYP3A4, Marker: 809114|664803|712037|869772;
Drug-Lipitor ™; Test-TC

| Genotype | n | d bar (+/−SE(d bar)) | | t₀ | p |
|---|---|---|---|---|---|
| 1. ATGC/ATGC | 41 | 31.8537 | (+/−08.8656) | 3.59* | 0.0005 |
| 2. ATGC/ATAC | 8 | 48.8750 | (+/−15.1344) | 3.23* | 0.0073 |
| 3. ATGC/AGAC | 5 | 09.2000 | (+/−10.89681) | 0.84 | 0.223 |
| 4. ATGC/AGAT | 4 | 23.5000 | (+/−32.6713) | 0.72 | 0.2619 |
| 5. ATGC/ATAT | 1 | 49.0 | (−) | — | — |
| 6. ATGC/TGAC | 1 | −13.0 | (−) | — | — |
| 7. ATGT/AGAT | 1 | 66.0 | (−) | — | — |
| 8. Total | 61 | 31.7868 | (+/−6.7254) | 4.73* | <0.00005 |

Table 6-4. Summary statistics for Lipitor™ response (as measured by TC change) within genotype classes of the CYP3A4C haplotype system. The genotype is shown on the far left, the number of patients in the second column (“n”), the average response in the third column, an effect statistic and associated p-value in the last two columns. (*=significant)

The results of this analysis indicate that there is an effect of the drug Lipitor™ in decreasing low cholesterol level in individuals with the ATGC/ATGC and ATGC/ATAC genotypes only. The effect on all patients is highly significant (<0.00005, row 8, Table 6-4), but the response seems to be focused in individuals of ATGC/ATGC and ATGC/ATAC genotypes. The mean TC decrease in these groups was 31.8537 and 48.875 respectively. The other genotypes with one minor allele, ATGC/AGAC, ATGC/AGAT, ATGC/ATAT, and ATGT/AGAT, the decrease in TC is not significant. This result was the same result obtained using TC levels as the indicator of Lipitor™ response.

In addition to the first haplotype system within the CYP3A4 described above, a second haplotype system, (HMGCRB, Table 6-1), this one in the HMGCR gene was identified. A total of two genetic features were identified in the HMGCR gene as capable of statistically resolving between Lipitor™ responders and non-responders. HMGCRB was discovered as the optimal haplotype system capable of resolving Lipitor™ responders and non-responders from a screen of 1,110 possible HMGCR SNP combinations in Lipitor™ patients. HMGCR is the molecular target for the Statin class of drugs. The null hypothesis (Ho) was tested for a genetic dependence between Lipitor™ response as measured with LDL readings, and HMGCRB genotypes (Table 6-5) or TC levels (Table 6-6).

First, the null hypothesis (Ho) that the LDL response to Lipitor™ was not associated with any particular HMGCRB genotype, was tested. In other words, whether the mean LDL difference (LDL level before Lipitor™−LDL level post Lipitor™) in Lipitor™ patients of the various genotype groups is zero, was tested (i.e. H0=d bar=0 against H1:d bar>0).

TABLE 6-5
Gene-HMGCR, Haplotype System: 809125|712050|712044|664793;
Drug-Lipitor ™; Test-LDL

| Genotype | n | D bar (+/−SE(d bar)) | | t₀ | p |
|---|---|---|---|---|---|
| 1. CGTA/CGTA | 42 | 32.9524 | (+/−06.5438) | 5.04* | <0.00005 |
| 2. CGTA/TGTA | 7 | 39.5714 | (+/−15.6948) | 2.52* | 0.0225 |
| 3. CGTA/CGCA | 3 | −24.3333 | (+/−52.4796) | −0.46 | 0.3442 |
| 4. CGTA/CGTC | 3 | 12.6667 | (+/−22.1008) | 0.57 | 0.3122 |
| 5. CGTA/CATA | 1 | 1. | (−) | — | — |
| 6. Total | 56 | 29.0179 | (+/−6.1161) | 4.74* | <0.00005 |

Table 6-5. Summary statistics for Lipitor™ response (as measured by LDL change) within genotype classes of the HMGCRB haplotype system. The genotype is shown on the far left, the number of patients in the second column (“n”), the average response in the third column, an effect statistic and associated p-value in the last two columns. (*significant.).

The results show a highly significant response to Lipitor™ in the patient population (“Total”, Row 7, Table 6-5). Specifically, Lipitor™ appears to affect a decrease in low cholesterol level for individuals of the CGTA/CGTA and CGTA/TGTA genotypes. The mean difference in LDL levels before the drug versus after the drug in individuals of the CGTA/CGTA and CGTA/TGTA genotypes are 32.9524 and 39.5714, respectively. These reductions are found to be highly significant (P<0.00005 and P=0.0225, respectively). The other genotypes, CGTA/CGCA, CGTA/CGTC and CGTA/CATA showed average LDL responses that were not significantly reduced by treatment. Individuals with the CGTA/CGCA actually showed an average increase in LDL levels after commencing Lipitor™ therapy.

The same result obtained when TC response was used instead of LDL response. The null hypothesis (Ho) that there was no effect of drug in decreasing total cholesterol level (tc) (i.e., the mean difference (before test date−after test date)) in cholesterol (tc) in each genotype group is zero, was tested (i.e. H0=d bar=0 against H1:d bar>0 (Table 6-6)).

TABLE 6-6
Gene-HMGCR, Marker: 809125|712050|712044|664793;
Drug-Lipitor ™; Test-TC

| Genotype | n | d bar (+/−SE(d bar)) | | t₀ | p |
|---|---|---|---|---|---|
| 1. CGTA/CGTA | 46 | 39.1957 | (+/−06.4773) | 6.05* | <0.00005 |
| 2. CGTA/TGTA | 7 | 34.7143 | (+/−16.7143) | 2.07* | 0.0416 |
| 3. CGTA/CGCA | 3 | −33.6667 | (+/−74.3064) | −0.45 | 0.3475 |
| 4. CGTA/CGTC | 3 | 13.6667 | (+/−24.3949) | 0.56 | 0.3159 |
| 5. CGTA/CATA | 2 | 35.5000 | (+/−36.5000) | 0.97 | 0.2590 |
| 6. Total | 61 | 33.6885 | (+/−06.4900) | 5.19* | <0.00005 |

Table 6-6. Summary statistics for Lipitor™ response (as measured by TC change) within genotype classes of the HMGCRB haplotype system. The genotype is shown on the far left, the number of patients in the second column (“n”), the average response in the third column, an effect statistic and associated p-value in the last two columns.(*significant).

The results show a highly significant response to Lipitor™ in the patient population (“Total”, Row 6, Table 6-6). Specifically, Lipitor™ appears to affect a decrease in low cholesterol level for individuals of the CGTA/CGTA and CGTA/TGTA genotypes. The mean of difference (before drug TC levels−post drug TC levels) for individuals with the CGTA/CGTA and CGTA/TGTA genotypes were 39.1957, 34.7143 and were found to be significantly reduced. The other genotypes, CGTA/CGCA, CGTA/CGTC and CGTA/CATA showed average TC responses that were not significantly reduced by treatment.

Feature Modeling for the Development of a Lipitor™ Classifier

Because the p-value for the resolution of Lipitor™ response in terms of HMGCRB haplotypes was greater than for the CYP3A4C haplotype system, the CYP3A4C haplotype system was used as the root for a classification tree analysis of variable Lipitor™ response in terms of CYP3A4C and HMGCRB haplotype pairs (genotypes). This method of modeling genetic features is described in T. Frudakis, U.S. patent application Ser. No. 10/156,995, filed May 28, 2002.

Although most CYP3A4C:ATGC/ATGC individuals responded to Lipitor™, there were several that did not. As a part of the construction process for the classification tree, CYP3A4C:ATGC/ATGC individuals were typed for haplotypes in the HMGCR gene. From the tree constructed, it was observed that the HMGCRB haplotype system effectively resolved between Lipitor™ responders and non-responders that harbored the CYP3A4C ATGC/ATGC genotype (FST P=0.081+/1+/−0.029). In contrast, haplotype systems for other genes did not show an ability to resolve between CYP3A4C:ATGC/ATGC responders and non-responders; F statistic P values for distribution of CYP2D6 haplotypes ranged, depending on the haplotype system, from 0.56 to 0.89.

The combined results from the classification tree developed using the CYP3A4 and HMGCR haplotype system features show that whereas 29 of 32 (91%) CYP3A4C:ATGC/ATGC, HMGCRB:CGTA/CGTA individuals responded to Lipitor™, only 6 of 10 (60%) CYP3A4C:ATGC/ATGC, HMGCRB:individuals responded to Lipitor™ (Table 6-5). This was a very important observation. It showed that individuals with minor haplotypes at EITHER the HMGCR or CYP3A4 genes showed a tendency not to respond to Lipitor™. For Example, consider Table 6-6 and Table 6-7, where the HMGCRB genotypes are counted for CYP3A4 ATGC/ATGC individuals (individuals who have two copies of the major CYP3A4 haplotype). Within this group, most of the non-responders harbor a minor HMGCR haplotype (not CGTA) and that the ratio of responders to non-responders is significantly lower for these individuals than for CGTA/CGTA individuals. This effect is highly specific for the HMBCRB and CYP3A4C haplotypes. Consider the CYP2D6 gene, thought to be the most prolific of the xenobiotic metabolizer genes; there is no dependence between genotypes in this gene and responses (Table 6-8). Although over 7,000 SNP combinations were tried, none of them significantly associated with response in this subgroup of patients or in Lipitor™ patients in general.

If we use “MAJOR” to indicate a major haplotype for either of the CYP3A4 or HMGCR genes with respect to the specific haplotype systems we have described; ATGC and CGTA, respectively; and “MINOR” to indicate a minor haplotype for either gene, the breakdown for the two gene analysis shows clearly that individuals that harbor two copies of a major haplotypes for both genes show a greater tendency to respond to Lipitor™ than individuals that do not.

Conclusion:

Thus, the classification tree “solution” (or the pharmacogenetic classifier) for Lipitor™ and Zocor™ response is quite simple. Table 6-8 shows the final counts. Patients who are compound homozygotes for the major CYP3A4C and HMGCRB haplotypes are responders about 91% of the time. Others respond only 66.7% of the time. Thus, if a patient is not a compound homozygote for the major CYP3A4C and HMGCRB haplotypes, they are relatively unlikely to respond favorably and may consider other treatment options. The example described here did not correct for other treatments, such as Niacin treatment (which is commonly administered in conjunction with Statins), or dietary change. It was assumed that statins were prescribed to the individuals in this study in a manner consistent with current FDA recommendations; dietary changes are almost always requested of patients. Though compliance is not possible to assess with our data, because compliance is the same regardless of which haplotype system or gene was analyzed, the finding of a haplotype system that is associated with statin response is significant notwithstanding the study participants, their diet, other medications they were taking, their sex, or their age.

TABLE 6-7

| | Total Cholesterol Increase in | | | | | |
|---|---|---|---|---|---|---|
| | CYP3A4C ATGC/ATGC individuals | | | | | |
| HMGCRB | >5% | <5% | <5% | 5-10% | 10-20% | >20% |
| GENOTYPE | INCR | INCR | DECR | DECR | DECR | DECR |
| CGTA/CGTA | 2 | 1 | 5 | 4 | 7 | 13 |
| CGCA/CGTA | 1 | 0 | 0 | 0 | 0 | 1 |
| CGTC/CGTA | 0 | 0 | 0 | 0 | 1 | 0 |
| CGTA/TGTA | 1 | 1 | 0 | 0 | 1 | 2 |
| OTHER | 1 | 0 | 0 | 0 | 0 | 1 |

Table 6-7. HMGCRB genotype counts of Lipitor™ patients with the CYP3A4C ATGC/ATGC genotype. Counts for each genotype exhibiting various total cholesterol (TC) responses {increase (INCR) or decrease (DECR) relative to baseline} are shown. Genotypes are diploid pairs of haplotypes shown in the first column, and the various responses are shown across the top of the table from poorest response (far left; >5% INCR) to best response (>20%DECR, far right).

TABLE 6-8

RESPONSE

| GENOTYPE | NEGATIVE | POSITIVE |
|---|---|---|
| CGTA/CGTA | 3 | 29 |
| CGCA/CGTA | 1 | 1 |
| CGTC/CGTA | 0 | 1 |
| CGTA/TGTA | 2 | 3 |
| OTHER | 1 | 1 |

Table 6-8. A condensation of the data presented in FIG. 5 showing HMGCRB genotype counts CYP3A4C:ATGC/ATGC patients based on Lipitor™ response. Responders are individuals who responded to Lipitor™ with a decrease in total cholesterol levels and non-responders as individuals who responded with an increase or no change in total cholesterol levels.

TABLE 6-9

| | Total Cholesterol Increase IN CYP3A4C individuals | | | | | |
|---|---|---|---|---|---|---|
| 2D6ST1105 | >5% | <5% | <5% | 5-10% | 10-20% | >20% |
| GENOTYPE | INCR | INCR | DECR | DECR | DECR | DECR |
| GTCT/GCAT | 1 | 0 | 2 | 0 | 3 | 2 |
| TTCT/GTAT | 0 | 0 | 0 | 0 | 0 | 1 |
| TCCT/GCAT | 2 | 1 | 2 | 2 | 2 | 4 |
| TTCT/GCAC | 0 | 0 | 0 | 0 | 0 | 1 |
| GTCT/TTCT | 0 | 0 | 1 | 0 | 0 | 1 |
| TCCT/TCCT | 0 | 0 | 0 | 0 | 1 | 2 |
| TTCT/GCAT | 1 | 1 | 0 | 1 | 3 | 2 |
| TTCT/TTCT | 0 | 0 | 0 | 0 | 0 | 2 |
| TCCT/TTCT | 0 | 0 | 0 | 1 | 0 | 0 |

Table 6-9. 2D6ST1105 genotype counts of Lipitor™ patients exhibiting various responses. Response is measured in terms of post-prescription total cholesterol (TC) increase (INCR) or decrease (DECR) relative to baseline. Genotypes are diploid pairs of haplotypes shown in the first column, and the various responses are shown across the top of the table from poorest response (far left; >5%INCR) to best response (>20%DECR, far right).

TABLE 6-10

RESPONSE

| GENOTYPE | NEGATIVE | POSITIVE |
|---|---|---|
| (+/+) : (+/+) | 3 | 29 |
| Other | 10 | 20 |

Table 6-10. Summary of Lipitor™ response in terms of major (+) and minor (−) CYP3A4C and HMGCRB haplotype counts. Response is measured in terms of a reduction in total cholesterol (TC) levels relative to baseline (a POSITIVE response) or an increase, or no change in TC levels relative to baseline (a NEGATIVE response).

### EXAMPLE 7

### Genetic Solution for a Zocor™ Response

This Example identifies haplotypes in the CYP3A4 gene that are related to a response to Zocor™. A similar, and even more dramatic tendency for patients taking Zocor™ was observed. SNP combinations in 24 genes were screened for association with a Zocor™ response (i.e. the ability of their constituent haplotype alleles to resolve Zocor™ patients based on the percent increase or decrease in total cholesterol (TC) and low density lipoprotein (LDL) levels). The methods used are those generally described in Example 2 along with primers as listed in Table 4 for the SNPs disclosed herein. The strategy for this analysis was identical as that already described for Lipitor™ patients in Example 6. Of the 1,434 candidate haplotype systems tested, alleles of the CYP3A4C haplotype system were the best at resolving Zocor™ patients based on their response (FST P=0.045+/−0.015) (Table 7-1). This is the same haplotype system that was identified for Lipitor™ in Example 6. The ATGC haplotype is the most frequent in the general population, and while ATGC/ATGC individuals responded to Zocor™ with decreases (DECR) in TC levels 41 of 45 times (91%), individuals with other haplotype combinations responded only 8 of 13 times (62%) (Table 7-1).

TABLE 7-1

| | Total Cholesterol Increase in Zocor patients | | | | | |
|---|---|---|---|---|---|---|
| CYP3A4C | <5% | 0-5% | <5% | 5-10% | 10-20% | >20% |
| GENOTYPE | INCR | INCR | DECR | DECR | DECR | DECR |
| ATGC/ATGC | 2 | 2 | 5 | 3 | 14 | 19 |
| AGGT/ATGC | 0 | 0 | 0 | 0 | 1 | 0 |
| ATGC/ATAC | 3 | 1 | 0 | 0 | 4 | 1 |
| AGAC/ATGC | 1 | 0 | 0 | 0 | 1 | 1 |

Table 7-1. CYP3A4C genotype counts of Zocor™ patients exhibiting various responses. Response is measured in terms of post-prescription total cholesterol (TC) increase (INCR) or decrease (DECR) relative to baseline. Genotypes are diploid pairs of haplotypes shown in the first column, and the various responses are shown across the top of the table from poorest response (far left; >5%INCR) to best response (>20%DECR, far right).

HMGCR Haplotypes and Zocor™ Response

A statistical analysis was performed of the HMGCR gene, to identify haplotypes that are associated with a response to Zocor™. A one-sided paired t-test was performed on LDL data looking at HMGCR haplotypes and a null hypothesis that there is no effect of drug in decreasing cholesterol level (LDL) in each HMGCR genotype (i.e., the mean of difference (before test date-after test date) in cholesterol (LDL) in each genotype group is zero).

TABLE 7-2

| Genotype | n | d bar (+/−SE(d bar)) | t₀ | p |
|---|---|---|---|---|
| 1. CGTA/CGTA | 42 | 41.8810 (+/−07.2364) | 5.79** | <0.00005 |
| 2. CGTA/CGTC | 6 | 27.6667 (+/−18.3805) | 1.51 | 0.0963 |
| 3. CGTA/TGTA | 4 | 43.0000 (+/−16.2327) | 2.65* | 0.0385 |
| 4. CGTA/CATA | 4 | 05.7500 (+/−19.4695) | 0.30 | 0.3935 |
| 5. CGTA/CGCA | 1 | 45.0 (−) | — | — |
| 6. Total | 57 | 37.9824 (+/−5.9657) | 6.37** | <0.00005 |

Table 7-2. Test of null hypothesis that for each HMGCRB (Marker:809125|712050|712044|664793) genotype there is no effect of Zocor™ in decreasing cholesterol (LDL) level (i.e., the mean of difference (before test date−after test date) in cholesterol (LDL) levels in each genotype group is zero)—i.e. H0=d bar=0 against H1:d bar>0. (*significant).

The analysis indicated that in the general population, the use of Zocor™ is associated with a significant (37.98, P<0.00005) response in terms of LDL readings (Row 6, Table 7-2). This decrease is related to the HMGCRB haplotype. Specifically, Zocor™ use is associated with a decrease in LDL cholesterol levels in individuals of the CGTA/CGTA and CGTA/CGTC genotypes. The mean LDL difference (before drug date-after drug date) in LDL cholesterol for individuals of the CGTA/CGTA and CGTA/TGTA genotypes are 41.8810 and 43.0, respectively. These values are significant (P<0.00005 and P=0.0385, respectively). The other genotypes, CGTA/CGTC, CGTA/CATA and CGTA/CGCA were found to not be significantly associated with LDL reduction in Zocor™ patients.

Next, a one-sided paired t-test was performed on total cholesterol (TC) data looking at HMGCR haplotypes and a null hypothesis that there is no effect of drug in decreasing total cholesterol (TC) in each HMGCR genotype (i.e., the mean of difference (before test date-after test date) in total cholesterol (TC) in each genotype group is zero).

TABLE 7-3

| Genotype | n | d bar (+/−SE(d bar)) | t₀ | p |
|---|---|---|---|---|
| 1. CGTA/CGTA | 46 | 38.9565 (+/−07.1171) | 5.47* | <0.00005 |
| 2. CGTA/CGTC | 7 | 22.5714 (+/−15.9177) | 1.42 | 0.1030 |
| 3. CGTA/TGTA | 5 | 18.0000 (+/−18.3439) | 0.98 | 0.1910 |
| 4. CGTA/CATA | 4 | 01.2500 (+/−17.2597) | 0.07 | 0.4734 |
| 5. CGTA/CGCA | 1 | 44. (−) | — | — |
| 6. Total | 63 | 33.1587 (+/−05.8455) | 5.92* | <0.00005 |

Table 7-3. Test of null hypothesis that for each HMGCRB (Marker:809125|712050|712044|664793) genotype there is no effect of Zocor™ in decreasing total cholesterol (TC) level (i.e., the mean of difference (before test date−after test date) in total cholesterol (TC) levels in each genotype group is zero)—i.e. H0=d bar=0 against H1:d bar>0. (*significant).

The analysis indicated that in the general population, the use of Zocor™ is associated with a significant (33.16, P<0.00005) response in terms of TC readings (Row 6, Table 7-3). This response is related to HMGCRB haplotype. Specifically, Zocor™ use is associated with a decrease in TC cholesterol levels in individuals of the CGTA/CGTA genotype. The mean TC difference (before drug date-after drug date) in LDL cholesterol for individuals of the CGTA/CGTA genotypes is 38.9565, and statistically significant. The other genotypes, CGTA/CGTC, CGTA/CATA, CGTA/CATA, and CGTA/CGCA were found to not be significantly associated with LDL reduction in Zocor™ patients.

CYP3A4 Haplotypes and Zocor™ Response

A statistical analysis was performed of the CYP3A4 gene, to identify haplotypes that are associated with a response to Zocor™. A one-sided paired t-test was performed on LDL data looking at CYP3A4 haplotypes and a null hypothesis that there is no effect of drug in decreasing cholesterol level (LDL) in each CYP3A4 genotype (i.e., the mean of difference (before test date−after test date) in cholesterol (LDL) in each genotype group is zero).

TABLE 7-4

| Genotype | n | d bar (+/−SE(d bar)) | t₀ | p |
|---|---|---|---|---|
| 1. ATGC/ATGC | 43 | 45.8605 (+/−07.0679) | 6.49* | <0.00005 |
| 2. ATGC/ATAC | 10 | 20.8000 (+/−12.7434) | 1.63 | 0.0686 |
| 3. ATGC/AGAC | 3 | 26.6667 (+/−24.0439) | 1.11 | 0.1915 |
| 4. ATGC/AGGT | 1 | 29.0 (−) | — | — |
| 5. Total | 57 | 40.1579 (+/−5.9792) | 6.72* | <0.00005 |

Table 7-4. Test of null hypothesis that for each CYP3A4C (Marker:809125|712050|712044|664793) genotype there is no effect of Zocor™ in decreasing cholesterol (LDL) level (i.e., the mean of difference (before test date−after test date) in cholesterol (LDL) levels in each genotype group is zero)—i.e. H0=d bar=0 against H1:d bar>0. (*significant).

The analysis indicated that in the general population, the use of Zocor™ is associated with a significantly significant decrease of 40.16 LDL units (Row 6, Table 7-4). This decrease is related to the CYP3A4C haplotype. Specifically, Zocor™ use is associated with a decrease in LDL cholesterol levels in individuals of the ATGC/ATGC genotype (P<0.00005). The mean LDL decrease in individuals harboring this genotype is 45.8605. In the case of genotypes with one minor allele, the decrease in LDL is not significant.

Next, a one-sided paired t-test was performed on total cholesterol (TC) data looking at CYP3A4C haplotypes and a null hypothesis that there is no effect of drug in decreasing total cholesterol (TC) in each CYP3A4C genotype (i. e., the mean of difference (before test date−after test date) in total cholesterol (TC) in each genotype group is zero).

TABLE 7-5

| Genotype | n | d bar (+/−SE(d bar)) | t₀ | p |
|---|---|---|---|---|
| 1. ATGC/ATGC | 47 | 41.5532 (+/−06.9587) | 5.97* | <0.00005 |
| 2. ATGC/ATAC | 11 | 07.7273 (+/−13.5211) | 0.57 | 0.2901 |
| 3. ATGC/AGAC | 3 | 26.3333 (+/−25.0000) | 1.05 | 0.2013 |
| 4. ATGC/AGGT | 1 | 41.0 (−) | — | — |
| 5. Total | 62 | 34.8065 (+/−6.0626) | 5.74* | <0.00005 |

Table 7-5. Test of null hypothesis that for each CYP3A4C (Marker:809114|664803|712037|869772) genotype there is no effect of Zocor™ in decreasing total cholesterol (TC) level (i.e., the mean of difference (before test date−after test date) in total cholesterol (TC) levels in each genotype group is zero)—i.e. H0=d bar=0 against H1:d bar>0. (*significant).

The analysis indicated that in the general population, the use of Zocor™ is associated with a significant (34.81, P<0.00005) response in terms of TC readings (Row 6, Table 7-5). This response is related to CYP3A4C haplotype. Specifically, Zocor™ use is associated with a decrease in TC cholesterol levels in individuals of the ATGC/ATGC genotype. The mean of decreasing TC in the genotype is 41.5532. In the case of genotypes with one minor allele, the decrease in LDL was not significant in Zocor™ patients.

Feature Modeling to Develop a Zocor™ Classifier

As with Lipitor™, a total of two genetic features were identified as capable of statistically resolving between Zocor™ responders and non-responders. The second feature was the HMGCRB haplotype system, which was discovered from a screen of 1,110 possible HMGCR SNP combinations. Haplotype systems in genes such as CYP2D6 and CYP2C9 did not make good features. Because the p-value for the resolution of Zocor™ response in terms of HMGCRB haplotypes was greater than for the CYP3A4C haplotype system, we used the CYP3A4C haplotype system as the root for a classification tree analysis of variable Zocor™ response In terms of CYP3A4C and HMGCRB haplotype pairs (genotypes). This method of modeling genetic features is described in T. Frudakis, U.S. patent application Ser. No. 10/156,995, filed May 28, 2002, which is incorporated herein in its entirety by reference.

As a part of construction process for the tree, we typed CYP3A4C:ATGC/ATGC individuals for haplotypes in the HMGCR gene. From the tree constructed, we observed that the HMGCRB haplotype system effectively resolved between Zocor™ responders and non-responders that harbored the CYP3A4C ATGC/ATGC genotype. Although most CYP3A4C:ATGC/ATGC individuals respond favorably to Zocor™, there are several that do not. The HMGCRB haplotype system showed the next best p-value for genetic distinction between responders and non-responders. Therefore HMGCRB genotypes were counted among CYP3A4C ATGC/ATGC individuals during construction of the genetic classification tree in an attempt to “explain” the heterogeneous component of the biased response in this group of patients (Table 7-6).

TABLE 7-6

| | Total Cholesterol Increase in Zocor patients | | | | | |
|---|---|---|---|---|---|---|
| | who are CYP3A4C ATGC/ATGC | | | | | |
| HMGCRB | | <5% | <5% | 5-10% | 10-20% | >20% |
| GENOTYPE | | INCR | DECR | DECR | DECR | DECR |
| CGTA/CGTA | 0 | 1 | 4 | 4 | 10 | 20 |
| CGTA/TGTA | 0 | 0 | 0 | 0 | 1 | 0 |
| CGCA/CGTA | 1 | 0 | 0 | 0 | 0 | 0 |
| CGTC/CGTA | 0 | 1 | 0 | 0 | 0 | 2 |
| CGTA/CATA | 1 | 1 | 0 | 1 | 1 | 0 |

Table 7-6. HMGCRB genotype counts of Zocor™ patients with the CYP3A4C ATGC/ATGC genotype. Counts for each genotype exhibiting various total cholesterol (TC) responses {increase (INCR) or decrease (DECR) relative to baseline} are shown. Genotypes are diploid pairs of haplotypes, shown in the first column, and the various responses are shown across the top of the table from poorest response (far left; >5%INCR) to best response (>20%DECR, far right).

The combined results from this two gene haplotype analysis of Zocor™ response is shown in Table 7-7. Individuals with two copies of the CYP3A4 major haplotype (ATGC) and two copies of the major HMGCR haplotype (CGTA) almost always respond favorably to Zocor™ (39/40 or 98% of the time), whereas individuals with a minor CYP3A4 or HMGCR haplotype respond favorably only half of the time (10/22 or 45% of the time).

TABLE 7-7

| | |
|---|---|
| (CYP3A4)/(HMGCR) | ZOCOR RESPONSE |

| GENOTYPE | NEGATIVE | POSITIVE |
|---|---|---|
| (+/+) : (+/+) | 1 | 38 |
| Other | 10 | 12 |

Table 7-7. Summary of Zocor™ response in terms of major (+) and minor (−) CYP3A4C and HMGCRB haplotype counts. Response is measured in terms of a reduction in total cholesterol (TC) levels relative to baseline (a POSITIVE response) or an increase, or no change in TC levels relative to baseline (a NEGATIVE response).

The combined results from the classification tree developed using the CYP3A4 and HMGCR haplotype system features show that whereas 38 of 39 (97%) CYP3A4C:ATGC/ATGC, HMGCRB:CGTA/CGTA individuals responded to Zocor™, only 10 of 22 (45%) other individuals responded to Zocor™ (Table 7-7).

Individuals with minor haplotypes at either the HMGCR or CYP3A4 genes showed a tendency to not respond to Zocor™. For Example, consider Table 7-7, where the HMGCRB genotypes are counted for CYP3A4 ATGC/ATGC individuals (individuals who have two copies of the major CYP3A4 haplotype). Within this group, most of the non-responders harbor a minor HMGCR haplotype (not CGTA) and the ratio of responders to non-responders is significantly higher for these individuals than for CGTA/CGTA individuals. This effect was not seen in other haplotype systems, for other genes. Consider the CYP2D6 gene (CYP2D6 is thought to be the most prolific of the xenobiotic metabolizer genes); there is no dependence between genotypes in this gene or responses (results not shown). Over 7,000 SNP combinations were tried, none of them significantly associated with response in this subgroup of patients or in Zocor™ patients in general.

If “MAJOR” is used to indicate a major haplotype for either of the CYP3A4 or HMGCR genes (with respect to the specific haplotype systems we have described; ATGC and CGTA, respectively), and “MINOR” is used to indicate a minor haplotype for either gene, the breakdown for the two gene analysis shows clearly that individuals that harbor two copies of a major haplotypes for both genes show a greater tendency to respond to Zocor™ than individuals that do not.

Conclusion:

Thus, the classification tree “solution” (or the pharmacogenetic classifier) for Zocor™ response is quite simple. Table 7-7 shows the final counts. Patients who are compound homozygotes for the major CYP3A4C and HMGCRB haplotypes are responders about 97% of the time. Others respond only 45% of the time. Thus, if a patient is not a compound homozygote for the major CYP3A4C and HMGCRB haplotypes, they are relatively unlikely to respond favorably and may consider other treatment options. The Example described here did not correct for other treatments, such as Niacin treatment (which is commonly administered in conjunction with Statins), or dietary change. We have assumed that Statins were prescribed to the individuals part of this study consistent with current FDA recommendations; dietary changes are almost always requested of patients. Though compliance is not possible to assess with our data, because compliance is the same regardless of which haplotype system or gene were examined, the finding of a haplotype system that is associated with Statin response is significant notwithstanding the study participants, their diet, other medications they were taking, their sex, or their age.

### EXAMPLE 8

### Genetic Solution for Provachol™ Response

The results described in the previous examples offer a method by which to predict patient response to Lipitor™ or Zocor™. An attempt was made to extend this method (i.e. using the haplotypes disclosed in Examples 7 and 8) to other statins. For Example, Pravachol™ response was analyzed using this method. However, Pravachol™ efficacy in the limited patient numbers analyzed, was not found to be correlated with CYP3A4C genotypes in a statistically significant manner (Table 8-1). Within CYP3A4C ATGC/ATGC individuals, HMGCRB genotypes were also not significantly correlated with Pravachol™ efficacy (Table 8-2). In fact, Pravachol™ response types were not significantly correlated with 2D6SG 1107 genotypes either, in the patients analyzed (not shown). Despite the lack of significance in these studies with a limited sample size, it is believed that subjects that are genotyped according to the present invention and found to have a genotype that is relatively unlikely to respond to Lipitor™ or Zocor™, is a good candidate for Pravacol™ treatment.

TABLE 8-1

| | Total Cholesterol Increase in Pravachol patients | | | | | |
|---|---|---|---|---|---|---|
| CYP3A4C | >5% | 0-5% | <5% | 5-10% | 10-20% | >20% |
| GENOTYPE | INCR | INCR | DECR | DECR | DECR | DECR |
| ATGC/ATGC | 4 | 1 | 1 | 0 | 2 | 6 |
| ATGC/ATAC | 0 | 0 | 0 | 1 | 0 | 1 |
| ATGC/AGAC | 0 | 0 | 0 | 0 | 1 | 1 |
| AGAC/ATGC | 0 | 0 | 1 | 0 | 0 | 0 |

Table 8-1. CYP3A4C genotype counts of Pravastatin™ patients exhibiting various responses. Response was measured in terms of post-prescription total cholesterol (TC) increase (INCR) or decrease (DECR) relative to baseline. Genotypes are diploid pairs of haplotypes shown in the first column, and the various responses are shown across the top of the table from poorest response (far left; >5%INCR) to best response (>20%DECR, far right).

TABLE 8-2

| | Total Cholesterol Increase in Pravachol patients with the CYP3A44B ATGC/ATGC genotype | | | | | |
|---|---|---|---|---|---|---|
| HMGCRB | >5% | 0-5% | <5% | 5-10% | 10-20% | >20% |
| GENOTYPE | INCR | INCR | DECR | DECR | DECR | DECR |
| CGTA/CGTA | 1 | 2 | 0 | 0 | — | 6 |
| CGTA/TGTA | 1 | 0 | 0 | 0 | 1 | 1 |
| CATA/CGCA | 1 | 0 | 0 | 0 | 0 | 0 |

Table 8-2. HMGCRB genotype counts of Pravachol™ patients with the CYP3A4C ATGC/ATGC genotype. Counts for each genotype exhibiting various total cholesterol (TC) responses {increase (INCR) or decrease (DECR) relative to baseline} are shown. Genotypes are diploid pairs of haplotypes shown in the first column, and the various responses are shown across the top of the table from poorest response (far left; >5%INCR) to best response (>20%DECR, far right).

The finding that Lipitor™ and Zocor™, but possibly not Pravachol™ patients can be resolved using CYP3A4 haplotypes is consistent with what is known from the literature about the metabolism of these drugs; though both Lipitor™ and Zocor™ are known to be metabolized by CYP3A4, Pravachol™ is know to not be metabolized by CYP3A4 (Igel et al., Eur. J. Clin. Pharmacol., 57(5):357 (2001); Chong et al., Am. J. Med. 111(5):390-400 (2001); Cohen et al., Biopharm. Drug Dispos. 21(9):353-64 (2000)). In fact, Pravachol™ is known to not be metabolized through the cytochrome P450 system at all. Thus, if the literature is correct, one would not expect to find genetic markers within the CYP3A4 or any other CYP gene to be associated with Pravachol™ response. However, the haplotypes disclosed herein are expected to be useful in inferring a response with respect to other statins that are metablolized by CYP3A4. The results presented in the Example were obtained systematically, without reference to these literature reports. The fact that they support conclusions drawn from previous works highlights their veracity.

### EXAMPLE 9

### Screening fot SNP Alleles Associated with Lipitor™ or Zocor Response

We screened the alleles of several hundred SNPs in order to identify those with a statistical association with LIPITOR or ZOCOR response. The strength of association is measured with a delta value (Shriver et al., Am. J. Genet., (2002), Shriver et al., Am. J. Genet., 60:1558 (1997)), which is inversely related to a chi-square statistic (the higher the value, the stronger the association). The delta value measures the difference in allele ratios between one group (in this case, responders) and another (in this case, non-responders). Generally, we select those SNPs with delta values greater than 0.15, though because the delta value is not very sensitive for sample size we discard those with delta values above 0.15 that have fewer than 20 counts for the minor allele in the overall sample (responders and non responders combined). In total, we surveyed 862 SNPs from xenobiotic metabolism and other genes and we present only the significant findings in tables below. Only SNPs with “significant” delta values are listed here, and their sequences appear in FIG. 3 and SEQ ID NOS:43-234 of the sequence listing. Because drug reaction is not a simple genetics trait, selecting an arbitrary p<0.05 criteria from a test such as a chi-square test is unreasonable because the marginal effects of loci that contribute towards genetic variance mainly or substantially through epistasis would be missed (only those that contribute through additivity and/or dominance would be recognized). In our experience (Frudakis et al., 2002b), choosing SNPs based on delta values greater than 0.10 produces better results for genetic classification than using a chi-square p<0.05 criteria (i.e. those selected based on the delta value criteria prove to be useful for constructing classifiers that generalize better than those selected based on the chi-square criteria). It is based on this experience (Frudakis et al., 2002b) that we justify claiming the SNPs presented here from our screen, even though their chi-square p-values may not be below 0.05 (in fact, those with delta values close to 0.10 usually have chi-square p-values of association approaching significance but not below 0.05).

For each of the tables in this Example, the Gene is shown with its GENBANK abbreviation, the Marker number is the unique identifier for the SNP. The counts for alleles are shown for the 20% Responder or Adverse Responder group (on the left side of the table) and the rest on the right side of the table. G1 A1 is the first allele and the “NO” following it is the count for this allele in that group, while G2 A2 is the second allele and the “NO” following it is the count for this allele in that same group. SAMPLE SIZE is also shown. At the far right of the table is the DELTA value for the distinction in counts between the Responder versus other groups, and an EAE value which is another statistical measure of how well an allele of the SNP is affiliated with one of the responder groups.

It appears that alleles of these three OCA2 SNPs are in linkage disequilibrium (notice the G2 A1 counts are similar for each of the three in the non responder group). Because these markers are good ancestry informative markers (AIMs), we conclude that there is likely a significant ancestry component to variable LDL response to ZOCOR. It may be that this ancestral component enables the detection of linkage with some as of yet unknown locus through admixture association (Shriver et al., 2002), or it may be that the ancestral component produces a so-called “false positive.” However, the literature suggests that there is little racial difference in ZOCOR or LIPITOR response. Also, most of the other 87 markers that did not have significant delta values are also excellent AIMs (Frudakis et al., 2002). In fact, the strongest OCA2, TYR AIMs are not on this list. That not all SNPs that are good AIMs are on this list (such as for the TYR gene, TYRP1 gene, MC1R gene, etc.) may suggest that certain chromosomal regions of ancestral distinction are important for the LDL response to this particular drug, particularly in the vicinity of the OCA2 locus, and that we detected this linkage through differential admixture in responders and non-responders. The locus liked with the OCA2 markers defined above do not seem to be associated with TC response as shown below, or with LDL response to LIPITOR™ shown above.

This raises a very important point for the development of a drug classifier. The OCA2 associations imply the presence of population substructure, and they also imply that there is an inter-populational (ancestral) component to variable LIPITOR™ response, at least in terms of LDL response. Thus, it is not known whether the genes and markers listed above are involved in LIPITOR™ metabolism, or whether they are associated with variable metabolism only by virtue of their association with ancestral group admixture. Thus, it cannot be concluded from this work that the genes and markers disclosed in this Example are actually relevant for variable response in a biochemical or cellular sense. However, the aim of the present Example is not to identify the genetic determinants of variable response—but rather to develop genetic classifiers predictive of response and if some of variable response is due to ancestral admixture then it is legitimate to consider markers of this admixture as legitimate classification tools for response in the general (mixed) population.

Another very important point is that not all of the AIMs make good markers for variable LDL response. Since the extent of linkage disequilibrium can be extreme in admixed populations—several megabases for example, (Shriver et al., 2002), it is possible that the present study is not just measuring ancestry with the OCA2 markers but measuring an admixture linkage effect in an admixed population. In this regard, adding all of the pigment gene SNPs associated with variable LDL response and calculating the percentage of variance they explain (through a regression analysis, for example) is likely to give that component of variance that cannot be explained with the battery of xenobiotic metabolism genes that have been tested, but which is explained by as yet unknown markers of differential ancestral proportions in the population. Since OCA2 is on chromosome 19, it is suspected that there are other LIPITOR™—LDL response genes on this chromosome.

TABLE 9-1
SNPs associated with LIPITOR RESPONSE in terms of LDL decrease: 20% responders (G1) versus others (G2).

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| UGT1A2008584_TC | 756 | T | 26 | C | 10 | 18 | T | 15 | C | 15 | 15 | 0.22222 | 0.09222 |
| UGT1A1875263_TC | 755 | C | 41 | T | 13 | 27 | C | 28 | T | 24 | 26 | 0.2208 | 0.09536 |
| PON3 | 869755 | C | 17 | T | 37 | 27 | C | 6 | T | 48 | 27 | 0.2037 | 0.11516 |
| SILV1052165_TC | 662 | C | 38 | T | 20 | 29 | C | 42 | T | 8 | 25 | 0.18483 | 0.08158 |
| CYP2D6 | 869777 | G | 23 | T | 31 | 27 | G | 30 | T | 20 | 25 | 0.17407 | 0.05322 |
| RAB27526213_TC | 844 | T | 27 | C | 25 | 26 | T | 16 | C | 30 | 23 | 0.1714 | 0.05252 |
| GSTM1414673_GA | 580 | G | 17 | A | 29 | 23 | G | 10 | A | 40 | 25 | 0.16957 | 0.06276 |
| CYP2E1RS2480257_TA | 37 | A | 10 | T | 10 | 10 | A | 20 | T | 10 | 15 | 0.16667 | 0.05017 |
| CYP4B1RS2405335_TC | 143 | C | 18 | T | 36 | 27 | C | 9 | T | 45 | 27 | 0.16667 | 0.06632 |
| ESD1923880_GA | 696 | A | 26 | G | 24 | 25 | A | 15 | G | 27 | 21 | 0.16286 | 0.04723 |
| CYP4B1RS681840_TC | 194 | T | 22 | C | 18 | 20 | T | 34 | C | 14 | 24 | 0.15833 | 0.04722 |
| ACE_4311_TC | 135 | T | 17 | C | 29 | 23 | T | 23 | C | 21 | 22 | 0.15316 | 0.04158 |
| AP3D12072304_GA | 906 | G | 11 | A | 35 | 23 | G | 4 | A | 42 | 23 | 0.15217 | 0.0789 |
| AHR2106728_GA | 599 | G | 10 | A | 30 | 20 | G | 20 | A | 30 | 25 | 0.15 | 0.04515 |

When only Caucasian samples were analyzed, the above SNPs showed the same association, but the following SNPs were identified as well:

TABLE 9-2
SNPs associated with LIPITOR RESPONSE in terms of LDL decrease: 20% responders (G1) versus others (G2).

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| GSTM1421547_TC | 527 | C | 17 | T | 7 | 12 | C | 6 | T | 12 | 9 | 0.375 | 0.25739 |
| CYP4B1RS2065996_TC | 137 | T | 9 | C | 15 | 12 | T | 2 | C | 22 | 12 | 0.29167 | 0.23903 |
| PON3 | 869755 | C | 14 | T | 22 | 18 | C | 3 | T | 27 | 15 | 0.28889 | 0.21896 |
| SILV1132095_GT | 704 | G | 15 | T | 11 | 13 | G | 19 | T | 3 | 11 | 0.28671 | 0.19122 |
| CYP2D6 | 554371 | T | 14 | C | 22 | 18 | T | 4 | C | 26 | 15 | 0.25556 | 0.15758 |
| GSTM1414673_GA | 580 | G | 11 | A | 17 | 14 | G | 4 | A | 24 | 14 | 0.25 | 0.14727 |
| CYP4B1RS681840_TC | 194 | C | 14 | T | 12 | 13 | C | 8 | T | 18 | 13 | 0.23077 | 0.09672 |
| UGT1A2008584_TC | 756 | T | 19 | C | 7 | 13 | T | 9 | C | 9 | 9 | 0.23077 | 0.10007 |
| CYP2D6 | 554365 | C | 19 | A | 13 | 16 | C | 11 | A | 19 | 15 | 0.22708 | 0.09133 |
| CYP2D6 | 869777 | G | 16 | T | 20 | 18 | G | 20 | T | 10 | 15 | 0.22222 | 0.08843 |
| ACE_4311_TC | 135 | T | 13 | C | 19 | 16 | T | 15 | C | 9 | 12 | 0.21875 | 0.08458 |
| ESD1923880_GA | 696 | A | 20 | G | 16 | 18 | A | 7 | G | 13 | 10 | 0.20556 | 0.07518 |
| UGT1A2008595_GA | 768 | G | 15 | A | 13 | 14 | G | 8 | A | 16 | 12 | 0.20238 | 0.0735 |
| ESD1923880_GA | 696 | A | 20 | G | 16 | 18 | A | 7 | G | 13 | 10 | 0.20556 | 0.07518 |

In the multi-racial sample of Table 9-1, the following genes (SNPs) were represented: UGT1A1 (2), PON3 (1), CYP2D6 (4), several pigment gene SNPs (5), GSTM1 (3), CYP2E1 (1), CYP4B1 (3), ESD (1), ACE (7), AHR (1), CYP2C8 (1), CYP2B6 (2), CYP3A5 (1) CYP1A2 (1). Genes such as CYP3A4, HMGCR, HMGCS1 were not detected for LDL response. Good AIMs like OCA2 and TYR were not detected. In the Caucasian analysis of Table 9-2, the associations were confirmed and most of the pigment gene associations disappear. GSTM1 (2), CYP4B1 (2), CYP2D6 (3), UGT1A1 (2) and ESD (2). The combined results illustrate that SNPs in five genes are associated with variable LDL response to LIPITOR™: CYP2D6, GSTM1, CYP4B1, ESD and UGT1A2.

LIPITOR™ response in terms of Total Cholesterol (TC) decrease in all patients irrespective of race (G1—20% responders versus G2—others). In this case, several SNPs with delta values less than 0.15 were allowed because the ratio of minor to major alleles for the two groups was close to 2:1:1:1, a quality of value that the delta value does not always (but usually does) capture.

TABLE 9-3

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| MYO5A1693494_TC | 836 | C | 15 | T | 17 | 16 | C | 12 | T | 34 | 23 | 0.20788 | 0.08272 |
| TYR | 217468 | C | 33 | A | 7 | 20 | C | 42 | A | 26 | 34 | 0.20735 | 0.09645 |
| CYP4B1RS2297810_GA | 350 | G | 25 | A | 13 | 19 | G | 51 | A | 9 | 30 | 0.19211 | 0.09016 |
| CYP4B1RS2405335_TC | 143 | C | 16 | T | 26 | 21 | C | 15 | T | 61 | 38 | 0.18358 | 0.07314 |
| MYO5A1724631_CA | 879 | A | 15 | C | 25 | 20 | A | 13 | C | 53 | 33 | 0.17803 | 0.06916 |
| CYP2B6 | 1002412 | G | 34 | A | 8 | 21 | G | 48 | A | 28 | 38 | 0.17794 | 0.07016 |
| MYO5A1669871_TC | 847 | C | 20 | T | 6 | 13 | C | 39 | T | 3 | 21 | 0.15934 | 0.09418 |
| NAT21041983_TC | 483 | C | 9 | T | 19 | 14 | C | 22 | T | 24 | 23 | 0.15683 | 0.04497 |
| PON1 | 869817 | G | 27 | A | 15 | 21 | G | 59 | A | 15 | 37 | 0.15444 | 0.05243 |
| GSTT22267047_TC | 464 | T | 7 | C | 29 | 18 | T | 25 | C | 47 | 36 | 0.15278 | 0.05242 |
| CYP2C8_RS1891071_GA | 369 | G | 5 | A | 17 | 11 | G | 21 | A | 35 | 28 | 0.14773 | 0.04574 |
| CYP4B1RS751027_GA | 343 | A | 16 | G | 28 | 22 | A | 16 | G | 58 | 37 | 0.14742 | 0.04662 |
| SILV1052165_TC | 662 | C | 28 | T | 16 | 22 | C | 58 | T | 16 | 37 | 0.14742 | 0.04662 |
| CYP2E1RS2480257_TA | 37 | A | 8 | T | 8 | 8 | A | 22 | T | 12 | 17 | 0.14706 | 0.03871 |
| AHR2106728_GA | 599 | A | 22 | G | 6 | 14 | A | 46 | G | 26 | 36 | 0.14683 | 0.04647 |
| MYO5A2899489_GT | 930 | G | 26 | T | 6 | 16 | G | 40 | T | 20 | 30 | 0.14583 | 0.04897 |
| GSTT2140184_GA | 568 | A | 28 | G | 14 | 21 | A | 41 | G | 37 | 39 | 0.14103 | 0.03617 |
| CYP2C8E2E3_397_TC | 134 | T | 18 | C | 24 | 21 | T | 22 | C | 54 | 38 | 0.1391 | 0.03687 |
| CYP2B6RS2279345_TC | 142 | T | 16 | C | 26 | 21 | T | 17 | C | 53 | 35 | 0.1381 | 0.03908 |
| MYO5A935892_GA | 898 | A | 23 | G | 17 | 20 | A | 47 | G | 19 | 33 | 0.13712 | 0.03593 |
| MYO5A1669870_CA | 877 | C | 9 | A | 27 | 18 | C | 7 | A | 55 | 31 | 0.1371 | 0.05733 |
| MYO5A1693512_GC | 821 | G | 22 | C | 6 | 14 | G | 30 | C | 16 | 23 | 0.13354 | 0.0389 |
| CYP2A131709081_TC | 503 | C | 27 | T | 11 | 19 | C | 54 | T | 10 | 32 | 0.13322 | 0.04562 |
| CYP4B1RS2065996_TC | 137 | T | 8 | C | 18 | 13 | T | 8 | C | 36 | 22 | 0.12587 | 0.03789 |
| MAOA909525_GA | 549 | A | 17 | G | 17 | 17 | A | 40 | G | 24 | 32 | 0.125 | 0.02773 |

Caucasian LIPITOR™ response in terms of Total Cholesterol (TC) decrease (G1—20% responders versus G2—others). In this case, several SNPs with delta values less than 0.15 were allowed, because the ratio of minor to major alleles for the two groups was close to 2:1:1:1, a quality of value that the delta value does not always (but usually does) capture.

TABLE 9-4

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| RAB271014597_GT | 932 | T | 12 | G | 10 | 11 | T | 25 | G | 5 | 15 | 0.28788 | 0.17842 |
| GSTM3 | 971882 | C | 17 | A | 9 | 13 | C | 16 | A | 26 | 21 | 0.27289 | 0.13292 |
| OCA2 | 886896 | A | 26 | G | 2 | 14 | A | 29 | G | 15 | 22 | 0.26948 | 0.22303 |
| MYO5A1693494_TC | 836 | T | 10 | C | 12 | 11 | T | 26 | C | 10 | 18 | 0.26768 | 0.13227 |
| GSTM1421547_TC | 527 | C | 11 | T | 5 | 8 | C | 12 | T | 16 | 14 | 0.25893 | 0.12101 |
| CYP2C8_RS1891070_GA | 357 | G | 1 | A | 7 | 4 | G | 6 | A | 10 | 8 | 0.25 | 0.15581 |
| PON1 | 869817 | G | 15 | A | 13 | 14 | G | 31 | A | 9 | 20 | 0.23929 | 0.11365 |
| TYR | 217468 | C | 20 | A | 6 | 13 | C | 25 | A | 21 | 23 | 0.22575 | 0.10095 |
| OCA2 | 886894 | T | 26 | C | 2 | 14 | T | 31 | C | 13 | 22 | 0.22403 | 0.165 |
| GSTT22267047_TC | 464 | T | 4 | C | 22 | 13 | T | 15 | C | 27 | 21 | 0.2033 | 0.09862 |
| CYP2C8 | 1004864 | G | 7 | A | 21 | 14 | G | 19 | A | 23 | 21 | 0.20238 | 0.07977 |
| CYP2C9 | 869797 | C | 11 | T | 17 | 14 | C | 8 | T | 34 | 21 | 0.20238 | 0.08891 |
| CYP2C8_RS1341159_GC | 94 | G | 7 | C | 21 | 14 | G | 19 | C | 23 | 21 | 0.20238 | 0.07977 |
| CYP2C8_RS1891071_GA | 369 | G | 2 | A | 12 | 7 | G | 11 | A | 21 | 16 | 0.20089 | 0.09991 |
| CYP2C8_1341159_GC | 95 | C | 21 | G | 7 | 14 | C | 22 | G | 18 | 20 | 0.2 | 0.07799 |
| POR17685_GA | 691 | G | 22 | A | 6 | 14 | G | 24 | A | 16 | 20 | 0.18571 | 0.07209 |
| CYP2C8_2071426_GA | 362 | G | 10 | A | 16 | 13 | G | 8 | A | 32 | 20 | 0.18462 | 0.07347 |
| CYP2B6 | 1002412 | G | 23 | A | 5 | 14 | G | 27 | A | 15 | 21 | 0.17857 | 0.07277 |
| CYP2C8_RS947173_GA | 342 | G | 7 | A | 21 | 14 | G | 18 | A | 24 | 21 | 0.17857 | 0.06289 |
| GSTT2140184_GA | 568 | A | 19 | G | 9 | 14 | A | 22 | G | 22 | 22 | 0.17857 | 0.05795 |
| GSTT2140185_GA | 783 | G | 15 | A | 11 | 13 | G | 17 | A | 25 | 21 | 0.17216 | 0.05203 |
| GSTT2140188_GC | 652 | C | 17 | G | 9 | 13 | C | 33 | G | 7 | 20 | 0.17115 | 0.06798 |
| CYP2D6 | 554371 | T | 10 | C | 18 | 14 | T | 8 | C | 34 | 21 | 0.16667 | 0.06219 |
| CYP4B1RS751028_GA | 292 | G | 11 | A | 11 | 11 | G | 24 | A | 12 | 18 | 0.16667 | 0.05017 |
| GSTP12370143_TC | 533 | C | 15 | T | 13 | 14 | C | 28 | T | 12 | 20 | 0.16429 | 0.05024 |

From the multi-racial data in Table 9-3, the following genes had more than one SNP on the list for association with variable TC response to LIPITOR™: MYO5A (8), CYP4B1 (4), CYP2B6 (2), GSTT2 (2), CYP2C8 (3), SILV (2) and CYP2E1 (2). From the analysis of Caucasians in Table 9-4, the following genes had more than one SNP associated with variable TC response to LIPITOR™: GSTMs (2), CYP2C8 (7), OCA2 (2), GSTT2 (4). It is therefore concluded that the CYP2C8, GSTM and GSTT2 genes exert the strongest control on variable TC response to LIPITOR™—so strong that their association can be detected at the level of the single SNP (unlike most of the haplotype associations we described earlier). When applying a test towards individuals without knowledge of their race, the MYO5A gene is also instructive. Interestingly, SNPs from the HMGCR, HMGCS1 or other xenobiotic metabolism genes such as CYP3A4 or CYP2C9 were not identified. Evidently the HMGCR and CYP3A4 alleles we identified using the HAPLOSCOPE method described earlier in the application are not significantly associated with LDL/TC response on their own, but are quite significant within the contexts of other loci in their respective genes.

LIPITOR™ SGOT 20% RESPONSE in individuals without respect to race: we compare genotypes from individuals that experienced at least a 20% increase in SGOT readings after taking LIPITOR™ (G1), versus everyone else (G2). For this screen SNPs with deltas less than 0.125 and those with deltas above 0.125 but with a minor allele sample size less than 10, not 20 (due to the scarcity of the adverse reaction in the population) were eliminated.

TABLE 9-5

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| CYP2E1RS2480257_TA | 37 | T | 8 | A | 2 | 5 | T | 10 | A | 20 | 15 | 0.46667 | 0.42144 |
| DCT2224780_TC | 674 | C | 16 | T | 0 | 8 | C | 29 | T | 23 | 26 | 0.43864 | 1.0323 |
| GSTM1421547_TC | 527 | C | 11 | T | 3 | 7 | C | 15 | T | 23 | 19 | 0.39098 | 0.2932 |
| CYP3A7RS2687140_GA | 287 | G | 2 | A | 10 | 6 | G | 14 | A | 12 | 13 | 0.37179 | 0.28476 |
| XO_RS1429374_GA | 295 | G | 12 | A | 10 | 11 | G | 11 | A | 41 | 26 | 0.33392 | 0.21724 |
| GSTT2140192_TC | 469 | T | 14 | C | 10 | 12 | T | 16 | C | 44 | 30 | 0.31667 | 0.1854 |
| CES22241409_TC | 658 | T | 9 | C | 11 | 10 | T | 8 | C | 50 | 29 | 0.31207 | 0.22117 |
| SILV1132095_GT | 704 | G | 12 | T | 0 | 6 | G | 36 | T | 16 | 26 | 0.30132 | 0.56644 |
| RAB271014597_GT | 932 | T | 8 | G | 10 | 9 | T | 31 | G | 11 | 21 | 0.29365 | 0.16059 |
| GSTT2140185_GA | 783 | A | 6 | G | 14 | 10 | A | 34 | G | 24 | 29 | 0.28621 | 0.14861 |
| AP3D125673_TC | 828 | C | 7 | T | 15 | 11 | C | 3 | T | 59 | 31 | 0.26979 | 0.25974 |
| CYP1A2E7_405_GC | 98 | G | 13 | C | 9 | 11 | G | 20 | C | 42 | 31 | 0.26833 | 0.12931 |
| CYP2A131709084_GA | 546 | G | 5 | A | 7 | 6 | G | 5 | A | 27 | 16 | 0.26042 | 0.15267 |
| GSTT2140184_GA | 568 | A | 19 | G | 5 | 12 | A | 33 | G | 29 | 31 | 0.25941 | 0.13584 |
| GSTA22290758_GA | 558 | G | 14 | A | 10 | 12 | G | 19 | A | 39 | 29 | 0.25575 | 0.11724 |
| CYP4B1RS632645_TC | 171 | T | 2 | C | 18 | 10 | T | 19 | C | 35 | 27 | 0.25185 | 0.17351 |
| GSTT2140187_GA | 562 | G | 17 | A | 1 | 9 | G | 43 | A | 19 | 31 | 0.2509 | 0.21972 |
| GSTT2140190_GC | 443 | C | 16 | G | 6 | 11 | C | 30 | G | 32 | 31 | 0.2434 | 0.1105 |
| DCT1325611_TC | 657 | T | 14 | C | 10 | 12 | T | 46 | C | 10 | 28 | 0.2381 | 0.12301 |
| AP3D12072304_GA | 906 | G | 7 | A | 15 | 11 | G | 5 | A | 55 | 30 | 0.23485 | 0.16684 |
| DCT2892680_GA | 699 | G | 10 | A | 6 | 8 | G | 48 | A | 8 | 28 | 0.23214 | 0.12914 |
| GSTA22290757_TC | 495 | T | 7 | C | 11 | 9 | T | 31 | C | 19 | 25 | 0.23111 | 0.0945 |
| CYP2C8 | 1004867 | A | 12 | G | 10 | 11 | A | 48 | G | 14 | 31 | 0.22874 | 0.10429 |
| CYP2C9 | 869797 | T | 12 | C | 10 | 11 | T | 48 | C | 14 | 31 | 0.22874 | 0.10429 |
| CYP2D6_RS2267444_GC | 93 | C | 10 | G | 12 | 11 | C | 14 | G | 48 | 31 | 0.22874 | 0.10429 |
| CYP4B1RS2297812_GC | 97 | G | 13 | C | 3 | 8 | G | 34 | C | 24 | 29 | 0.22629 | 0.10988 |
| MYO5A722436_GT | 929 | G | 11 | T | 9 | 10 | G | 48 | T | 14 | 31 | 0.22419 | 0.10043 |
| MYO5A1724630_GC | 806 | G | 4 | C | 6 | 5 | G | 6 | C | 28 | 17 | 0.22353 | 0.11018 |
| GSTA21051775_TC | 456 | T | 9 | C | 11 | 10 | T | 39 | C | 19 | 29 | 0.22241 | 0.08885 |
| POR8509_GA | 689 | G | 12 | A | 8 | 10 | G | 46 | A | 10 | 28 | 0.22143 | 0.10776 |
| AP3D12238593_TC | 834 | C | 7 | T | 15 | 11 | C | 6 | T | 56 | 31 | 0.22141 | 0.14149 |
| AP3D12238594_TC | 838 | T | 16 | C | 8 | 12 | T | 55 | C | 7 | 31 | 0.22043 | 0.13099 |
| CYP4B1RS681840_TC | 194 | C | 6 | T | 14 | 10 | C | 28 | T | 26 | 27 | 0.21852 | 0.08744 |
| GSTA21051536_GC | 440 | G | 14 | C | 4 | 9 | G | 28 | C | 22 | 25 | 0.21778 | 0.09568 |
| GSTT2678863_GA | 786 | A | 15 | G | 7 | 11 | A | 28 | G | 32 | 30 | 0.21515 | 0.08369 |
| TYR_RS1851992_GA | 278 | G | 7 | A | 15 | 11 | G | 33 | A | 29 | 31 | 0.21408 | 0.08287 |
| CYP2C9RS2860905_GA | 367 | A | 3 | G | 19 | 11 | A | 21 | G | 39 | 30 | 0.21364 | 0.11382 |
| CYP2C82071426_GA | 596 | G | 10 | A | 12 | 11 | G | 15 | A | 47 | 31 | 0.21261 | 0.08862 |
| UGT1A2008584_TC | 756 | T | 8 | C | 8 | 8 | T | 27 | C | 11 | 19 | 0.21053 | 0.0821 |
| CYP2C8 | 1004864 | G | 5 | A | 17 | 11 | G | 27 | A | 35 | 31 | 0.20821 | 0.08719 |
| CYP2C8_RS1341159_G | 94 | G | 5 | C | 17 | 11 | G | 27 | C | 35 | 31 | 0.20821 | 0.08719 |
| C | | | | | | | | | | | | | |
| MYO5A935892_GA | 898 | A | 14 | G | 10 | 12 | A | 49 | G | 13 | 31 | 0.20699 | 0.08903 |
| CYP2C8_1341159_GC | 95 | C | 17 | G | 5 | 11 | C | 34 | G | 26 | 30 | 0.20606 | 0.08551 |
| DCT727299_GA | 682 | G | 3 | A | 9 | 6 | G | 20 | A | 24 | 22 | 0.20455 | 0.0814 |
| MYO5A1724631_CA | 879 | C | 16 | A | 8 | 12 | C | 54 | A | 8 | 31 | 0.2043 | 0.10793 |
| OCA2 | 886894 | T | 21 | C | 3 | 12 | T | 42 | C | 20 | 31 | 0.19758 | 0.10331 |
| GSTA22144696_TC | 455 | T | 11 | C | 9 | 10 | T | 22 | C | 40 | 31 | 0.19516 | 0.06768 |
| DCT2296498_GA | 701 | G | 7 | A | 17 | 12 | G | 6 | A | 56 | 31 | 0.19489 | 0.11395 |
| AP3D12072305_GC | 820 | G | 7 | C | 17 | 12 | G | 6 | C | 56 | 31 | 0.19489 | 0.11395 |
| GSTT2140188_GC | 652 | G | 9 | C | 13 | 11 | G | 13 | C | 47 | 30 | 0.19242 | 0.07667 |
| CYP2C8_RS947173_GA | 342 | G | 5 | A | 17 | 11 | G | 26 | A | 36 | 31 | 0.19208 | 0.07494 |
| GSTA22180319_GA | 577 | G | 11 | A | 9 | 10 | G | 46 | A | 16 | 31 | 0.19194 | 0.0713 |
| MYO5A1724639_TC | 843 | T | 10 | C | 14 | 12 | T | 14 | C | 48 | 31 | 0.19086 | 0.07424 |
| GSTT22719_GT | 611 | G | 8 | T | 14 | 11 | G | 31 | T | 25 | 28 | 0.18994 | 0.06391 |
| GSTA22894803_CA | 435 | A | 22 | C | 0 | 11 | A | 47 | C | 11 | 29 | 0.18768 | 0.39324 |
| CYP2C8E8_92_GA | 265 | G | 11 | A | 5 | 8 | G | 17 | A | 17 | 17 | 0.1875 | 0.0642 |
| AIM35415_GT | 937 | G | 16 | T | 8 | 12 | G | 30 | T | 32 | 31 | 0.1828 | 0.06015 |
| CYP2C9RS2298037_TC | 248 | T | 1 | C | 21 | 11 | T | 13 | C | 45 | 29 | 0.17868 | 0.1399 |
| CYP4B1RS1572603_TC | 176 | C | 13 | T | 1 | 7 | C | 36 | T | 12 | 24 | 0.17857 | 0.11372 |
| FDPS | 756238 | C | 7 | T | 13 | 10 | C | 10 | T | 48 | 29 | 0.17759 | 0.07324 |
| CYP2C8_RS1891071_G | 369 | G | 3 | A | 13 | 8 | G | 16 | A | 28 | 22 | 0.17614 | 0.06936 |
| A | | | | | | | | | | | | | |
| GSTT2140194_GC | 442 | G | 17 | C | 5 | 11 | G | 37 | C | 25 | 31 | 0.17595 | 0.06356 |
| CYP2D6 | 554371 | C | 15 | T | 7 | 11 | C | 53 | T | 9 | 31 | 0.17302 | 0.07596 |
| GSTA22608677_GC | 451 | G | 18 | C | 4 | 11 | G | 40 | C | 22 | 31 | 0.17302 | 0.0681 |
| GSTA22608679_GA | 570 | G | 12 | A | 12 | 12 | G | 39 | A | 19 | 29 | 0.17241 | 0.05385 |
| OCA2 | 886896 | A | 20 | G | 4 | 12 | A | 41 | G | 21 | 31 | 0.17204 | 0.07026 |
| CYP2C9 | 869803 | G | 3 | T | 9 | 6 | G | 3 | T | 35 | 19 | 0.17105 | 0.10089 |
| GSTT2140196_GA | 605 | G | 14 | A | 8 | 11 | G | 27 | A | 31 | 29 | 0.17085 | 0.05177 |
| CYP4B1RS751028_GA | 292 | A | 10 | G | 8 | 9 | A | 17 | G | 27 | 22 | 0.16919 | 0.05039 |
| MYO5A752864_TC | 835 | T | 13 | C | 9 | 11 | T | 47 | C | 15 | 31 | 0.16716 | 0.05622 |
| XO_RS2295475_TC | 150 | C | 12 | T | 8 | 10 | C | 46 | T | 14 | 30 | 0.16667 | 0.05676 |
| AHR2106728_GA | 599 | G | 8 | A | 10 | 9 | G | 14 | A | 36 | 25 | 0.16444 | 0.05151 |
| CYP2B6RS707265_GA | 283 | G | 18 | A | 4 | 11 | G | 38 | A | 20 | 29 | 0.16301 | 0.06104 |
| CYP2D6_RS2267446_TC | 172 | T | 6 | C | 14 | 10 | T | 8 | C | 50 | 29 | 0.16207 | 0.06935 |
| PON3 | 869790 | G | 11 | T | 11 | 11 | G | 21 | T | 41 | 31 | 0.16129 | 0.04687 |
| AHR2237299_GA | 540 | G | 11 | A | 3 | 7 | G | 30 | A | 18 | 24 | 0.16071 | 0.05503 |
| RAB27526213_TC | 844 | T | 9 | C | 15 | 12 | T | 33 | C | 29 | 31 | 0.15726 | 0.04371 |
| CYP2C181042194_GT | 712 | G | 18 | T | 0 | 9 | G | 42 | T | 8 | 25 | 0.15708 | 0.29022 |
| CYP1A2_RS2069524_T | 206 | T | 11 | C | 1 | 6 | T | 32 | C | 10 | 21 | 0.15476 | 0.08299 |
| C | | | | | | | | | | | | | |
| PON3 | 869755 | T | 19 | C | 3 | 11 | T | 44 | C | 18 | 31 | 0.15396 | 0.06365 |
| CYP2D6_RS2856960_T | 193 | C | 13 | T | 5 | 9 | C | 33 | T | 25 | 29 | 0.15326 | 0.04512 |
| C | | | | | | | | | | | | | |
| ESD1216958_GT | 706 | G | 9 | T | 13 | 11 | G | 16 | T | 46 | 31 | 0.15103 | 0.04515 |
| CYP2B6RS2279345_TC | 142 | C | 18 | T | 2 | 10 | C | 45 | T | 15 | 30 | 0.15 | 0.07157 |
| CYP2A6RS1061608_TA | 41 | T | 1 | A | 17 | 9 | T | 11 | A | 43 | 27 | 0.14815 | 0.09457 |
| CYP4B1RS837400_GA | 336 | G | 16 | A | 6 | 11 | G | 36 | A | 26 | 31 | 0.14663 | 0.04174 |
| CYP2A6RS1137115_GA | 284 | G | 17 | A | 7 | 12 | G | 53 | A | 9 | 31 | 0.14651 | 0.05636 |
| CYP4B1RS2297810_GA | 350 | G | 19 | A | 1 | 10 | G | 45 | A | 11 | 28 | 0.14643 | 0.09766 |
| GSTT2140186_GA | 545 | G | 7 | A | 15 | 11 | G | 25 | A | 29 | 27 | 0.14478 | 0.03859 |

Table 9-5 shows no fewer than 104 SNPs are associated with SGOT increases greater than 20% elicited by LIPITOR™. About 700 others tested were not associated. Of the 104 associated SNPs, those SNPs in the GSTA2 (11 SNPs), GSTT2 (11 SNPs), CYP2C9 (4 SNPs), CYP2C8 (9 SNPs), CYP4B1 (5) and CYP2D6 (4 SNPs) genes are exceptionally strong markers of adverse SGOT response in terms of delta values, estimates of affiliation (EAEs) and in terms of the numbers of SNPs in each of these genes on the list. Not only were numerous SNPs in each gene identified with delta values greater than 0.20, but many had alleles that were absolutely indicative of response in that certain alleles were ONLY present in the responder or non-responder group (see bold print in above table). For example, 19/20 individuals with the GSTT2140187 minor allele experience a 20% increase in SGOT levels and 22/26 individuals with the GSTA21051536 minor allele respond the same way. CYP2C9 also seems to play important role—21 of 24 individuals with a minor CYP2C9RS2860905 allele respond to LIPITOR with no 20% increase and this minor allele may contribute a protective effect. Restricted to Caucasians (Table 9-6), the analysis shows far fewer SNPs associated, with the following genes have multiple SNPs associated with SGOT elevations: GSTT2 (8), GSTA2 (11), CYP2C8 (4), CYP2C9 (2), DCT (3), CYP4B1 (2). Combining the two screens it can be asserted with good confidence that GSTT2, GSTA2, CYP2C8 CYP2C9 and CYP4B1 alleles are associated with SGOT elevations in LIPITOR™ patients in a manner that is biologically meaningful. In individuals of unknown ancestry, the DCT, MYO5A, AP3D and AIM genes also contain useful markers.

LIPITOR™ SGOT 20% RESPONSE in individuals of Caucasian descent only: we compare genotypes from individuals that experienced at least a 20% increase in SGOT readings after taking LIPITOR™ (G1) versus everyone else (G2). For this screen, due to the sample of adverse responders, we eliminated those with deltas less than 0.23 and those with deltas above 0.23 but with a minor allele sample size less than 15.

TABLE 9-6

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| CYP2E1RS2480257_TA | 37 | T | 6 | A | 2 | 4 | T | 8 | A | 18 | 13 | 0.44231 | 0.36681 |
| CYP2C9 | 869803 | G | 3 | T | 3 | 3 | G | 2 | T | 26 | 14 | 0.42857 | 0.4774 |
| GSTM1421547_TC | 527 | C | 9 | T | 3 | 6 | C | 9 | T | 19 | 14 | 0.42857 | 0.34356 |
| DCT2224780_TC | 674 | C | 12 | T | 0 | 6 | C | 24 | T | 18 | 21 | 0.4222 | 0.88637 |
| CYP3A7RS2687140_GA | 287 | A | 7 | G | 1 | 4 | A | 10 | G | 12 | 11 | 0.42045 | 0.38862 |
| GSTT2140185_GA | 783 | G | 11 | A | 3 | 7 | G | 17 | A | 27 | 22 | 0.39935 | 0.30558 |
| GSTT2140192_TC | 469 | T | 12 | C | 6 | 9 | T | 14 | C | 34 | 24 | 0.375 | 0.25739 |
| CES22241409_TC | 658 | T | 8 | C | 8 | 8 | T | 6 | C | 40 | 23 | 0.36957 | 0.30449 |
| XO_RS1429374_GA | 295 | G | 9 | A | 7 | 8 | G | 9 | A | 33 | 21 | 0.34821 | 0.23449 |
| MYO5A1724630_GC | 806 | G | 3 | C | 3 | 3 | G | 4 | C | 22 | 13 | 0.34615 | 0.25628 |
| DCT2892680_GA | 699 | G | 5 | A | 5 | 5 | G | 37 | A | 7 | 22 | 0.34091 | 0.24651 |
| AP3D125673_TC | 828 | C | 6 | T | 10 | 8 | C | 2 | T | 46 | 24 | 0.33333 | 0.37996 |
| GSTA21051536_GC | 440 | G | 10 | C | 2 | 6 | G | 20 | C | 18 | 19 | 0.30702 | 0.20055 |
| GSTA22290758_GA | 558 | G | 11 | A | 7 | 9 | G | 14 | A | 32 | 23 | 0.30676 | 0.17035 |
| POR8509_GA | 689 | G | 7 | A | 7 | 7 | G | 37 | A | 9 | 23 | 0.30435 | 0.18686 |
| GSTA22290757_TC | 495 | T | 4 | C | 8 | 6 | T | 28 | C | 16 | 22 | 0.30303 | 0.16487 |
| SILV1132095_GT | 704 | G | 10 | T | 0 | 5 | G | 29 | T | 13 | 21 | 0.30051 | 0.524 |
| GSTA22144696_TC | 455 | T | 10 | C | 6 | 8 | T | 16 | C | 32 | 24 | 0.29167 | 0.15251 |
| GSTT2140184_GA | 568 | A | 15 | G | 3 | 9 | A | 26 | G | 22 | 24 | 0.29167 | 0.18271 |
| GSTA21051775_TC | 456 | T | 6 | C | 8 | 7 | T | 33 | C | 13 | 23 | 0.28882 | 0.15293 |
| DCT1325611_TC | 657 | T | 10 | C | 8 | 9 | T | 37 | C | 7 | 22 | 0.28535 | 0.17869 |
| GSTT2678863_GA | 786 | A | 11 | G | 5 | 8 | A | 19 | G | 27 | 23 | 0.27446 | 0.13586 |
| GSTT2140190_GC | 443 | C | 12 | G | 4 | 8 | C | 23 | G | 25 | 24 | 0.27083 | 0.13903 |
| GSTA22180319_GA | 577 | G | 8 | A | 8 | 8 | G | 37 | A | 11 | 24 | 0.27083 | 0.14268 |
| MYO5A722436_GT | 929 | G | 7 | T | 7 | 7 | G | 37 | T | 11 | 24 | 0.27083 | 0.14268 |
| CYP2A6RS1061608_TA | 41 | A | 12 | T | 0 | 6 | A | 31 | T | 11 | 21 | 0.25554 | 0.45654 |
| MAOB1799836_TC | 465 | T | 5 | C | 9 | 7 | T | 28 | C | 18 | 23 | 0.25155 | 0.11248 |
| CYP2C8 | 1004864 | G | 3 | A | 13 | 8 | G | 21 | A | 27 | 24 | 0.25 | 0.13192 |
| CYP2C8_RS1341159_GC | 94 | C | 13 | G | 3 | 8 | C | 27 | G | 21 | 24 | 0.25 | 0.13192 |
| GSTA22608677_GC | 451 | G | 14 | C | 2 | 8 | G | 30 | C | 18 | 24 | 0.25 | 0.15581 |
| GSTT2140187_GA | 562 | G | 15 | A | 1 | 8 | G | 33 | A | 15 | 24 | 0.25 | 0.20842 |
| RAB271014597_GT | 932 | T | 8 | G | 8 | 8 | T | 24 | G | 8 | 16 | 0.25 | 0.11928 |
| AIM35415_GT | 937 | G | 12 | T | 6 | 9 | G | 20 | T | 28 | 24 | 0.25 | 0.11179 |
| CYP2C8_1341159_GC | 95 | C | 13 | G | 3 | 8 | C | 26 | G | 20 | 23 | 0.24728 | 0.1293 |
| GSTT2140188_GC | 652 | G | 7 | C | 9 | 8 | G | 9 | C | 37 | 23 | 0.24185 | 0.12209 |
| CYP4B1RS681840_TC | 194 | T | 10 | C | 4 | 7 | T | 20 | C | 22 | 21 | 0.2381 | 0.1046 |
| GSTA22144697_TC | 474 | T | 8 | C | 0 | 4 | T | 33 | C | 11 | 22 | 0.2363 | 0.34505 |
| CYP2C9RS2298037_TC | 248 | C | 16 | T | 0 | 8 | C | 35 | T | 11 | 23 | 0.23547 | 0.46197 |
| GSTA22894803_CA | 435 | A | 16 | C | 0 | 8 | A | 35 | C | 11 | 23 | 0.23547 | 0.46197 |
| CYP4B1RS632645_TC | 171 | C | 15 | T | 1 | 8 | C | 31 | T | 13 | 22 | 0.23295 | 0.18605 |
| GSTA22180315_TC | 500 | T | 12 | C | 2 | 7 | T | 30 | C | 18 | 24 | 0.23214 | 0.12914 |
| GSTT22719_GT | 611 | G | 5 | T | 11 | 8 | G | 25 | T | 21 | 23 | 0.23098 | 0.09658 |
| CYP1A2E7_405_GC | 98 | G | 9 | C | 7 | 8 | G | 16 | C | 32 | 24 | 0.22917 | 0.094 |
| CYP2C8_RS947173_GA | 342 | G | 3 | A | 13 | 8 | G | 20 | A | 28 | 24 | 0.22917 | 0.11245 |
| GSTA22749019_GA | 583 | G | 8 | A | 8 | 8 | G | 35 | A | 13 | 24 | 0.22917 | 0.09857 |

LIPITOR™ ALTGPT 20% RESPONSE: we compare genotypes from individuals that experienced at least a 20% increase in ALTGPT readings after taking LIPITOR™ (G1) versus everyone else (G2).

TABLE 9-7

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| AHR2237299_GA | 540 | G | 16 | A | 4 | 10 | G | 14 | A | 16 | 15 | 0.33333 | 0.22002 |
| XO_RS2295475_TC | 150 | T | 12 | C | 14 | 13 | T | 5 | C | 33 | 19 | 0.32996 | 0.24833 |
| ACE_4343_GA | 349 | A | 22 | G | 2 | 12 | A | 25 | G | 15 | 20 | 0.29167 | 0.23903 |
| MAOB1799836_TC | 465 | T | 17 | C | 7 | 12 | T | 15 | C | 21 | 18 | 0.29167 | 0.15501 |
| GSTM1421547_TC | 527 | T | 9 | C | 7 | 8 | T | 5 | C | 13 | 9 | 0.28472 | 0.14923 |
| ACE_4335_GA | 291 | G | 11 | A | 11 | 11 | G | 8 | A | 28 | 18 | 0.27778 | 0.15113 |
| MAOA909525_GA | 549 | G | 11 | A | 7 | 9 | G | 13 | A | 25 | 19 | 0.26901 | 0.1292 |
| CYP2B6 | 1002412 | G | 23 | A | 3 | 13 | G | 25 | A | 15 | 20 | 0.25962 | 0.17206 |
| HMGCS1 | 886899 | T | 11 | C | 13 | 12 | T | 28 | C | 12 | 20 | 0.24167 | 0.10646 |
| POR2868178_TC | 669 | T | 5 | C | 21 | 13 | T | 18 | C | 24 | 21 | 0.23626 | 0.11773 |
| TUBB1054332_GA | 763 | A | 17 | G | 7 | 12 | A | 17 | G | 19 | 18 | 0.23611 | 0.10239 |
| CYP2D6_RS1467874_GA | 293 | A | 15 | G | 11 | 13 | A | 13 | G | 25 | 19 | 0.23482 | 0.09832 |
| CYP2B6RS2099361_CA | 81 | A | 16 | C | 10 | 13 | A | 34 | C | 6 | 20 | 0.23462 | 0.12885 |
| AP3D125672_CA | 873 | A | 14 | C | 6 | 10 | A | 15 | C | 17 | 16 | 0.23125 | 0.09766 |
| ACE | 971861 | G | 19 | A | 7 | 13 | G | 20 | A | 20 | 20 | 0.23077 | 0.10007 |
| CYP1B1RS1056837_TC | 151 | T | 16 | C | 6 | 11 | T | 20 | C | 20 | 20 | 0.22727 | 0.09681 |
| ACE_4320_GA | 321 | G | 11 | A | 15 | 13 | G | 26 | A | 14 | 20 | 0.22692 | 0.09157 |
| CYP2B6RS707265_GA | 283 | G | 13 | A | 13 | 13 | G | 26 | A | 10 | 18 | 0.22222 | 0.09222 |
| MYO5A1724631_CA | 879 | C | 19 | A | 7 | 13 | C | 40 | A | 2 | 21 | 0.22161 | 0.19222 |
| CYP2B6RS2279345_TC | 142 | C | 16 | T | 10 | 13 | C | 30 | T | 6 | 18 | 0.21795 | 0.10785 |
| RAB271014597_GT | 932 | T | 12 | G | 4 | 8 | T | 15 | G | 13 | 14 | 0.21429 | 0.08892 |
| AHR2158041_GA | 593 | G | 24 | A | 2 | 13 | G | 27 | A | 11 | 19 | 0.21255 | 0.14649 |
| ACE_1987692_TA | 48 | T | 10 | A | 14 | 12 | T | 25 | A | 15 | 20 | 0.20833 | 0.07666 |
| AHR1476080_CA | 640 | C | 17 | A | 7 | 12 | C | 20 | A | 20 | 20 | 0.20833 | 0.08028 |
| DCT2224780_TC | 674 | C | 17 | T | 5 | 11 | C | 17 | T | 13 | 15 | 0.20606 | 0.08551 |
| MAOA2283725_GA | 585 | G | 12 | A | 12 | 12 | G | 24 | A | 10 | 17 | 0.20588 | 0.07828 |
| GSTM1412302_TC | 461 | T | 9 | C | 17 | 13 | T | 22 | C | 18 | 20 | 0.20385 | 0.07407 |
| ACE_4329_GA | 322 | G | 15 | A | 11 | 13 | G | 15 | A | 25 | 20 | 0.20192 | 0.072 |
| ACE_4331_GA | 338 | G | 15 | A | 11 | 13 | G | 15 | A | 25 | 20 | 0.20192 | 0.072 |
| ACE_4973_GA | 341 | G | 15 | A | 11 | 13 | G | 15 | A | 25 | 20 | 0.20192 | 0.072 |
| ACE_4344_GA | 354 | G | 11 | A | 15 | 13 | G | 25 | A | 15 | 20 | 0.20192 | 0.072 |
| ESD1216967_GA | 690 | G | 14 | A | 12 | 13 | G | 31 | A | 11 | 21 | 0.19963 | 0.07646 |
| ASIP8818ag_GA | 859 | G | 9 | A | 17 | 13 | G | 6 | A | 34 | 20 | 0.19615 | 0.09359 |
| CYP2A131709081_TC | 503 | C | 17 | T | 1 | 9 | C | 27 | T | 9 | 18 | 0.19444 | 0.14648 |
| CYP1A2E7_405_GC | 98 | C | 19 | G | 7 | 13 | C | 22 | G | 18 | 20 | 0.18077 | 0.06264 |
| AHR2237298_GA | 600 | G | 19 | A | 7 | 13 | G | 22 | A | 18 | 20 | 0.18077 | 0.06264 |
| MVK | 886917 | A | 13 | C | 13 | 13 | A | 13 | C | 27 | 20 | 0.175 | 0.05555 |
| ACE_4309_TC | 256 | T | 13 | C | 13 | 13 | T | 13 | C | 25 | 19 | 0.15789 | 0.04484 |
| MYO5A1615235_GA | 919 | G | 18 | A | 8 | 13 | G | 34 | A | 6 | 20 | 0.15769 | 0.06326 |
| GSTP12370143_TC | 533 | T | 5 | C | 21 | 13 | T | 13 | C | 25 | 19 | 0.1498 | 0.05082 |
| GSTA22290757_TC | 495 | T | 10 | C | 10 | 10 | T | 22 | C | 12 | 17 | 0.14706 | 0.03871 |
| MYO5A752864_TC | 835 | C | 10 | T | 16 | 13 | C | 10 | T | 32 | 21 | 0.14652 | 0.04411 |
| OCA2 | 712054 | G | 10 | A | 12 | 11 | G | 13 | A | 29 | 21 | 0.14502 | 0.03905 |
| TYR | 217468 | C | 21 | A | 5 | 13 | C | 28 | A | 14 | 21 | 0.14103 | 0.04544 |
| GSTT2678863_GA | 786 | A | 12 | G | 14 | 13 | A | 24 | G | 16 | 20 | 0.13846 | 0.03365 |
| GSTA22608678_GA | 542 | G | 16 | A | 10 | 13 | G | 30 | A | 10 | 20 | 0.13462 | 0.03675 |
| GSTA22749019_GA | 583 | G | 16 | A | 10 | 13 | G | 30 | A | 10 | 20 | 0.13462 | 0.03675 |
| CYP2C9RS1200313_GT | 413 | T | 15 | G | 11 | 13 | T | 27 | G | 11 | 19 | 0.1336 | 0.03411 |
| CYP2D6 | 554365 | A | 14 | C | 10 | 12 | A | 18 | C | 22 | 20 | 0.13333 | 0.0311 |
| CYP2B6E7E8_610_TC | 165 | T | 13 | C | 13 | 13 | T | 14 | C | 24 | 19 | 0.13158 | 0.0308 |
| CYP2D6_RS2267447_TC | 259 | T | 17 | C | 9 | 13 | T | 19 | C | 17 | 18 | 0.12607 | 0.02873 |
| CYP2B6 | 1002413 | G | 13 | T | 13 | 13 | G | 25 | T | 15 | 20 | 0.125 | 0.02773 |
| CYP2B6RS2054675_TC | 149 | C | 13 | T | 13 | 13 | C | 15 | T | 25 | 20 | 0.125 | 0.02773 |

Those genes with more than one SNP on the list for association with elevated ALTGPT include GSTM1 (2), GSTA2 (4), ACE (10), MAOA (2), AHR (2) and CYP2B6 (7). When we restrict the analysis to Caucasian group (Table 9-8), we see that the only genes with more than one SNP associated with elevations in ALTGPT are the ACE gene (8 SNPs) and CYP2B6 (2). The results suggest that the ACE and CYP2B6 genes are the most important for ALTGPT elevations in LIPITOR™ patients, but all of the SNPs on the list would be useful for classifications. Haplotype analysis will reveal the extent to which the other genes with SNPs on the list will be helpful for classification.

LIPITOR ALTGPT 20% RESPONSE in Caucasians only: we compare genotypes from individuals that experienced at least a 20% increase in ALTGPT readings after taking LIPITOR (G1) versus everyone else (G2).

TABLE 9-8

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| CYP1B1RS1056837_TC | 151 | T | 14 | C | 2 | 8 | T | 14 | C | 14 | 14 | 0.375 | 0.31691 |
| AHR2237299_GA | 540 | G | 13 | A | 3 | 8 | G | 10 | A | 12 | 11 | 0.35795 | 0.2563 |
| XO_RS2295475_TC | 150 | T | 10 | C | 10 | 10 | T | 4 | C | 22 | 13 | 0.34615 | 0.25628 |
| ACE_4343_GA | 349 | A | 16 | G | 2 | 9 | A | 16 | G | 12 | 14 | 0.31746 | 0.24703 |
| MVK | 886917 | A | 12 | C | 8 | 10 | A | 8 | C | 20 | 14 | 0.31429 | 0.18041 |
| POR2868178_TC | 669 | T | 4 | C | 16 | 10 | T | 15 | C | 15 | 15 | 0.3 | 0.18062 |
| TUBB1054332_GA | 763 | A | 13 | G | 5 | 9 | A | 11 | G | 15 | 13 | 0.29915 | 0.16443 |
| ESD1923880_GA | 696 | G | 9 | A | 7 | 8 | G | 7 | A | 19 | 13 | 0.29327 | 0.15919 |
| CYP2B6 | 1002412 | G | 18 | A | 2 | 10 | G | 17 | A | 11 | 14 | 0.29286 | 0.22409 |
| ACE | 971861 | G | 14 | A | 6 | 10 | G | 12 | A | 16 | 14 | 0.27143 | 0.13379 |
| ACE_4320_GA | 321 | G | 9 | A | 11 | 10 | G | 20 | A | 8 | 14 | 0.26429 | 0.1282 |
| AP3D125672_CA | 873 | A | 13 | C | 5 | 9 | A | 11 | C | 13 | 12 | 0.26389 | 0.12865 |
| CYP2B6RS2099361_CA | 81 | A | 13 | C | 7 | 10 | A | 25 | C | 3 | 14 | 0.24286 | 0.15834 |
| ACE_1987692_TA | 48 | T | 9 | A | 11 | 10 | T | 19 | A | 9 | 14 | 0.22857 | 0.09409 |
| ACE_4329_GA | 322 | G | 11 | A | 9 | 10 | G | 9 | A | 19 | 14 | 0.22857 | 0.09409 |
| ACE_4331_GA | 338 | G | 11 | A | 9 | 10 | G | 9 | A | 19 | 14 | 0.22857 | 0.09409 |
| ACE_4973_GA | 341 | G | 11 | A | 9 | 10 | G | 9 | A | 19 | 14 | 0.22857 | 0.09409 |
| ACE_4344_GA | 354 | G | 9 | A | 11 | 10 | G | 19 | A | 9 | 14 | 0.22857 | 0.09409 |
| TYR_RS1827430_GA | 386 | G | 9 | A | 11 | 10 | G | 19 | A | 9 | 14 | 0.22857 | 0.09409 |
| GSTM1412302_TC | 461 | T | 7 | C | 13 | 10 | T | 16 | C | 12 | 14 | 0.22143 | 0.0872 |

ZOCOR™ RESPONSE in terms of LDL decrease in all patients regardless of race: 20% responders (decrease) (G1) versus others (G2).

TABLE 9-9

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| CYP2D6_RS2267444_GC | 93 | G | 38 | C | 6 | 22 | G | 4 | C | 12 | 8 | 0.61364 | 0.78469 |
| CYP2D6_RS2743456_GA | 347 | G | 39 | A | 3 | 21 | G | 7 | A | 7 | 7 | 0.42857 | 0.4774 |
| ESD1216961_TC | 677 | C | 28 | T | 8 | 18 | C | 6 | T | 10 | 8 | 0.40278 | 0.30849 |
| CYP2D6 | 554371 | C | 39 | T | 7 | 23 | C | 10 | T | 10 | 10 | 0.34783 | 0.25947 |
| CYP2C9RS2298037_TC | 248 | C | 29 | T | 15 | 22 | C | 18 | T | 0 | 9 | 0.33799 | 0.76627 |
| CYP2C9RS2860906_GA | 286 | A | 27 | G | 17 | 22 | A | 13 | G | 1 | 7 | 0.31494 | 0.28754 |
| CYP2D6_RS2856960_TC | 193 | T | 17 | C | 23 | 20 | T | 2 | C | 16 | 9 | 0.31389 | 0.24226 |
| CYP2E1RS2480257_TA | 37 | A | 19 | T | 5 | 12 | A | 4 | T | 4 | 4 | 0.29167 | 0.1691 |
| CYP2C8_RS947173_GA | 342 | G | 19 | A | 23 | 21 | G | 3 | A | 15 | 9 | 0.28571 | 0.176 |
| CYP2D6_RS2267447_TC | 259 | T | 30 | C | 12 | 21 | T | 7 | C | 9 | 8 | 0.27679 | 0.14035 |
| CYP2C181042194_GT | 712 | G | 26 | T | 10 | 18 | G | 16 | T | 0 | 8 | 0.27411 | 0.561 |
| CYP2D6_RS2267446_TC | 172 | C | 35 | T | 5 | 20 | C | 11 | T | 7 | 9 | 0.26389 | 0.17121 |
| CYP2D6 | 756251 | G | 42 | A | 4 | 23 | G | 13 | A | 7 | 10 | 0.26304 | 0.1979 |
| CYP2C8_RS1341159_GC | 94 | G | 18 | C | 24 | 21 | G | 3 | C | 15 | 9 | 0.2619 | 0.15034 |
| ABC11045642_TC | 665 | T | 28 | C | 18 | 23 | T | 7 | C | 13 | 10 | 0.2587 | 0.11919 |
| CYP2C8_RS947172_GA | 371 | G | 17 | A | 29 | 23 | G | 2 | A | 16 | 9 | 0.25845 | 0.17346 |
| CYP2C8 | 1004864 | G | 17 | A | 23 | 20 | G | 3 | A | 15 | 9 | 0.25833 | 0.14665 |
| CYP2C8_1341159_GC | 95 | G | 17 | C | 23 | 20 | G | 3 | C | 15 | 9 | 0.25833 | 0.14665 |
| ACE_4309_TC | 256 | T | 20 | C | 22 | 21 | T | 4 | C | 14 | 9 | 0.25397 | 0.12766 |
| OCA2 | 886896 | G | 19 | A | 29 | 24 | G | 3 | A | 17 | 10 | 0.24583 | 0.14005 |
| ACE_4311_TC | 135 | C | 20 | T | 16 | 18 | C | 5 | T | 11 | 8 | 0.24306 | 0.10678 |
| ABC12373589_GA | 681 | G | 37 | A | 7 | 22 | G | 12 | A | 8 | 10 | 0.24091 | 0.13178 |
| CYP2D6 | 554365 | A | 27 | C | 17 | 22 | A | 6 | C | 10 | 8 | 0.23864 | 0.10089 |
| CYP2C9RS1934969_TA | 39 | A | 18 | T | 24 | 21 | A | 12 | T | 6 | 9 | 0.2381 | 0.10142 |
| CYP2C8E93UTR_221_TC | 155 | T | 13 | C | 33 | 23 | T | 1 | C | 17 | 9 | 0.22705 | 0.18752 |
| CYP2C8_RS1058932_TC | 164 | T | 13 | C | 33 | 23 | T | 1 | C | 17 | 9 | 0.22705 | 0.18752 |
| MVKE7E8_197_GA | 578 | G | 23 | A | 7 | 15 | G | 10 | A | 0 | 5 | 0.22432 | 0.35578 |
| UGT1A2008584_TC | 756 | T | 22 | C | 14 | 18 | T | 10 | C | 2 | 6 | 0.22222 | 0.11171 |
| GSTM11296954_GA | 565 | G | 26 | A | 14 | 20 | G | 6 | A | 8 | 7 | 0.22143 | 0.0872 |
| CYP2B6RS2873265_TC | 120 | T | 9 | C | 23 | 16 | T | 6 | C | 6 | 6 | 0.21875 | 0.08914 |
| CYP2C8 | 1004863 | G | 30 | A | 12 | 21 | G | 8 | A | 8 | 8 | 0.21429 | 0.08527 |
| ACE_4344_GA | 354 | G | 21 | A | 25 | 23 | G | 12 | A | 6 | 9 | 0.21014 | 0.07917 |
| CYP2C8_RS1926705_TC | 122 | T | 26 | C | 8 | 17 | T | 10 | C | 8 | 9 | 0.20915 | 0.08679 |
| ACE | 971861 | G | 26 | A | 18 | 22 | G | 7 | A | 11 | 9 | 0.20202 | 0.07192 |
| CYP2A132545782_GA | 556 | G | 40 | A | 8 | 24 | G | 14 | A | 8 | 11 | 0.19697 | 0.0898 |
| CYP4B1RS837395_TA | 550 | G | 18 | A | 30 | 24 | G | 4 | A | 18 | 11 | 0.19318 | 0.08333 |
| POR8509_GA | 689 | G | 27 | A | 15 | 21 | G | 9 | A | 11 | 10 | 0.19286 | 0.06604 |
| CYP1A1_RS2515900_GA | 385 | G | 18 | A | 26 | 22 | G | 12 | A | 8 | 10 | 0.19091 | 0.06411 |
| PON3 | 869755 | T | 36 | C | 6 | 21 | T | 12 | C | 6 | 9 | 0.19048 | 0.09088 |
| CYP4B1RS751028_GA | 292 | G | 20 | A | 14 | 17 | G | 4 | A | 6 | 5 | 0.18824 | 0.0623 |
| OCA2 | 217458 | C | 17 | T | 29 | 23 | C | 4 | T | 18 | 11 | 0.18775 | 0.07909 |
| MAOB1799836_TC | 465 | T | 25 | C | 15 | 20 | T | 7 | C | 9 | 8 | 0.1875 | 0.06206 |
| UGT1A1042605_GA | 788 | G | 13 | A | 31 | 22 | G | 2 | A | 16 | 9 | 0.18434 | 0.0969 |
| TYR_RS1851992_GA | 278 | A | 30 | G | 18 | 24 | A | 9 | G | 11 | 10 | 0.175 | 0.05407 |
| ABC12235067_GA | 685 | G | 42 | A | 6 | 24 | G | 14 | A | 6 | 10 | 0.175 | 0.0835 |
| PON3 | 869790 | T | 31 | G | 15 | 23 | T | 10 | G | 10 | 10 | 0.17391 | 0.05483 |
| CYP2C9 | 869806 | A | 35 | G | 7 | 21 | A | 12 | G | 6 | 9 | 0.16667 | 0.06632 |
| ACE_4329_GA | 322 | G | 23 | A | 23 | 23 | G | 6 | A | 12 | 9 | 0.16667 | 0.05017 |
| ACE_4331_GA | 338 | G | 22 | A | 22 | 22 | G | 6 | A | 12 | 9 | 0.16667 | 0.05017 |
| TYR_RS1827430_GA | 386 | G | 23 | A | 23 | 23 | G | 12 | A | 6 | 9 | 0.16667 | 0.05017 |
| PON1 | 869817 | G | 32 | A | 12 | 22 | G | 16 | A | 2 | 9 | 0.16162 | 0.07711 |
| PON1 | 886930 | A | 29 | T | 15 | 22 | A | 9 | T | 9 | 9 | 0.15909 | 0.04555 |
| NAT21799929_TC | 530 | T | 15 | C | 29 | 22 | T | 9 | C | 9 | 9 | 0.15909 | 0.04555 |
| SILV1132095_GT | 704 | G | 20 | T | 8 | 14 | G | 10 | T | 8 | 9 | 0.15873 | 0.04778 |
| NAT21208_GA | 598 | G | 16 | A | 30 | 23 | G | 10 | A | 10 | 10 | 0.15217 | 0.04154 |
| CYP2B6 | 1002412 | G | 30 | A | 12 | 21 | G | 9 | A | 7 | 8 | 0.15179 | 0.04384 |
| ACE_4320_GA | 321 | A | 22 | G | 24 | 23 | A | 6 | G | 12 | 9 | 0.14493 | 0.03815 |
| ACE_4973_GA | 341 | G | 21 | A | 23 | 22 | G | 6 | A | 12 | 9 | 0.14394 | 0.03764 |
| GSTM3 | 971882 | A | 27 | C | 15 | 21 | A | 9 | C | 9 | 9 | 0.14286 | 0.03647 |
| GSTP12370143_TC | 533 | C | 27 | T | 15 | 21 | C | 8 | T | 8 | 8 | 0.14286 | 0.03647 |
| ESD1216967_GA | 690 | G | 26 | A | 20 | 23 | G | 14 | A | 6 | 10 | 0.13478 | 0.03424 |
| NAT21495744_GA | 588 | A | 32 | G | 14 | 23 | A | 9 | G | 7 | 8 | 0.13315 | 0.03327 |
| CYP2D6 | 869777 | G | 29 | T | 17 | 23 | G | 10 | T | 10 | 10 | 0.13043 | 0.03025 |

Next SNPs related to the efficacy of Zocor™ were identified. The results from this screen are quite clear. Of the top 25 delta scores (reading from the top of the table down), 8 belong to CYP2D6 SNPs, 6 to CYP2C8 SNPs. Half of them are therefore CYP2D6 and CYP2C8 SNPs, which is far from random given the number and diversity of SNPs surveyed (p <0.0001). Further, the rest of the top 25 SNPs were found in the CYP2C9 gene (2 SNPs), the ABC1 (3 SNPs) and ACE (2 SNPs) gene. Only one pigmentation gene SNP was part of the top 25 scores. When we restrict the analysis to Caucasians we observe 27 associated SNPs and the following genes had more than one SNP on the list: CYP2D6 (6), CYP2C8 (8), CYP2C9 (3) and ACE (4). We therefore conclude that the CYP2D6, CYP2C8, CYP2C9 and ACE genes are important for LDL response in ZOCOR™ patients.

ZOCOR RESPONSE in terms of LDL decrease in Caucasians only: 20% responders (decrease) (G1) versus others (G2).

TABLE 9-10

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| CYP2D6_RS2267444_GC | 93 | G | 36 | C | 6 | 21 | G | 4 | C | 10 | 7 | 0.57143 | 0.67205 |
| CYP2D6_RS2743456_GA | 347 | G | 37 | A | 3 | 20 | G | 6 | A | 6 | 6 | 0.425 | 0.46371 |
| ACE_4309_TC | 256 | T | 20 | C | 20 | 20 | T | 2 | C | 12 | 7 | 0.35714 | 0.27791 |
| CYP2C8_RS1926705_TC | 122 | T | 25 | C | 7 | 16 | T | 6 | C | 8 | 7 | 0.35268 | 0.23904 |
| CYP2C9RS2298037_TC | 248 | C | 27 | T | 15 | 21 | C | 14 | T | 0 | 7 | 0.3524 | 0.7382 |
| ESD1216961_TC | 677 | C | 28 | T | 8 | 18 | C | 6 | T | 8 | 7 | 0.34921 | 0.23362 |
| CYP2D6 | 554371 | C | 37 | T | 7 | 22 | C | 8 | T | 8 | 8 | 0.34091 | 0.24651 |
| ACE_4311_TC | 135 | C | 20 | T | 14 | 17 | C | 3 | T | 9 | 6 | 0.33824 | 0.21377 |
| CYP2C8_RS947173_GA | 342 | G | 19 | A | 21 | 20 | G | 2 | A | 12 | 7 | 0.33214 | 0.24402 |
| CYP2C8 | 1004863 | G | 29 | A | 11 | 20 | G | 5 | A | 7 | 6 | 0.30833 | 0.17487 |
| CYP2D6_RS2267447_TC | 259 | T | 29 | C | 11 | 20 | T | 5 | C | 7 | 6 | 0.30833 | 0.17487 |
| CYP2C8_RS1341159_GC | 94 | G | 18 | C | 22 | 20 | G | 2 | C | 12 | 7 | 0.30714 | 0.21224 |
| CYP2C8 | 1004864 | G | 17 | A | 21 | 19 | G | 2 | A | 12 | 7 | 0.30451 | 0.20901 |
| CYP2C8_1341159_GC | 95 | G | 17 | C | 21 | 19 | G | 2 | C | 12 | 7 | 0.30451 | 0.20901 |
| CYP2C9RS2860906_GA | 286 | A | 26 | G | 16 | 21 | A | 11 | G | 1 | 6 | 0.29762 | 0.24718 |
| CYP2E1RS2480257_TA | 37 | A | 19 | T | 5 | 12 | A | 4 | T | 4 | 4 | 0.29167 | 0.1691 |
| CYP2C9RS1934969_TA | 39 | A | 17 | T | 23 | 20 | A | 10 | T | 4 | 7 | 0.28929 | 0.1531 |
| CYP2D6 | 554365 | A | 26 | C | 16 | 21 | A | 4 | C | 8 | 6 | 0.28571 | 0.14625 |
| ACE_4344_GA | 354 | G | 19 | A | 25 | 22 | G | 10 | A | 4 | 7 | 0.28247 | 0.14607 |
| CYP2D6_RS2856960_TC | 193 | T | 16 | C | 22 | 19 | T | 2 | C | 12 | 7 | 0.2782 | 0.178 |
| CYP2A6RS696839_GC | 91 | G | 27 | C | 3 | 15 | G | 5 | C | 3 | 4 | 0.275 | 0.20141 |
| CYP2C181042194_GT | 712 | G | 26 | T | 10 | 18 | G | 12 | T | 0 | 6 | 0.27141 | 0.49393 |
| CYP2A132545782_GA | 556 | G | 38 | A | 8 | 23 | G | 10 | A | 8 | 9 | 0.27053 | 0.15685 |
| ACE | 971861 | G | 26 | A | 16 | 21 | G | 5 | A | 9 | 7 | 0.2619 | 0.12208 |
| CYP2C82275622_TC | 459 | C | 30 | T | 14 | 22 | C | 6 | T | 8 | 7 | 0.25325 | 0.11546 |
| CYP2C8_RS947172_GA | 371 | G | 17 | A | 27 | 22 | G | 2 | A | 12 | 7 | 0.24351 | 0.14056 |

ZOCOR response in terms of Total Cholesterol (TC) decrease in all patients (G1—20% responders versus G2—others). Given the total sample (about 70), those with deltas less than 0.15, or those with deltas above 0.15 but with a sample less than 15 for the minor allele were eliminated.

TABLE 9-11

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| UGT1A2008595_GA | 768 | G | 8 | A | 18 | 13 | G | 19 | A | 13 | 16 | 0.28606 | 0.14789 |
| DCT2224780_TC | 674 | C | 22 | T | 6 | 14 | C | 14 | T | 14 | 14 | 0.28571 | 0.16122 |
| CYP4B1RS837400_GA | 336 | G | 20 | A | 18 | 19 | G | 34 | A | 8 | 21 | 0.28321 | 0.16501 |
| CYP2D6_RS2267444_GC | 93 | G | 28 | C | 8 | 18 | G | 20 | C | 20 | 20 | 0.27778 | 0.15113 |
| CYP2C9RS1934969_TA | 39 | T | 19 | A | 13 | 16 | T | 14 | A | 28 | 21 | 0.26042 | 0.12131 |
| NAT21799930_GA | 603 | G | 24 | A | 14 | 19 | G | 35 | A | 5 | 20 | 0.24342 | 0.14873 |
| HMGCS1 | 886899 | C | 21 | T | 19 | 20 | C | 12 | T | 30 | 21 | 0.23929 | 0.10562 |
| CYP4B1RS2297812_GC | 97 | G | 22 | C | 14 | 18 | G | 32 | C | 6 | 19 | 0.23099 | 0.12259 |
| NAT21041983_TC | 483 | T | 23 | C | 13 | 18 | T | 9 | C | 13 | 11 | 0.2298 | 0.09364 |
| CYP2C9RS2298037_TC | 248 | T | 13 | C | 25 | 19 | T | 5 | C | 35 | 20 | 0.21711 | 0.12182 |
| ESD1216961_TC | 677 | C | 24 | T | 10 | 17 | C | 14 | T | 14 | 14 | 0.20588 | 0.07828 |
| CYP2B6RS707265_GA | 283 | G | 19 | A | 21 | 20 | G | 28 | A | 14 | 21 | 0.19167 | 0.06603 |
| NAT21799929_TC | 530 | T | 12 | C | 26 | 19 | T | 19 | C | 19 | 19 | 0.18421 | 0.06186 |
| CYP2C8_RS947172_GA | 371 | G | 14 | A | 24 | 19 | G | 8 | A | 34 | 21 | 0.17794 | 0.07016 |
| ESD1216967_GA | 690 | G | 22 | A | 18 | 20 | G | 32 | A | 12 | 22 | 0.17727 | 0.06006 |
| CYP3A4_RS2246709_GA | 384 | G | 11 | A | 15 | 13 | G | 6 | A | 18 | 12 | 0.17308 | 0.05927 |
| GSTT2140188_GC | 652 | C | 31 | G | 7 | 19 | C | 27 | G | 15 | 21 | 0.17293 | 0.06762 |
| CYP2B6RS2279345_TC | 142 | T | 17 | C | 19 | 18 | T | 12 | C | 28 | 20 | 0.17222 | 0.05505 |
| CYP4B1RS2405335_TC | 143 | T | 29 | C | 11 | 20 | T | 37 | C | 5 | 21 | 0.15595 | 0.0699 |
| CYP2B6 | 1002412 | G | 29 | A | 7 | 18 | G | 26 | A | 14 | 20 | 0.15556 | 0.0542 |
| CYP2B6RS2099361_CA | 81 | C | 15 | A | 21 | 18 | C | 11 | A | 31 | 21 | 0.15476 | 0.04702 |
| NAT21495744_GA | 588 | G | 12 | A | 28 | 20 | G | 18 | A | 22 | 20 | 0.15 | 0.04212 |
| NAT21208_GA | 598 | A | 26 | G | 14 | 20 | A | 22 | G | 22 | 22 | 0.15 | 0.04033 |
| UGT1A2008584_TC | 756 | T | 18 | C | 16 | 17 | T | 19 | C | 9 | 14 | 0.14916 | 0.04077 |

For the first of any of our screens, we see NAT2 as a major contributor towards variable Statin response, in this case Zocor™ response in terms of TC level reduction in individuals without regard to race (Table 9-11). NAT2 SNPs appear 5 times in this group of 25 SNPs associated with outcome for this particular drug/test combination. The CYP2B6 gene has 4 SNPs in this list of 25. Neither NAT2 nor CYP2B6 were significant components of variable LIPITOR™ response using any response metric, nor ZOCOR™ response using the LDL metric, which suggests a certain specificity to these results. Looking at TC response in Caucasians only (Table 9-12), we see the following genes with more than one SNP on the list of significant SNPs: CYP4B1 (3), UGT1A2 (3), NAT2 (3) and CYP2B6 (2) genes. We therefore conclude that variants in the CYP4B1, UGT1A2, NAT2 and CYP2B6 genes are associated with TC outcome in ZOCOR™ patients.

ZOCOR™ response in terms of Total Cholesterol (TC) decrease in Caucasians only (G1—20% responders versus G2—others). Given the total sample (about 70), those with deltas less than 0.15, or those with deltas above 0.15 but with a sample less than 15 for the minor allele were eliminated.

TABLE 9-12

| | | | | | | SAM- | | | | | SAM- | | |
|---|---|---|---|---|---|---|---|---|---|---|---|---|---|
| | | G1 | | G1 | | PLE | G2 | | G2 | | PLE | | |
| Gene | Marker | A1 | NO | A2 | NO | SIZE | A1 | NO | A2 | NO | SIZE | DELTA | EAE |
| CYP4B1RS837400_GA | 336 | G | 18 | A | 18 | 18 | G | 24 | A | 4 | 14 | 0.35714 | 0.27791 |
| UGT1A2008584_TC | 756 | C | 16 | T | 16 | 16 | C | 3 | T | 17 | 10 | 0.35 | 0.26366 |
| DCT2224780_TC | 674 | C | 22 | T | 6 | 14 | C | 9 | T | 11 | 10 | 0.33571 | 0.21869 |
| UGT1A2008595_GA | 768 | G | 7 | A | 17 | 12 | G | 12 | A | 8 | 10 | 0.30833 | 0.17311 |
| NAT21041983_TC | 483 | T | 21 | C | 13 | 17 | T | 5 | C | 11 | 8 | 0.30515 | 0.16804 |
| CYP2B6RS2873265_TC | 120 | C | 25 | T | 5 | 15 | C | 9 | T | 7 | 8 | 0.27083 | 0.15974 |
| CYP4B1RS2297812_GC | 97 | G | 20 | C | 14 | 17 | G | 24 | C | 4 | 14 | 0.26891 | 0.1676 |
| PON1 | 886930 | A | 27 | T | 9 | 18 | A | 14 | T | 14 | 14 | 0.25 | 0.11928 |
| CYP2C9RS1934969_TA | 39 | T | 18 | A | 12 | 15 | T | 10 | A | 18 | 14 | 0.24286 | 0.10476 |
| CYP4B1RS2297809_TC | 219 | C | 26 | T | 10 | 18 | C | 27 | T | 1 | 14 | 0.24206 | 0.24603 |
| RAB27526213_TC | 844 | T | 13 | C | 25 | 19 | T | 18 | C | 14 | 16 | 0.22039 | 0.08665 |
| GSTP12370143_TC | 533 | C | 19 | T | 15 | 17 | C | 20 | T | 6 | 13 | 0.21041 | 0.08842 |
| UGT1A1875263_TC | 755 | C | 22 | T | 12 | 17 | C | 24 | T | 4 | 14 | 0.21008 | 0.10817 |
| NAT21799930_GA | 603 | G | 23 | A | 13 | 18 | G | 22 | A | 4 | 13 | 0.20726 | 0.10209 |
| CYP2B6RS707265_GA | 283 | G | 18 | A | 20 | 19 | G | 19 | A | 9 | 14 | 0.20489 | 0.07586 |
| MAOB1799836_TC | 465 | T | 15 | C | 17 | 16 | T | 16 | C | 8 | 12 | 0.19792 | 0.07034 |
| NAT21799929_TC | 530 | T | 11 | C | 25 | 18 | T | 14 | C | 14 | 14 | 0.19444 | 0.06933 |
| CYP2D6_RS2267444_GC | 93 | G | 26 | C | 8 | 17 | G | 16 | C | 12 | 14 | 0.19328 | 0.07479 |

A comparison of genotypes from individuals that experienced at least a 20% increase in SGOT readings after taking ZOCOR (G1) versus everyone else (G2) was not possible because the sample size for adverse responders was only 4 for this drug.

A comparison of genotypes from individuals that experienced at least a 20% increase in ALTGPT readings after taking ZOCOR™ (G1) versus everyone else (G2) was not possible because the sample for adverse responders was only 4 for this drug.

Summary

The results of this SNP screen are shown in Table 9-13. From Table 9-13 it is evident that many different genes impact variable Statin response. For most of the outcomes, there were SNPs from at least four different genes associated. It is also clear that the gene compliments are highly unique for each end point and each gene. The GSTs (GSTM1, GSTT2, GSTA2) were quite strongly associated with LIPITOR™ response, linked with LDL, TC and SGOT outcome, but not ZOCOR™ response. The NAT2 gene was only found to be relevant for ZOCOR™ response, and only had impact on the TC lowering effect of the drug, not the LDL lowering effect. CYP2C8 was an important determinant for both LIPITOR™ and ZOCOR™, for the former, impacting both TC and SGOT outcome. Of significant interest, no SNPs, or only weakly associated SNPs in the HMGCS1, MVK or HMGCR gene were identified, though we previously described HMGCR haplotypes associated with response. Usually, the ability to identify associations at the level of the SNP indicates the gene contribution towards response is relatively strong compared to genes with associations only apparent at the level of the haplotype. The HMGCS 1, MVK and HMGCR genes are part of the cholesterol synthesis pathway inhibited by Statins, yet our results suggest that most of Statin variable response is attributed by xenobiotic metabolism gene sequences, not target pathway sequences. The associations we have described earlier in this application therefore are a function of haplotype, not SNP sequences. With these haplotypes, and SNPs from genes below, a linear or quadratic discriminate classifier (as we have described elsewhere, (T. Frudakis, U.S. patent application Ser. No. 10/156,995, filed May 28, 2002), Fru fdakis et al., J. Forensic Science, (2002); Frudakis 2002a) is possible to predict each outcome.

TABLE 9-13
Genes with SNPs most strongly associated with each test for both LIPITOR ™ and ZOCOR ™.

| DRUG | TEST | RESPONSE | GENES1 | | | | | | | | |
|---|---|---|---|---|---|---|---|---|---|---|---|
| LIPITOR | LDL | 20% DECREASE | CYP2D6 | CYP4B1 | GSTM1 | ESD | UGT1A2 | | | | |
| LIPITOR | TC | 20% DECREASE | CYP2C8 | GSTT2 | GSTM1 | MYO5A* | OCA2* | | | | |
| LIPITOR | SGOT | 20% INCREASE | GSTA2 | GSTT2 | CYP2C8 | CYP4B1 | CYP2C9 | DCT* | MYO5A* | AP3D1* | AIM* |
| LIPITOR | ALTGPT | 20% INCREASE | ACE | CYP2B6 | | | | | | | |
| ZOCOR | LDL | 20% DECREASE | CYP2C8 | CYP2D6 | ACE | CYP2C9 | | | | | |
| ZOCOR | TC | 20% DECREASE | CYP4B1 | UGT1A2 | NAT2 | CYP2B6 | | | | | |

| | | | |
|---|---|---|---|
| ZOCOR | SGOT | 20% INCREASE | incidence is too low to measure with our sample |
| ZOCOR | ALTGPT | 20% INCREASE | incidence is too low to measure with our sample |

1-(RANKED IN ORDER OF ASSOCIATION STRENGTH AT THE LEVEL OF THE INDIVIDUAL SNP)
*Also useful for classification if race is not known

TABLE 9-14
List of SNPs identified in Example 9 as
being related to a statin response.

| SNP name or Gene | | |
|---|---|---|
| name | Marker number | SEQ ID NO: |
| UGT1A2008584 | 756 | SEQ ID NO: 43 |
| UGT1A1875263 | 755 | SEQ ID NO: 44 |
| SILV1052165 | 662 | SEQ ID NO: 45 |
| RAB27526213 | 844 | SEQ ID NO: 46 |
| GSTM1414673 | 580 | SEQ ID NO: 47 |
| CYP2E1RS2480257 | 37 | SEQ ID NO: 48 |
| CYP4B1RS2405335 | 143 | SEQ ID NO: 49 |
| ESD1923880 | 696 | SEQ ID NO: 50 |
| CYP4B1RS681840 | 194 | SEQ ID NO: 51 |
| ACE_4311 | 135 | SEQ ID NO: 52 |
| AP3D12072304 | 906 | SEQ ID NO: 53 |
| AHR2106728 | 599 | SEQ ID NO: 54 |
| GSTM1421547 | 527 | SEQ ID NO: 55 |
| CYP4B1RS2065996 | 137 | SEQ ID NO: 56 |
| SILV1132095 | 704 | SEQ ID NO: 57 |
| UGT1A2008595 | 768 | SEQ ID NO: 58 |
| MYO5A1693494 | 836 | SEQ ID NO: 59 |
| CYP4B1RS2297810 | 350 | SEQ ID NO: 60 |
| MYO5A1724631 | 879 | SEQ ID NO: 61 |
| MYO5A1669871 | 847 | SEQ ID NO: 62 |
| NAT21041983 | 483 | SEQ ID NO: 63 |
| GSTT22267047 | 464 | SEQ ID NO: 64 |
| CYP2C8_RS1891071 | 369 | SEQ ID NO: 65 |
| CYP4B1RS751027 | 343 | SEQ ID NO: 66 |
| MYO5A2899489 | 930 | SEQ ID NO: 67 |
| GSTT2140184 | 568 | SEQ ID NO: 68 |
| CYP2C8E2E3_397 | 134 | SEQ ID NO: 69 |
| CYP2B6RS2279345 | 142 | SEQ ID NO: 70 |
| MYO5A935892 | 898 | SEQ ID NO: 71 |
| MYO5A1669870 | 877 | SEQ ID NO: 72 |
| MYO5A1693512 | 821 | SEQ ID NO: 73 |
| CYP2A131709081 | 503 | SEQ ID NO: 74 |
| MAOA909525 | 549 | SEQ ID NO: 75 |
| RAB271014597 | 932 | SEQ ID NO: 76 |
| CYP2C8_RS1891070 | 357 | SEQ ID NO: 77 |
| CYP2C8_RS1341159 | 94 | SEQ ID NO: 78 |
| CYP2C8_1341159 | 95 | SEQ ID NO: 79 |
| POR17685 | 691 | SEQ ID NO: 80 |
| CYP2C8_2071426 | 362 | SEQ ID NO: 81 |
| CYP2C8_RS947173 | 342 | SEQ ID NO: 82 |
| GSTT2140185 | 783 | SEQ ID NO: 83 |
| GSTT2140188 | 652 | SEQ ID NO: 84 |
| CYP4B1RS751028 | 292 | SEQ ID NO: 85 |
| GSTP12370143 | 533 | SEQ ID NO: 86 |
| DCT2224780 | 674 | SEQ ID NO: 87 |
| CYP3A7RS2687140 | 287 | SEQ ID NO: 88 |
| GSTT2140192 | 469 | SEQ ID NO: 89 |
| CES22241409 | 658 | SEQ ID NO: 90 |
| AP3D125673 | 828 | SEQ ID NO: 91 |
| CYP1A2E7_405 | 98 | SEQ ID NO: 92 |
| CYP2A131709084 | 546 | SEQ ID NO: 93 |
| GSTA22290758 | 558 | SEQ ID NO: 94 |
| CYP4B1RS632645 | 171 | SEQ ID NO: 95 |
| GSTT2140187 | 562 | SEQ ID NO: 96 |
| GSTT2140190 | 443 | SEQ ID NO: 97 |
| DCT1325611 | 657 | SEQ ID NO: 98 |
| DCT2892680 | 699 | SEQ ID NO: 99 |
| GSTA22290757 | 495 | SEQ ID NO: 100 |
| CYP2D6_RS2267444 | 93 | SEQ ID NO: 101 |
| CYP4B1RS2297812 | 97 | SEQ ID NO: 102 |
| MYO5A722436 | 929 | SEQ ID NO: 103 |
| MYO5A1724630 | 806 | SEQ ID NO: 104 |
| GSTA21051775 | 456 | SEQ ID NO: 105 |
| POR8509 | 689 | SEQ ID NO: 106 |
| AP3D12238593 | 834 | SEQ ID NO: 107 |
| AP3D12238594 | 838 | SEQ ID NO: 108 |
| GSTA21051536 | 440 | SEQ ID NO: 109 |
| GSTT2678863 | 786 | SEQ ID NO: 110 |
| TYR_RS1851992 | 278 | SEQ ID NO: 111 |
| CYP2C9RS2860905 | 367 | SEQ ID NO: 112 |
| CYP2C82071426 | 596 | SEQ ID NO: 113 |
| DCT727299 | 682 | SEQ ID NO: 114 |
| GSTA22144696 | 455 | SEQ ID NO: 115 |
| DCT2296498 | 701 | SEQ ID NO: 116 |
| AP3D12072305 | 820 | SEQ ID NO: 117 |
| GSTA22180319 | 577 | SEQ ID NO: 118 |
| MYO5A1724639 | 843 | SEQ ID NO: 119 |
| GSTT22719 | 611 | SEQ ID NO: 120 |
| GSTA22894803 | 435 | SEQ ID NO: 121 |
| CYP2C8E8_92 | 265 | SEQ ID NO: 122 |
| AIM35415 | 937 | SEQ ID NO: 123 |
| CYP2C9RS2298037 | 248 | SEQ ID NO: 124 |
| CYP4B1RS1572603 | 176 | SEQ ID NO: 125 |
| GSTT2140194 | 442 | SEQ ID NO: 126 |
| GSTA22608677 | 451 | SEQ ID NO: 127 |
| GSTA22608679 | 570 | SEQ ID NO: 128 |
| GSTT2140196 | 605 | SEQ ID NO: 129 |
| MYO5A752864 | 835 | SEQ ID NO: 130 |
| CYP2B6RS707265 | 283 | SEQ ID NO: 131 |
| CYP2D6_RS2267446 | 172 | SEQ ID NO: 132 |
| AHR2237299 | 540 | SEQ ID NO: 133 |
| CYP2C181042194 | 712 | SEQ ID NO: 134 |
| CYP1A2_RS2069524 | 206 | SEQ ID NO: 135 |
| CYP2D6_RS2856960 | 193 | SEQ ID NO: 136 |
| ESD1216958 | 706 | SEQ ID NO: 137 |
| CYP2A6RS1061608 | 41 | SEQ ID NO: 138 |
| CYP4B1RS837400 | 336 | SEQ ID NO: 139 |
| CYP2A6RS1137115 | 284 | SEQ ID NO: 140 |
| GSTT2140186 | 545 | SEQ ID NO: 141 |
| MAOB1799836 | 465 | SEQ ID NO: 142 |
| GSTA22144697 | 474 | SEQ ID NO: 143 |
| GSTA22180315 | 500 | SEQ ID NO: 144 |
| GSTA22749019 | 583 | SEQ ID NO: 145 |
| ACE_4343 | 349 | SEQ ID NO: 146 |
| ACE_4335 | 291 | SEQ ID NO: 147 |
| POR2868178 | 669 | SEQ ID NO: 148 |
| TUBB1054332 | 763 | SEQ ID NO: 149 |
| CYP2D6_RS1467874 | 293 | SEQ ID NO: 150 |
| CYP2B6RS2099361 | 81 | SEQ ID NO: 151 |
| AP3D125672 | 873 | SEQ ID NO: 152 |
| CYP1B1RS1056837 | 151 | SEQ ID NO: 153 |
| ACE_4320 | 321 | SEQ ID NO: 154 |
| AHR2158041 | 593 | SEQ ID NO: 155 |
| ACE_1987692 | 48 | SEQ ID NO: 156 |
| AHR1476080 | 640 | SEQ ID NO: 157 |
| MAOA2283725 | 585 | SEQ ID NO: 158 |
| GSTM1412302 | 461 | SEQ ID NO: 159 |
| ACE_4329 | 322 | SEQ ID NO: 160 |
| ACE_4331 | 338 | SEQ ID NO: 161 |
| ACE_4973 | 341 | SEQ ID NO: 162 |
| ACE_4344 | 354 | SEQ ID NO: 163 |
| ESD1216967 | 690 | SEQ ID NO: 164 |
| AHR2237298 | 600 | SEQ ID NO: 165 |
| ACE_4309 | 256 | SEQ ID NO: 166 |
| MYO5A1615235 | 919 | SEQ ID NO: 167 |
| GSTA22608678 | 542 | SEQ ID NO: 168 |
| CYP2C9RS1200313 | 413 | SEQ ID NO: 169 |
| CYP2B6E7E8_610 | 165 | SEQ ID NO: 170 |
| CYP2D6_RS2267447 | 259 | SEQ ID NO: 171 |
| CYP2B6RS2054675 | 149 | SEQ ID NO: 172 |
| TYR_RS1827430 | 386 | SEQ ID NO: 173 |
| CYP2D6_RS2743456 | 347 | SEQ ID NO: 174 |
| ESD1216961 | 677 | SEQ ID NO: 175 |
| CYP2C9RS2860906 | 286 | SEQ ID NO: 176 |
| ABC11045642 | 665 | SEQ ID NO: 177 |
| CYP2C8_RS947172 | 371 | SEQ ID NO: 178 |
| ABC12373589 | 681 | SEQ ID NO: 179 |
| CYP2C9RS1934969 | 39 | SEQ ID NO: 180 |
| CYP2C8E93UTR_221 | 155 | SEQ ID NO: 181 |
| CYP2C8_RS1058932 | 164 | SEQ ID NO: 182 |
| MVKE7E8_197 | 578 | SEQ ID NO: 183 |
| GSTM11296954 | 565 | SEQ ID NO: 184 |
| CYP2B6RS2873265 | 120 | SEQ ID NO: 185 |
| CYP2C8_RS1926705 | 122 | SEQ ID NO: 186 |
| CYP2A132545782 | 556 | SEQ ID NO: 187 |
| CYP4B1RS837395 | 550 | SEQ ID NO: 188 |
| CYP1A1_RS2515900 | 385 | SEQ ID NO: 189 |
| UGT1A1042605 | 788 | SEQ ID NO: 190 |
| ABC12235067 | 685 | SEQ ID NO: 191 |
| NAT21799929 | 530 | SEQ ID NO: 192 |
| NAT21208 | 598 | SEQ ID NO: 193 |
| NAT21495744 | 588 | SEQ ID NO: 194 |
| CYP2A6RS696839 | 91 | SEQ ID NO: 195 |
| CYP2C82275622 | 459 | SEQ ID NO: 196 |
| NAT21799930 | 603 | SEQ ID NO: 197 |
| CYP3A4_RS2246709 | 384 | SEQ ID NO: 198 |
| CYP4B1RS2297809 | 219 | SEQ ID NO: 199 |
| PON3 | 869755 | SEQ ID NO: 200 |
| CYP2D6 | 869777 | SEQ ID NO: 201 |
| CYP2D6 | 554371 | SEQ ID NO: 202 |
| CYP2D6 | 554365 | SEQ ID NO: 203 |
| TYR | 217468 | SEQ ID NO: 204 |
| CYP2B6 | 1002412 | SEQ ID NO: 205 |
| PON1 | 869817 | SEQ ID NO: 206 |
| CYP2C8E2E3_397 | null | SEQ ID NO: 207 |
| GSTM3 | 971882 | SEQ ID NO: 208 |
| OCA2 | 886896 | SEQ ID NO: 209 |
| OCA2 | 886894 | SEQ ID NO: 210 |
| CYP2C8 | 1004864 | SEQ ID NO: 211 |
| CYP2C9 | 869797 | SEQ ID NO: 212 |
| CYP2C8_1341159 | null | SEQ ID NO: 213 |
| CYP2C8_2071426 | 1004857 | SEQ ID NO: 214 |
| CYP2C8_RS947173 | 1004864 | SEQ ID NO: 215 |
| CYP1A2E7_405 | null | SEQ ID NO: 216 |
| CYP2C8 | 1004867 | SEQ ID NO: 217 |
| CYP2C8E8_92 | null | SEQ ID NO: 218 |
| FDPS | 756238 | SEQ ID NO: 219 |
| CYP2C9 | 869803 | SEQ ID NO: 220 |
| PON3 | 869790 | SEQ ID NO: 221 |
| HMGCS1 | 886899 | SEQ ID NO: 222 |
| ACE | 971861 | SEQ ID NO: 223 |
| MVK | 886917 | SEQ ID NO: 224 |
| OCA2 | 712054 | SEQ ID NO: 225 |
| CYP2B6E7E8_610 | null | SEQ ID NO: 226 |
| CYP2B6 | 1002413 | SEQ ID NO: 227 |
| CYP2D6 | 756251 | SEQ ID NO: 228 |
| CYP2C8E93UTR_221 | null | SEQ ID NO: 229 |
| CYP2C8 | 1004863 | SEQ ID NO: 230 |
| OCA2 | 217458 | SEQ ID NO: 231 |
| CYP2C9 | 869806 | SEQ ID NO: 232 |
| PON1 | 886930 | SEQ ID NO: 233 |
| CYP3A4_RS2246709 | null | SEQ ID NO: 234 |

| | |
|---|---|
| GSTM1421547 (SNP in brackets) | SEQ ID NO:55 |

GTGTTCTTCAGTATGAGACGGTGGCTCCAGTGGCCTTTGAAGTCACACCG
TGATATGTGACCCATGGTACAACCTCCACGAGAACAATGTCCAACCTGCC
AACTTTCTTCTTTCAAGGTAGAAGGAAGACTTTCAAAAGAGTTGTGCAAT
GGATTAGCCTGGGGTTGACTGCTTTAAAGGATATTGCAAATAATAATGGA
{C/T}ATATGGAAATAGATGATAGACCTTTAATGAGAAATCATTTTGCAA
TGTAAACCAGGCTGTTGTGCTGCAAAAAAAGTAGTTTTTTTGTTTTGTTT
TGTTTTGTTTTGTTTTGTTTTGTTTTTTGTAAATTAGCTAAAACATTGTT
AGGACTCCAGAGGATGAACCCAGTATATCAAAAAAGTTTCAAACCACCTG
GATAA

What is claimed is:

1. A method for inferring a statin response of a human subject from a nucleic acid sample of the subject, the method comprising identifying, in the nucleic acid sample, at least one haplotype allele indicative of a statin response, wherein the haplotype allele comprises
a) nucleotides of the cytochrome p450 3A4 (CYP3A4) gene, corresponding to
i) a CYP3A4A haplotype, which comprises
nucleotide 808 of SEQ ID NO:8 {CYP3A4E10-5_{—}292}, and
nucleotide 227 of SEQ ID NO:9 {CYP3A4E12_{—}76}; or
ii) a CYP3A4B haplotype, which comprises
nucleotide 1311 of SEQ ID NO:7 {CYP3A4E7_{—}243},
nucleotide 808 of SEQ ID NO:8 {CYP3A4E10-5_{—}292}, and
nucleotide 227 of SEQ ID NO:9 {CYP3A4E12_{—}76}; or
iii) a CYP3A4C haplotype, which comprises
nucleotide 425 of SEQ ID NO:10 {CYP3A4E3-5_{—}249},
nucleotide 1311 of SEQ ID NO:7 {CYP3A4E7_{—}243},
nucleotide 808 of SEQ ID NO:8 {CYP3A4E10-5_{—}292}, and
nucleotide 227 of SEQ ID NO:9 {CYP3A4E12_{—}76}; or
b.) nucleotides of the 3-hydroxy-3-methylglutaryl-coenzyme A reductase (HMGCR) gene, corresponding to:
i) an HMGCRA haplotype, which comprises
nucleotide 1757 of SEQ ID NO:2 {HMGCRE7E11-3_{—}472}, and
nucleotide 1430 of SEQ ID NO:3 {HMGCRDBSNP_{—}45320};
ii) an HMGCRB haplotype, which comprises
nucleotide 519 of SEQ ID NO:11 {HMGCRE5E6-3_{—}283},
nucleotide 1757 of SEQ ID NO:2 {HMGCRE7E11-3_{—}472},
nucleotide 1430 of SEQ ID NO:3 {HMGCRDBSNP_{—}45320}, and
nucleotide 1421 of SEQ ID NO:12 {HMGCRE16E18_{—}99}; or
iii) an HMGCRC haplotype, which comprises
nucleotide 1757 of SEQ ID NO:2 {HMGCRE7E11-3_{—}472},
nucleotide 1430 of SEQ ID NO:3 {HMGCRDBSNP_{—}45320}, and
nucleotide 1421 of SEQ ID NO:12 {HMGCRE16E18_{—}99},
whereby the haplotype allele is associated with a decrease in total cholesterol or low density lipoprotein in response to administration of a statin to the subject, thereby inferring the statin response of the subject.

2. The method of claim 1, wherein the haplotype allele comprises
a) a CYP3A4A haplotype alleles, a CYP3A4B haplotype allele, or a CYP3A4C haplotype allele;
b) an HMGCRA haplotype allele, or an HMGCRB haplotype allele; or
c) a combination of a) and b).

3. The method of claim 1, comprising identifying a diploid pair of haplotype alleles.

4. The method of claim 3, wherein the diploid pair of haplotype alleles comprises
a) a diploid pair of CYP3A4A haplotype alleles, CYP3A4B haplotype alleles, or CYP3A4C haplotype alleles;
b) a diploid pair of HMGCRA haplotype alleles, HMGCRB, or HMGCRC haplotype alleles; or
c) a combination of a) and b).

5. The method of claim 1, comprising identifying at least one CYP3A4C haplotype allele and at least one HMGCRB haplotype allele.

6. The method of claim 1, comprising identifying
a diploid pair of CYP3A4C haplotype alleles;
a diploid pair of HMGCRB haplotype alleles; or
a diploid pair of CYP3A4C haplotype alleles and a diploid pair of HMGCRB haplotype alleles.

7. The method of claim 6, wherein the diploid pair of CYP3A4C haplotype alleles is ATGC/ATGC or ATGC/ATAC.

8. The method of claim 6, wherein the diploid pair of HMGCRB haplotype alleles is CGTA/CGTA or CGTA/TGTA.

9. The method of claim 6, wherein the diploid pair of CYP3A4C haplotype alleles is ATGC/ATGC, and wherein the diploid pair of HMGCRB haplotype alleles is CGTA/CGTA or CGTA/TGTA.

10. The method of claim 1, wherein the statin is Atorvastatin or Simvastatin.

11. The method of claim 6, wherein the diploid pair of CYP3A4C haplotypes alleles is a diploid pair of one minor and one major haplotype allele or a diploid pair of minor haplotype alleles.

12. The method of claim 6, wherein the diploid pair of HMGCRB haplotype alleles is a diploid pair of major haplotype alleles or a diploid pair of minor haplotype alleles.

13. The method of claim 6, wherein the diploid pair of CYP3A4C haplotype alleles is ATGC/ATGC, ATGC/ATAC, ATGC/AGAC, ATGC/AGAT, ATGC/ATAT, ATGC/TGAC or ATGT/AGAT.

14. The method of claim 6, wherein the diploid pair of HMGCRB haplotype alleles is CGTA/CGTA, CGTA/TGTA, CGTA/CGCA, CGTA/CGTC, or CGTA/CATA.

15. The method of claim 1, comprising identifying
a diploid pair of CYP3A4C haplotype alleles;
a diploid pair of HMGCRC haplotype alleles; or
a diploid pair of CYP3A4C haplotype alleles and a diploid pair of HMGCRC haplotype alleles.

16. The method of claim 15, wherein the diploid pair of CYP3A4C haplotype alleles is ATGC/ATGC, and wherein the diploid pair of HMGCRC haplotype alleles is GTA/GTA.

17. A method for inferring a statin response of a Caucasian subject from a nucleic acid sample of the subject, the method comprising identifying, in the nucleic acid sample, a diploid pair of alleles indicative of a statin response, wherein the diploid pair of alleles is identified for:
a) nucleotides of the cytochrome p450 3A4 (CYP3A4) gene, corresponding to a CYP3A4C haplotype, which comprises
nucleotide 425 of SEQ ID NO:10 {CYP3A4E3-5_{—}249},
nucleotide 1311 of SEQ ID NO:7 {CYP3A4E7_{—}243},
nucleotide 808 of SEQ ID NO:8 {CYP3A4E10-5_{—}292}, and
nucleotide 227 of SEQ ID NO:9 {CYP3A4E12_{—}76}; and
b.) nucleotides of the 3-hydroxy-3-methylglutaryl-coenzyme A reductase (HMGCR) gene, corresponding to an HMGCRB haplotype, which comprises
nucleotide 519 of SEQ ID NO:11 {HMGCRE5E6-3_{—}283},
nucleotide 1757 of SEQ ID NO:2 {HMGCRE7E11-3_{—}472},
nucleotide 1430 of SEQ ID NO:3 {HMGCRDBSNP_{—}45320}, and
nucleotide 1421 of SEQ ID NO:12 {HMGCRE16E18_{—}99},
wherein the diploid pair of CYP3A4C haplotype alleles is ATGC/ATGC, ATGC/ATAC, ATGC/AGAC, ATGC/AGAT, ATGC/ATAT, ATGC/TGAC or ATGT/AGAT, and the diploid pair of HMGCRB haplotype alleles is CGTA/CGTA, CGTA/TGTA, CGTA/CGCA, CGTA/CGTC, or CGTA/CATA, and wherein the diploid pair of haplotype alleles is associated with a decrease in total cholesterol or low density lipoprotein in response to administration of Atorvastatin or Simvastatin to the subject, thereby inferring the statin response of the subject.

18. A method for inferring a statin response of a human subject from a nucleic acid sample of the subject, the method comprising identifying, in the nucleic acid sample, a nucleotide occurrence of at least one statin response-related single nucleotide polymorphism (SNP) corresponding to
nucleotide 1757 of SEQ ID NO:2 {HMGCRE7E11-3_{—}472},
nucleotide 1430 of SEQ ID NO:3 {HMGCRDBSNP_{—}45320},
nucleotide 1311 of SEQ ID NO:7 {CYP3A4E7_{—}243},
nucleotide 808 of SEQ ID NO:8 {CYP3A4E10-5_{—}292},
nucleotide 227 of SEQ ID NO:9 {CYP3A4E12_{—}76};
nucleotide 425 of SEQ ID NO:10 {CYP3A4E3-5_{—}249},
nucleotide 519 of SEQ ID NO:11 {HMGCRE5E6-3_{—}283}, or
nucleotide 1421 of SEQ ID NO:12 {HMGCRE16E18_{—}99},
whereby the nucleotide occurrence is associated with a decrease in total cholesterol or low density lipoprotein in response to administration of the statin, thereby inferring the statin response of the subject.

19. The method of claim 18, wherein the at least one statin response-related single nucleotide polymorphism (SNP) corresponds to:
nucleotide 1311 of SEQ ID NO:7 {CYP3A4E7_{—}243},
nucleotide 808 of SEQ ID NO:8 {CYP3A4E10-5_{—}292},
nucleotide 227 of SEQ ID NO:9 {CYP3A4E12_{—}76}; or
nucleotide 425 of SEQ ID NO:10 {CYP3A4E3-5_{—}249}.

20. The method of claim 18, wherein the at least one statin response-related single nucleotide polymorphism (SNP) corresponds to:
nucleotide 1757 of SEQ ID NO:2 {HMGCRE7E11-3_{—}472},
nucleotide 1430 of SEQ ID NO:3 {HMGCRDBSNP_{—}45320},
nucleotide 519 of SEQ ID NO:11 {HMGCRE5E6-3_{—}283}, or
nucleotide 1421 of SEQ ID NO:12 {HMGCRE16E18_{—}99}.

21. The method of claim 18, wherein the at least one statin response-related single nucleotide polymorphism (SNP) corresponds to:
nucleotide 1311 of SEQ ID NO:7 {CYP3A4E7_{—}243},
nucleotide 808 of SEQ ID NO:8 {CYP3A4E10-5_{—}292},
nucleotide 227 of SEQ ID NO:9 {CYP3A4E12_{—}76}; and
nucleotide 425 of SEQ ID NO:10 {CYP3A4E3-5_{—}249}.

22. The method of claim 18, wherein the at least one statin response-related single nucleotide polymorphism (SNP) corresponds to:
nucleotide 1757 of SEQ ID NO:2 {HMGCRE7E11-3_{—}472},
nucleotide 1430 of SEQ ID NO:3 {HMGCRDBSNP_{—}45320},
nucleotide 519 of SEQ ID NO:11 {HMGCRE5E6-3_{—}283}, and
nucleotide 1421 of SEQ ID NO:12 {HMGCRE16E18-99}.

23. The method of claim 21, wherein the nucleotide occurrences comprise a minor allele of a CYP3A4C haplotype.

24. The method of claim 22, wherein the nucleotide occurrences comprise a minor allele of a HMGCRB haplotype.


SEQUENCE LISTING (234 sequences)

SEQ ID NO: 1 - DNA, 2170 bp, Homo sapiens CYP2D6E7_339
  misc_feature (1274)..(1274): n = a or c
gacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcrtgtctgtccaggccgtgtccaacaggagatcgacracgtgatagggcaggtgyggygaccagagatgggtgaccwggctcrcatgccctrcaycactgccgtgattcaygaggtgcagcgctttggggacatcgtccccctgggtgtgacccatatgacatcccgtgacatcgaagtacagggcttccgcatccctaaggtaggcctggcrccctcctcaccccagctcagcaccagcmcctggtgatagccccagcatggcyactgccaggtgggcccastctaggaancctggccaccyagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctc

SEQ ID NO: 2 - DNA, 3220 bp, Homo sapiens HMGCRE7E11-3_472
  misc_feature (1757)..(1757): n = g or a
tatcatttcctagaggtactactttgggaaattaaacatattggagcctcaatgttctcatctgaaaaatagataattgtacctacctctcaaggttgtgtgaaataaagaaagaagatagtatctgttagtcatgttacaccgtgcccagaacttagctgtagtgttcagcagatactagatttttcttccttgaaaaagctcctttgtaatgaaaaatgccacattggagtttatgttatcatcctcacctctgcattcccaagtatctgtagacattcttcattaggccgaggttccctgggaagttcaatttcaggttcctgtgtcaccagtactgatgaagtatcgagtaaggaggagttaccaaccacaaatgtagctctgtttggggtatctgtttcagccactaagggttttataacctcaactgttcaagaaatcaaaagaaatctcacaacacggcacataattttgaaaacagactactttttgacaaagcaatgaaggattgaaagcgaagagaataacaagttaccttttctttctcggtttatccctgtctcttcctctactgaatcacatttctggttatttctgaccagcataggttcacgtctacaacaattgtctgggactttcttttgtgtcactacaggagatgtgatagggttttttaatgagagtgtagattctgtctctgtttgttcaaagaagatgtacttgacagccagaaggagagctaaacttagggtaataacttgttcaatatccatgctgatcattctaaataaaagaaagcaaattaaaatcttattcagaaatgtaaaggacacaatctaaacttacattagcatagagtgttatgattaatatatcacaaagtagatttcaattaacttacttagagagataaaactgccagagggaaacacttggttcaattctcttggacacattttcatccagtcctaatgaaaccttagaagtatctgctgtactgttttgaggagaaggatcagctatccagcgactgtgagcatgaacaagaaccaagcctagagactgaaataaaatttttaaagtaatgtatcctctgcatatcaatagaacttaaatttcttatccctagcaactggacagccagacattatctctcatagcttccccttaccatgaaaaaaaaaaagccccaagcttgctatgcaactaactaaagtagtgaccccactgaactactgaaaacaccccaaagaacaggctttcaacgagagataaggtgggggaacttaaaaagtctgtttaggagagaggggctaataaagaccaggagcctcaaagaaatgaacacattaagaaaaaaaggaagaagggggtgcaatatcattgaatgggccaaaattgtagaaaaaaagaaatcttawaaataatgagattggaactgaggatactaaaagaagaagaaaaccatgtcattaccataatcatcttgaccctctgagttacaggattcggcttattttcttcttcttctaaaactcgggcaaaatggctgagctgccaaattggacgaccctcgcggctttcccgagaaagctacaaattaagtcagtgtgacattagaaggtattgattctgtttaggtaaactgtgtaagcagaaatcttactcttctactagtgccatatgtaagaattggtcttacctctaataccaaggacacacaagctgggaagaaagtcatgaacacgaagtanttggcaagaactgacatgcagccaaagcagcacataatttcaagctgacgtacccctggttagagaaaaattaaagatacaactagtaaagtctacgttattttttatgctgcatatatcacaaggatatatttgtcttcctgcaggtggacaaaacatgaaaatattagtacattaagctccttgccacttactggaaatagaattagcaacttagaggtgatgccaagccttaggaaacagcaggaataacaacagaactctaagtccctcaagagcaaagctcttgccttctctcatgtccccatatcacaatgccaggcagaatggtagtgtttaatacacaattgataaaacatatgtgtttccaattcctttttgtcaagcagtagtgccttattattaaataaggagaggacaggctactctaacagagcaaaaccccagtgacactattgatctaatcccacttgttaggagaaaagcaatttgccaattaaccaaggattttttatataagtagaatatcctgccagttatgccagtcctgatataaggtcaaaataatattttttgtgtaccaacaaactctaatttcaatataacctctatgatgcctactgaagttcttataattttggttatttcattgatctgtttggaaaatgatataatacaaaatgccaacttaagactaaaaattaacaaacatattgtactacaactgtttaaggtatcatttggtgatctcaaatagaaaacagaattatagaggctagaagagaccttagatgaccaccaaatccttctccctcactttacaaataaacttgagtataatataaaacatatttctgatgtcgctcaaacataacaatgctttgaaacacaaatttgaattgtcctataaaaactgctcacaaaaactcaaagttttattttattacctacattttaagatataatttgactgactagaacaagcatatattgtatttttttaattcccaatgctttgaaacataaatttaaattgtcctataaaaactgctcacaaaaactcaaagttgtattttattacctaaattttaagatatcatttgactgactagaacaagcatatattgtatttttttaattccacgattaccctcaaatgtggaaattcaagagactacaaaatcacaataacaaaagcattataaatcaaactacattttaaatagtagctgaatataatcttttcaaacttgaggccattaaaaccatacttgaccaatgctttcatgacttgactatctactatttctctctgcacaatattcactcatgttgttgccatatgctctccaggcattcttccttactgtgtccccagataagtctctctacacacaaagaaacagcaccaatccacagacatccaagaagctaagactttcttctttttgtactggcttttt

SEQ ID NO: 3 - DNA, 2870 bp, Homo sapiens HMGCRDBSHP_45320
  misc_feature (1430)..(1430): n = t or c
ccatgtgttctcattgttcaattcccacttacaagtgaaaaggtgtggtgtttggttttctgttcctgtgttagtttgctgagaatgatatttccaggctcatccatgtccctacaaaggacatgatctcattccttttttatggctgcacagtattgcatgctgtatatgtgccacattttctttatccggtctatcattgatgggcatttgggttggttccatgtctttgctcccagaactcttttcatctcacaaaacaaaaactctgtacttactaaataacaaccacccattttccccttccccaaacccatgataaccaccattctactttctgtctctatgaatttgactattctagacacctcattcaagtggaatgatacagtatttgtctttttgtgactggtttatttcatttagcataatgtcatcaatgtttatccatgctgtatcatgtgctataatttccttcctttttaatgctgaataatattccattttatatagacatatacatacacacacatacaaatacatctaatttgttgtccattcatccaacaacgaacactaaggttgattccaattcatggctattatgaaaaatgctgctacaaacatagctgtacaaatatctctctgagaactgctttcagttcttttgggtatatgcccggaagtgcaactgctggatcatatggtaatttcatgtttaagttgttgaactgccatactgtttattggcaattttaaagcttatacagatgctccttgacttttgataggtttattaggctgtaaaccccacataagcagaaaatatcctaaattgaaaacagacagacggacggacggacagacggatggatgaatggagtcagggtcttgctacattgccgaagctggcctcaagctcctaggctcaagctaacttcctgccccagcctaccgtgtagcgaggaccacaggtgtgtgccactatgcacaactatttttttttattgtttgtagagatagcatctcactgtgttgcccaggctggtcttaaactccagaccccaagcaatcttcttgccttggcctcccaaagtactgaaattatattggtgttcttaaagacaaatcttgaagaggtcagcttcaaaggtggtctcttgactggataaagttttgaaatgtcaatactaagattgttcccagtcctaagtaaactcaggatatgtgtaatgccaagtctaaattaaatctatcaaatgtaaggaataccaatcaacaaatgcctgatttgttttttataaaagtactttcattttaataaaagtactttcagatactctgcctacacttacctttgaaatcatgttcatccccatggcatcccctgacctggactggaaacggatataaaggttgcgtccagctanacttgtatgaagtttctgtagacgtgcaaatctataaataaaagatgcaaagactgtgttttattcttttattattattatttctttgttttttgtttttttttgagacggagtctcactctgtggcccaggctggagtgcagtggcttgatcttggctcactgcaacatccacctcccgggttcaagaaattctccagcctcagcctcccgagtagctgggattacaggcgcgggccaccatgcccagccaatttttgtattttgagtagagacagggtttcgccatgctggccaggctggtctcgaactcctggcctcaagtgatctgcccgcgttggcctccccaaagtgttgggattacaggcgtgagccactgcgcccagtcacaattatttcttaataaacttacacagttcacataaaaacaaatgtgttagcttgaactatactatggttatcatttgtgttgattatgctactttattaattttctttatttgaagtaagtcttattatactaatatttctctcctatttgaaaaatctttttttctaagacagttctctcctaggcaaagtaacatctaatcaaaattactagctcacacttttttttttttcttactaatttacctctgtggagctattcatttgaatcaacattctttttttccccccaaccaagcataaatattactcattttaaatgaatggctttaaagttgatattctgttatgtgctctttagcaggtaatatgttaacaattatgtttggtaatcacagaaaatgacactggttctaaaataaacaaatagatataactgtacatacaaatccactcacacacctgctagtgctgtcaaatgcctcctttatcactgcgaacccttcagatgtttcgagccaggctttcacttctgcagagtcacaagcacgtggaagacgcacaactgggccacgagtcatcccatctgcaaggactcggctgctggcacctccaccaagctacacagtatatgttagagaagcaagcacatgttacccaaaaatgctcatgcttgacccaaaaggtatcactaattgtccttaaaactcttctcattgccttacttatgatgtatttttaaactggcaaatatataaatgccaacttacacctattgctctgcagcctctattggtgctggccacaagacaaccttctgttgttgccattggaacctgaaattctttttcatctaagcaaaggggtcctgccactccaacagggatgggcatatatccaataacattctcacaacaagctcccatcacctaaaaggtaaagtcaggcaccaaatgaaaatctatatagtaaatgcacaaaattttatctcagcttgtcagtataactatcttcaaacttaatcctttagtatgtattctttttaaacaaa

SEQ ID NO: 4 - DNA, 2240 bp, Homo sapiens CYP2D6PE1_2
  misc_feature (1159)..(1159): n = t or c
aacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgccctaagtgtcagtgtgagtctgtgtatgtgtgaatattgtctttgtgtgggtgattttctgcgtgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacncaccaggccccctgccactgcccgggctgggcaacctgctgcatgtggacttccagaacacaccatactgcttcgaccaggtgagggaggaggtcctggagggcggcagaggtgctgaggctcccctaccagaagcaaacatggatggtgggtgaaaccacaggctggaccagaagccaggctgagaaggggaagcaggtttgggggacgtcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgcctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccacgatcaggaggctgggtctcctccttccacctgctcactcctggtagccccgggggtcgtccaaggttcaaataggactaggacctgtagtctggggtgatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcggg

SEQ ID NO: 5 - DNA, 2170 bp, Homo sapiens CYP2D6E7_150
  misc_feature (1093)..(1093): n = t or c
gacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcrtgtctgtccaggccgtgtccaacaggagatcgacracgtgatagggcaggtgyggygaccagagatgggtgaccwggctcrcatgccctrcaycactgccgtgattcangaggtgcagcgctttggggacatcgtccccctgggtgtgacccatatgacatcccgtgacatcgaagtacagggcttccgcatccctaaggtaggcctggcrccctcctcaccccagctcagcaccagcmcctggtgatagccccagcatggcyactgccaggtgggcccastctaggaamcctggccaccyagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctc

SEQ ID NO: 6 - DNA, 2170 bp, Homo sapiens CYP2D6E7_286
  misc_feature (1223)..(1223): n = a or c
gacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcrtgtctgtccaggccgtgtccaacaggagatcgacracgtgatagggcaggtgyggygaccagagatgggtgaccwggctcrcatgccctrcaycactgccgtgattcaygaggtgcagcgctttggggacatcgtccccctgggtgtgacccatatgacatcccgtgacatcgaagtacagggcttccgcatccctaaggtaggcctggcrccctcctcaccccagctcagcaccagcncctggtgatagccccagcatggcyactgccaggtgggcccastctaggaamcctggccaccyagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctc

SEQ ID NO: 7 - DNA, 2590 bp, Homo sapiens CYP3A4E7_243
  misc_feature (1311)..(1311): n = g or t
tctcccaagatggggcagctccgatgaggaggtggggcagctggaggaaaaggatcttctcccctgtgcacaggggccagggtttacatatccattaaattgtcaccttggatattctagaagactaaatatatcctttagggggaaaaagtgtgattgtaccaaagttttaagcatggagtgtatgggatggtggaaggggaaggcacttggtatctgttggttggcagtgagtaggttgggagagttataatggagaacttagaataactttgatcatttcatgtttttttctgaggatatcagtagaatactaaatattaaaattcctaccatttctttttcctccagtctcaaagagagagggtggtaaaaacactataggtagggcaagcctattatttgctatctacacttatgcagtaaaaacaggtgtaatctgagtttgtcctgggcagaccagggatatgtggtcactcactatagaaatttccaaatcaaattttgagagatttttttttaaccaggacattattggtcattatattttacaaaaataattctgctgtcagggcaacctcagctcaccacagctggggatagtggaattttccaaagcttgagcagggagtatagagaataaggatgatatttctaggagctcagaacagggtactgttgctttgtaaagtgctgaagaggaatcggctctgggcatagagtctgcagtcaggcaatatcacctgtcttgagccccttaggaagagttaattattctactcttgttctgctgaagcacagtgcttacccatcttgtatcatccacaatcaatacatgctactgtagttgtctgatagtgggtctctgtcttcctatgatgggctccttgatctcagaggtaggtctaattcagttcagtgtctccatcacacccagcgtagggccagctgcatcactggcacctgataacaccttctgatggagtgtgatagaaggtgatctagtagatctgaaagtctgtggctgtttgtctgtcttgactggacatgtgggtttcctgttgcatgcatagaggaaggakggtaaaaaggtgctgattttaattttccacatctttctccactcagcgtctttggggcctacagcatggatgtgatcactagcacatcatttggagtgaacatygactctctcaacaatccacaagacccctttgtggaaaacaccaagaagcttttaagatttgattttttggatccattctttctctcaataagtatgtggactactatttcctttaatttatcttnctctcttaaaaataactgctttattgagatataaatcaccatgtaattcakccacttwaaatatacagttcagtgatttgtagtacatttgaagatatgtgtgaccatcatcattttaaactttaaaactttttttgtcaatctagagacctcatacatttttagctatcagccccctgtcacaaaccctgtcatcatatgcaaccactaatcaactttctgcttctatggatttgcctattctggacacttcatagaaatgatattaattcatcagggttttttattctctagttcatgaatttgtactttagtctgtatcattttctttcttctgctggcttcaggcttagtttgcccttcttcgtttactatgttgtggcatgaacatagattactgatttgtgatttttttgttcctctaaatttagacattacagctgtaactttccctctgagcacttcctttgctaaatcccatgagattgtggcctatcacatcttagttttgttcacctcaaaacagtttctatttgccctttgggtttctactttgactcattgggtacttaaatgtttattatttaacttccacatatgtgtgagtttctcaattttctttcccttattgattttatctttattccatgataggtgacagagatatgctgtgttatttctatcttgactacctactatttcttgaacagcaagattaattttgagcttcagattatgatttgggttattctaggagactgtagtccaatagataaaggcaaagagattagggcattgaattttgttccttttatccttcaaaagatgcacaaggggctgctgatctcactgctgtagcggtgctccttatgcatagacctgcccttgctcagccactggcctgaaagaggggcaaaagtcatagaaggaatggcttccagttgagaaccttgatgtcttttactcttctggttggtagagaaaactagaattgctccaggtaaattttgcacattcacaatgaatttctttttctgtttttgttttgtttttcctacagcagtctttccattcctcatcccaattcttgaagtattaaatatctgtgtgtttccaagagaagttacaaattttttaagaaaatctgtaaaaaggatgaaagaaagtcgcctcgaagatacacaaaaggtaaaatgtggtggtagttataggaggatgtttagtttttcataattttttagataatatacatatgatcagtgcagttacctgtatgttt

SEQ ID NO: 8 - DNA, 1820 bp, Homo sapiens CYP3A4E10-5_292
  misc_feature (808)..(808): n = g or a
agattttgaatcagtagttcaagggtggggtttgagattttgcatttctaaatgagctctcaagatgcttctgacccatggaccacactttgaataccaagaagtggtctgtagaccaatattggtcccttaagttccctcaaacatatcttcgggaaacgtcctttgattttccctacatttaaccattagtgttgcaaattctctcaaagtttgtcaagatatattgtagctaaaataaattacatttttcttgggggagagtactacctcatattaacttacaataaagtacttttaggatcattcaaggaacacacccataacactgagtatgttatgcggaaatgctctctctggaaattacacagctgtgcaggtggcgggggtggcatgaggaggagtggatggcccacattctcgaagaccttggggaaaactggattaaaatgatttgccttattctggttctgtaagatacacatcagaatgaaaccacccccagtgtacctctgaattgcttttctattcttttcccttagggatttgagggcttcacttagatttctcttcatctaaactgtgatgccctacattgatctgatttacctaaaatgtctttcctctcctttcagctctgtccgatctggagctcgtggcccaatcaattatctttatttttgctggctatgaaaccacgagcagtgttctctccttcattatgtatgaactggccactcaccctgatgtccagcagaaactgcaggaggaaattgatgcagttttacccaataaggtgagtggatgntacatggagaaggagggaggaggtgaaaccttagcaaaaatgcctcctcaccacttcccaggaraatttttataaaaagcataatcactgattctttcactgactctatgtaggaaggctctgaaaagaaaaagaaagaaacatagcaaatggttgctactggcagaagcgtaagatctttgtaaaacgtgctggctctggttcatctgctttctattactacaataatgctaagtaaaaaacctccaaaaacctcagtggcatctaacaataagcatttgttgctcacactcatttcaattggttttggttgtgaattacatgtttgcagcaggcaccatagtggtgtgtgatgtccccttagctgtatccacatatggacacaggaattggctctttttatctctttttattttcttggttacagacatgtgacttttttttttgaaaggtaacaatcactttctcatatgttatttgatgctagtggtcatagcctatagtcacatttgtttcaatgagaaagaaaaaccagtacacggttatgctaaggatttcagtccctggggtgagagccgtctcgaatgtctccccacttcataactcctccacacatcatagttggatagtgagctctgctgatattggcaggacttgctctggtctggctgtagtctgacggagcctggccctgggtgtgctgtgcaggctgactcagctctccccacacctatctcatgttccagtcaggcagtaactggtgaagaagccaagctaggaaccaggatatctggctcctgagctaaagtcttaaaacactatcatattgccttccaaatataacaccaaatactaggtgcatatcaccctcactgttttcagacctctgccaaaattgggattctttgtggtatgaagagacacggctttggggctggcccggctgtgacagtgaggtgaacacaaagggatgttcttcagagattacagtccagccctgaagcaacaacta

SEQ ID NO: 9 - DNA, 490 bp, Homo sapiens CYP3A4E12_76
  misc_feature (227)..(227): n = t or c
cactatttatctcatctcaacaagactgaaagctcctatagtgtcaggagagtagaaaggatctgtagcttacaattctcatagcaaaataagcatagcaggatttcaatgaccagcccacaaaagtatcctgtgtactactagttgaggggtggcccckaagtaagaaaccctaacatgtaactcttaggggtattatgtcattaactttttaaaaatctaccaangtggaaccagattcrgcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacaggttagtcaattttctataaaaataatgttgtattaataattcttttaactgagtggtctgtattttttaaaaagaatatgcttgtttaat

SEQ ID NO: 10 - DNA, 840 bp, Homo sapiens CYP3A4E3-5_249
  misc_feature (425)..(425): n = a or t
gaaagacaaagaggtacttagtatttatacacaaggataagtcattcagtatccacaacacttggagagaattcaagagtgattttaaatttcccttttcaaatacctcctctgttttctcttatttcctttatgacgtctccaaataagcttcctctaactgccagcaagtctgatttcattggcttcgactgttttcatcccaattagaggcagggttaagtacattaaaaataataatcaaatattattttgtttctcctcccagggcttttgtatgtttgacatggaatgtcataaaaagtatggaaaagtgtgggggtgagtattctggaaacttccattggatagacttgtttctatgatgagtttacyccactgcacagaggacagtctcagcccaaagcctcttgggatraagctcntgtcaaccyaactacaaacagagagaagttctctgaaagaagaagatatttatttgggtgtagagtattgcaatgggaatctgcatgcctttataaactatgtgcaaattcagggaagtaaagcaagacaaagaggctccaaggaaaatatgaggaggatttcttatcagttttgaaataattatccttcgctacaaagatcagtaacaagggtgacgcctcaccaaggttggacaggcagttgctgggcaggtgtccttgcagaaatattttttttaatgttgggatggcctttgtgcaagcttgtagttttgcggagtcttttgtgatagttttgttatcaggcacacaagcatgagaatcctctcttcatagccttctttgatttatttgtcagggtttttacacacacaca

SEQ ID NO: 11 - DNA, 910 bp, Homo sapiens HMGCRE5E6-3_283
  misc_feature (519)..(519): n = t or c
tcacttgaggtcagaaattaaagaccagtctggccaacatggcaaaactccgtctctactgaaaacacaaaaattagccggggatggtggtgcacatgtgtaatcccagctactcaggtggctgaggcagaagaatccctcgaaaccaggaggcgaaggttgtggtgagccaagatctcgccactgcactccagcctgggtgacagagtgagactacatctcaaatcaatcaatcaatcaatctaccctgggtttctcttccattagatcttgttctgctctctgatgtgtttcactaggaaaatactcttatttacccaaaaattattattaccataagttctgaaaactttcaaaaagaaaaatgggggyaattccaaattccagtagctacagaatcataattgagttgttagatacaggggactgttcctggggcacttatggagaccagtcttgggacttragaattaaacttaaaactttgggcaattcttaaatcttgtgctatgaagaaangctattaatccttcctattaatgtaaactgaaaaaaggaatactattcacattcctatcttataaataatacttacctgtgagttggaactgagggcaaactttgctaatgtgcttgctctggaaaggtcaatcaaaagtaggaaaaagggcaaagcttcactggaaaagaacaaaatgatcagataaatttaacgggaaaaagtatgattttaaaaaaattctttttagaacaaaacctttccccctccatactgtatgatcctgtagtatgtgtacctttctgcagacaaaaaagtataccctatatttctttggcatcctcaaagctaaacatagtagttgctcaaaatatttgttaaaaatatttttaatgttaaaatgtaagtata

SEQ ID NO: 12 - DNA, 2380 bp, Homo sapiens HMGCRE16E18_99
  misc_feature (1421)..(1421): n = a or c
agccaatccagacaaacatttatatttaaacatttatatttaaacaaaaggcctctctgaacaaatagcctgcggagataaatacagtgatttgttttcctgatagaactatttagcatgtttaacacattattctgtagtttgggaataagagtgtttcttcccttgaagaaaacaggtccccttctgaagaataatgctgattaccccccaaaatcaaaatagaccagcaccaaatgaagtattaatttacaaacatgaacttagaacttagctcttacttcttgaagttctacatcccagacttaataaattaactacaaaatcaggagtttcatcagctacagtataatttaaaaatccattttcaactggcaggagtgagggagaaggtcaattgcactgatcaccatgaacttcaagaatttcatcaaaacttttttcccagcttatatttgccttcagaggtgagctgtagattaccatctctgatgctttaacatacaatattcttgttgaaatctcttcaaagagcacagcatgtaaagcactaaactgtgttcagatctgaggagtctgcatggaaagaacctgagacctctctgaaagagccaaaaaccaagtggctgtctcagtgatcacatctattcatcctccacaagacaatgcattgagcttttttaattcacagattttatgttagtcctttagaacccaatgcccatgttccagttcagaactgtcgggctattcaggctgtcttcttggtgcaagctccttggaggtcttgtaaattgatcttcgacctatggtagaaaatgacaaagtagcaatatataaatatcaggagtgtagaattttaacttggaactacagtagatgaatagtaagtttttacactgcatattttttgaagtatagggggaacatgttaaatatatctttgagtcttacctgttgtgaatcatgtgacttttgacaagatgtcctgctgccaatgctgccataagtgacaattccccagccattacggtcccacacacaattcgggcaagctgccgggcattttccccaggattatctttgcatgctccttgaacacctagcatctgtagccagggagagacacaacaagattcacccttaaaatcatgaccaatttcttactaaatcaactaaaaacagggcaactgtaatggcatcagaatagaactagactccactggaagcactaactttccaagacttgacagccacacctgacagtgcataataccatagctaacataatattcacagcctgactggcagtacccttaactcagtagatgaacattcatttgctctcttcatctactttcttatctaagcataagcttaaacatgcttatttggacacaatggattaggctgatatgacnaaagagtttggaaaagaccaattaaaatagaggtgagtgatacatartctcagatagaaagagaaacccagagagtcagaactaggcttgtggactctatgcctgatacatcatacctgcaaacaggcttgctgaggtagtaggttggtcccaccacccaccgttcctatctctatagatggcatggtgcagctgatatataaatcttcatttgtgggaccacttgcttccattaaagtaatacagtttgaactaccaacattctgtgctgcatcctgsaaacaagaaaagaaaaaatatacaatatacttctttcacttagaaagacgtmacacaagagaagtggaggctggagagctcacctgtccacaggcaatgtagatggcggtgacaatgtttgctgcatgggcgttgtagcctcctatgctcccagscatggcagagcccactaaattcttgttaatgttgacctcaatcatagcctctgtggtagtctttaatacctacaaaacagagctgtgtacatttagatgttcctccagaaggttcaggggaatgttacccaaatctatctttctgaacctccagaaaacaaagtttagatgtggccccatttaagccctgtcctccattaaaaaataaaaaaaattaaaaaaaatcagtaaagtttgttcctatggatgatacacacagacagatgggcaaggtacaacagtcatctttgatggaaaacactgtcccatatatttaactttatttaaaatgttaatactcctttcccccatttttaaatacaattaaagattacaaaataaaaaagataaattatccatccagtcactcacttctctgacaaccttggctggaatgacagcttcacaaacaacagattttcctcttccctctatccaatttatagcagcaggtttcttgtcagtacaatagttaccactaacggc

SEQ ID NO: 13 - DNA, 20 bp, Artificial sequence
  PCR primer
aggcaagaaggagtgtcagg

SEQ ID NO: 14 - DNA, 20 bp, Artificial sequence
  PCR primer
cagtcagtgtggtggcattg

SEQ ID NO: 15 - DNA, 20 bp, Artificial sequence
  PCR primer
gtggggacagtcagtgtggt

SEQ ID NO: 16 - DNA, 18 bp, Artificial sequence
  PCR primer
agcmcctggtgatagccc

SEQ ID NO: 17 - DNA, 98 bp, Homo sapiens
agcmcctggtgatagccccagcatggcyactgccaggtgggcccastctaggaamcctggccaccyagtcctcaatgccaccacactgactgtcccca

SEQ ID NO: 18 - DNA, 27 bp, Artificial sequence
  Amplification primer
yactgccaggtnggcccastctaggaa

SEQ ID NO: 19 - DNA, 26 bp, Artificial sequence
  PCR primer
tattctggaaacttccattggataga

SEQ ID NO: 20 - DNA, 32 bp, Artificial sequence
  PCR primer
caaataaatatcttcttctttcagagaacttc

SEQ ID NO: 21 - DNA, 23 bp, Artificial sequence
  PCR primer
catygactctctcaacaatccac

SEQ ID NO: 22 - DNA, 29 bp, Artificial sequence
  PCR primer
acatggtgatttatatctcaataaagcag

SEQ ID NO: 23 - DNA, 19 bp, Artificial sequence
  PCR primer
tgcaggaggaaattgatgc

SEQ ID NO: 24 - DNA, 24 bp, Artificial sequence
  PCR primer
ataaaaattytcctgggaagtggt

SEQ ID NO: 25 - DNA, 29 bp, Artificial sequence
  PCR primer
cckaagtaagaaaccctaacatgtaactc

SEQ ID NO: 26 - DNA, 22 bp, Artificial sequence
  PCR primer
gtccacttccaaagggtgtgta

SEQ ID NO: 27 - DNA, 20 bp, Artificial sequence
  PCR primer
tacaggggactgttcctggg

SEQ ID NO: 28 - DNA, 35 bp, Artificial sequence
  PCR primer
gaatagtattccttttttcagtttacattaatagg

SEQ ID NO: 29 - DNA, 33 bp, Artificial sequence
  PCR primer
ttactcttctactagtgccatatgtaagaattg

SEQ ID NO: 30 - DNA, 23 bp, Artificial sequence
  PCR primer
cttgaaattatgtgctgctttgg

SEQ ID NO: 31 - DNA, 25 bp, Artificial sequence
  PCR primer
ttacctttgaaatcatgttcatccc

SEQ ID NO: 32 - DNA, 29 bp, Artificial sequence
  PCR primer
ctttgcatcttttatttatagatttgcac

SEQ ID NO: 33 - DNA, 30 bp, Artificial sequence
  PCR primer
gctctcttcatctactttcttatctaagca

SEQ ID NO: 34 - DNA, 33 bp, Artificial sequence
  PCR primer
tctatctgagaytatgtatcactcacctctatt

SEQ ID NO: 35 - DNA, 20 bp, Artificial sequence
  Probe sequence
agcctcttgggatraagctc

SEQ ID NO: 36 - DNA, 20 bp, Artificial sequence
  Probe sequence
tatttcctttaatttatctt

SEQ ID NO: 37 - DNA, 20 bp, Artificial sequence
  Probe sequence
cccaataaggtgagtggatg

SEQ ID NO: 38 - DNA, 20 bp, Artificial sequence
  Probe sequence
actttttaaaaatctaccaa

SEQ ID NO: 39 - DNA, 20 bp, Artificial sequence
  Probe sequence
aatcttgtgctatgaagaaa

SEQ ID NO: 40 - DNA, 20 bp, Artificial sequence
  Probe sequence
aaagtcatgaacacgaagta

SEQ ID NO: 41 - DNA, 20 bp, Artificial sequence
  Probe sequence
ataaaggttgcgtccagcta

SEQ ID NO: 42 - DNA, 20 bp, Artificial sequence
  Probe sequence
atggattaggctgatatgac

SEQ ID NO: 43 - DNA, 875 bp, Homo sapiens UGT1A2008584   756
  misc_feature (398)..(398): n = c or t
ctgcagtctgaggagtcaccattgccctagcccccagtcatatgctctctgctggatgtgaccctatttgctaacccttgaaactttcttctcatgtatgagtaacgcagcgcatccaaaaaccatacaacattgctaagccagctttctctgaacagcgtggaggctggctatgtggttttggtcgtttttcgcaccaggatatttcttgtaaggatcaaagaggactctctctgaagtggctgtcaatatagaaaaagctgcagggaggctgagctcaagatgtgatgatcagattcttgggaagactttgttgaggattctccatatacaccatggaagaagaggtgctgctatctaaagcacacatgggcaagacaaccctagcaaagacccanggtgggttcagggagctcctgctcaccaagggactgggggagctggagcctggcctcagatcttgcacagtctgaattcctcttctgaatgccgaaggctccagtctttatttcagtcatccaggcctttatttcatcctgaagattatggtcaggctcttctccaagtcaatccagcaccaagttctgataagaatggttcctccagaacagaatgggctgcacaatttgtgggggccctagtccaatttcaagatggagaaataaatatgtgccctttctaaaggagagaccctgagcactcacaagggcctcaagccccatgaggcttatcctactccagaaagtcactcccacatgtgctgtgctcacaagattgctgatgccaccctgtccctgggcacctccagatgaagtgggcaagctgtcttgggtgtgcaccttttatctcactggccctggggtttttgcctggcc

SEQ ID NO: 44 - DNA, 1190 bp, Homo sapiens UGT1A1875263   755
  misc_feature (594)..(594): n = c or t
acagacactaagcttaaagtgaaacccacatcttattcaaaacttggaggttttccttcatttggccatttaaatttaatttttgtttctgtcctccgtagtcttctattctccaggcttcagagctctcagcttaccattcaattatctcctttctctccttatattccttttttcattttttaaaaacaatactttcaaagagcaaaaattttagaatttgatgaggtttattctagtgaagtttttatcgtttgtactttttgtaccctaaggaagctttgtttaccccaagatatagtttctacattctcttaaaaacactaaagagttccagtttatatgtttagctctgtgagattgggagaggggagctagatcactcaggtcaggctttcgggatgcctttttctgtctctggacgttgctggggtgacctcactgacacccatggcttcagctaccacatatgctgatggctccaagtctatctgtgcagcccagacccctcctcatctccagaccctggaagctgatgccttgggcagctcctcctatttcccaggcaccaggaatgtgagcttctccctcccnacagtcctgctgtcctcagggcccctatgtctctgaaggcaccaccatcttccaagatacatgggcctccgcagggtctaggagtgccagacacgtaaccagaaatcagatgacatcactatctaaataaaacaccactacatggaaatagaacaccactacatggaaatagaacatgggagccccttgaatgtggcaagagcaccctcccaggcatgttccaccctcaccccgggctcatcaggagggttcttaagatgcagacagttttaagggggttggaggaatagttgagaagctgagatgttgcacccacagctgagaatccctttctagcactctgtgtcctcacaaatccccagaaatcgtcctcccctggggagttctcaagcccttacagacctgccctctctgtgccatcctgcatatgcctcccttgagctgggtgtccctctgatggacgcatccattcactgcctgtcccatgggttgtgtccaaaggtggaatctgttatcaatgtggatttctaatgggagtaacttcctccataagggaagcctcagcctcaccagcaatggcagacatggccaggcat

SEQ ID NO: 45 - DNA, 121 bp, Homo sapiens SILV1052165   662
  misc_feature (61)..(61): n = c or t
agggaacaagcacttcctgagaaatcagcctctgacctttgccctccagctccatgacccnagtggctatctggctgaagctgacctctcctacacctgggactttggagacagtagtgga

SEQ ID NO: 46 - DNA, 401 bp, Homo sapiens RAB27526213   844
  misc_feature (201)..(201): n = c or t
gaatagtctgaaataaaccagctagttagacatggggctaatctagatctcatgtctcctaaattctagtcatgtatgcatatttttcttctgtctgtattccttccttcagagtgagaaacctcagttacagctgcttgaattcaagaatcaaaagttttcttcagtgccagcagttatagcagtgataaaatgctttanaattaaggcttgtgctctcagagaggttgggttgagggatctgtacttctgtgctcagagtccctctatcaagaagcccttcatcctgatgggctctgccaaggacagaagtcataaattgggagcttgtctgcttcttgctggtcattgcaaatccaagaaaaaaaaataactcttcttaatacttgtaccatatccca

SEQ ID NO: 47 - DNA, 401 bp, Homo sapiens GSTM1414673   580
  misc_feature (201)..(201): n = a or g
gtcactgtaagagcaggacttcctctgatccgaaagctactccgagggcttagtctcccctctagccccgcctacacaggaacagtgtcagtggtataggaaggacccccaggaaaagggccagagtaaaggaaatgtggtctgtgttttctgttaggggcctttggatactgagtccttcggtcatctggctaagtactntgtaaattagccacttcgtattttggcacaatttatgaatcgaaatccaaggatcagatccagcaagttggtgaggtatctgaggtgccccctagaaatgtccagccagtccactaggtgaacctcacagagaccggaacagcaacagcaacagtgagtgcagtggccacagagagcaggcgagggaggatggaagaact

SEQ ID NO: 48 - DNA, 401 bp, Homo sapiens CYP2E1RS2480257   37
  misc_feature (201)..(201): n = a or t
tcaaaaaaaaaacaaaaataaaaaaaaaaaaaacctctctgtgagaatcacttaaacaattaagatattccatgacttaacatatagttaaaataatcatgtgatgatttatttatattctgggaaaatatttattttcaaatactcatatgcaaagaaaggaatcagtttgagaaatcctgacctcaaacaatttgaaancttgtttgaaagcggggggttcagggtgtcctccacacactcatgagcggggaatgacacagagtttgtaacgtggtgggatacagccaaacccaatatgtatagggctgaggtcgatatcctttgggtcaacgagaggcttcaaattaaaatgctgcaaaatggcacacaacaaaagaaacaactccatgcgagccagg

SEQ ID NO: 49 - DNA, 985 bp, Homo sapiens CYP4B1RS2405335   143
  misc_feature (487)..(487): n = c or t
atggggaaggctatgggcaggtgtctttcactttagcgactaagaagccttttgaggaattaagactgtccaaagatggttggggtattttgaaaagaagtttcctgtcactagagatgtttgagcacttgggcagggattcttactgggtgaaggtgctgggcaggcaccctgtgtgctgggaatccgagaccgacatgacctgccttgagggaactatgacatgctgccgaaaaggaaaccccttcgtctttctcctccctcacctttgttttcgaaactcttatccccattttgtgagcagactgattttcatctgaagcagcccctggggcactgtagctgcagcacactggtcaagggtagcttgtttcttggacactggccccactgagcttcgggtcaactggctgatggcttgagcaggcttgctaacaagggctgcagaaagagactaagccaggatccctggtctcccttaactcangctggactgttccctttggtctttgtaccgcacgtttccacctttaaagagaggtcagggttccactaggcaatgttgaaatccttttccgatctgattcttggagcaaagcttactttaggctcttgagaagcagagaggaaagtggtgacgtatgcttgctcttcccaatccctccagtggtttatttctggtgaggtcaacaaggagtgccaatgtgagggtgggaggtggggctggggagctgggatcagtgaattattttcataccctgctctcttcctgtttccttccttctggagaaaaaacaagccgtcccagaacttccagtcctgtaagtgtcccagctcaggcattctgagggcgagtctgaggcatgcagttgtgtaatccagaggaactgtccaggcagtcacgtgtaccccacagggaggatgcctcagccctcttgaggggctgcagctgcaatagtgggaccaggtgctgggagctgtcctc

SEQ ID NO: 50 - DNA, 625 bp, Homo sapiens ESD1923880   696
  misc_feature (335)..(335): n = a or g
ataatattaagaaactgattaacgctcatacatgcatttacacttgttactcacttttaaaagctttcatactttggtatatcattactaatttattaacaagttttttcgttgtggtggactgccccaaattatttttaaaagaggcagaaaatacataagttaccaattaattaccttagtttagaagattaaaaattgttaatagtctgcctatcaactgcaacaaagtagtcaaggaattggtatgactgattatagttgattttaaaatggaagaaagatgtgataatgattttggtaataataatatgtaacattactgaacacttacngtgtgatacacatttttctagacactaaaccagatccaattactcatcccatgaatttttaagagaaagtgatatgaaactttaagatcaacatttatttctccttttaaaacaaaccaataagtaaaacaagcctgttaacttcttttctgtccaacaaatcttttgttttcatgtaaaaataattttaaacttacatgtaaaatgacatattttattgcttttaatgataaatacaggaaaaaagttggaaaactatctttatcataaatgcctattaagcaaaggga

SEQ ID NO: 51 - DNA, 401 bp, Homo sapiens CYP4B1RS681840   194
  misc_feature (201)..(201): n = c or t
gccccagggtgttttgtagcaagaatccctctatgatgatttaacacctgacgtttatctgcccagtagtttgcaaaatacctatactttatctctttctatccccttctaacccagtaaggtaaaaatgattatctccactttgttgacagggaaacagaagcacagagaggtaaagtggcttagctaactgggcacagngaaggcctgtaatcaatgtttactgaatgaaaatgagccacagccctgtcttcactacacagcacaaggtcaagggcactctcacagcaaagggcatggctgggaaagaggcctctgcagtctcagagtatctcatttccttgatctgggagctggcattctacacacacacacacacccacacacaccaatttgttggc

SEQ ID NO: 52 - DNA, 121 bp, Homo sapiens ACE_4311   135
  misc_feature (61)..(61): n = c or t
cttccccagttcctcaggatggggaagggttgccgggtggaaatgccttttctacaaaagntaaatccatctgtttgcaacctctaggccctaagacaatttaaccatccttttccagaac

SEQ ID NO: 53 - DNA, 121 bp, Homo sapiens AP3D12072304   906
  misc_feature (61)..(61): n = a or g
agtcccacaggtaccctccagattcaacctcaacctggcctaaggcccggcgtcagccccncccaccaatcaaagcccaggagggaaacaagcatggcccacatggggcactggagacaag

SEQ ID NO: 54 - DNA, 612 bp, Homo sapiens AHR2106728   599
  misc_feature (172)..(172): n =a or g
ctggtttctataactgctagtagaaaaaagaaaaataaaaaggaaggacatgaagatgtatagctctgtagagttttatagattacagagcctttatattttagttagtggacgttctggaaactttaaaacagattaaaatcatatccttaattgcttcaaataaaatctncctttgtaaagcctacataactggcttcagtaatcaaaatgttaattacttcacagatcctccaaaacatatataaaatctatagtaaaaatcatataacctgtatcttcaatgaatggcaacactgtaaattctttaaataaaaagttagtattacctggccagataatggtgagtttaaaatgattttttctcattactcataacacaattcatgtcaccattctctttaaacatactgtacacagcagtgtaatccaatagaaatataatgtgagccacatacagaacttaaatttttggctacagtagattaaataaaatacatttctaaagttaattttacctgtttctttttacttttgtaatgcggctactgaaaaaactaatcatttcaacatgaaatcaataataaaagatgagatattttacattctttt

SEQ ID NO: 55 - DNA, 158 bp, Homo sapiens GSTM1421547   527
gtgttcttcagtatgagacggtggctccagtggcctttgaagtcacaccgtgatatgtgacccatggtacaacctccacgagaacaatgtccaacctgccaactttcttctttcaaggtagaaggaagactttcaaaagagttgtgcaatggattagc

SEQ ID NO: 56 - DNA, 937 bp, Homo sapiens CYP4B1RS2065996   137
  misc_feature (600)..(600): n = c or t
acaacatttaaggtgaaacttgaaggatggtgagtctctggccatgtgaagcataaaggaaaagcattccaggtgagaaaacagaaagcacaaacgcgctgggttgggaaagatcatggtgtcttccaggagctgccccatggtgggacagtcacctgggatgagcttgaataggtcatccagagggtggtacatgtcctttggaggtttttctagtcacgtttactcacccagaggtgttttaagtaaatgaagttatacaataaatatttttgcaaaaccagccatttttcattgaacataacttgtcctcctttccaagctagtgcacttaactgtcattactatttttaacagttgtgtagtatttcactataaggatgtaccataatgtatttaactatttttccccatttagtgggagtctttcaaagatacacttaacactcctagtggcctgcccaggggccttggaaaaggtccaagctctggagtttgtggggaggcatggtaatgactaatctttattaagcacttgctgtgtgctagaccttgttctaagcaccttacattttgacctgattgaatcctcacaacaantctaagtggtcccattttagggaagaggagcctaaggaatagagaggttaggttacagtcccataaaattcctgttgaggcctgagcctgtgctgggcctgctttttctctccgtaaaattctctctcacaattgccacctattcactgatagtatccaaacaaattaggaagggaaaaatgtccaaaccttaataatttggacatttgtcccctcctaatctcattttgaaatctgatctccaatgttggaggtggggcctcatgggaggtgttggcatcataggggtggatccctcatgaatggccctggggccattctggcaggattgagtgag

SEQ ID NO: 57 - DNA, 101 bp, Homo sapiens SILV1132095   704
  misc_feature (51)..(51): n = g or t
gtattgccagatgggcaggttatctgggtcaacaataccatcatcaatggnagccaggtgtggggaggacagccagtgtatccccaggaaactgacgatgc

SEQ ID NO: 58 - DNA, 875 bp, Homo sapiens UGT1A2008595   768
  misc_feature (221)..(221): n = a or g
ctgcagtctgaggagtcaccattgccctagcccccagtcatatgctctctgctggatgtgaccctatttgctaacccttgaaactttcttctcatgtatgagtaacgcagcgcatccaaaaaccatacaacattgctaagccagctttctctgaacagcgtggaggctggctatgtggttttggtcgtttttcgcaccaggatatttcttgtaaggatcanagaggactctctctgaagtggctgtcaatatagaaaaagctgcagggaggctgagctcaagatgtgatgatcagattcttgggaagactttgttgaggattctccatatacaccatggaagaagaggtgctgctatctaaagcacacatgggcaagacaaccctagcaaagacccacggtgggttcagggagctcctgctcaccaagggactgggggagctggagcctggcctcagatcttgcacagtctgaattcctcttctgaatgccgaaggctccagtctttatttcagtcatccaggcctttatttcatcctgaagattatggtcaggctcttctccaagtcaatccagcaccaagttctgataagaatggttcctccagaacagaatgggctgcacaatttgtgggggccctagtccaatttcaagatggagaaataaatatgtgccctttctaaaggagagaccctgagcactcacaagggcctcaagccccatgaggcttatcctactccagaaagtcactcccacatgtgctgtgctcacaagattgctgatgccaccctgtccctgggcacctccagatgaagtgggcaagctgtcttgggtgtgcaccttttatctcactggccctggggtttttgcctggcc

SEQ ID NO: 59 - DNA, 401 bp, Homo sapiens MYO5A1693494   836
  misc_feature (201)..(201): n = c or t
ataacacacaggcagagaaaaagaaagactatgttacaaaggagagcagtattacactttctcctttgttgcttcactaaaggcaaagaggagtgcggccaattctaaaggccaagggtgtacacctgcctgaccacatcatgcccactggaatcatcaaaggcttacaactgaggctctatagaagattcccacgagganacacccatgtcataggcacacgggtgaaggaaacactcagagctagaaatgtcagcataagggatatgggctttcttaagaaaagaatgagtatttgtgttatgtacaaatgcttcttaaatatctttttagaagactctgtgatacaagttttacgttgatactaaaaatttggaagctttcaaagaggagaaataggt

SEQ ID NO: 60 - DNA, 839 bp, Homo sapiens CYP4B1RS2297810   350
  misc_feature (439)..(439): n = a or g
agcaggggagacagtaggtagatgttgaccaaaatgtcttcctctggcaggtggtattctatgtgcccagtgagcagaacagtcagggctggacaaggtcacaagtcatttggccagagcatttagggagggctttgtggaggaggaggcatccaggctgagctttgaaggaatataggaatctgggggaaggtcctaatccaccctctgaaaaggagctttcactccctcccagagaccttcatttgacaaccaccattatgagttcttcttgttacccacctccaatacctcaagctgtcctattatgccttccctaacccaggatgaagatgacatcaaactgtcagatgcagacctccgggctgaagtggacacattcatgtttgaaggccatgacaccaccaccagtggtatctcctggtttctctactgcatngccctgtaccctgagcaccagcatcgttgtagagaggaggtccgcgagatcctaggggaccaggacttcttccagtggtgagtctgagggtgggcccggtttatcctgctcagcccttgggaagggcgatgcccatcctgtcctgaaccatcctggaaatcaggtgaggtggcggctgctatcctgttacccccagtgtcataccttttgtggtggtggggggtggggagagtggttggctgggcacagatgcacccctgagcccattctcccaaaatggagcctttcagaagtgttatgtagagaaagttgtcaacaagaggctgatattttgtgtgctaacttcttctgacggtagaacctgattacccttctgggtccctggatagagtagggagag

SEQ ID NO: 61 - DNA, 401 bp, Homo sapiens MYO5A1724631   879
  misc_feature (201)..(201): n = a or c
atttttgacctaccagcaaaattgttgcccctaaataccaggcagcaattaagtttccttctcctcattaactgactcatgtctattgttcctttccttgtaccagaagtagaagttactcatttcccccaagtcctgagttcttatttatgctccttctaaaacataagatttgttatgctcaagtatgaatgagtttcnttattcctttgcccatatttcattttctgtacttagtccgacctcagttggcttaaattttaatatagctaaagttttaatttttttccagatttgaactacaaacttaaactatgatctttataagctttttgttgttgttgttgttctgtcaagcacataacacagctataggttcataaaagacagtaactgtaagg

SEQ ID NO: 62 - DNA, 401 bp, Homo sapiens MYO5A1669871   847
  misc_feature (201)..(201): n = c or t
actggatgggtcaagagcggtgtaccatttttgctttggtttgcattttttaaaatctcaagtgaggctgagaaccttttcatcacatgcatcctcaatgttgtaggaagtctggatgcttaggagggtacacctgtgctaaaatgtaacttactgcatataaactaaagagaaataaagcgaaagaataggtttgcctangatcactcagtggtaaagctggggatctaatgcttttttcccacatggtgcatctttaaatattaaagatacaactccatccctctcaaatatgtcagtaataaggaacaactagttagttttacaattataactatttagaactgttattcaaaatatcttctgcacagtttcttgcatttctttaaattctatcgctg

SEQ ID NO: 63 - DNA, 1120 bp, Homo sapiens NAT21041983   483
  misc_feature (572)..(572): n = c or t
gtttaccatttggctccttatttaatctggatttccaactcctcatgcttaaaagacggaagatacaataatactttccttacagggttctgagactactaagagaacttatgcatgtaaaagggattcatgcagtagaaatactaacaaaagaattactatgacagatacttataaccattgtgtttttacgtatttaaaatacgttatacctataattagtcacacgaggaaatcaaatgctaaagtatgatatgtttttatgttttgtttttcttgcttaggggatcatggacattgaagcatattttgaaagaattggctataagaactctaggaacaaattggacttggaaacattaactgacattcttgagcaccagatccgggctgttccctttgagaaccttaacatgcattgtgggcaagccatggagttgggcttagaggctatttttgatcacattgtaagaagaaactggggtgggtggtgtctccaggtcaatcaacttctgtactgggctctgaccacaatcggttttcagaccacaatgttaggagggtatttttanatccctccagttaacaaatacagcactggcatggttcaccttctcctgcaggtgaccattgacggcaggaattacattgtcgatgctgggtctggaagctcctcccagatgtggcagcctctagaattaatttctgggaaggatcagcctcaggtgccttgcattttctgcttgacagaagagagaggaatctggtacttggaccaaatcaggagagagcagtatattacaaacaaagaatttcttaattctcatctcctgccaaagaagaaacaccaaaaaatatacttatttacgcttgaacctcaaacaattgaagattttgagtctatgaatacatacctgcagacgtctccaacatcttcatttataaccacatcattttgttccttgcagaccccagaaggggtttactgtttggtgggcttcatcctcacctatagaaaattcaattataaagacaatacagatctggtcgagtttaaaactctcactgaggaagaggttgaagaagtgctgagaaatatatttaagatttccttggggag

SEQ ID NO: 64 - DNA, 121 bp, Homo sapiens GSTT22267047   464
  misc_feature (61)..(61): n = c or t
tggccttggagggatcacagcctctctgaaccttagcttgccttctgaaaaggaggataangttaccttctgctctgtagggatggaaagaaaatactgaatggagttgacagagttcttg

SEQ ID NO: 65 - DNA, 631 bp, Homo sapiens CYP2C8_RS1891071   369
  misc_feature (75)..(75): n = a or g
taatctgtctaaatttgatgacacaatttaaaatgacatctttgtacaatggaggaggatgacagagatcagtanaaacagtatggcagtagcaaaataagtaaagcactgatgaagtgtctggatttcagcaaaggtaatttgtggtaaggagagccagcataaattgccctagtattgaatgttggttttattatgaaaagtccactttgaacagtaggttcatttctcattttaaaaattccatgctctaatgctgtggtggggagatgaaaacaatctttattgaagcataagtggaaattctagaattgtactgaggcatcctgacataaattccagtctgggaagtaatctaaaagttagtctcttacaaaggtgtttctatttaagcagaggccatacctaaaggaattttattattctaggagtgtgtttcataaaaatgctatttgaccaaatagggacatttggaagggggtttaataattgcatctttcacatacaacttttctttagaatttacaatttacccttagagtaaactctacatttccttagaatttacatttaaatagttcctgctttgcagcagataacatacctttttccctgtgtcaggatcccactg

SEQ ID NO: 66 - DNA, 401 bp, Homo sapiens CYP4B1RS751027   343
  misc_feature (201)..(201): n = g or a
gtttatatcaatatgaagagggatgccctgtggaggcagcccctcctccctgcaggcccaccaagtgatttttacattgaaatcagcaaaccagagcaaaaagaaccagatgtagcaggtcatgggaggagactctgccacggaattctccaaatccagtactcgaggatcccatacccagacactgacaggtgctgcccnccactgagcctcctctttctgggtctcagatgcccacattttaaaattgagacatagaaattcattcctcctgtttacaatccagtcttatggctctcctttgatgactttcgctagattctgctctagtccagctgtgttgatgcctccaattaacagagctccaagggtaatccttgtttccttttcctgctacatgg

SEQ ID NO: 67 - DNA, 619 bp, Homo sapiens MYO5A2899489   930
  misc_feature (137)..(137): n = g or t
atggtagcaatgccttagaaatttcaggagtagggaagagaaaaagtgacatctgggaggcagtagagactcagcaatatatttaaaaacaacaacaacaacaaaaaactatgggtgcaaggacacgcctagaaatntctaaaaaacccattctcagcactcagttttccttccccatccattccaatccataatttggaaaaacatgtcccactccccaacattctatggattaaggtattttaattgagcaaatgttatgttaaaagatatcctggcaagaatgcgaaggtccatccccagactagatgaacttcttccttagcttatggctatgtccacacccttatgacagccctaattgtggttcctgaacttctattattataacacaacgatttagaaaaacagtctgctctatacagctaatgtcacactgtggaaatgaatcattctgaccaaaagccttgcttctggcagtgccaatcccttcctaaagcaactgcaagccgtccacctcccagtcaggaagcatctggtagctggggctctagaagtatccctgggaagtcagtcagaactagagagaccactaacatttgttagggggcactggaat

SEQ ID NO: 68 - DNA, 775 bp, Homo sapiens GSTT2140184   568
  misc_feature (695)..(695): n = g or a
taaccttaagcaaaaaactgggaaggatctcggtaaaagatacgtcagaaggaagtaatgtcctttaagatgttcttagaaacccatcagacaggtggggtgtggtggttcacgcctgtaatccctgtactttgggaggcagagatgggcggatcagttgaggtcaggagtttgagaccagcctgggcaacacggtgaagccccgtctctactaaaaatacaaaaattagctgggtgcggtggcacactcgggaggctgagacaggagaatcacttgaaccttggaggcagaggtttcagtgagctgagatcataccactgcactccagccgggccactgagcgagactgtctcaaaacaaacaaacgaacaaacaaaaagaagagaaactcatcagacgaagacacaggaaaaaaatgagcgaaggaaatcagcagaggtttcattgaaggacaaagagaaatggtcaatacatggatgaaaacatgtttaacttcagtaataatcaaggaagcacacaccaacacaacatgcacatactgtttttatttatcaaaggcacacatatttttgaaatgagtactcctaattaatatgtacagagcacttacccagtgcccagcacaggggtggcaccctgtgtgtgagacagcatgaaacaggtagacacgcgccctgctgaaagtaagggaccncctctctggaggatccatcgggcaataaggaggtttccacaccttaactgtgtctgccttgacctctggggcctgggagc

SEQ ID NO: 69 - DNA, 400 bp, Homo sapiens CYP2C8E2E3_397   134
  misc_feature (200)..(200): n = c or t
aactcctccacaaggcagtgagcttcctcttgaacacggtcctcaatgctcctcttccccatcccaaaattccgcaaggttgtgagggagaaacgccggatctccttccatctctttccattgctggaaatgattcctaataaaaaaaggggcagaaactgggagaattcacagccaaggaagaaagtgctgcaacactnggcagccatgcagataggctaagctctgctgagaagctttttagggctctgttttccatccccctcaccccagttaccaaagctgacacagaaatatgtgcacctaccaagtcctttagtaattctttgagatattggggaattgcctcttccagaaaactcctctccattatcaatcagggcttccttcactgcctcat

SEQ ID NO: 70 - DNA, 1050 bp, Homo sapiens CYP2B6RS2279345   142
  misc_feature (557)..(557): n = c or t
tcttgaaatactttcctggggcacacaggcaagtttacaaaaacctgcaggaaatcaatgcttacattggccacagtgtggagaagcaccgtgaaaccctggaccccagcgcccccaaggacctcatcgacacctacctgctccacatggaaaaagtggggtctgggagaggaaaaagggaagggaggggagggagggcaagatggagaggtgagaagagggagggaaaaggggtagggaaggggaagatggggagggaagaagaaagactagggaggggagaatagggaaagggaggagagaacatgaggaaggaaagaaagatgaggtgaaaggagggagaaaatagggaggaggaactgagacagggagagaggggaggtgggaagacagaatgaaagacagagggagagagagagaagactggctgaggaaggaattcggggcaagggacaaaaatacagcaacaagagaaaaaactcacagaggcagaaagagacggggacaaaaagagagaaacacatcaaagagatgtggagagagatagaaacagagtnaggaagactaaagagaggctgagagagatgagttagagatacgcggttggatgtgtagaggacagagaaaagcaaactgggccagatagtgtcaaagacctttaggccaacggagggcagccagggagatgggcgtatacacagcaaggctacagcctcccctgaccctccccttccttccctactgtggacgcaggagaaatccaacgcacacagtgaattcagccaccagaacctcaacctcaacacgctctcgctcttctttgctggcactgagaccaccagcaccactctccgctacggcttcctgctcatgctcaaataccctcatgttgcaggtgggccagggacagccagtcaagggggtcttctgacctccttctgagctgcagaaatggggctatgggtaccacctggatgagagaggggatgctggcttcctattctgggagcactgtaggctctgggctagattccaaccaagccaattctg

SEQ ID NO: 71 - DNA, 603 bp, Homo sapiens MYO5A935892   898
  misc_feature (122)..(122): n = a or g
acgtgagtaagagctggctagggaagaagagcaatgtatgtaccccagcagggagaatctgagcaaagccaggagagagaaacagcctggcatggaaatggctggatgtagagtgtgagaancaatgagaaagaagctcaataggaagtcaggggtcagtcatgaaggccctttcatgtcatatcatgccaagtaaggtgttgggtctgcttctgaaggcagtggggagtcctgaaacatttcagtaagggcatgatatgatccctaatagctttaacttccatgagtttctgatcttgtggggccacatctgcgttatagtcactaatctcatctatccaaccctctctctttctctctctctctgtcacacacacacacacacacacacacacacacacacacaatcactatgcaccagagattagaatccatatttctttattctcagtttgagattctcctttggataacatatgtctttgataattatgtttctgataatattgatagagagacttaaactatattgcttctttaaacaatctacaaatctaaaacataattcaactgccatcccactaaagcttattctgaacttac

SEQ ID NO: 72 - DNA, 401 bp, Homo sapiens MYO5A1669870   877
  misc_feature (201)..(201): n = c or a
gggaaatattgtatgatttggtaaagcaggactacttgagaatggactatttcttttccaaaactccagcaacttcagttgtctgccactcaagggggtcaaagcttgcatgacaaaacctttaggtggccctagggtcatctcaaggcttctagatggaattagggcagacttagaaagtcctcaatccctaaaggagancctgtgaaacttaccccaaagcctacatcacagtctccttgaaatataaattactctcattgcttgtgattttctaagtacactatagaacttgtgaagcagtaagacagtatgccttattagaagggacccagtgaatcaattgcagagggtgacacaagtccacaattgctattctgaaacccttgagatcagctatg

SEQ ID NO: 73 - DNA, 401 bp, Homo sapiens MYO5A1693512   821
  misc_feature (201)..(201): n = g or c
agagaacccatccgatctactggagcaagcatctcccacccgccgggaattttccaaagccaagcaggaggggaggcacgcccgccctgctaaatccacatgggccccctttccactccgaagcccgctctgcccccagctcgagcagcgcggcaggggcctgggagacccccgaggcgggccaccttccgccgccttcancatctcgcccgaaagaggaaggtgccgcagcgggcgaccggctggtagggccgagggttctgaggcgctgaaggggatggcgctggtggggctcgcctgggcccggcgctcccgccccctccccagcctgacagctggcggcgagggccgcacagccccagtcctcgacgccggccgcggggtgccttacctttgtgtag

SEQ ID NO: 74 - DNA, 401 bp, Homo sapiens CYP2A131709081   503
  misc_feature (201)..(201): n = t or c
cattaggccttttgccttagggacacaaatctcaggtccctcaaacaccctgcctagtggaacatggaccccatgtctcccaaacttcctgtttcagagacatgaaacttctatcccccaaagctcctccctcagaggtccccaactcctccatgcctgccactcccctcacctggggcaccctagttccccctgcagccnctgtgtattttcaccaatccccccaacctgcctcattacacacaccttcctcctccctcccagggcactgaagtgttccctatgctgggctccgtgctgagagaccccaggttcttctccaacccccgggacttcaatccccagcacttcctggataagaaggggcagtttaagaagagtgatgcttttgtgcccttttc

SEQ ID NO: 75 - DNA, 509 bp, Homo sapiens MAOA909525   549
  misc_feature (86)..(86): n = a or g
gtttaaacaatctcttgtttaaacagtaggaaactgcacaaacaagctagacttcccaagagtgaaggccaggtacagaggaaatnaagcattccaaataatgccaggtaagaatgaggatgaataaccagttcaaaggctaaagaagtggcctcaaactcttgtgttccttggagctctagggttgctccctaggttgctcagggattgcctgtagctgggggaggggagtgtgcgtgtgtgtgtgtgtgtgtgtgtgtacatgtctgttgcagcacctgccacccccacccctgtggccttcaaatgcattaggagggaacccagagcctcccattctgggagtgaggatagcttgtgagggccactgagggtgacagggaggaaggtcaagctgagtcataatatcctgcagtgattccaggagactttagagatttttttcaaaagaaaaagaaaaaagaaaacaagaaaagaaaggcaaatactacttcaaagtcaagagccta

SEQ ID NO: 76 - DNA, 966 bp, Homo sapiens RAB271014597   932
  misc_feature (328)..(328): n = g or t
tctcacttataagtgggaactaaacaatgggtacgcatggacataaagacagaaacagtagacactagggactccaaaagggggaagggagtgggagaggggctgaaaaactacttattgggtactacattcactgtttgggtgatgaattcaatagatgctcaaaccccagcattatgcaatctatctacgtaacaagcctgcacatgtaccccctggttaaacaaacaaaaaagatgagattgggaaaagccatagaaaagtaatatttcctgaaactagtgacccaagagaactttgaggaaaggtaaactaattattatatttnatacaagttgttttgtattcatattttacaatatatttataagatatatatgtatatatttcaatataacttattacattacctggatataattatttgacataaatacaaacatggttgtattccagatggcattctctttggaattttagatgcctttgggatttgtactgaacaaaaaggtgagaaggttcgctggtgttagaagttctcttttgttttctctttcactttgctgttttaggtgtacagagccagtgggccggatggagccactggcagaggccagagaatccacctgcagttatgggacacagcagggcaggagaggtatgagatcttcagttatgtgctccttactgaaggaaagggaaaaatagttacattcttcaaacagtgacctgagcaggaaaaagccagccaattcgttggtttgcacttgaatgctgccaaattagcaggaaatttgtcaagtctgagatgagaatggtggccttatttcatacaaggtcaagggagaggttatgactcttacttgtggacttttttcttttccttcttttaatttttttttgcttagatactttgctccatttccttttgctatttactcaaccacaagaaagtggccaagttac

SEQ ID NO: 77 - DNA, 611 bp, Homo sapiens CYP2C8_RS1891070   357
  misc_feature (281)..(281): n = a or g
tattcggatttttttcttgctgttttgagtttcttgtagactctggaaaatagtcctttgttgaaggtatattttgcaaatattttctcccattctgtaggttgtatgtttactctgcttgtcatttcttttactgtgcagaagctctttagtttaattaggtcccattgtcaactgtttttgttgaaattgcttttaaacattgagtcataaatccttagcctacaccaatgctcagaagagttttttataggttttttctagaatttttatgatttcangtctcatatttaagtctttagtccatcttgagttaatttttgtatgtggtgagatataagaatcatatttcattcttctacatgttcccctgggtaatatcagccaagcacaaatcccacagctaccagcgtaggtggctctttcctgcaagaaccacctcctagctggaagccaataggcacagcctattacaacatctgctggcaaaataacatagcatttgggaaggagaaaacttttatcgtatctcagctaacaccatacccacatcaccccagctaatcggaaggtcttgagtgtgttcacaaacccaatacattgctagtaca

SEQ ID NO: 78 - DNA, 531 bp, Homo sapiens CYP2C8_RS1341159   94
  misc_feature (406)..(406): n = g or c
attatagccaatatttgtaattttctgttttttgtgtcagtgcaaagtggtatttcattgtggttttgacttgacctatgatattaattagctttttaccatttttacatgttctttagagaaatgttattcaaggcccttgttcatttttattttattttatttatttattttggagacaaggcctctctgtgttgctcaggttggagtacagtgctgtcatcttggctcactgcaacctctgactcttggtctcaagtgattctcctacctcagcctcccaagtagctaggagcacaggcacaaacccccacacccagctaatttttgtattttttttgtacaaacttggtttcaccatgtttcctaggctggtctcaaactcctgagctcaagcagtccaccnatgttggccctcccaaagcactgggattgcagttgtgaggcaccacacctggccctttgcttatttctatactgggttgcttgtcatttgttgttgaactgtaggtaattgtttatggattctgg

SEQ ID NO: 79 - DNA, 1470 bp, Homo sapiens CYP2C8_1341159   95
  misc_feature (703)..(703): n = g or c
ttcaataatattccattttcatgtacacatatgacaatttgcttatcattcatctgttgatgatcatttgtgttattttcaccttttggctcttataaataatgttgctatgaacatttgtatacaagttacttcatgaatatattttcatttttccagggtatagtcctaggagtgttatttctgggtcatatggtaattttatgtttaactttttgagaaacaactaaacatttctacagtaaatgcaccattttaaaatcccatcagcaatgtttgagggttcctcttttccatattatagccaatatttgtaattttctgttttttgtgtcagtgcaaagtggtatttcattgtggttttgacttgacctatgatattaattagctttttaccatttttacatgttctttagagaaatgttattcaaggcccttgttcatttttattttattttatttatttattttggagacaaggcctctctgtgttgctcaggttggagtacagtgctgtcatcttggctcactgcaacctctgactcttggtctcaagtgattctcctacctcagcctcccaagtagctaggagcacaggcacaaacccccacacccagctaatttttgtattttttttgtacaaacttggtttcaccatgtttcctaggctggtctcaaactcctgagctcaagcagtccaccnatgttggccctcccaaagcactgggattgcagttgtgaggcaccacacctggccctttgcttatttctatactgggttgcttgtcatttgttgttgaactgtaggtaattgtttatggattctgggcattaaacccttactaaatacgtatgaaatacaaatattttctcccattctacaggttgtcatttcacatttttaattttgtcctttgatgaacaaacattttaatttggtgaggcccagtttatctctcttattttagttgttttggtgtcaaatctatgcatccacttccaattctgaaggcattaatatttaaccgatgttttattctaagaattgtatagttttagttcacatttaagtttttcgttcactttcagttatattttgcataagagtgagataggggttcaacttcattcttttgtatgtggctacccagttgtcccagcactgtttgttgaagaaacgcttcctttatttatttatttttttttgaaactcttcctttagattaaatgatcttggtacatttgttgaaaatgaaccgggcatagatgattaggtttatgtttggatttcaattttattccactggtctttatttctttccttttgccagtaccatgctgttttgactactatagttttgttttgaagtctggaaatttggaaattgagtcctctccctgtagttgcatataaattcaggattggcttttccatttttgcacaaataaaaattttaaaaaggacattgg

SEQ ID NO: 80 - DNA, 121 bp, Homo sapiens POR17685   691
  misc_feature (61)..(61): n = g or a
ccctcggtggctgcacagaagggctctttctctctgctgagctgggcccagcccctccacntgatttccagtgagtgtaaataattttaaataacctctggcccttggaataaagttctgt

SEQ ID NO: 81 - DNA, 1050 bp, Homo sapiens CYP2C8_2071426   362
  misc_feature (459)..(459): n = a or g
gtggcactatctccactcactgcaagctctgcctcccaggttcacaccattctcctgcctcagcctcccccgagtaactgggactacaggtgccctccaccatgcccggctaattttttgtatgttttagtagagacagggtttcaccgtgttagccagaatggtctcagtctcctgacctcatgatctgcctgccttggcctcccaaagtgctgggattacatgtgtgagccaccgtacctggcctcttttgtctttctaagtctgtcattgtcagaaatagcggagtgagttgatgcattttgtgaatacagaaacattggggtcattgtattatataatcatttaatacagtggcaaaagtttaaagtgctgtttctcctctttgtttcacagtgttttgctatgatttttgactgaaggtgaagggaagtgtgtgtgattagaaatttcatccantaagttctctactatagtagtcatgtgttttattcagaatggtcatgaaaattgaacttctctgaagattcatttgatggctgatgtgaaataaatatctgtgggttcagggcaaacataagtgcatgaaagaaagaagtaatcagtcagggcccaataggtagttaacagaattcttttggattctgaagaaagccactgtctgtggccaaggttgctggagaatggaagaaattgttcttccaggagatgctgaatgtcctgattctaactttgtggtgcttcatcgttccatattggtaataccagcagttacaaactggactgggcattagaatcacctggggtgacactgtaaatacagatttctagggttcatcacaggactgctgtatcagaatcctcatgttaagagctttacaagtgaccctgaagtctttagctgggtagtggcctcaaggtggacatgggggattgattaattgctcaagcatcagtttaaattagcagagattccagtttggagcttctacatattacctgtgggactctgagaatgaatctgcaattctctggcctcagtttcttcat

SEQ ID NO: 82 - DNA, 1540 bp, Homo sapiens CYP2C8_RS947173   342
  misc_feature (761)..(761): n = g or a
taacagaactgcactaattttatgctctataattgctgtgctctccctccctcagtactcagaaatactctctgaaccatgccactgctgccaggggaagagaggatgtcagtaattcaatagtattttttctatctcttcatttcctctttcagtgatatatatttaaatcaaggtgcatgctcatctgattttggttcttatgaaggtacattttgtgtagatagctgttaaactgatgtctttgcttggggaataatcaatgaagcattcaattctgtcatcttgctccactctcccatttgtatatcttcttttgagaaatttctgttcatgttgtttgcccactttctaatgggattattcggatttttttcttgctgttttgagtttcttgtagactctggaaaatagtcctttgttgaaggtatattttgcaaatattttctcccattctgtaggttgtatgtttactctgcttgtcatttcttttactgtgcagaagctctttagtttaattaggtcccattgtcaactgtttttgttgaaattgcttttaaacattgagtcataaatccttagcctacaccaatgctcagaagagttttttataggttttttctagaatttttatgatttcaagtctcatatttaagtctttagtccatcttgagttaatttttgtatgtggtgagatataagaatcatatttcattcttctacatgttcccctgggtaatatcagccaagcacaaatcccncagctaccagcgtaggtggctctttcctgcaagaaccacctcctagctggaagccaataggcacagcctattacaacatctgctggcaaaataacatagcatttgggaaggagaaaacttttatcgtatctcagctaacaccatacccacatcaccccagctaatcggaaggtcttgagtgtgttcacaaacccaatacattgctagtacagctggcatttgagaaaattaccacactaaacctatttataaccaagtaaatcttacaaagtctatgtcactctcttgccacctcgatcagaggtggtgcttgcacctgctgctaggagaccagaggacagttaggcccagttaagccccattcaacattgccctcctttgtagcaaagagtggaacccaagcactgtacatctctcaaacctttccacagcctgaggcatcagagatttccagttggctgacaaagatgtcaatgttcaattatcctcagaaggaagagccaatattacaggtgaataatcataagccaaatggactgttaaagagagagcaatggagcttattggtcaattcataagaagaaagaatctacaactcacaaacacgcacacacacatacacacaaataaagtaaaaagaagctggcagaggtgaaaccctgagagacttagtaatccatggaaagggcaggtaggagtgttctcagcctccctacccaatttggcagactgctgggatctgaactcaaggtgaccttccttgccttcatgagcacaag

SEQ ID NO: 83 - DNA, 401 bp, Homo sapiens GSTT2140185   783
  misc_feature (201)..(201): n = a or g
gtgccccctggtgagatgccagggctgggattcagggagaagaaaggaggttcccggacagtcattcctgcctcccgcggctgcgggctccctgcccccatcctgtgcacgaagtgggagctcccgctgtctggcagctcccgctgtctggcagcagctgctctgcaggggacagtctggacggcagaaagttcatccttnaccccagccttccagtcaaggttcccaccagtttgggacacctgcaagtgtcacatcccactgggtgaaactctaagatcccttttaggggatcccattcgctccctcccttccgccaccatgcagcgccgagaaacagagctctgaacgaaccctcagatgtccgtgcgctggggcctttccaggacggcggcgcccag

SEQ ID NO: 84 - DNA, 401 bp, Homo sapiens GSTT2140188   652
  misc_feature (201)..(201): n = c or g
ggaggcggagcttgcaatgagcagaggtcgcgccactgcactccagcctgggtgacagagggagcccactccagcctgggcgacagagggagactccgtctcaaaaaaaaaggaaagaaagaaaggagaggtatctggggagaaggtacagcttggggtgtgtccgggatgagcaggggctgacagaacatgtccccccanctctcatcttcagccttttctgagccgcagggcctctccactcccagactgaagggtattagaagagaagacaagggaacatttttccactgttgcgcatttgttcaacaaatgctagctgaaaagagcctctagtgacttgtcgcagactacccaatctacccaggccgggcctagaggccaatgccatggcccaaggg

SEQ ID NO: 85 - DNA, 401 bp, Homo sapiens CYP4B1RS751028   292
  misc_feature (201)..(201): n = a or g
tttctccatggtttatatcaatatgaagagggatgccctgtggaggcagcccctcctccctgcaggcccaccaagtgatttttacattgaaatcagcaaaccagagcaaaaagaaccagatgtagcaggtcatgggaggagactctgccacggaattctccaaatccagtactcgaggatcccatacccagacactgacangtgctgcccaccactgagcctcctcttcctgggtctcagatgcccacattttaaaattgagacatagaaattcattcctcctgtttacaatccagtcttatggctctcctttgatgactttcgctagattctgctctagtccagctgtgttgatgcctccaattaacagagctccaagggtaatccttgtttccttttcc

SEQ ID NO: 86 - DNA, 993 bp, Homo sapiens GSTP12370143   533
  misc_feature (499)..(499): n = c or t
gaggcttcgctggagtttcgccgccgcagtcttcgccaccagtgagtacgcgcggcccgcgtccccggggatggggctcagagctcccagcatggggccaacccgcagcatcaggcccgggctcccggcaggctcctcgcccacctcgagacccgggacgggggcctaggggacccaggacgtccccagtgccgttagcggctttcagggggcccggagcgcctcggggagggatgggaccccgggggcggggagggggggcagactgcgctcaccgcgccttggcatcctcccccgggctccagcaaacttttctttgttcgctgcagtgccgccctacaccgtggtctatttcccagttcgaggtaggagcatgtgtctggcagggaagggaggcaggggctggggctgcagcccacagcccctcgcccacccggagagatccgaacccccttatccctccgtcgtgtggcttttaccccgggcctccttcctgttncccgcctctcccgccatgcctgctccccgccccagtgttgtgtgaaatcttcggaggaacctgtttccctgttccctccctgcactcctgacccctccccgggttgctgcgaggcggagtcggcccggtccccacatctcgtacttctccctccccgcagccgcggccctgcgcatgctgctggcagatcagggccagagctggaaggaggaggtggtgaccgtggagacgtggcaggagggctcactcaaagcctcctgcgtaagtgaccatgcccgggcaaggggagggggtgctgggccttagggggctgtgactaggatcgggggacgcccaagctcagtgcccctccctgagccatgcctcccccaacagctatacgggcagctccccaagttccaggacggagacctcaccctgtaccagtccaataccatcctgcgtcacctgggccgcacccttggtgagtcttgaacctccaagtccagggcagg

SEQ ID NO: 87 - DNA, 636 bp, Homo sapiens DCT2224780   674
  misc_feature (599)..(599): n = c or t
ggagaaagaaccaaggtgatgctagaagagattctagacagagactaagctacctctcaggccattcttgactaaacaatcatgaaaactctaggagagagttgctcaactcaatgctagaaccatcttagatttgtatgtaagttgtggtttgttattatattcatattttatcagaatgaattggatgtaattcataggtttagttcttctcaatatagtatgcatttatccttataaattctagagttgaagagaatccattcaggtgacatttagcacctgtgaaattaaagaaaacaagccagcccccagcctagtccatagaaacactgccaccctggggaaccagagaggggtccagccaccctctctgattcctcagctcttataaaactcatcaagatgttatgccacttaggaggtagtaactgtgtacctgctatttaaaaactagtattgaataagtaaatgtgacatttaaaaagcataaatacatgctcacaatgaaagcaatgactatcatttcaaaagctgtgcaaaattagtcagatctgcccttcaccaattagtgttaattcctattaatatgatctaangggacttaatttcctcagctatagtgaatgcaattgt

SEQ ID NO: 88 - DNA, 401 bp, Homo sapiens CYP3A7RS2687140   287
  misc_feature (201)..(201): n = a or g
gggagaggggggaggtcaggatgacatttcagtcactccaggttaaatcgcaggactgagttaaatattggaattcctgtatatatttagtggggtctgatgaaaaagagcctaaacgctgactgatctgggagaggtcgatagagaaaaaggcacatgtaccttgactatgccttcagctccagccacctgactaagagnaaattgttgggcaggtggaggagggctagtctcggaatgaaactgtaaggtggagtgggtgtgaggaggggaggtgatacttctattatagggtgggggagcagaggatgaggaagaattgggacctggcttggcctggtgaggagcagcctggtggggaggggagaggtcagatgggttcatagaaaaggaggattcaa

SEQ ID NO: 89 - DNA, 401 bp, Homo sapiens GSTT2140192   469
  misc_feature (201)..(201): n = c or t
cagccagtgtcacctgctggccagcgaggaagggcctgtcccccaggaacttgtcctccagccattgcagggcctggtccatggcagtcctgttgcgttccaccttctcctcgggcacctggaccccaatgaggggccccaacacctgatgggggcagagagtgggtcagtctatggccccggcctactgccaactactcnctgatggccaatcactctccagatggctctcctcacctggacccacaggggtataccaaaggtgccacggatgcagtcggcatgccagcccaggtactcatgaacacgggcacgagcctgcaggtcagatggataccagtggtccggcgtctggtacttacagctcaggtaaatcaggatggccgagctgggaacaaatg

SEQ ID NO: 90 - DNA, 121 bp, Homo sapiens CES22241409   658
  misc_feature (61)..(61): n = c or t
agcatcccaggtggagctcgtccttggcccccgagacctcggtccccagtcctgcttctcngctttttcttccactgccctcagaagccagccctccccttttccaaactttccctgtaga

SEQ ID NO: 91 - DNA, 201 bp, Homo sapiens AP3D125673   828
  misc_feature (101)..(101): n = a or g
caggctgcacttcagctgcagctcctacttgatcaccactccctgctacagtgacgcctttgctaagttgctggagtctggggacttgagcatgagctcantcaaagtcgatggcattcggatgtccttccagaatcttctggcgaagatctgttttcaccaccatttttccgttgtggagcgagtggactcctgcgcctc

SEQ ID NO: 92 - DNA, 364 bp, Homo sapiens CYP1A2E7_405   98
  misc_feature (174)..(174): n = g or c
ctagagtataccagtccactccagggaagattggagctgaggctgcttgagggctatacacactctgggaactagggggtctccaaacccttgagaggtttgcaggaggaaaactgcaaggagactggcagaaagcaggctgaagtggaagcttcctggcccgtgctgggctcntcagtgcttgagaacatagatgaagggcagacagtggccgcagacgagggacgctgtgaggaggaggcctggcatgtcttggggccaggaagagctccctgatcattttttccttcaggatgggtaggctcttggtctgactgcccggaaaacaggtgatggaaggaccaaggactacaaaggtcaggac

SEQ ID NO: 93 - DNA, 401 bp, Homo sapiens CYP2A131709084   546
  misc_feature (201)..(201): n = g or a
aatctttgaacacagatctgtgcccatagccctctagatagattcttaaaaagcaccccttcctcacgtaaaatagcttagtatagcatcacatggcctgaacatccctgtcctggggagttttccagagacctggcgggcggctgtcctgccttctctgcacactttcctactcggcacgctttgaacaccagggtgtantctgagctcgctaccaggtaaggccactgtggcccaatcagaatcagtctaggacacaaagagacatgaatggacatacagagtcagtccattgacaattcctttgcagagcagaagtttttaattttaatgacattctgtcattgtatctcttaatgaagaagttgaaggagagaaccactttaatgccgcgagaactc

SEQ ID NO: 94 - DNA, 121 bp, Homo sapiens GSTA22290758   558
  misc_feature (61)..(61): n = a or g
gatgaccaccactcattcatggtgccccaagcatgaaaacaaagaaagggctttctcagcngtgggagattgttcctctagcaaatactctgagaggtctggtccttttaacctggagaga

SEQ ID NO: 95 - DNA, 401 bp, Homo sapiens CYP4B1RS632645   171
  misc_feature (201)..(201): n = t or c
ctcacattttatacccatttcccagataatgaagctgaggctcagcttgcctatgctttctctcaagaaagcttccagtgtccccttcaatgtgaacccttaagagagctggcatttatgctaggctcgcaatgctttgagttcttttttgtgaggcaccttcagagacaaggttctagccccaaaagggaaataccaggncagaaaggggccaactccacccctaaaacaatagtgccattttgacacttaggaacatagaactttcagggtgatgagaggtcatcaaattcaatccctcctggctggatggagccacttaagctcaagagaggtagggaaatatccaaagttgcacagcaagaaattggcagctcccagtgccacagcccaggcttctg

SEQ ID NO: 96 - DNA, 401 bp, Homo sapiens GSTT2140187   562
  misc_feature (201)..(201): n = a or g
ccagagaatgctgtggactgagtggccttgaagggatcacagcctctctgaaccttagcttgccttctgaaaaggaggataacgttaccttctgctctgtagggatggaaagaaaatactgaatggagttgacagagttcttgcgtggaatgcacgcatataaattcacaaagcccagaagacctcgggaagaaggacatnctgttgtgagaattaagagatgggaagagatgagccaccccagtttgcctcccctcccctggcccaccagagtccggctagaaaacttctctttatccacctgctgcacctggccccacccaccaaaaccccccagctgccccggaatgtggcagggcagggaggcccagccagggagtgaggctgatccaggcctctag

SEQ ID NO: 97 - DNA, 413 bp, Homo sapiens GSTT2140190   443
  misc_feature (269)..(269): n = c or g
tgctgggcaggaaaaggacaagaggtcaggtggctgcagaggtgatggctgggggcctgtcagacgggggccaaagacattcctcccctcgtgatccctgacccaagcgcgtggacatgcaagggactccacggagcatccactgtgtgccagccccatgcagggttccaggggtccagggagcctattctgagctgcaccgcctcggacaagtcacttgaccattctgaccttgagttttctcttgtgctaaaaggctaacaggagtntctacctcacagggcggctgctggcatatcacagagatgaggttctcaaaatgcaaagcagaaggtccagccaagagtcggtgcccaaggcaacaaagacaggaggagactcgtaggaggagggggtggtgttggggagctgga

SEQ ID NO: 98 - DNA, 540 bp, Homo sapiens DCT1325611   657
  misc_feature (356)..(356): n = c or t
ttggctattgtaagtaatgctgctatgaacatgggtgtgcaaatatctctgctggaccttgctttcagttcttttaggtataccagaagtataattgctgggtcatatggtaattatttgttccatttttttgaggaatccccatactgttttccatagtggctgcaccattttacattcccaccagcaatgcacaaggattccaatttctctacaccctcagcaacacttactattttctatttttttttgatagcagtcatcccaatgggtatgaggtggtatcttattggggtttctgcctggcatctaaggccctctgtacctaggctctttcataaatttgaacttaattngaggtaattctctgcccaagcgtcccactacagccaggcttgaaagactcaggtcaaagagagagagactgagctctgaaatcatcttgattgctttctaggctgagactttgggtaaataggctgtgtgatttttcaccttcttgattaagattttttaaattgttttgtttttgtttttttg

SEQ ID NO: 99 - DNA, 615 bp, Homo sapiens DCT2892680   699
  misc_feature (115)..(115): n = a or g
aggtgtaaaagcagatagttcatttaataagatctatttaaaactttcagttttctaaaacaagagtcagctcattagtccctttctgatgtttaattggtagtttaaaggctcnttttgctaaatacgtatatgctagaaaaatggtcaataaacttaactagacctgaaagcttcggctaaaggtcttggtttacattataaagaaagaaagcaagaaagctagcttctttagaagatgaatgaatcacccatttggaagttgtgttagttacctggcagatcgatggcatagctgtagccaagttggtctgaggttaaaaagagttcttcattagtcactggagggaagaaaggaaccatgttgtacatccgattgtgaccaataggggccagctcctgaggccaggcatctgcaggaggattaaatcttttcatccactcatcaaagatggcatcagtaaaggaatgaagaacctgcaaaacagttggacacagcatttaacataaatcagtctgttccgatcacaccaacctgactgttgctttctctaaagtggaataacttcttcttgacataggaacttctgtatacccatgtgtggaataccccct

SEQ ID NO: 100 - DNA, 121 bp, Homo sapiens GSTA22290757   495
  misc_feature (61)..(61): n = t or c
atgttcactgttccctcatctacatgggactctgcaatactggacctcagcgtacatgccnaaggcccagcctgctgctggtcatgatgccctgccatcgtcccacccactcaaggaagga

SEQ ID NO: 101 - DNA, 121 bp, Homo sapiens CYP2D6_RS2267444   93
  misc_feature (61)..(61): n = c or g
gccagcgctgggatgtgcgggaggacggggacagcattcagcacctacaccagacagaacngggtctcaatccctcctgtgctctgcgttcatctggaccagtctcaggccccagccatct

SEQ ID NO: 102 - DNA, 1190 bp, Homo sapiens CYP4B1RS2297812   97
  misc_feature (649)..(649): n = g or c
acgatacggcaggggtggaattgaggccatttctctattgcacaatgagcaaatattacctatctttataattaaagagctttaatgtggagaagaaagcactttctggctgcagtgcagtagagagttggtggcaggtgctaaatggaagcaggttggctactttggggatgttgagctaaccccggtgtgagaagagggcggcttggaattagagcagtggttgtggggctggagaggggactaagcccagggatgtttaggagaagagggtgcaggagaggtaaggaagcaaccagggcctgcctgggcagccttctcccctgccctcccagggacttggggcctagctggtgacaatgtgttcctgagtgaccttggccttctgtcatcatttcagatccaggagacggggagcctggacaaagtggtgtcctgggcccaccagttcccgtatgcccacccactctggttcggacagttcattggcttcctgaacatctatgagcctgactatgccaaagctgtgtacagccgtgggggtgaggagagaggatggggatctcaggagagggtggggcttcctgagaacaaagggctcagggcatgatatggggaggaagcctgggcctgtgtactaagtctgcgnagctgaggttcccaccctactcataaatgagcctcctctaggaaccccgggtccctgcttgacctgattgtctcctcctgcagaccctaaggcccctgatgtgtatgacttcttcctccagtggattggtgagtgagcacctgccttccctgccctgccaacctcagacccgtggtgctgggtgactaggatcctggcctgtcccactcaattggttatcccagatggcctagttctcgggtgcccatcctaagctcagctgctcagtggagagacaagggaggagcaggagagcccccagctgtggggccaaggcatcttctctggcagggcctgattctctagacagggcaaaggctttggagaatgtgtgagtgcgaagaacagtttcctgaaggaggtggcactagagttgatccaaaaggagatttaaataggtagtgctggccagggcaagggcctaaggaaatggtgtggtggtaggaccacggctggtcaccagaggctgtgaggctgtcggggtggctacaaggcagagccc

SEQ ID NO: 103 - DNA, 480 bp, Homo sapiens MYO5A722436   929
  misc_feature (460)..(460): n = t or g
tttttgggattgaaaaacaagatatgatggggaagttgagaggcttcatgggctggatgtgctatggaaaaaaatactgggaatactttctgaaataggatgaacaggaagaaacagatgtttgcaccagaaagagagtagtgaaaattttaagcaaatgttaagattttgaaagaatgtaagctacagatacactgctggatgtcactagtgttaatactggggaaatagaatctgtggagccagtttttggtttggggtcagaaaacaacatggcttcttttcttgctgttgttgttttgactggttttggtcatgacaaattcttggtattagacaacagttccagccattttagtccctagcattagaatatcccaaagtcttgaatgtgatggcatgaggtaatcccatttcttccgggcctggaccatataatactaggatttgaagttgtctntgaagataacttaagtgtaa

SEQ ID NO: 104 - DNA, 401 bp, Homo sapiens MYO5A1724630   806
  misc_feature (201)..(201): n = g or c
aaaaatctaggaaaagtaaaaacttggtaaggtagacatttttagtaaagcccagctatagcggaatctgttttagtctataatcctgaatttttgacctaccagcaaaattgttgcccctaaataccaggcagcaattaagtttccttctcctcattaactgactcatgtctattgttcctttccttgtaccagaagtanaagttactcatttcccccaagtcctgagttcttatttatgctccttctaaaacataagatttgttatgctcaagtatgaatgagtttcattattcctttgcccatatttcattttctgtacttagtccgacctcagttggcttaaattttaatatagctaaagttttaatttttttccagatttgaactacaaacttaaa

SEQ ID NO: 105 - DNA, 121 bp, Homo sapiens GSTA21051775   456
  misc_feature (61)..(61): n = t or c
acactgaactgcttcacttactttttcaaaggcagggaagtagcgattttttattttctcnttgatcaaggcaagcttggcatctttttcctcaggtggacatacgggcagaaggaggatc

SEQ ID NO: 106 - DNA, 121 bp, Homo sapiens POR8509   689
  misc_feature (61)..(61): n = g or a
actgtgaaacttgtggtgcacaaccctcagggtggtgaagaaattgccgaggaaaaggagnaggaagggaaagccgcacataagcacctgccggaggaatagggtgagggctggacatggg

SEQ ID NO: 107 - DNA, 121 bp, Homo sapiens AP3D12238593   834
  misc_feature (61)..(61): n = t or c
cttcccacccagccagtgcagggaagaacgcagagaagactttccagcagcagcagcaganacccttggcccaaggcagggtctcacctgtacccagtactcactgttcacccgactagcc

SEQ ID NO: 108 - DNA, 121 bp, Homo sapiens AP3D12238594   838
  misc_feature (61)..(61): n = t or c
acccagccagtgcagggaagaacgcagagaagactttccagcagcagcagcagataccctnggcccaaggcagggtctcacctgtacccagtactcactgttcacccgactagcccaaacc

SEQ ID NO: 109 - DNA, 101 bp, Homo sapiens GSTA21051536   440
  misc_feature (51)..(51): n = g or c
gagaggaacaaagagcttataaatacattaggacctggaattcagttgtcnagccaggacggtgacagcgtttaacaaagcttagagaaacctccaggaga

SEQ ID NO: 110 - DNA, 401 bp, Homo sapiens GSTT2678863   786
  misc_feature (201)..(201): n = g or a
tagagtttcacccagtgggatgtgacacttgcaggtgtcccaaactggtgggaaccttgactggaaggctggggttaaggatgaactttctgccgtccagactgtcccctgcagagcagctgctgccagacagcgggagctgccagacagcgggagctcccacttcgtgcacaggatgggggcagggagcccgcagccgcnggaggcaggaatgactgtccgggaacctcctttcttctccctgaatcccagccctggcatctcaccagggggcacagtgatggtccagggctgggcccgggactctagctgaatctttcagagtatcccatccctctggccagtggcccaagcgagtgaaccagaatgcttccttgggagttttgaaactggaactggag

SEQ ID NO: 111 - DNA, 425 bp, Homo sapiens TYR_RS1851992   278
  misc_feature (93)..(93): n = g or a
taagtaggaaaagaatttgctgagaggctattgagtagctcacaaaatcatggagcagcaggctcagaaacaggtgagaataagcaagaaggncatcagctaagacagctgccaaaaccatgctatagaacacagggcacttgctgggcaatggattcctttgctggtacatctggctttgctgaccctgaaaactgaatattgttataccaactgccactgcccatttctaggatggtttctgattatccctgcttctttgtgtcactatctcctgtttcgaagtcatgaatgagtatgtcagattggcagaatatttatcatatggtcatactctaactttagaaaaagccgagaaacaaagtttaagtatctaaaccattgtcattggaggtaagctctgtctcccatcaagactcattaag

SEQ ID NO: 112 - DNA, 708 bp, Homo sapiens CYP2C9RS2860905   367
  misc_feature (455)..(455): n = a or g
ttacagagctcctcgggcagagcttggcccatccacatggctgcccagtgtcagcttcctctttcttgcctgggatctccctcctagtttcgtttctcttcctgttaggaattgttttcagcaatggaaagaaatggaaggagatccggcgtttctccctcatgacgctgcggaattttgggatggggaagaggagcattgaggaccgtgttcaagaggaagcccgctgccttgtggaggagttgagaaaaaccaagggtgggtgaccctactccatatcactgaccttactggactactatcttctctactgacattcttggaaacatttcaggggtggccatatctttcattatgagtcctggttgttagctcatgtgaagcgggggtttgaagctgagagccaagggaatttgcacatatttgtgctgtgtgtgtacaggcatgattgtgcntacagtgtgggtataaaaggttcatttaatcccatgttctcctgaactttgcttttttgctttcaaataagaaatgatgaatatagattttgagttcattttttgaaagagttaaagagcagtgtttttcccattacctattccagaacatgtcaccagagaatacttgacaagtcaacatggtgggaatggccctatcatacccatatggagcatgaaccaaatggcatgtgcttttatttaattggactgt

SEQ ID NO: 113 - DNA, 121 bp, Homo sapiens CYP2C82071426   596
  misc_feature (61)..(61): n = a or g
ttttgctatgatttttgactgaaggtgaagggaagtgtgtgtgattagaaatttcatccantaagttctctactatagtagtcatgtgttttattcagaatggtcatgaaaattgaacttc

SEQ ID NO: 114 - DNA, 520 bp, Homo sapiens DCT727299   682
  misc_feature (365)..(365): n = a or g
caatacaaatgttatcataacaataataatgtgttttataatggtcagaattagagaaccatatgttaggtttagattttcaaaccttaattaatatttcttatcttgttctccaaagcagacagtaatgccctaagacattttactaataagcacaaagtcagaagtatttcacaggtgatttattattgttactcaacccagggtacaaaaaaaggagctatgaagagggagagtaaaggtatagctttcaatgattctaagcttgcctgatgggtgtgaagtagctttctggggctcctagatagattaaagcatagtcaggtgagcttcaagaaatcctgagacggaattagttggtaganttgttctttccttctaaaaaatgtttctttcctcatatttgcatagtagcaataaatgaaggggttgtcagaagtctgaattaatggtcccagcctctaacaaggtgggggcttatctgtgacgccgccacagcgatctttgcttttctctagaa

SEQ ID NO: 115 - DNA, 391 bp, Homo sapiens GSTA22144696   455
  misc_feature (158)..(158): n = t or c
cttgcaactgtaattttctcttctgaagtacgtgagacacaatagggtaaaattctcaatttaataaaggaattagggtcccacactagcattatttttaaggaaaacctctggtttctgatgtggttttgtggcattggggaatgcttgtgtgttcnagaagcctcctcccctcattttaaccacgtgtttatttctctgcatcctcatagacacgtaggctgccccagggcagggactgtgtctgtcttgttcactatctccatgaccgagtacagaacctggaattaataagtgctcaagtaaataattgctgtgaatgtagtcaatctttaataggtagtttgttacaatccactcccttccatctctcatttgtagtttgcatttt

SEQ ID NO: 116 - DNA, 121 bp, Homo sapiens DCT2296498   701
  misc_feature (61)..(61): n = a or g
gcccaaatcaactcatatagagtgactatgatggcgaggatcaagatttcgggaagaaaancagttaagttttcaacgatgtatgaatctctctctccaagcaggactataaacccctttg

SEQ ID NO: 117 - DNA, 121 bp, Homo sapiens AP3D12072305   820
  misc_feature (61)..(61): n = g or c
ggatcccaagcaggctcgggtaggtagtgcacacaggacgcggctgtgccctcccaagccncaagatggcgtggggggaccagcaccttggtcacagggtgggcaagctcccgcctgtgga

SEQ ID NO: 118 - DNA, 550 bp, Homo sapiens GSTA22180319   577
  misc_feature (114)..(114): n = a or g
atgaggcatgacatgggctaatggccatcaaatattctgccaccaaggagcctctgctgtaatttgtatcgccccacttctcaggaaccctgctaagggtgacataggtcgccnctgttgcacagctttcacacttgcaactgtaattttctcttctgaagtacgtgagacacaatagggtaaaattctcaatttaataaaggaattagggtcccacactagcattatttttaaggaaaacctctggtttctgatgtggttttgtggcattggggaatgcttgtgtgttctagaagcctcctcccctcattttaaccacgtgtttatttctctgcatcctcatagacacgtaggctgccccagggcagggactgtgtctgtcttgttcactatctccatgaccgagtacagaacctggaattaataagtgctcaagtaaataattgctgtgaatgtagtcaatctttaataggtagtttgttacaatccactcccttccatctctcatttgtagtttgcattttacctctaattacaatcattttttaat

SEQ ID NO: 119 - DNA, 401 bp, Homo sapiens MYO5A1724639   843
  misc_feature (201)..(201): n = t or c
ctggaccaatcatgtatactctccctggctggagaggacaaaataaaaacctctgcagtattagttttctttccaccttataaattactcgtgggtttcccatattatatttataatgtgttctgctttgtaggctggagaaatgaattaaacttaaactattcttctacacattcacagttttatattttattatattantaagagcataatctagtcctgaaagtaacatttttctcccattttccaccctcaaaatgttagggttccatggttaatataagagacattttgcagatgctgttcaggataactgatgccctatcatataatactaatgttaaaattcacactttcagttgggcatggtagtgtatgcctgcagtaccaactactcagga

SEQ ID NO: 120 - DNA, 401 bp, Homo sapiens GSTT22719   611
  misc_feature (201)..(201): n = g or t
ccagaggcctatcaggctatgctgcttcgaatcgccaggatcccctgaagggtctgggatgggggccaggagattagcaacaaggattcattctgttacttacttgcccctttttatctttccctcttgccccagtcccttctctccagcttcatgtgaagctctgcacagacaagacactcagtgtccttggcagtgctnctactcctcaggtgcagcatacataaccagtaagagactaaatctgcaatatataaagagctcctacaaatcagtaacatgaagaacactcaaaaattggcaaatgtcatcagtgttttaaacagaataaagattccaaacactttgaatagagaaccaagagttattggttttactacattgttgtgttatacatatgg

SEQ ID NO: 121 - DNA, 562 bp, Homo sapiens GSTA22894803   435
  misc_feature (62)..(62): n = a or c
tgcatcctcttctagaatcacccttgcctgaaccctccccatgttcactgttccctcatctncatgggactctgcaatactggacctcagcgtacatgcccaaggcccagcctgctgctggtcatgatgccctgccatcgtcccacccactcaaggaaggacctaaatcactctgtgttctctgtggatggaagaacagaaaatataccgtacagggctctctcctttatgtctttcccatagaggttgtatttgctggcaatgtagttgagaatggctctggtctgcaccagcttcatcccatcaatctcaaccattggcacttgctggaacatcaaatatccatctttagaaggaagaaaaaaaaggagagtgaagtgtctatgaaacccacccttttgggatgaacaaatggttgtggaaatgactaaatttgtaaaatggcaaagaaattactgcctggtaagatttcacttgaaacaaaaactatatatatatatatatatatatatatatatatatgtgtgtgtgtgtgtgtgtgtgtgtgtgtgtgtgtgtgtgt

SEQ ID NO: 122 - DNA, 280 bp, Homo sapiens CYP2C8E8_92   265
  misc_feature (83)..(83): n = a or g
ttcttataatcagattatctgttttgttacttccagggcacaaccataatggcattactgacttccgtgctacatgatgacanagaatttcctaatccaaatatctttgaccctggccactttctagataagaatggcaactttaagaaaagtgactacttcatgcctttctcagcaggtaatagaaactcgtttccatttgtatttaaaggaaagagagaactttttggaattagttggaatttacatggcacctcctctggggctggtagaattgcta

SEQ ID NO: 123 - DNA, 401 bp, Homo sapiens AIM35415   937
  misc_feature (201)..(201): n = t or g
acacaggtttatagttcaatttaagctctagcgtcttagaatcagccactttctaacctttcatgatgccttcctgctttgcaaaaccatgatcagcatgaattgcaaacatacctccttcagtacaagcatttctcaggtctcctccattaaagtcatctgcaaacttcacaattactccataattaatttcaccatacnttgtaaggggacttgcatggattttcaacgtgtctaatcttgctcgttcatttggcaaatcaatatgtatttttttctatctaatcttcctggatgcagcaaagcaagatcccgtgtatttggtctgttgtattttaactctgcgcagagtatcaaatccatccatttgattcagtaactccattaaagttctctgaatc

SEQ ID NO: 124 - DNA, 121 bp, Homo sapiens CYP2C9RS2298037   248
  misc_feature (61)..(61): n = c or t
ggtacaattactctttgtacatgatcaagagcactgttctgaatgcctgtgtacaccctgntcatgatacatcctaattattgggccagattagtggactttggggagttaatccaattct

SEQ ID NO: 125 - DNA, 519 bp, Homo sapiens CYP4B1RS1572603   176
  misc_feature (285)..(285): n = c or t
gaaggaggacagctcccagcacctggtcccactattgcagctgcagcccctcaagagggctgaggcatcctccctgtggggtacacgtgactgcctggacagttcctctggattacacaactgcatgcctcagactcgccctcagaatgcctgagctgggacacttacaggactggaagttctgggacggcttgttttttctccagaaggaaggaaacaggaagagagcagggtatgaaaataattcactgatcccagctccccagccccacctcccaccctcanattggcactccttgttgacctcaccagaaataaaccactggagggattgggaagagcaagcatacgtcaccactttcctctctgcttctcaagagcctaaagtaagctttgctccaagaatcagatcggaaaaggatttcaacattgcctagtggaaccctgacctctctttaaaggtggaaacgtgcggtacaaagaccaaagggaacagtccagcatgagttaagggagacc

SEQ ID NO: 126 - DNA, 401 bp, Homo sapiens GSTT2140194   442
  misc_feature (201)..(201): n = c or g
aagtaccagacgccggaccactggtatccatctgacctgcaggctcgtgcccgtgttcatgagtacctgggctggcatgccgactgcatccgtggcacctttggtatacccctgtgggtccaggtgaggagagccatctggagagtgattggccatcagggagtagttggcagtaggccggggccatagactgacccactntctgcccccatcaggtgttggggccactcattggggtccaggtgcccaaggagaaggtggaacgcaacaggactgccatggaccaggccctgcaatggctggaggacaagttcctgggggacaggcccttcctcgctggccagcaggtgacactggctgatctcatggccctggaggagctgatgcaggtgtgagctcag

SEQ ID NO: 127 - DNA, 401 bp, Homo sapiens GSTA22608677   451
  misc_feature (201)..(201): n = g or c
caccccttacaaaactgagggatccatagcagacagaaaggttgtttgaaaatgcataagaaaaagtgttttcatgagaaataaaatggatgtcaaggaaaaaagaaaatttcattttgccattccccagaatgataactgttttcttgtgcagaatgtcagaagtaaatttctatacatgactttctgataggccatttnacaaatgttgcaggacaattcttgaaaaagtcaaacaaaccacatagtctacattttacttttttactaaatttttaattccaagaaaatttatggggagtttggaaatctgatttcatatcagatactaatgaaaagaaatcaattttaactaaatctggtcataggcattttactatgtaattagcacataattttta

SEQ ID NO: 128 - DNA, 401 bp, Homo sapiens GSTA22608679   570
  misc_feature (201)..(201): n = a or g
ggtattgcatgttcttggcatccatgcctgttttatcaaaccttgaaaatctttgttgcttcttctaaacctttcgcatttatgggaagctccctcaggctgccaggctgcaaaaacttcttcactgtggggagtttgctgattctgattttcagggcccgcaatgcacaaagcacagcctcagagtgaagccaagggctnacaccaccattaacacaacccagggaatctgcgcccctcctacacaaagaccaactaagttccctctatcagcaccagtagggaggcagaaagagacactacgtgagaaatgaagataagaggaagaacatgcagctcactagcattttcccaaaaaatgtctttaagactttattcagttcagtcttcctaccctcttc

SEQ ID NO: 129 - DNA, 401 bp, Homo sapiens GSTT2140196   605
  misc_feature (201)..(201): n = a or g
gaaccaagagttattggttttactacattgttgtgttatacatatggagtaaaagtatgtgctagtaatcctcatcatggttaataacaaagtaacctcacaataacgagtcaacataattgtatcaccagggcaacaaaatgttaagtaagtaaccaattcgaattgcaaactgttaaaggatataggcgatgtttcacngggcatagcaacggtctttgaagtctaggaaacttaaaagatttcttttaacaagcattcatgtcttctaggacagttttgtaataactgcaaatagtaagattatacattgtcacacagacctccatgtatatccatgggatggaccccaccacaatgattttaacggagagaacttgatataaagaattggtaaccag

SEQ ID NO: 130 - DNA, 793 bp, Homo sapiens MYO5A752864   835
  misc_feature (98)..(98): n = c or t
atcctctgccatcctaggtttattatgtgattgtcacaactgagactggggacaggcttcctttacctttggtccccaagcagccactgaggccatcncaggctccacctctagcagacaagtagggttagagttgaggttgagaaaggcggttggcaaaactaagccggggtttttggttgatgttggatggagctcccaggtcagggaggtgtgtgttaggattcaggtttacatattttctgttcttttaaacagaacttgtataacaggtgaggaaattgtctttttccttctcagattctaaaccctggcatggagtctgactgttttcttttcttttttttgccttgttcctttttttttttcttttgccaaaaaaattttctaaaatatttctcagtcacataataacttttattactgatttacagttttttgacaaatatatttactatattaaaaatccttaaaaagtgataatttcatatttctatataaagtctatattgtagtgctagcccgattttgagaaaaattgtttaggattggtgggggatttgtgtcttttttaaattaacatttttaatgacctatataaaaaagtatgaattttctctgaagatttaacaatctgaattatccctccattactccatttgtggtaattacattacaaaacagtttctagtgtcatttattcaaccattttttttttgatttagaaaccaaacgcaagggtttcttttttacagtctgtcttgtatgaagcttccaccatgggaatgaggga

SEQ ID NO: 131 - DNA, 732 bp, Homo sapiens CYP2B6RS707265   283
  misc_feature (272)..(272): n = a or g
tggtgcaatttttgttcactgcaacctctgccttccaagatcaagagattctccagtctcagctcccaagtagctgggattacaggcatgtactaccatgcctggctaattttcttgtagttttagtagggacatgttggccaggctggtggtgagctcctggcctcaggtgatccacccacctcagtgttccaaagtgctgatattacaggcataatatgtgatcttttgtgtctggttgctttcatgttgaatgctatttttgaggttcntgcctgttgtagaccacagtcacacactgctgtagtcttccccagtcctcattcccagctgcctcttcctactgcttccgtctatcaaaaagcccccttggcccaggttccctgagctgtgggattctgcactggtgctttggattccctgatatgttccttcaaatctgctgagaattaaataaacatctctaaagcctgacctccccacgtcaagaggtgatctgtgccattttgtgtgtgattcttttattgtcgggtctctagggatttttctggaaggaatgttggtgagaatgcctctctcacctcaatgccaactctgtgaagggccaaaccattgtcttgctcatccctgtactctcaacacagcgtgtggcatatgacaggtgttcaaaatatttggtgaggaatgaatgaatgagtggctaaatcagccaccccctacccccacagccca

SEQ ID NO: 132 - DNA, 121 bp, Homo sapiens CYP2D6_RS2267446   172
  misc_feature (61)..(61): n = c or t
tgtgcgggaggacggggacagcattcagcacctacaccagacagaacggggtctcaatccntcctgtgctctgcgttcatctggaccagtctcaggccccagccatctccaggaagaccca

SEQ ID NO: 133 - DNA, 121 bp, Homo sapiens AHR2237299   540
  misc_feature (61)..(61): n = a or g
gtatctgagatggatcttgagtgagggacaggatttcatgaagaggcataactaaggattngtgaactgtaagaattcccccacatgaagggagaggcacagggtgaaagagagaaaagaa

SEQ ID NO: 134 - DNA, 101 bp, Homo sapiens CYP2C181042194   712
  misc_feature (51)..(51): n = g or t
cacatatgctaatacctatctactgctgagttgtcagtatgttatcactanaaaacaaagaaaaatgattaataaatgacaattcagagccatttattctc

SEQ ID NO: 135 - DNA, 1001 bp, Homo sapiens CYP1A2_RS2069524   206
  misc_feature (501)..(501): n = c or t
catatatcctcacgtaagtccatgaatatctgacatttctcatatctactttctctcgatttattgatagataggtatacattgttttaattttatgggtacatagtaggtgtatatatgtatggggtacatgaaatgttttgatacaggcatgcaatatgaaataagcattcatggagaatggagtatccatcccctcaagcaaggataaacctttgagttacaaacaatccaattacactctttaaaggtgtacattttttttttttttgagacggagtctcactctgtcgcccaggctggagtggagtggcacgatcttggctcactgcagcctccacctcccaagttcaagccattctcctgcctcagcctcccgagtagctgggatcacaggcacatgccaccatgcctggctaatttttgtatttttagtagagacggagtttcaccaggttggccaggctggtcttgaacacctgatctcaggtgatccgcccntctcggcctctcaaagtgctgggattacaggtgcgagccatcgcgcctggcctagaggtgtacattttttaacagaaccattcaaaaggaggttgtggggatcatgacacttccatgctacagcattaatctcctaagaataaggatacactcccacataccatgacactctgttcacacctaaaaaaatttacatttattccagaatatcatctaatctccagtccgtgcttacatgtccccaattgtccccaaaacatcttttatagatttttttaaaattttgtttaaatgccatatccaatcgatatggcaatcaaatgcaaatccatattgcatttggttatgtctcttagtctttttgcataaggggggcctctctttaggatgcaaaatctttatcatctcttcttttccacttggggacttgggctgaaaatcaggagtggctggaacacgcccatttactgtttggttttgcaggttgttggagggtacta

SEQ ID NO: 136 - DNA, 401 bp, Homo sapiens CYP2D6_RS2856960  193
  misc_feature (201)..(201): n = c or t
ggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggncatgttctggaggaggggttgtgactacattacggtgtatgagcctagctgggaggtggatggccgggtccactgagaccctggttatcccagaagcctgtgtgggcttggggagcttggagtggggagagggggtgacttctccgaccaggcctttctaccaccctaccctgggtaagggcctggagcaggaagcagcg

SEQ ID NO: 137 - DNA, 401 bp, Homo sapiens ESD1216958   706
  misc_feature (201)..(201): n = t or g
catgggcaagacatttcaattcacaaatgcagcacatcagtaagacagtgactattaaaacacaaaataagcacacaaacaatatagaaaaagaacataaaaatgcccaaatgttctatcattgttgggaagtcaaacacagccatcataaatcctgttaacaattcctcctacacagtaaaagcatgttgtatctttatntgaggagaaatatgtcattaaggcctgacatcccttagcaaattaggtaaaaagtcagtaatcctttggaaaactaacatgaaacagagaaaaatgcaatgtgctaagcagttaagttgaaagagatttctatctatccagtcatttaaaaccattgttgtaggtaaatggagaaataatccctttctgctgactctgac

SEQ ID NO: 138 - DNA, 387 bp, Homo sapiens CYP2A6RS1061608   41
  misc_feature (312)..(312): n = a or t
cttcaccaccgtcatgcagaacttccgcctcaagtcctcccagtcacctaaggacattgacgtgtcccccaaacacgtgggctttgccacgatcccacgaaactacaccatgagcttcctgccccgctgakcgagggctgtgccggtgcaggtctggtgggcggggccagggaaagggcagggccaagaccgggcttgggagaggggcgcagctaagactgggggcaggatggcggaaaggaaggggcgtggtggctagagggaagagaagaaacagaagsggctcagttcaccttgataaggtgcttccgngwtgggatgagaggaaggaaacccttacattatgctatgaagagtagtaataatagcagctcttatttcctgagc

SEQ ID NO: 139 - DNA, 401 bp, Homo sapiens CYP4B1RS837400   336
  misc_feature (201)..(201): n = a or g
gtattggggatctagaccagaggtttctaattagacacagggcagggaattggaatcaactgggaggagcaaattcctggacctccagttcctggagattccaatttgtcagattggaactaggagtttacatttataaacctgtccaagtgttagtgttttgatctgcagccagcttggggaatccaggtagagatgccngagacttttctttctttccctctctttttcctttcctttccctttccctttctttctttctttttttcttctttctttctctttctttctttctctttctttctttctttctctttctctctttctttctttctttccttccttccttccttccttccttccttccttccttccttccttccttctttctttctttcctt

SEQ ID NO: 140 - DNA, 560 bp, Homo sapiens CYP2A6RS1137115   284
  misc_feature (60)..(60): n = a or g
actaccaccatgctggcctcagggatgcttctggtggccttgctggtctgcctgactgtnatggtcttgatgtctgtttggcagcagaggaagagcaaggggaagctgcctccgggacccaccccattgcccttcattggaaactacctgcagctgaacacagagcagatgtacaactccctcatgaaggtgtcccaaggcagggagatgggtggcacggggtgggggctgcctagttggctggggctttgtggcagggggttgaccagtgtggaccagagtcttaggaaatggagttttggagtttcagcatcagaaagacaggatcttgggatgtccagctccctgactgtgagaacctgggtgcgaagcatcccagcacatgacatctcggtgctgggccccattcagagtggaggcttctccctctaaccactcccacccacctccatcagatcagtgagcgctatggccccgtgttcaccattcacttggggccccggcgggtcgtggtgctgtgtggacatgatgccgtcagggaggctctggtggaccaggct

SEQ ID NO: 141 - DNA, 401 bp, Homo sapiens GSTT2140186   545
  misc_feature (201)..(201): n = g or a
ccctgcccccatcctgtgcacgaagtgggagctcccgctgtctggcagctcccgctgtctggcagcagctgctctgcaggggacagtctggacggcagaaagttcatccttaaccccagccttccagtcaaggttcccaccagtttgggacacctgcaagtgtcacatcccactgggtgaaactctaagatcccttttagnggatcccattcgctccctcccttccgccaccatgcagcgccgagaaacagagctctgaacgaaccctcagatgtccgtgcgctggggcctttccaggacggcggcgcccagtcgtttctgggtcagggcgacgcctggaactgggcagggtccctggcaccgggatcccgaaaagcagacctgcttctccctgtccagcc

SEQ ID NO: 142 - DNA, 565 bp, Homo sapiens MAOB1799836   465
  misc_feature (65)..(65): n = c or t
tctggaatcttccccatggcatgcaggatctgaaatgaaagaacacactggcaaatagcaaaagngacaccatctttcttctaatctgctccctaaaggactaagtaactgtctcttgagatatccatcaaaaacaccaaggtaaaggccagaacactccatacaacctttaagaggcgccaaaaggtctttctcctgtggtataagaggttccaatcattaggcttaccataaattttaatagttatgaacacacctcaaatagggatgacatcagaggaactgtcagaaaatctttcctgtgaaatgaattgaattttgagtcctaatagaagcatgtttggttctagaatatcagacctaggttacctttcaagtatagtcaggttgatcgtttgccccatgggctctttttacttcgggtgacatgttaggtattttcattattacttcgggctaaggtgttcacttcacctgagacaatagttaaaatagtattttatgggtcttaagatgggggttactggagagttggtctccaggctctcagatatctttgacctac

SEQ ID NO: 143 - DNA, 849 bp, Homo sapiens GSTA22144697   474
  misc_feature (492)..(493): n = c or t
ctcaatcttctagttcacttcacctccaccttgatatgaatgtctatgattaggtcatgttttgagagggacatcactggagaaaaggcactgagcagttcttctagttatggtgttgtcatatcttaggaaagcctgtgtctccaagtgagatcagaccacaaccttgtgtgtccccagcatgaggcatgacatgggctaatggccatcaaatattctgccaccaaggagcctctgctgtaatttgtatcgccccacttctcaggaaccctgctaagggtgacataggtcgccactgttgcacagctttcacacttgcaactgtaattttctcttctgaagtacgtgagacacaatagggtaaaattctcaatttaataaaggaattagggtcccacactagcattatttttaaggaaaacctctggtttctgatgtggttttgtggcattggggaatgcttgtgtgttctagaagcctcctcccctcatnttaaccacgtgtttatttctctgcatcctcatagacacgtaggctgccccagggcagggactgtgtctgtcttgttcactatctccatgaccgagtacagaacctggaattaataagtgctcaagtaaataattgctgtgaatgtagtcaatctttaataggtagtttgttacaatccactcccttccatctctcatttgtagtttgcattttacctctaattacaatcattttttaatattatgcatttttatttttttattgtggaaattatgaacatgaataaatataaacagaaaagcataatgattctccttatacttctcacctagaatcaataattatcaatcaaaacca

SEQ ID NO: 144 - DNA, 551 bp, Homo sapiens GSTA22180315   500
  misc_feature (68)..(68): n = t or c
agtgagatcagaccacaaccttgtgtgtccccagcatgaggcatgacatgggctaatggccatcaaanattctgccaccaaggagcctctgctgtaatttgtatcgccccacttctcaggaaccctgctaagggtgacataggtcgccactgttgcacagctttcacacttgcaactgtaattttctcttctgaagtacgtgagacacaatagggtaaaattctcaatttaataaaggaattagggtcccacactagcattatttttaaggaaaacctctggtttctgatgtggttttgtggcattggggaatgcttgtgtgttctagaagcctcctcccctcattttaaccacgtgtttatttctctgcatcctcatagacacgtaggctgccccagggcagggactgtgtctgtcttgttcactatctccatgaccgagtacagaacctggaattaataagtgctcaagtaaataattgctgtgaatgtagtcaatctttaataggtagtttgttacaatccactcccttccatctctcatttgtagtt

SEQ ID NO: 145 - DNA, 401 bp, Homo sapiens GSTA22749019   583
  misc_feature (201)..(201): n = a or g
agttagggaaaagccactcccacacatttcatggccaaggggccacctactggattctaagacatgaggcaagtgatctgcttatcagaagacactggttaatatgttccttttcaaggttggtaatcaaagtttaaacaatacatttcacctagattttgctctttttgcaagtcagcagaaactggctttttaaagatnctttttttcatgagttggatgcaaagactagggcaactgaaaaaaccctattgtgagcatagctgggagaggatgtctgtgaagggcaagctgatgccaccgttttcttactgggttgccaaataaaatataggacatccatgtaaatgtgaatttcaggcaaacaatcaacaattttttagttatagctatgttccaaa

SEQ ID NO: 146 - DNA, 141 bp, Homo sapiens ACE_4343   349
  misc_feature (71)..(71): n = a or g
cagaggtgagctaagggctggagctcaagccattcaaccccctaccagatctgacgaatgtgatggccacntcccggaaatatgaagacctgttatgggcatgggagggctggcgagacaaggcggggagagccatcctcc

SEQ ID NO: 147 - DNA, 572 bp, Homo sapiens ACE_4335   291
  misc_feature (389)..(389): n = a or g
ctgcccctccctcagaaccgccctctgcttaagggtgtccactctctcctgtcctctctgcatgccgcccctcagagcagcgggatctcaaagttatatttcatgggcttggactccaaatggggggaactcggggacactagctccccccggcctcctttcgtgaccctgcccttgacttcctcaccttctctgtctttcctgagcccctctcccagcatgtgactgataaggaaattgagtcacacagcccctgaaagcgccagactagaacctgagcctctgattcctctcacttccctcacctaccctgccacttcctactggatagaagtagacagctcttgactgtcctcttttctccccactggctggtccttcttaccccngcccgtttgaaagagctcacccccgacacaaggacccgcacacagatacctcccagctccctctcaacccaccctttccagggttggagaacttgaggcataaacttgcttccatgaggaatctccacccagaaatgggtctttctggcccccagcccagctcccacattagaacaatgacaa

SEQ ID NO: 148 - DNA, 780 bp, Homo sapiens POR2868178   669
  misc_feature (280)..(280): n = c or t
tctcgctctgttgcccaggctgggtcttaacctcctggcctcaagcagtctccctgcctcagccttgcaaagttctgagatcactcactgtgtaggatccaaagtcccacaggatccagggctcctgtgggctctctctcctgtgggcctggcagctttgctcacccttatggaacaacaccgcatggcgtgccctcttggtgatggaatttgtatttttgcctcccatggtgcagagagcgtcccatttccatctgggtccctaccttagtgcggggcngcctgtcggagggaagcttctcagagaatggccgttgaattaaccaaggctaaatctgtatgtgtggctgcctctagggaaacctgtggcctccaggctgggtttggcttacacagtatttttttaaaaaatattttaattagaacattaaaaagtgtggcagtatcaggcccagcgtggtggctgacacctgtaatcccagcactctgggaggccaaagcaggtggatcacatgaggccaggagttcaagaccagcctggccaacatggcaaaaccctgtctctacaaaaagtacaaaaattagctgggcatggtggtgcgtgcctgtaatcccagctactcgggaggctgaggcaggagaattgcttgaatccaggaggtggaagttgcagtgagctgagatcacgctactgcattccagcctgggcgatgaagtgagaccaaaaaaaaaaaggcagtatcaaataaaaacttagacttacagcttctttaaaaaaaa

SEQ ID NO: 149 - DNA, 401 bp, Homo sapiens TUBB1054332   763
  misc_feature (201)..(201): n = g or a
aagccgggcatgaagaagtgcaggcgagggaagggcaccatgttcaccgccagcttgcgcaggtctgcgttcagctggcccgggaagcgcaggcaggtggtgaccccgctcatggtggccgacaccaggtggttgaggtccccgtaggtgggggtggtcagcttcagggtgcggaagcagatgtcatacagggcctcgttntcaatgcagtaggtttcatctgtgttttccaccagctggtggaccgagagggtggcgttgtagggctccaccaccgtgtctgacaccttgggtgagggcatgacgctgaaggtgttcatgatgcggtctgggtactcttcccggatcttgctgatgagcagggtgcccatcccggaccccgtgccgccccccagagagtg

SEQ ID NO: 150 - DNA, 788 bp, Homo sapiens CYP2D6_RS1467874   293
  misc_feature (483)..(483): n = a or g
gcctgtagtcccagctacttgggaggcagggggtccacttgatgtcgagactgcagtgagccatgatcctgccactgcactccggcctgggcaacagagtgagaccctgtctaaagaaaaaaaaaataaagcaacatatcctgaacaaaggatcctccataacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagaggtcccatccaggaaacctcgggcatggctgggaagtggggtacttggtgccgggtctgtatgtgtgtgtgactggtgtgtgtgagagagaatgtgtgcyctnagtgtcagtgtgagtctgtgtatgtgtgaatattgtctttgtgtgggtgattttctgcatgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaagg

SEQ ID NO: 151 - DNA, 700 bp, Homo sapiens CYP2B6RS2099361   81
  misc_feature (283)..(283): n = a or c
cactgcactccatcctgggtgacagagtgagactccttctcaaaaaaaaaaaaaaaaaaaagaatatactcccaagttaggttgcagttcactctacagagagagctttaggtcaaatttaatttaattaaacaattctccccttttggtcagcctcaaaattttgagattgaccaaaaccttgggcatcaacattacttctgtcaccatcataatggacttgtctgctctcagtatggaattcacaatggacaatgtcaacgtagttgagtgattctttacnttttcttcatgtttttgttgttcccactgtaatgagcccactggatgtacaaagaatggctgcatatgagcatttaagactctttttttttctgagacagggcctcactctgtcagccaggctgaagtgctgtggcatgatcacgtctcactgcagccttgacctcccaaggctcaagtgatcctcctgcctcagccccccaagtagctggaactacaggtgcatgccaccacgcccagctaatttttgtattttttgtagagacagggttttgccatgttgcccagactggtcttaaactcctgggctcaagcaatccacctgcctcggcctcccaaagtgctaggattacatgtgtgagccaccgcacccggccaagactcttgagaaaatacaacacatcagggagactgttat

SEQ ID NO: 152 - DNA, 201 bp, Homo sapiens AP3D125672   873
  misc_feature (101)..(101): n = a or c
aggacttggtccgcggcatccgtaaccacaaggaggacgaggcaaaatacatatctcagtgcattgatgagatcaagcaggagctgaagcaggacaacatngcggtgaaggcgaacgcggtctgcaagctgacgtatttacagatgttgggatacgacatcagctgggccgccttcaacatcatagaagtgatgagtgcct

SEQ ID NO: 153 - DNA, 401 bp, Homo sapiens CYP1B1RS1056837   151
  misc_feature (201)..(201): n = t or c
gccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccagtgaagtggcctaacccggagaactttgatccagctcgattcttggacaagganggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatt

SEQ ID NO: 154 - DNA, 121 bp, Homo sapiens ACE_4320   321
  misc_feature (61)..(61): n = a or g
gcagggtacaagggagtgcgagagggataatggcttctggtgagaccacaaacctggagangggaggcagaggtttgtctgtttccctgcactctgtcccacagacctggtgactgatgag

SEQ ID NO: 155 - DNA, 514 bp, Homo sapiens AHR2158041   593
  misc_feature (436)..(436): n = a or g
aacaactaaaaaacagtttgatagtgatccactgtctcgactgcatacccatgagcgagtcataacttgcagttttaaaaatggtgtcttaatctaccacttgcgtcagatatgaatattcttttaggaaaaaaatccatgtgtatatgcgagaaaaaaatggattggaaaatggctaatctctctgattttcttactaatataaatcacaatatcaaatcctgattaagacagttatataatacattaggcttcaaacaggcttttctatggacattaatgtattttctttagaatgcaagaaatcaggagtttagactttgtggtagcagtagtagctagataccactactttagtcatctacacttaaatcttcctaggaacctaatcttctagtattaaatcatcttgatgccaaccattacacaatttccnaaagttgcatctgaaacacaagttgaaatatacacaccccctgtaacaaaacccttcagtggtttccactgggtatat

SEQ ID NO: 156 - DNA, 616 bp, Homo sapiens ACE_1987692   48
  misc_feature (273)..(273): n = a or t
ggacagggtttggcctacaagttgtggatgtgggtacccatgccaagtgtgaggggaggctggccgggtgtggtggctcatgcctctaatcccagcactttgggaggccaaggtgagtagatcacttgaggccgggagtttgagaccagcctggccaacatggtgaaaccccatctgtactaaaaatacaaaagttagctgggcgtggtggtagatgcctgtagtcccagctacttgggaggctgaggcatgagaatcgcttgagcccagccngggcaatacagcaagaccccgtctctacaaataaaatacaaaaaattagttggatgtggtggtgcatgcctgtagtcctagctgctagggaggctgagatggaaggattgcttgagcctgggaggtcaaggctgcagtgagccgagatggcgccactgcactccagcctgggcaacagagtgagaccctgtctcagaaaaaaaaaaaaaaaaaaaaaaaggagaggagagagactcaagcatgcccctcacaggactgctgaggccctgcaggtgtctgcagcatgtggccccaggccggggactctgtaagccactgctggagagccactcccatcctttct

SEQ ID NO: 157 - DNA, 579 bp, Homo sapiens AHR1476080   640
  misc_feature (145)..(145): n = a or c
atcaaatggtcataccacagatgactcaagttttattcccacatgcatttaaccatatccatgtttcctaatacggtactttccttccttagacatctaagctcagatttcccctactccctaaatgccacatctcttcatatcncatgctttgaattggtcaaggaaaaaaaaattcatgaggaataccgaatagtggtaaactatattttgtattttgttactatagtttgcatttaaacagaaagatgatcttgccagggaaagcatatccccttttagtccctaactgctgctataaattcaattaatttcataatatttaccatcccctttactactttggcagggtagtccttaaaagtttgttctatccaaactcttacataaaaactgacttccaaagatgtttaacattcttccagttactcattggtacgcactcagttttatacaattcctctataactatatgacaaatctctgcattaatggaacaagagcagttaattgaccttttcagtggggaatgagttgcacttagcttttcatatatacaagagtagtatttaaattgca

SEQ ID NO: 158 - DNA, 121 bp, Homo sapiens MAOA2283725   585
  misc_feature (61)..(61): n = g or a
atactaaatctggaggtctaaggcatggtttgaaattgctggctatatattatttttgttnaatgatccatgtaaacttattattcaaagtatggcccaagtattggccagtattttatgt

SEQ ID NO: 159 - DNA, 401 bp, Homo sapiens GSTM1412302   461
  misc_feature (201)..(201): n = c or t
aggctccacgagtccatccagcccttgctaggtccacagcgacttggctgtgcgcttgagacaccagccagatcctaacggagcaaagctcttctcccttctcctccctgcccgcggtgtccctcagccttctctccgctgccgagttcccaagggctctgggagactccggctgcaggggtcagactaaaaagtggtggncccaacctgggaatttaattcagcccctgtcactgtaagagcaggacttcctctgatccgaaagctactcccagggcttagtctcccctctagccccgcctacacaggaacagtgtcagtggtataggaaggacccccaggaaaagggccagagtaaaggaaatgtggtctgtgttttctgttaggggccctcgggtact

SEQ ID NO: 160 - DNA, 121 bp, Homo sapiens ACE_4329   322
  misc_feature (61)..(61): n = g or a
cctctgtttgtctcctctacaaaaggggctacacttcctctttaccctcattccctgcctntttggctgagcacaaattatgccactgagccacacactgttactgttccttggcactttg

SEQ ID NO: 161 - DNA, 121 bp, Homo sapiens ACE_4331   338
  misc_feature (61)..(61): n = g or a
gttgcagaacaccactatcaagcggatcataaagaaggttcaggacctagaacgggcagcnctgcctgcccaggagctggaggaggtgtgtggctcgcaaggtacagggagaggggaatcc

SEQ ID NO: 162 - DNA, 139 bp, Homo sapiens ACE_4973   341
  misc_feature (71)..(71): n = g or a
atgtgaaccagttgcagaacaccactatcaagcggatcataaagaaggttcaggacctagaacgggcagcnctgcctgcccaggagctggaggagtacaacaagatcctgttggatatggaaaccacctacagcgtggc

SEQ ID NO: 163 - DNA, 121 bp, Homo sapiens ACE_4344   354
  misc_feature (61)..(61): n = g or a
ccccacttgcatctggtgccacattcactgcagatctatgtcgggcaagtcaccatggatnggggaagaagttaataatcttgtccaggagaccacggcacccatcacaacattgtgtgat

SEQ ID NO: 164 - DNA, 1057 bp, Homo sapiens ESD1216967   690
  misc_feature (857)..(857): n = a or g
aaatttcataaaattaaaaccatgcaggatcactgaaaaaatagatgaataaaactccatataaacttcagtcacactctacctgtgtgcaaacacagtcttttttttcttctttttttttttttgagacagggtcaaactctgtggcccagattggagtgcagtggcctgatctcggctcactgcaatcatctcggcccccgggctcaggtgatcctcccgcctcagcctctggagtagttgggaccacaggtggcaccaccatgcccaggtaattttttgtagaggaggttttgccatgctgcctaggctgggtctcttttccttcataaaaattcacatggcttgcctaggtaaggatttctattaaactctggggaaaattttttttaaaaaatatagcatctataaacgccaaacaccttcacatggctctaaatatggtctataatttggtcccagattatttttctggtctcactccctattttgttagttaaactcaactatgcataattctaggatggcctggatgttgctatcgggtggctttactcaatgccacggtacaatggtttcagagcaggttttggggccagaccctccgggttcaaaccctgacttcaccacttattactacatgacactgggcaagctaatctatgcctcatttgcccatctgcaaaatgggtatacagtaatatatatcaatttgatagggttgctctgggaattaaatgagttaattcacgtagtgcttagaatgctgcttctaccacacacacaaacatgagctattatttttcctgcaacctgaatgccctctccttccaggtgcgctcaatcccatttccctnatgaccccatcagaaatgacttccagtcccccacagcgctctgagagcattttacgacccgaacagattttgtcaaactccaaagacacgtgctcccctcggcaccatcaggtgagtgctcctgaccgcgccattggctcgtgccccgacgcggacgctgcctagagcagcaggtccacactcccccgaaccaggcgccg

SEQ ID NO: 165 - DNA, 121 bp, Homo sapiens AHR2237298   600
  misc_feature (61)..(61): n = a or g
ctccacttaaccaactgaatttcaaactttacctataatgcttgtctacctcattgtgtanttctctactggttaattaataacttgctgacaagtatttatttatatccatcacatcctg

SEQ ID NO: 166 - DNA, 302 bp, Homo sapiens ACE_4309   256
  misc_feature (151)..(151): n = c or t
ggaagtggtgtgccacgcctcggcttgggacttctacaacaggaaagacttcaggatcaagcagtgcacacgggtcacgatggaccagctctccacagtgcaccatgagatgggccatatacagtactacctgcagtacaaggatctgccncgtctccctgcgtcggggggccaaccccggcttccatgaggccattggggacgtgctggcgctctcggtctccactcctgaacatctgcacaaaatcggcctgctggaccgtgtcaccaatgacacggaaagtgacatcaattacttgcta

SEQ ID NO: 167 - DNA, 1001 bp, Homo sapiens MYO5A1615235   919
  misc_feature (501)..(501): n = a or g
agccatcacagtagggagaagaggagaataaaaataacagattttttaaaactgaagaaacacggttgttcagcattttgccttcattattttataaagatgagtgggaaatggtaaaaatggcacagccagtcactaataagcttagctcctgtagcctcaacatcatgaaaaaggcatggtaatcatcagggattcccaaagcagcaaatattttgcctcaactagatactccctggacataagccagaagattatgtggtcaattttgaagaaaaagaacaaaatatactaacatgcacaaatctttcgaataccacattgaagaagtgtttttatgtgcttggactctctgggaagaaatttccctttaaaacacacatacactaaacctaaatgcccattttcatgaacaaatcacatatgaaaacaggaatcctagattcagccagcaagggaaagacatgacacaatcaatcatggctggcatgggccagactnaacccggtctctggcctgtatctgtatcctcatttcatgtaaattcacctctagatttcatttttaacaataatcagggtactaatttacttggagctttgagaaggagcatttggatttgagtatgttaaatatggggcaacttcccaaagaactgtgataatgtaatgactattctaagtactgggcacaaagttctggaagcttgtaacaaggtcattcccaagtacaagagaaatttatatgacccagtgatgaacttacagataggagaaagcgaaaagaaagtgggtgaacgaaagtacaggtagatctgagattcaagataatttaaattaagaaaaacatttcagcatgccatgccactgtcctggatattacgttaacctctttttttttttttttttttttgagacggagtctcactctgtcactctgtcacccaggctggagtgcagtggcacgatctcggctcactgcaacctccgcctcctgggttc

SEQ ID NO: 168 - DNA, 401 bp, Homo sapiens GSTA22608678   542
  misc_feature (201)..(201): n = g or a
attggtattgcatgttcttggcatccatgcctgttttatcaaaccttgaaaatctttgttgcttcttctaaacctttcgcatttatgggaagctccctcaggctgccaggctgcaaaaacttcttcactgtggggagtttgctgattctgattttcagggcccgcaatgcacaaagcacagcctcagagtgaagccaaggnctaacaccaccattaacacaacccagggaatctgcgcccctcctacacaaagaccaactaagttccctctatcagcaccagtagggaggcagaaagagacactacgtgagaaatgaagataagaggaagaacatgcagctcactagcattttcccaaaaaatgtctttaagactttattcagttcagtcttcctaccctc

SEQ ID NO: 169 - DNA, 685 bp, Homo sapiens CYP2C9RS1200313   413
  misc_feature (325)..(325): n = t or g
gaagtagaagacccatctcagacctagaagacccaattcagacctagaaaaccctaaagaactttccaaaaactcctggaactgacatacaacttcagtgagatttcagtacacaaaataaatatgcaaaaatctgtagcatttttaaacaccaaaaatgttcatcctgaaagccaatcaagaactcaatcccatttacaatagccacaagaaaaaacctaggaatacaactaacaaagggggcaaaaaggtctctacaaggagagctacaaaatactgatgaacaaaatcatagatgacacaaacaaatggaaaaacagttcangcttatggattgaaacaatcaatatcattaaaatggctagactgcccaaagctatctacagattcaatgctattcctatcaaactaccaatattattttattctaaaattcatatggaaccaaagaagagcccaaatccaaagcaatcctaaacaaaaagaacagtgggaagcttcacattgacttcaaactatactgtaagctacagtaaccaagagagcatgtaactggtacaaaacacaaattgaacagaagtagagaagccagaaataaagccacacacctatagccatctgatcttcaacaaagttgacaaaaaataatcaatgaggaaagaattctctatttagtaaattgaat

SEQ ID NO: 170 - DNA, 326 bp, Homo sapiens CYP2B6E7E8_610   165
  misc_feature (201)..(201): n = c or t
cctcaggacacagaagtatttctcatcctgagcactgctctccatgacccacactactttgaaaaaccagacgccttcaatcctgaccactttctggatgccaatggggcactgaaaaagactgaagcttttatccccttctccttaggtaagctggacccacaatttctttcccagacaccagagggcaggtactatccncaacttgagaaaaacaacgagagatactgattatttgagcacttaatatattctgattgcttcacctgccttatcccattccatcttcactacaaccctataaggaggcttgagaaagaagatat

SEQ ID NO: 171 - DNA, 121 bp, Homo sapiens CYP2D6_RS2267447   259
  misc_feature (61)..(61): n = c or t
cctcctccaggcccttcttacagtggggtctcctggaatgtcctttcccaaacccatctangcaaatcctgcccttcggaggccccagtccagccccggcacctctcaggagctcgccctg

SEQ ID NO: 172 - DNA, 611 bp, Homo sapiens CYP2B6RS2054675   149
  misc_feature (333)..(333): n = c or t
gacacagcacagcaagaccgaggcccttggttcaggaaagtccatgctgccacctcttcagggtcaggaaagtacagtttccacctcttacaaataggactgtttgtctgctcctcctgggtcaaagtaacttcgggttcaggtcctggatccagcaaagggtttgcttaacattgcaagaaagatgttgcctcatggtcaaaagtcaggcgtaggatgagacaggcagacacgcacacattcacacccacgttttgcaaagatggactgaccctgtcagaggatgtgtgggtgaaggtgcacagtgaggatagagacatatgggagtccagnagacatcaatcaaactggactcagtttgcacacacctggagctcaagagtctccagggggaaaacagagacacaaagtcagacagagagagagccagagaaatttcctgcaccgtgaagatagtcagaggcagggaagaaactccttagcactagttagagtgatcagaaaccaagaggacctgatcgctgtacctgccaggtctcagtttctgtctccttccaactgaccacctcttcctctgagactcaccagttctgcatctcttgctcctcctt

SEQ ID NO: 173 - DNA, 361 bp, Homo sapiens TYR_RS1827430   386
  misc_feature (114)..(114): n = a or g
ataggccattttgtacatggcaaccatgtgaagagcagtagaatcagaagaagaaaaaaaaaggttttgagacatgactctatcaactgactgtaaggtgacctgggaaattcnctctacatccctgaatctcagtttattcacctgaaatactgggaccagaacacattaaagaattatttagaatgatacattaatgagcctagtacagtgtaacacagggtaaacatccagcagttttggaatcatttttggaagtttcttgctagggttaccaagaaaatttgtagaaatcttgaacttaagtgtagttaataataatagctattataatgtttattgctctatgatgacgatagta

SEQ ID NO: 174 - DNA, 401 bp, Homo sapiens CYP2D6_RS2743456   347
  misc_feature (201)..(201): n = a or g
gtctcaaatgcggccaggcggtggggtaagcaggaatgaggcaggggtggggttgccctgaggaggatgatcccaacgagggcgtgagcaggggacccaagttggaactaccacattgctttattgtacattagagcctctggctagggagcaggctggggactaggtaccccattctagcggggcacagcacaaagctcntagggggatggggtcaccagaaagctgacgacacgagagtggctgggccggggctgtccggcggccacggagaagctgaagtgctgcagcagggaggtgaagaagaggaagagctccatgcgggccaggggctccccgaggcatgcacggcggcctgtggggaggggaggggcgtcagtgagcctggctcctgggtgata

SEQ ID NO: 175 - DNA, 989 bp, Homo sapiens ESD1216961   677
  misc_feature (702)..(702): n = c or t
cagagtaaacacctatctttttatcaaagttatgtaagtacatagtttaaagtaaactgtaactaaatagtaaatacttccacacactgttatgaaaaactaaatatatataaaaactaaatatataaaaaactctccaaccttctcattttccatattccagagataattaatttacctcttttagttgatttttctggtatttatgtccctatctacataatatgactttattactgcttctagatttttcagtttttagttattatctattggcttcccattatagaaggattttagttaactttcagctccctgtctttgctctctgcatgcccctacatttctccttccactatcgtatcacatataattttggttaatcaatattaagaagttataaatgctattcagagataagccatgtagtatattagcattactttcctttttctttcagtgttttttgttttcattgagctggtaattctctcctttttgttactggttatccttccttgttaggaatagactcccacaaaggccccatcatgggaagctttctgtatgctcctttccattctctgcatattggtgattttctttttccacattattctaagcaacttttcttgttgatgactgacggcacaaaaaatttctgacatttgtttttaggttttcaacttttgttagggaagntgaaacatttactttcattataatactatatcaaatccatattcaatgcagggatatctgttatcaatacgtaaaatcaagagaacataatcttgttacagaatggttggtgccttaagacctttcttgcacacttaaacatttgttaagagggtatatttcatgttgttttgttattacaacaaaatttttaaaagaggacctttctcaagcagttgtggaataaatcaggtctaaagtatgttcaacagtggtatgattaacacttatgtaaaagcaaaaaaaaa

SEQ ID NO: 176 - DNA, 828 bp, Homo sapiens CYP2C9RS2860906   286
  misc_feature (328)..(328): n = a or g
gtgaatttgggagctctttaactataaagtttaatatctcaaaataataagagctatttatgacaaacccatagccaatatcatattgaatgggcaaaagctggaagcattccctttgaaaaccagcacaagacaaggatgccctctctcaccactcttattcaacatagtattggaagtcctggccagagcaatcagtcaagggggtattcaaatgggaagagaagaagtcaaattgtctctgtttgcaggtgacatgactgtatacttagaaaccccatcatctcagccccaaaactgtttaagctgataagcaatttcagcaagncctcaggatacaaaatcaatgtgcaaaaataacaagcattcctataaaccaataatagacaagcagagagccaaatcatgagtggactcccattcacaattgctacaaagggaataaaatgcctacttacacaacttacaagcgatgtgaaggacctcttcaaggagaactacaaaccacttctcaaggaaataagaggtgacacaaatggaaaaaaattccgtgctcatgaatagaaagaatcaatactgtgaaaatagccatactgcccaaagtaattcatagattcaatgctatacccgtcaaactatcattgactttcttcacagaactagaaaagaataatttaaatttcatatgaagcaaaaaaagagcctgtatagccaagacaatcctaagcaaaaacaaagctggaggcatcattctacctgacttcaaacaatactacaaggctacagtaaccaaacagcatggtactgggaaaactggctagccatatg

SEQ ID NO: 177 - DNA, 430 bp, Homo sapiens ABC11045642   665
  misc_feature (212)..(212): n = c or t
attgtgctacattcaaagtgtgctggtcctgaagttgatctgtgaactcttgttttcagctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatngtgagggcagcaaaggaggccaacatacatgccttcatcgagtcactgcctaatgtaagtctctcttcaaataaacagcctgggagcatgtggcagcctctctggcctatagkttgatttataaggggctggtytcccagaagtgaagagaaattagcaaccaaatcacacccttacctgtatacaagcatctggccacacttcctgtttgggttagt

SEQ ID NO: 178 - DNA, 612 bp, Homo sapiens CYP2C8_RS947172   371
  misc_feature (251)..(251): n = a or g
ttattcggatttttttcttgctgttttgagtttcttgtagactctggaaaatagtcctttgttgaaggtatattttgcaaatattttctcccattctgtaggttgtatgtttactctgcttgtcatttcttttactgtgcagaagctctttagtttaattaggtcccattgtcaactgtttttgttgaaattgcttttaaacattgagtcataaatccttagcctacaccaatgctcagaagagttttttntaggttttttctagaatttttatgatttcaagtctcatatttaagtctttagtccatcttgagttaatttttgtatgtggtgagatataagaatcatatttcattcttctacatgttcccctgggtaatatcagccaagcacaaatcccacagctaccagcgtaggtggctctttcctgcaagaaccacctcctagctggaagccaataggcacagcctattacaacatctgctggcaaaataacatagcatttgggaaggagaaaacttttatcgtatctcagctaacaccatacccacatcaccccagctaatcggaaggtcttgagtgtgttcacaaacccaatacattgctagtaca

SEQ ID NO: 179 - DNA, 1000 bp, Homo sapiens ABC12373589   681
  misc_feature (501)..(501): n = a or g
atagagtttcattccaatctttttaaatatatttatgcacttaggaaaaaaacaatatggaaatgtgtaaaatatactttttttttaaaaaaaaggacacatttattcagcattatgatcagactattacatttaacaatcaacagtatgggtgccaaaaaaaatctacattaaaaccctttgttgtaatgctttacactttccacagaacagaaactaaaagaatctgttacacaattagtcacaaatatagtcctcgagttttttacccatacacatgagtatttgtctaaaacatgtcttcttgtagcacttaggccctgccaccactgtgcttgtctgagttcacaaatctgttgtaaactgtagcttccctgtcacttctctggctcttatctcctgctaagatttgtttcctggcagtaatttaaaatcttctgccactgctgtagctactgctgctactggaactgccatagccaccttggtttcatggtttgncaaagtactggcctgtaccagcataggggccagagcttctgcctccaaagtttcctcccttcatgggtccaaaatgtaaaactaattgttgtaattgccaaaatcattacaccacctccaaaattgcttccatgattaccaaatccattatagccatccccactgccactatatccaccaccaccacagctgccaccaaagccacaatgaccactgaagtttcctccacgaccaaagttgtcattcccaccgaaactacctccacgaccaccaccaaagttcccagagctgctttgcctctttggctggatgaagcactcaccatctcttgctttgacagggctttcctaacttcacaagtgtggccattcacagtatgggtatttctcagtgacagtcttacccatggagtcatggtcgtcaaaagttactaaagcaaagccccttttcttatcactgccttggtcagtcatgatttcaatcacttcaatttttccat

SEQ ID NO: 180 - DNA, 533 bp, Homo sapiens CYP2C9RS1934969   39
  misc_feature (122)..(122): n = a or t
gtccattcatttttcagttgcctatacatccatccattcatccatttatccatccactcatccatccattcattcatgcatgcacccatccacccatctatctcttcatctcttctacgatncactgaacagttattgcatattctgtttgtgccagttacagagacagtgtttgtcactgtcacagttacgcatgaggagtaactgctctctgtgtttgctattttcaggaaaacggatttgtgtgggagaagccctggccggcatggagctgtttttattcctgacctccattttacagaactttaacctgaaatctctggttgacccaaagaaccttgacaccactccagttgtcaatggatttgcctctgtgccgcccttctaccagctgtgcttcattcctgtctgaagaagagcagatggcctggctgctgctgtgcagtccctgcagctctctttcctctggggcattatccatctttcactatctgtaatgccttttctcacctgtcatctcacattttcccttc

SEQ ID NO: 181 - DNA, 401 bp, Homo sapiens CYP2C8E93UTR_221   155
  misc_feature (201)..(201): n = c or t
cgaatttgtgcaggagaaggacttgcccgcatggagctatttttatttctaaccacaattttacagaactttaacctgaaatctgttgatgatttaaagaacctcaatactactgcagttaccaaagggattgtttctctgccaccctcataccagatctgcttcatccctgtctgaagaatgctagcccatctggctgcngatctgctatcacctgcaactctttttttatcaaggacattcccactattatgtcttctctgacctctcatcaaatcttcccattcactcaatatcccataagcatccaaactccattaaggagagttgttcaggtcactgcacaaatatatctgcaattattcatactctgtaacacttgtattaattgctgcatatgc

SEQ ID NO: 182 - DNA, 823 bp, Homo sapiens CYP2C8_RS1058932   164
  misc_feature (491)..(491): n = c or t
tgttatggagctgataatcaatgaatatttgttgaatgaagggtgcctattgagattagatgttagacagatagcaaatatatctctttttgtacatttgtttgtcccaccatccattaatcaatccatcatgtcatccatccattcatccacatgttcattcatctacccaatcattaatcaattatttactgcatattctgtttgtgcaagtcacaaatgactgtttgtcacagtcacagttaaacacaaggagtaactacttcctttctttgttatcttcaggaaaacgaatttgtgcaggagaaggacttgcccgcatggagctatttttatttctaaccacaattttacagaactttaacctgaaatctgttgatgatttaaagaacctcaatactactgcagttaccaaagggattgtttctctgccaccctcataccagatctgcttcatccctgtctgaagaatgctagcccatctggctgcngatctgctatcacctgcaactctttttttatcaaggacattcccactattatgtcttctctgacctctcatcaaatcttcccattcactcaatatcccataagcatccaaactccattaaggagagttgttcaggtcactgcacaaatatatctgcaattattcatactctgtaacacttgtattaattgctgcatatgctaatacttttctaatgctgactttttaatatgttatcactgtaaaacacagaaaagtgattaatgaatgataatttagatccatttcttttgtgaatgtgctaaataaaaagtgttattaattgctggttca

SEQ ID NO: 183 - DNA, 384 bp, Homo sapiens MVKE7E8_197   578
  misc_feature (184)..(184): n = g or a
accccgggttcctgcagacaggctcttacttccagcacgaggtactgctccggggctggggcttcccccatctctcccagcacgcgctcacactccagggagatggcatctattgaggtcaggaggggggccacgatctctgggaactggaaaaaaaaagaaggaacggctggtgaggcctggnggcaggcagatgcaggacagctgccccagcagtggggtggagggaggaggtgttcacacagcccgtgcccatcctctggggaaaccacctctcttctgagcctgtttttttgccttcccagctgcaaagtcagtgtgtctaacgagcccgaccactgtcattttcctggccaggctacgggcacggacggtaccttcttt

SEQ ID NO: 184 - DNA, 401 bp, Homo sapiens GSTM11296954   565
  misc_feature (201)..(201): n = g or a
ggctgtgcgcttgagacaccagccagatcctaacggagcaaagctcttctcccttctcctccctgcccgcagaatccctcagccttctctccgctgccgagttcccaagggctctgggagactccggctgcaggggtcagactaaaaagtggtggtcccaacctgggaatttaattcagcccctgtcactgtaagagcagnacttcctctgatccgaaagctactccgagggcttagtctcccctctagccccgcctacacaggaacagtgtcagtggtataggaaggacccccaggaaaagggccagagtaaaggaaatgtggtctgtgttttctgttaggggcctttggatactgagtccttcggtcatctggctaagtactatgtaaattagccactt

SEQ ID NO: 185 - DNA, 889 bp, Homo sapiens CYP2B6RS2873265   120
  misc_feature (479)..(479): n = c or t
tgtatgagagcatatgatggggacctgggggtcaggaagtcttctcaagggagctgctgcctgcctgaatagctaaggaaccccaataatgaggagcagacaccgttcttgcactgacattaaccaagatgacccacccacatcctcaaataacaataacaacgacaaaaacaatttgccccaagtcctccctgtgagaaaatggaaatctttgctgcaataaaggaagggagagggtcaaacatgtctatgcaaaacttatcccaatgctttgggaggttgaggcaggaggattgcttgagtccaggagttcgagaccagcctaggcaacatagtgagaccgtcccccaaacacatctctacaaaaataaatagtggggcatggtggctcacacttttagtcccagctactctggaggctaaggtgggagaatctcctgagcacaggagttcaaggctgcagtgagctatgactgtgncattgtactccagcctgggtaacagactgagaccctgtctctaaaacaattaaaatgaaaaaaattctttaatatcattccagacaagcctccactttctatgaactaataaggtagccacaaagatccttttgaaaactcattttagtatacaaaatcaattcaagatggattaaagacttaaacgttagacctaaaaccataaaaaccctagaagaaaacctaggcattaccattcaggacataggcatgggcaaggacttcatgtctaaaaaaccaaaaccaatggcaacaaaagacaaaattgacaaatgggatctaattaaactaaagagcttctgcacagcaaaagaaactaccatcagagtgaacaggcaacctacaaaatgggagaaaattttcgcaaccta

SEQ ID NO: 186 - DNA, 579 bp, Homo sapiens CYP2C8_RS1926705   122
  misc_feature (337)..(337): n = c or t
aatatcttacctgctccattttgatcaggaagcaatcgataaagtcccgaggattgttaacatccagtgatgcttggtgttcttttactttctccctaatgtaacttcgtgtaagagcaacatttttaagcactttgttgtgagttcctgggaaacaatcaatgagtagagggaaattattgcagacctaaaagagaaaagaatattaaatataaacatgtcataagatatatgtatcttacaccaagccctgattgaaattataactataaatatgaataagacatcatgtccattttgaagggaaatttttatacatatatacattttttattanactttaagttctagggtatatgtgcacaaagtgcaggtttgtcacatatgtatacatgtgccaggttggtgtactgcacccaataactcgtcatatacattaggtatatctcccaatgctatcccttccccctccccccaccacacaacagaccccagtgtgtaatgttccccttcctgtgttcaagtgttctcattgttcaattcccacctatgaatgagaatatgcagtgtttggttttt

SEQ ID NO: 187 - DNA, 401 bp, Homo sapiens CYP2A132545782   556
  misc_feature (201)..(201): n = g or a
gtagatgtgaaatgattatgatgggctggattttgcagcaccaggtgttcaggtatgcaggaggccggttgacgcagtcacttgtccaggtgttaaaatattcagaagaactgggtagggagcatctgttagaaattacggtaagttggggatgggaacacgtgcccaggtgagagagctgagctgagggtatgcatcctnccagcaggctctgttttggggagcatctgttgagctatccaggtgtccttggagacagggtattggacatccatcctgggttctggtgcaactgtccagttgtccaatatcggggactgattttgaggggacactgtctggagggcggtgggagtttggggcacctgtctccaggtaggggagcagttggcaggttgtgg

SEQ ID NO: 188 - DNA, 401 bp, Homo sapiens CYP4B1RS837395   550
  misc_feature (201)..(201): n = t or a
cccacccaaaaactagccaggaatatggaatgtcgttttctccatggtttatatcaatatgaagagggatgccctgtggaggcagcccctcctccctgcaggcccaccaagtgatttttacattgaaatcagcaaaccagagcaaaaagaaccagatgtagcaggtcatgggaggagactctgccacggaattctccaaanccagtactcgaggatcccatacccagacactgacaggtgctgcccgccactgagcctcctcttcctgggtctcagatgcccacattttaaaattgagacatagaaattcattcctcctgtttacaatccagtcttatggctctcctttgatgactttcgctagattctgctctagtccagctgtgttgatgcctccaatt

SEQ ID NO: 189 - DNA, 276 bp, Homo sapiens CYP1A1_RS2515900   385
  misc_feature (201)..(201): n = a or g
aatgtttgtacacaacaatccttctattctagcctgcattgagcttgcatgcttgcataagagcttaagaaccattgatttaatgtaatagggaaaattctaacccaggtatccaaaaatgtgtaagaacaactacctgagctaaataaagatattgttcagaaaatccatatggtggagattttttggaatcataaatanttcatcactcgtctaaatactcaccctgaaccccattctgtgttgggtttactgtagggaggaagaagaggaggt

SEQ ID NO: 190 - DNA, 101 bp, Homo sapiens UGT1A1042605   788
  misc_feature (51)..(51): n = g or a
gtcacggcatatgatctctacagccacacatcaatttggttgttgcgaacngactttgttttggactatcccaaacccgtgatgcccaatatgatcttcat

SEQ ID NO: 191 - DNA, 351 bp, Homo sapiens ABC12235067   685
  misc_feature (320)..(320): n = g or a
accactatttactcttgtgcctcttggtgatcggtgctgtctgttacagatcgctactgaagcaatagaaaacttccgaaccgttgtttctttgactcaggagcagaagtttgaacatatgtatgctcagagtttgcaggtaccatacaggtaataaccgctgaagagtgggaggagagtgtgaataatttttcaatcatcatatttgttttcagagggattactttggctagaaggtagggagcaagtggagaaagtgctcgaaggtaaaccattgagaaacagttgtaattatgcaggagagaaagtacaagaccctnaactaaggcagggacatctctgaggtagaac

SEQ ID NO: 192 - DNA, 866 bp, Homo sapiens NAT21799929   530
  misc_feature (491)..(491): n = t or c
ttaggggatcatggacattgaagcatattttgaaagaattggctataagaactctaggaacaaattggacttggaaacattaactgacattcttgagcaccagatccgggctgttccctttgagaaccttaacatgcattgtgggcaagccatggagttgggcttagaggctatttttgatcacattgtaagaagaaaccggggtgggtggtgtctccaggtcaatcaacttctgtactgggctctgaccacaatcggttttcagaccacaatgttaggagggtatttttacatccctccagttaacaaatacagcactggcatggttcaccttctcctgcaggtgaccattgacggcaggaattacattgtcgatgctgggtctggaagctcctcccagatgtggcagcctctagaattaatttctgggaaggatcagcctcaggtgccttgcattttctgcttgacagaagagagaggaatctggtacntggaccaaatcaggagagagcagtatattacaaacaaagaatttcttaattctcatctcctgccaaagaagaaacaccaaaaaatatacttatttacgcttgaacctcaaacaattgaagattttgagtctatgaatacatacctgcagacgtctccaacatcttcatttataaccacatcattttgttccttgcagaccccagaaggggtttactgtttggtgggcttcatcctcacctatagaaaattcaattataaagacaatacagatctggtcgagtttaaaactctcactgaggaagaggttgaagaagtgctgagaaatatatttaagatttccttggggagaaatctcgtgcccaaacctggtgatg

SEQ ID NO: 193 - DNA, 887 bp, Homo sapiens NAT21208   598
  misc_feature (521)..(521): n = g or a
atccctccagttaacaaatacagcactggcatggttcaccttctcctgcaggtgaccattgacggcaggaattacattgtcgatgctgggtctggaagctcctcccagatgtggcagcctctagaattaatttctgggaaggatcagcctcaggtgccttgcattttctgcttgacagaagagagaggaatctggtacttggaccaaatcaggagagagcagtatattacaaacaaagaatttcttaattctcatctcctgccaaagaagaaacaccaaaaaatatacttatttacgcttgaacctcaaacaattgaagattttgagtctatgaatacatacctgcagacgtctccaacatcttcatttataaccacatcattttgttccttgcagaccccagaaggggtttactgtttggtgggcttcatcctcacctatagaaaattcaattataaagacaatacagatctggtcgagtttaaaactctcactgaggaagaggttgaagaagtgctganaaatatatttaagatttccttggggagaaatctcgtgcccaaacctggtgatgaatcccttactatttagaataaggaacaaaataaacccttgtgtatgtatcacccaactcactaattatcaacttatgtgctatcagatatcctctctaccctcacgttattttgaagaaaatcctaaacatcaaatactttcatccataaaaatgtcagcatttattaaaaaacaataactttttaaagaaacataaggacacattttcaaattaataaaaataaaggcattttaaggatggcctgtgattatcttgggaagcagagtgattcatgctagaaaacatttaatattgatttattgttgaattc

SEQ ID NO: 194 - DNA, 531 bp, Homo sapiens NAT21495744   588
  misc_feature (324)..(324): n = a or g
actgcatggaacaatcctcctcacacatatccacagaacttattctctagcatccttaaagtcttagtgagcctttctttaaccaccttgtttgaattcagtgctctccctgtgcacccactaacccctctttttgttttcaccaggcacttaccacaatctaacagactgcatgttttatccatttattcagtttcctatttgtgtcccttcaactcccattaaaatataatatttttgagggcaagcaagtactagaacaataggaaacacatcaagagtattctgtaaactatttcttgaatcaatcagtgaatgaatganttaatcaatatattttttgagtgaggagctttgtgttaggtacagctaaatgggaaatcaagtgggtcatgtaccatgaataccatatactctactgtataattatcctgcttatatcagaaactgtttataagcctattataattgataccaattggaatctcttttttactcatcaccaagaacaccacaaacaagttgtttacc

SEQ ID NO: 195 - DNA, 317 bp, Homo sapiens CYP2A6RS696839   91
  misc_feature (211)..(211): n = g or c
aaacacgtgggctttgccacgatcccacgaaactacaccatgagcttcctgccccgctgakcgagggctgtgccggtgcaggtctggtgggcggggccagggaaagggcagggccaagaccgggcttgggagaggggcgcagctaagactgggggcaggatggcggaaaggaaggggcgtggtggctagagggaagagaagaaacagaagnggctcagttcaccttgataaggtgcttccgwgwtgggatgagaggaaggaaacccttacattatgctatgaagagtagtaataatagcagctcttatttcctgagc

SEQ ID NO: 196 - DNA, 121 bp, Homo sapiens CYP2C82275622   459
  misc_feature (61)..(61): n = t or c
taaaaaaaaggggcagaaactgggagaattcacagccaaggaagaaagtgctgcaacactnggcagccatgcagataggctaagctctgctgagaagctttttagggctctgttttccatc

SEQ ID NO: 197 - DNA, 726 bp, Homo sapiens NAT21799930   603
  misc_feature (460)..(460): n = a or g
gtgggcaagccatggagttgggcttagaggctatttttgatcacattgtaagaagaaaccggggtgggtggtgtctccaggtcaatcaacttctgtactgggctctgaccacaatcggttttcagaccacaatgttaggagggtatttttacatccctccagttaacaaatacagcactggcatggttcaccttctcctgcaggtgaccattgacggcaggaattacattgtcgatgctgggtctggaagctcctcccagatgtggcagcctctagaattaatttctgggaaggatcagcctcaggtgccttgcattttctgcttgacagaagagagaggaatctggtacctggaccaaatcaggagagagcagtatattacaaacaaagaatttcttaattctcatctcctgccaaagaagaaacaccaaaaaatatacttatttacgcttgaacctcnaacaattgaagattttgagtctatgaatacatacctgcagacgtctccaacatcttcatttataaccacatcattttgttccttgcagaccccagaaggggtttactgtttggtgggcttcatcctcacctatagaaaattcaattataaagacaatacagatctggtcgagtttaaaactctcactgaggaagaggttgaagaagtgctgagaaatatatttaagatttccttggggagaaatctcgtgcccaaacctggtgatg

SEQ ID NO: 198 - DNA, 987 bp, Homo sapiens CYP3A4_RS2246709   384
  misc_feature (501)..(501): n = a or g
caaaattaatcttgctgttcaagaaatagtaggtagtcaagatagaaataacacagcatatctctgtcacctatcatggaataaagataaaatcaataagggaaagaaaattgagaaactcacacatatgtggaagttaaataataaacatttaagtacccaatgagtcaaagtagaaacccaaagggcaaatagaaactgttttgaggtgaacaaaactaagatgtgataggccacaatctcatgggatttagcaaaggaagtgctcagagggaaagttacagctgtaatgtctaaatttagaggaacaaaaaaatcacaaatcagtaatctatgttcatgccacaacatagtaaacgaagaagggcaaactaagcctgaagccagcagaagaaagaaaatgatacagactaaagtacaaattcatgaactagagaataaaaaaccctgatgaattaatatcatttctatgaagtgtccagaataggcaaatccatagangcagaaagttgattagtggttgcatatgatgacagggtttgtgacagggggctgatagctaaaaatgtatgaggtctctagattgacaaaaaaagttttaaagtttaaaatgatgatggtcacacatatcttcaaatgtactacaaatcactgaactgtatattttaagtggatgaattacatggtgatttatatctcaataaagcagttatttttaagagagaaagataaattaaaggaaatagtagtccacatacttattgagagaaagaatggatccaaaaaatcaaatcttaaaagcttcttggtgttttccacaaaggggtcttgtggattgttgagagagtcgatgttcactccaaatgatgtgctagtgatcacatccatgctgtaggccccaaagacgctgagtggagaaagatgtggaaaattaaaatcagcacctttttaccatccttcctctatgcatgcaacaggaaacccaca

SEQ ID NO: 199 - DNA, 909 bp, Homo sapiens CYP4B1RS2297809   219
  misc_feature (533)..(533): n = c or t
gccaaagggaaaagacacacacacacacacacacacacttcacctcattaaataatgcatcaactttggttggttcattcaaccaacgtgtatcagccccagtttctttcattcagctcagtaggggaaaccaagctgaaacctaaagaactgtcctttgtaggcttccatgggggtcctgagagttgcagaagtgaccttgtctttgatggctgggtgcaccttcatctctagggtcctgtgccttcttctcctaccagtgaagatgagaaagggatggagaaaagtggaaccagatcctttagatctgagtgttcacaccatgtcaggcctagcctggccaggggcacttggaactctgtgcctctgactcttgagtgtgtggtggtggtgagggaggaaaactggggctggggtctgctttctcgccaaatcctgttgcttcccattccaagaatgttctggttgtgttgctggcagggatgatctgggcaaaatgacttatctgaccatgtgcatcaaggagagcttcngcctctacccacctgtgccccaggtgtaccgccagctcagcaagcctgtcacctttgtggatggccggtctctacctgcaggtgggatgggtggatttgggggtggaaaaggagtccctgcatgctcctctggcaccctctgtgcctttagtcaaatctttgcacttttggggaagagctcaggctttgcgttcagagagccctgggtctaaatcccagccccacaacatattgatcaattcttctacctctgagcctccatttccccttctgtaaaatggggatgagaagagtatctatgtttctaggctgctgtcaggattaaagagaatgttcaccaaggctgccatgtaccaccatgcaggtcatgccctga

SEQ ID NO: 200 - DNA, 630 bp, Homo sapiens PON3   869755
tttaatcttagcttcatgttggacagtgtgaaagaagatggtaccatatacaactctctctttgtctaactgcaagacctacctgctcagagactatctcctgattggaaatacgatatggcaatctgggtcaatatgaataatcgagcttatgtttttccattattcccaacagcaagagttattgaaaagttaatggtggcctaaaagagttaacgaaccttatctccctacctgtcaaaaactttacttttctcatacgtaaaatttctttagttctataacaccaaatgtgactggcttaaattcttccaagtcaccccaacaaatttgttcytgcagctttgcctgtgagctcagagaggtataggaacttacttctgatcctggagggtcctcagggttatagttcagtagcttcataggattaggatggcatcctgccaaaatgtctcctgtggcaggatcgacagtcaggttatccactaaggtgcccaactgtatcaccttacaacaaagagggagtagtgaagacattgtctcaatgattcctcgctttcttccmtattcatccccacaacccctacagcttcttctggcacctaaagttggtgtttttagggggctttg

SEQ ID NO: 201 - DNA, 490 bp, Homo sapiens CYP2D6   869777
tgagtgcaaaggcggtcagggtgggcagagacgaggtggggcaaagcctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgwtyctggcgcgctatgggcccgcgtggcgcgagcagaggcgcttctccrtstccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggygaccccttacccgcatctcccacccccargacgcccctttcgccccaacggtctcttggacaaa

SEQ ID NO: 202 - DNA, 2240 bp, Homo sapiens CYP2D6   554371
aacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgccctaagtgtcagtgtgagtctgtgtatgtgtgaatattgtctttgtgtgggtgattttctgcgtgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacycaccaggccccctgccactgcccgggctgggcaacctgctgcatgtggacttccagaacacaccatactgcttcgaccaggtgagggaggaggtcctggagggcggcagaggtgctgaggctcccctaccagaagcaaacatggatggtgggtgaaaccacaggctggaccagaagccaggctgagaaggggaagcaggtttgggggacgtcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgcctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccacgatcaggaggctgggtctcctccttccacctgctcactcctggtagccccgggggtcgtccaaggttcaaataggactaggacctgtagtctggggtgatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcggg

SEQ ID NO: 203 - DNA, 2170 bp, Homo sapiens CYP2D6   554365
gacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcrtgtctgtccaggccgtgtccaacaggagatcgacracgtgatagggcaggtgyggygaccagagatgggtgaccwggctcrcatgccctrcaycactgccgtgattcaygaggtgcagcgctttggggacatcgtccccctgggtgtgacccatatgacatcccgtgacatcgaagtacagggcttccgcatccctaaggtaggcctggcrccctcctcaccccagctcagcaccagcmcctggtgatagccccagcatggcyactgccaggtgggcccastctaggaamcctggccaccyagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctc

SEQ ID NO: 204 - DNA, 906 bp, Homo sapiens TYR   217468
atcactgtagtagtagctggaaagagaaatctgtgactccaattagccagttcctgcagaccttgtgaggactagaggaagaatgctcctggctgttttgtactgcctgctgtggagtttccagacctccgctggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatscatatatattatgtgtcaatggatgcactgcttgggggatmtgaaatctggagagacattgatttttctgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggca

SEQ ID NO: 205 - DNA, 278 bp, Homo sapiens CYP2B6   1002412
agctgttacggttattctcatgtttaccattactgagtgatggcagacaatcacacagagataggtgacagcctgatgttccccaggcacttcagtctgtgtcsttgayctgctgcttcttcctaggggccctcatggaccccaccttcctcttccaktccattaccgccaacatcatctgctccatcgtctttggaaaacgattccactaccaagatcaagagttcctgaagatgctgaacttgttctaccagactttttcactcatcagctctgta

SEQ ID NO: 206 - DNA, 350 bp, Homo sapiens PON1   869817
cttcctctcacatacataccgattcctttaactaaattacagttagraagttctacgggttgtacctctcggagagcattaagtcgtgttctgtgggggagaaagaaataaaacacacacaaaactattcagaaattataataagttgcaaaggtaggcagtccacaaaagttctccttcactrtactttgcctgagtttcaactccagagtttttcctgcaatttttccaggcaaggccagggcaagacaagtggggamctctgggtacaatatttagtttttatttagcagaggtggataggtacacataagagatacataatatcctctctagggctctgtgtactt

SEQ ID NO: 207 - DNA, 299 bp, Homo sapiens CYP2C8E2E3_397   null
atcccaaaattccgcaaggttgtgagggagaaacgccggatctccttccatctctttccattgctggaaatgattcctaataaaaaaaggggcagaaactgggagaattcacagccaaggaagaaagtgctgcaacactyggcagccatgcagataggctaagctctgctgagaagctttttagggctctgttttccatccccctcaccccagttaccaaagctgacacagaaatatgtgcacctaccaagtcctttagtaattctttgagatattggggaattgcctcttccagaaaa

SEQ ID NO: 208 - DNA, 350 bp, Homo sapiens GSTM3   971882
ggtcccgcctcggggtcgccaggccctgaaccccaacgccggcattagtcgcgcctgcgcacggccctgtggagccgcggaggcaagggacggagaacggggcggaggcggagtcagggcgcccgcgcgtgggccccgcccccttatgtmgggyataaagcccctcccgctcacagtttccctagtcctcgaaggctcggaagcccgtcaccatgtcgtgcgagtcgtctatggttctcgggtactgggatattcgtggggtgagtgccgtctcaacggtagagccgctcggtcaaagagactgacgcggagagggcgggtctctgggtccgcgatctccagcaggagca

SEQ ID NO: 209 - DNA, 420 bp, Homo sapiens OCA2   886896
tggccaggcataccggctctcccggggacgggtgtgggccatgatcatcatgctctgtctcatcgcggccgtcctctctgccttcttggacaacgtcaccaccatgctcctcttcacgcctgtgaccataaggtacgcaaagcacctctgccgtgggrgttgcggccaggttctggcaggcaggggctctgcctgcactgcctggctccaggttccattctcaggtgcatgaaaaggtgggggcrgttgagcccacagctcactgcattccagtccagctcgtgtctgctttgtgtgactgcagtacatgctacaagcagtggggcctcagaagctggtggcagaaatgcctgcaggaggtggaagacataggccttgctttcctggagattgtggtctcatggggagacatgtggacaa

SEQ ID NO: 210 - DNA, 350 bp, Homo sapiens OCA2   886894
tgcgtcgcccggaggctgcacaccttccacaggtaccgggcggggtcctgctcagactgtgcttggtgtgcagcagaacattccatgggcctacaaaatagcgacattagctgtatactaatacrtgatatttaggtgacgcacactgtgctaagcctcttatagtacattttatctaaccctcactgagctytgcagggggtacacagccgagtttaaggaccaaagaaacaacacaaaaccagaggctcagagaatttgagcggcgtgcccagggttgtgcagctcggaaggagtggcactggggatggggctctcactgtcaaccgctgggctgtcccatctctcta

SEQ ID NO: 211 - DNA, 350 bp, Homo sapiens CYP2C8   1004864
acattgagtcataaatccttagcctacaccaatgctcagaagagttttttataggttttttctagaatttttatgatttcaagtctcatatttaagtctttagtccatcttgagttaatttttgtatgtggtgagatataagaatcatatttcattcttctacatgttcccctgggtaatatcagccaagcacaaatcccrcagctaccagcgtaggtggctctttcctgcaagaaccacctcctagctggaagccaataggcacagcctattacaacatctgctggcaaaataacatagcatttgggaaggagaaaacttttatcgtatctcagctaacaccataccca

SEQ ID NO: 212 - DNA, 350 bp, Homo sapiens CYP2C9   869797
tgattgatcttggagaggagttttctggaagaggcattttcccactggctgaaagagctaacagaggatttggtaggtgtgcawgtgcctgtttcagcatctgtcttggggatggggaggatggaaaacagagacttacagagcycctcgggcagagcttggcccatccacatggctgcccagtgtcagcttcctctttcttgcctgggatctccctcctagtttcgtttctcwtcctgttaggaattgttttcagcaatggaaagaaatggaaggagatccggcgtttctccctcatgacgctgcggaattttrggatggggaagaggagcattgaggacmgtgttcaa

SEQ ID NO: 213 - DNA, 420 bp, Homo sapiens CYP2C8_1341159   null
gttgctcaggttggagtacagtgctgtcatcttggctcactgcaacctctgactcttggtctcaagtgattctcctacctcagcctcccaagtagctaggagcacaggcacaaacccccacacccagctaatttttgtattttttttgtacaaacttggtttcaccatgtttcctaggctggtctcaaactcctgagctcaagcagtccaccsatgttggccctcccaaagcactgggattgcagttgtgaggcaccacacctggccctttgcttatttctatactgggttgcttgtcatttgttgttgaactgtaggtaattgtttatggattctgggcattaaacccttactaaatacgtatgaaatacaaatattttctcccattctacaggttgtcatttcacatttttaattttg

SEQ ID NO: 214 - DNA, 350 bp, Homo sapiens CYP2C8_2071426   1004857
tagcggagtgagttgatgcattttgtgaatacagaaacattggggtcattgtattatataatcatttaatacagtggcaaaagtttaaagtgctgtttctcctctttgtttcacagtgttttgctatgatttttgactgaaggtgaagggaagtgtgtgtgattagaaatttcatccartaagttctctactatagtagtcatgtgttttattcagaatggtcatgaaaattgaacttctctgaagattcatttgatggctgatgtgaaataaatatctgtgggttcagggcaaacataagtgcatgaaagaaagaagtaatcagtcagggcccaataggtagttaacag

SEQ ID NO: 215 - DNA, 350 bp, Homo sapiens CYP2C8_RS947173   100486; identical to SEQ ID NO: 211

SEQ ID NO: 216 - DNA, 300 bp, Homo sapiens CYP1A2E7_405   null
ctagagtataccagtccactccagggaagattggagctgaggctgcttgagggctatacacactctgggaactagggggtctccaaacccttgagaggtttgcaggaggaaaactgcaaggagactggcagaaagcaggctgaagtggaagcttcctggcccgtgctgggctcstcagtgcttgagaacatagatgaagggcagacagtggccgcagacgagggacgctgtgaggaggaggcctggcatgtcttggggccaggaagagctccctgatcattttttccttcaggatgggta

SEQ ID NO: 217 - DNA, 350 bp, Homo sapiens CYP2C8   1004867
atgcactttaagatcccaaaaaattgctgctgttttaagtattttagtagatattctttatcagaaatctttaagattttagtagatatcctttatcagattagggaagttttttctcttctctcatttttatataaatttgtgtcatgaatgtgtgtgcaattttatcaaattgattctctgcatctrtttatatgattgtatatactggcctcacatccctcactaaatatacataagtatacacaaacagctggatttgttctgttacttattgttcaggacttctgtattcataagatattttggtctgcaatttttcttcctcaaaattttcttttcagagcttt

SEQ ID NO: 218 - DNA, 240 bp, Homo sapiens CYP2C8E8_92   null
ttcttataatcagattatctgttttgttacttccagggcacaaccataatggcattactgacttccgtgctacatgatgacaragaatttcctaatccaaatatctttgaccctggccactttctagataagaatggcaactttaagaaaagtgactacttcatgcctttctcagcaggtaatagaaactcgtttccatttgtatttaaaggaaagagagaactttttggaattagttgg

SEQ ID NO: 219 - DNA, 770 bp, Homo sapiens FDPS   756238
cctgcgctgcgcagctgcaccccttcgcgcatgggcgtggcgtagctcagacccgcccccagcgtttagcgtcttttgtcacccacctagagggtttgatatatcctaagcttttggcccctgggtcctggttccgtgcagcgagtcctcccagcaccccaccctgcacattctggaaagagccagactctggctgggccgagcaagaacagaaccacaagaaggttacacgattatttattgagagcctcctctccccgcccttgcaatctctaggtcactttctccgcttgtagattttgcgcgcaagccccaraaagacggctgggggcaggggtgctgcgtactgttcaatgagagccataargtggctgtaactgtcttcctcatattgcaagaacactgctggcagatccagctcctcatatagcgccttcacccgggccactttctcagcctccttctgcccgtaattttcctggaagaggttgaaagacaggaaaacgggcttggccttccccagagcctccaggacccctccactcccctcattcacatattccagaacatctccaaagccacccactcctttcctccctccaattttcaagtgtctctacgtagctaaaatcccaagcttcccttccctatcccaaatattgcctcataccaggcatcctctactccagggtttctccaccttggcactattgaaatttgggaccagataatcctgtctgggggagctgttctgtgtactacatgtttgg

SEQ ID NO: 220 - DNA, 350 bp, Homo sapiens CYP2C9   869803
gataatttctaaactactattatctsttaacaaatacagtgttttatatctaaagtttaatagtattttaaattgtttctaattatttagcctcaccctgtgatcccactttcatcctgggctgtgctccctgcaatgtgatctgctccattattttccakaaacgttttgattataaagatcagcaatttcttaacttaatggaaaagttgaatgaaaacatcaagattttgagcagcccctggatccaggtaaggccaagatttttgcttcctgagaaaccacttaytctcttttttttctgacaaatccaaaattctacatggatcaagctctgaagtgcatttttg

SEQ ID NO: 221 - DNA, 490 bp, Homo sapiens PON3   869790
tcatgttttatagtttycaggtataccagagaacgttgttgttcctcaaatttaaatatctccacagtggacttcatgtgggratgattcacaacataaagatayacagtattgtctacatggaaaaaagggataatttccaagaaagttacccctatcacaactaatattagacttgttttaaaacttggtcacttccaaaagttttcttcttacatcttgcatttkaccctcacagtctacatgataggtaagtcagacaaatgccagaatccagatttagttgaggaaattgaagctcaggaggcgaatgatccatcagcaatttcatcaccacaaagtggcagagccaagatgttttgccatagtcacttcacccttatatgcataacctgtctaacaggagcctacagaactataatgatgcataaacagggatgtggtttccccagatggcccttcagcaagagaagtgagtgcaagcaaacaa

SEQ ID NO: 222 - DNA, 490 bp, Homo sapiens HMGCS1   886899
ttaaaatacctctctcaaaagatgaacaaaattcaacttaccttcttttctccactttgattttcttacaaggggtaaaccgacctaaakatttttgcagagatacacctcagttccaaagtttacctcttctagtaaattacactttcccagaaactatgggaaaatgttgtttggtgaatgaggaattaatggtaaaaggatattaaatgaagaatgcccacaaagtagttgcytaccaaagatacctagtacattagactgctctcaactcataccaaaaccaaggaaaatgggtaaaacttacctttctgccactgggcatggatctttttgcagtagacagartagcagcggtctaatgcactgaggtagcactgtatggagagttttccatctactataggatattcagatagcatatcaggcttgtaaaaatcataggcatgttgcatatgtgtcccacgaagccctattagaaccaaaaagg

SEQ ID NO: 223 - DNA, 350 bp, Homo sapiens ACE   971861
  misc_feature (1)..(350) <: n is any nucleotide
tgttcccactttacaggtggggaccctgaggcttagggtcgtgagggacttagtggtcagagagctaggggccaaaccaaaggctctggccctgggtccagtgggggagccatcagcctagctcatgcccnaaggaaacaagcactgtggccctgcctcaggattgagtggctggggcctggcrcagccagaaatgacagtggcagcatcttgcagccccaggacatgtggccctcggaggagtgtgggtgggactgatgtgtgagatttctggccctaagccaggcctgncagcccttgagggccccagggtacaggtgccggccccagggtgccactcagcgatgcat

SEQ ID NO: 224 - DNA, 350 bp, Homo sapiens MVK   886917
gaggaatgttctcaagttcaaggatacagccagtgctacctatagaataaatgacaaaagcaataagcctgagggtgagtggcaaaggggccaggacccacgtgctaagaagagagcaaacataagcacagaggccactcctagccatgcccttgccagacactgctaatcaaccttggcatgctctcccactaagcctgggmacagccaccatcgatcagctaaaagttaaaatccactttgccttctgcctgcaaaatttcagaggttctcaataccaaggaatcacttccccataatcaccatgttttcaatgagaaatataagaacataaagaatagcagtgagaa

SEQ ID NO: 225 - DNA, 1032 bp, Homo sapiens OCA2   712054
cattgacttatttttaaaaatattgctccattgtcgttttgtttatatcttgattttggaagacctgatgtcagtctgattgttttgcgtgcggccttgatgatttttatcttcttccttgaaatcttatagttttactagaacatgtaacagagattttagttttaaatattagcttcattctactatttgtttttttcccttaaggactccaataaacaaatattattccttcattgcccgggttccatttccactactatctctgcccttttaatttatctatttacttattcatttttattctcttacttgctttgatatctttatttagtgacccttgttatattttcatttttgtctattgtcttttgggcatcttttaatttatttctcatttcttttgtaaagtgatttctctgagtacataatagttgttgcatatttatgggggacatgtgatattttgatacaataatacaatatgtaatgatgaaatcagggtaattaggatatccataacctcaaacatttrttgttacttgttttgggaacattccaagtcctttcttccagttattttaaaatatacaataagttattgttaattatagtggccctatcatgctatcaaacactagaacttattacttctaactaaccctattttttgtacccattaaacaaccccttatttctgagaaaacttggttacctcatccttgagttcaatcaactttttatttctccctgttatttgcccatttctgttttcaaatctctgatttaaggtgggtttgtatttttgatgcttgcttgaggcgtgggcatggcgaattcattttgaagtgtgggcttgtagttttcttctacatgcttcatggttattttcagaggggattttcctcagctgatacatgtgacatttccgctcctgatagcgtttgcactagctctgtaggtgtgacttcatttttctcttgttcatttaatgccgttgggcttgtttgtgttttgtaggattcctggcgc

SEQ ID NO: 226 - DNA, 266 bp, Homo sapiens CYP2B6E7E8_610   null
gaaaaaccagacgccttcaatcctgaccactttctggatgccaatggggcactgaaaaagactgaagcttttatccccttctccttaggtaagctggacccacaatttctttcccagacaccagagggcaggtactatccycaacttgagaaaaacaacgagagatactgattatttgagcacttaatatattctgattgcttcacctgccttatcccattccatcttcactacaaccctataaggaggcttgagaaagaagatat

SEQ ID NO: 227 - DNA, 348 bp, Homo sapiens CYP2B6   1002413
agctgttacggttattctcatgtttaccattactgagtgatggcagacaatcacacagagataggtgacagcctgatgttccccaggcacttcagtctgtgtcsttgayctgctgcttcttcctaggggccctcatggaccccaccttcctcttccaktccattaccgccaacatcatctgctccatcgtctttggaaaacgattccactaccaagatcaagagttcctgaagatgctgaacttgttctaccagactttttcactcatcagctctgtattcggccaggtcagggagacggagagggacagggggtgtgggggtgaggtgaacacccagaacacacgag

SEQ ID NO: 228 - DNA, 1190 bp, Homo sapiens CYP2D6   756251
tgagtgcaaaggcggtcagggtgggcagagacgaggtggggcaaagcctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccstgtccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccargacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatttgcatagatgggtttgggaaaggacattccaggagaccccactgtaagaagggcctggaggaggaggggacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccga

SEQ ID NO: 229 - DNA, 300 bp, Homo sapiens CYP2C8E93UTR_221   null
ttacagaactttaacctgaaatctgttgatgatttaaagaacctcaatactactgcagttaccaaagggattgtttctctgccaccctcataccagatctgcttcatccctgtctgaagaatgctagcccatctggctgcygatctgctatcacctgcaactctttttttatcaaggacattcccactattatgtcttctctgacctctcatcaaatcttcccattcactcaatatcccataagcatccaaactccattaaggagagttgttcaggtcactgcacaaatatatctgcaat

SEQ ID NO: 230 - DNA, 490 bp, Homo sapiens CYP2C8   1004863
acatctctgtttctccaggattggtccctggtgccttatttagttcgtgtggtgaggtcatgttttcctggattttctttatacttgtagatattcatcgggggctgggcattctagagttaggtatttttgttgtctttgtagtctggggttttttttgtacacatccttcttgttaggctttccagatattaaaaaggacttaacctattttcgatttgcccctagaatactgcaccrgcagtgaactgcactttttttaataaatgggaaatgagttaagtgttgtgatctaagctgtatctgctttaggggcactccaagcccaataatgcagtggttcttgaagacttgtagaggtactgccttgatggtcttggacaagatccaagagaattctctggattaccagaaactcttgttcccttcccttaatttctcccaaataaacaaagtctctctctctgttctgagtcaattgaaactgggg

SEQ ID NO: 231 - DNA, 435 bp, Homo sapiens OCA2   217458
gatcgacccacctcggaaagtgctgggattacaggcgtgagccaccatgcctgggctgccatttcatttccccttgtttatttccagggcctggactttgccggattcactgcacacatgttcattgggatttgycttgttctcctggtctgctttccgctcctcagactcctttactggaacagaaagctttataacaaggaacccagtgagattgttggtgagtacaagtgcaacctcatgtaggctcagatttcatgaccataatattgtttgtttaccaggagaagttcttattaggaagtatctgttgatgggttgctggatgctcaataccagtgactctccacgtccaccttctagtatacactgttttcagggctgctatcatgagctgtgcctctttagttttcgtgaagtgtactgtccctaaaa

SEQ ID NO: 232 - DNA, 350 bp, Homo sapiens CYP2C9   869806
aaaaaaaaatatgctgtgtgactcagctagctgcaaagagcctgatgaatggaatttttaggcaagcatggaataagggagtaggaaataaagtttgggcaagttggtctacagcctctgctatacaagcagtattttttttctagtactgtactttccagtttctatgttggtaactatataactatgtgartaattttgaattcactgtaatcaaatatgctggtaaataatttgtcagataattgcatcaaatcattcctaggaaaagcacaaccaaccatctgaatttactattgaaagcttggaaaacactgcagttgacttgtttggagctgggacagagacga

SEQ ID NO: 233 - DNA, 420 bp, Homo sapiens PON1   886930
gaacacrcatgatcataattayagcacaagtrtaaatgcatacacaatttgtcttttaaaccatgactgttcattttatttgaaagtgggcatgggtatacagaaagcctaagtgaaagacttaaactgccagtcctagaaaacgttctagaacacagaaaagtgaaagaaaacactcacagagctaatgaaagccagtccattaggcagtatctccawgtcttcagagccagtttctgccagaaaagagaacagaaagtacaggttgtttcatattattgcaggatgtggatccatttctttatcacacctcacttgaaactggggctatacatcactcttctttaataggttcagaataattcattctttcatttattcaaattgatkaatgcgattatatggaaattaaaaatatat

SEQ ID NO: 234 - DNA, 300 bp, Homo sapiens CYP3A4_RS2246709   null
agaagggcaaactaagcctgaagccagcagaagaaagaaaatgatacagactaaagtacaaattcatgaactagagaataaaaaaccctgatgaattaatatcatttctatgaagtgtccagaataggcaaatccatagargcagaaagttgattagtggttgcatatgatgacagggtttgtgacagggggctgatagctaaaaatgtatgaggtctctagattgacaaaaaaagttttaaagtttaaaatgatgatggtcacacatatcttcaaatgtactacaaatcactgaactgt